SEQUENCE LISTING (39 sequences)

SEQ ID NO: 1 - protein, 133 aa, Homo sapiens [sequence omitted]

SEQ ID NO: 2 - DNA, 507 bp, Artificial Sequence
  Synthetic Construct
atgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccag

SEQ ID NO: 3 - protein, 169 aa, Artificial Sequence
  Synthetic Construct
MYSMQLASCVTLTLVLLVNSAPTSSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFCQSIISTSPQ

SEQ ID NO: 4 - DNA, 1962 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaat

SEQ ID NO: 5 - DNA, 1954 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacgtataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaat

SEQ ID NO: 6 - DNA, 5869 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggatcccagaccctgaactttgatctgctgaaactggcaggcgatgtggaaagcaacccaggcccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 7 - DNA, 3943 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 8 - DNA, 3935 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacgtataaaaattgaaattttattttttttttttggaatataaatatccctatcagtgatagagatctccctatcagtgatagagagccaccatgtacagcatgcagctggccagctgcgtgacactgaccctcgtgctgctggtgaacagcgctcctacctcctccagcaccagcagcagcaccgctgaggcccagcagcagcagcagcaacagcaacagcagcaacaacatttagaacagctgctgatggatttacaagaactgctgtctcgtatggagaactatcgtaatttaaagctgcctcgtatgctgaccgccaagttcgctttacccaagcaagctacagagctgaaggatttacagtgtttagaggacgagctgggccctctgaggcatgtgctggacggcacccagagcaagagcttccagctggaggacgccgagaactttatcagcaacattcgtgtgaccgtggtgaagctgaagggcagcgacaacaccttcgagtgccagttcgacgacgagagcgccacagtggtggactttttaagaaggtggatcgccttctgccagtccatcatcagcaccagcccccagtaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgaattc

SEQ ID NO: 9 - protein, 133 aa, Artificial Sequence
  Synthetic Construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 10 - DNA, 399 bp, Artificial Sequence
  Synthetic Construct
gcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 11 - DNA, 399 bp, Artificial Sequence
  Synthetic Construct
gcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacc

SEQ ID NO: 12 - DNA, 459 bp, Artificial Sequence
  Synthetic Construct
atgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 13 - DNA, 459 bp, Artificial Sequence
  Synthetic Construct
atgtatcgaatgcaattactttcctgtatcgcactttcattagcccttgtgaccaactcagcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacc

SEQ ID NO: 14 - protein, 153 aa, Artificial Sequence
  Synthetic Construct
MYRMQLLSCIALSLALVTNSAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 15 - DNA, 1914 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaat

SEQ ID NO: 16 - DNA, 3895 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccgccaagttcgctatgcccaagaaggccaccgagctgaagcacctccagtgtttagaggaggagctgaagcctttagaggaggtgctgaatggagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 17 - DNA, 1914 bp, Artificial Sequence
  Synthetic Construct
gattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgaatgcaattactttcctgtatcgcactttcattagcccttgtgaccaactcagcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaat

SEQ ID NO: 18 - DNA, 3895 bp, Artificial Sequence
  Synthetic Construct
tgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcactgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacgaattcgattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactctcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacgggcgcgcctataaaaattgaaattttattttttttttttggaatataaatatccctatcagtgatagagatctccctatcagtgatagagagccaccatgtatcgaatgcaattactttcctgtatcgcactttcattagcccttgtgaccaactcagcgccaacatcaagttcgaccaagaagacgcagttgcagctagagcatttgcttttggatcttcaaatgatccttaatggtataaataattataagaaccccaaattgacgcgaatgctaacagctaaattcgcaatgccaaagaaggcaaccgagttaaagcacctacaatgcttggaagaagaactaaaaccccttgaggaggtattaaatggtgctcagtcgaagaattttcatcttcgacctcgagacctaatttcaaatattaacgtaattgttttggaattaaagggttcggaaactacttttatgtgtgagtacgcagacgagacagctacaatagtggagtttcttaaccgttggataaccttttgtcaatcaatcatttcgactttgacctaatgagcgatcgcgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggaaatgaacctagtacggcggatttactaagtaattacgctctacaaaatgtcatgattagtattttaaaccccgtggcatctagtcttaaatggagatgcccgtttccagatcaatggatcaaggacttttatatcccacacggtaataaaatgttacaaccttttgctccttcatattcagctgaaatgaattc

SEQ ID NO: 19 - DNA, 507 bp, Artificial Sequence
  Synthetic Construct
atgtactcgatgcagttagcttcctgcgtgaccctaaccttagtcttgctagtgaattcggcgcccacctcatcctcaacgtcatcttccacagcggaggctcaacagcagcagcaacagcagcaacaacaacagcagcatttggaacaattgctaatggacttacaggaactactatcaagaatggagaattatcgaaacctaaagttacctcgaatgttgacagcaaaatttgcgttgccaaagcaggccacagagctaaaggacctacagtgtcttgaagatgagctaggaccacttcgtcacgttttagacggaacacagtccaagtcttttcagttggaagacgccgagaactttatatctaacatacgtgttactgtcgtaaaacttaaaggatcggacaatactttcgaatgccaattcgatgatgaaagtgcaaccgtcgtggacttcttgcgacgttggatcgccttctgtcaaagtataatttccacttcgccacag

SEQ ID NO: 20 - DNA, 2204 bp, Artificial Sequence
  Synthetic Construct
actttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcggcgcgcctataaaaattgaaattttattttttttttttggaatataaatatccctatcagtgatagagatctccctatcagtgatagagagccaccatgtactcgatgcagttagcttcctgcgtgaccctaaccttagtcttgctagtgaattcggcgcccacctcatcctcaacgtcatcttccacagcggaggctcaacagcagcagcaacagcagcaacaacaacagcagcatttggaacaattgctaatggacttacaggaactactatcaagaatggagaattatcgaaacctaaagttacctcgaatgttgacagcaaaatttgcgttgccaaagcaggccacagagctaaaggacctacagtgtcttgaagatgagctaggaccacttcgtcacgttttagacggaacacagtccaagtcttttcagttggaagacgccgagaactttatatctaacatacgtgttactgtcgtaaaacttaaaggatcggacaatactttcgaatgccaattcgatgatgaaagtgcaaccgtcgtggacttcttgcgacgttggatcgccttctgtcaaagtataatttccacttcgccacagtattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaa

SEQ ID NO: 21 - protein, 153 aa, Artificial Sequence
  Synthetic Construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 20 aa, Homo sapiens
MYRMQLLSCIALSLALVTNS

SEQ ID NO: 23 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 24 - protein, 167 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTS

SEQ ID NO: 25 - protein, 376 aa, human herpesvirus 1
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 26 - protein, 376 aa, Artificial Sequence
  Synthetic Construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHVPPPALTILADRHPIAYFLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 27 - protein, 376 aa, Artificial Sequence
  Synthetic Construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 28 - protein, 376 aa, Artificial Sequence
  Synthetic Construct
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 29 - protein, 153 aa, Artificial Sequence
  Synthetic Construct
MYRMQLLSCIALSLALVTNSAPTSSSTKKTQLQLEHLLLDLQMILNGINNYKNPKLTNMTTFNFTMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - DNA, 459 bp, Artificial Sequence
  Synthetic Construct
atgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactaatatgaccaccttcaacttcactatgcccaagaaggccaccgagctgaagcacctccagtgtttagaggaggagctgaagcctttagaggaggtgctgaatttagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 31 - protein, 133 aa, Artificial Sequence
  Synthetic Construct
APTSSSTKKTQLQLEHLLLDLQMILNGINNYKNPKLTNMTTFNFTMPKKATELKHLQCLEEELKPLEEVLNLAQSKNFHLRPRDLISNINVIVLELKGSETTFMCEYADETATIVEFLNRWITFCQSIISTLT

SEQ ID NO: 32 - DNA, 399 bp, Artificial Sequence
  Synthetic Construct
gcccctaccagctcctccaccaagaagacccagctgcagctggagcatttactgctggatttacagatgattttaaacggcatcaacaactacaagaaccccaagctgactaatatgaccaccttcaacttcactatgcccaagaaggccaccgagctgaagcacctccagtgtttagaggaggagctgaagcctttagaggaggtgctgaatttagcccagagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 33 - protein, 153 aa, Artificial Sequence
  Synthetic Construct
MYRMQLLSCIALSLALVTNSAPTSSSTKKTQLQLEHLLLDLQMILNGINNYKNPKLTRMLTFNFTMPKKATELKHLQCLEEELKPLEEVLNNATSKNFHLRPRDLISNINVIVLELKGSETTFMCEYADETATIVEFLNRWITFCQSIISTLT

SEQ ID NO: 34 - DNA, 459 bp, Artificial Sequence
  Synthetic Construct
atgtatcgtatgcagctgctgagctgcatcgctttatctttagctttagtgaccaacagcgcccctaccagctcctccaccaagaagacccagctgcagctggagcatttactgctggatttacagatgattttaaacggcatcaacaactacaagaaccccaagctgactcgtatgctgaccttcaacttcactatgcccaagaaggccaccgagctgaagcacctccagtgtttagaggaggagctgaagcctttagaggaggtgctgaataacgccaccagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 35 - protein, 133 aa, Artificial Sequence
  Synthetic Construct
APTSSSTKKTQLQLEHLLLDLQMILNGINNYKNPKLTRMLTFNFTMPKKATELKHLQCLEEELKPLEEVLNNATSKNFHLRPRDLISNINVIVLELKGSETTFMCEYADETATIVEFLNRWITFCQSIISTLT

SEQ ID NO: 36 - DNA, 399 bp, Artificial Sequence
  Synthetic Construct
gcccctaccagctcctccaccaagaagacccagctgcagctggagcatttactgctggatttacagatgattttaaacggcatcaacaactacaagaaccccaagctgactcgtatgctgaccttcaacttcactatgcccaagaaggccaccgagctgaagcacctccagtgtttagaggaggagctgaagcctttagaggaggtgctgaataacgccaccagcaagaatttccatttaaggcctcgtgatttaatcagcaacatcaacgtgatcgtgctggagctgaaaggctccgagaccaccttcatgtgcgagtacgccgacgagaccgccaccatcgtggagtttttaaatcgttggatcaccttctgccagagcatcatcagcactttaacc

SEQ ID NO: 37 - protein, 10 aa, Artificial Sequence
  Synthetic Construct
GGGGSGGGGS

SEQ ID NO: 38 - protein, 168 aa, Artificial Sequence
  Synthetic Construct
MKSLNRQTVSMFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNNKWLDINNDKDIDISKLTNFKQLNSTTDAEACYIYKSGKLVETVCKSTQSVLCVKKFYK

SEQ ID NO: 39 - DNA, 507 bp, Artificial Sequence
  Synthetic Construct
atgaaatcgcttaatagacaaactgtaagtatgtttaagaagttgtcggtgccggccgctataatgatgatactctcaaccattattagtggcataggaacatttctgcattacaaagaagaactgatgcctagtgcttgcgccaatggatggatacaatacgataaacattgttatctagataccaacattaaaatgtccacagataatgcggtttatcagtgtcgtaaattacgagctagattgcctagacctgatactagacatctgagagtattgtttagtattttttataaagattattgggtaagtttaaaaaagaccaataataaatggttagatattaataatgataaagatatagatattagtaaattaacaaattttaaacaactaaacagtacgacggatgctgaagcgtgttatatatacaagtctggaaaactggttgaaacagtatgtaaaagtactcaatctgtactatgtgttaaaaaattctacaagtga

(19) United States
(12) Patent Application Publication
(10) Pub. No.: US 2022/0033784 A1
(43) Pub. Date: Feb. 3, 2022

(54) RECOMBINANT VACCINIA VIRUS

(71) Applicant: Pfizer Inc., New York, NY (US)

(72) Inventors: Joseph John Binder, San Diego, CA (US); Michael Dale Eisenbraun, San Diego, CA (US); Clare Lees, Del Mar, CA (US); Jeremy Shawn Myers, Farmington, CT (US); James Travis Patterson, San Diego, CA (US)

(73) Assignee: Pfizer Inc., New York, NY (US)

(21) Appl. No.: 17/371,206

(22) Filed: Jul. 9, 2021

Related U.S. Application Data
(60) Provisional application No. 63/051,890, filed on Jul. 14, 2020.
(60) Provisional application No. 63/051,628, filed on Jul. 14, 2020.

(51) Int. Cl. C12N 7/00; C07K 14/55; A61K 35/768; A61P 35/00

(52) U.S. Cl. CPC C12N 7/00; C07K 14/55; A61K 38/00; A61P 35/00; A61K 35/768

(57) ABSTRACT

The present disclosure provides human IL-2 variants, recombinant oncolytic viruses comprising the IL-2 variant, compositions comprising the IL-2 variant or recombinant oncolytic virus, and use of the IL-2 variants, recombinant oncolytic virus, or compositions for treating cancer in an individual.

### REFERENCE TO RELATED APPLICATIONS

This application claims the benefit of U.S. Provisional Patent Application No. 63/051,628 filed on Jul. 14, 2020 and U.S. Provisional Patent Application No. 63/051,890 filed on Jul. 14, 2020. The content of each of the provisional applications is incorporated herein by reference in its entirety.

### REFERENCE TO SEQUENCE LISTING

This application is being filed electronically via EFS-Web and includes an electronically submitted sequence listing in .txt format. The .txt file contains a sequence listing entitled “PC72649A_SequenceListing_ST25.txt” created on Jun. 22, 2021 and having a size of 80 KB. The sequence listing contained in this .txt file is part of the specification and is herein incorporated by reference in its entirety

### BACKGROUND

Interleukin-2 (IL-2) is a cytokine that is important for multiple functions of the mammalian immune system, such as stimulation of T cell and natural killer cell growth and has been approved as an immunotherapeutic agent for cancer. However, various factors (e.g. narrow therapeutic window; potential serious side effects) have limited its clinical use. One of the limitations of use of IL-2 as an anti-cancer agent is that while it can stimulate and expand effector T cells and NK cells (which have anti-tumor activity), it can also expand regulatory T cells (Tregs), which repress the immune response.

IL-2 signaling is mediated through the IL-2 receptor (IL-2R), which exists in different formats on different cell types. The high affinity IL-2 receptor contains three polypeptide chains, referred to as IL-2R alpha (“IL-2Rα”; also known as CD25), IL-2R beta ((“IL-2Rβ”; also known as CD122), and IL-2R gamma (“IL-2Rγ”; also known as CD132). The high affinity IL-2R is constitutively expressed on Tregs and transiently expressed on activated T cells and NK cells. The intermediate affinity IL-2R contains the IL-2Rβ and IL-2Rγ polypeptide chains, and is expressed, for example, on resting CD4+ and CD8+ T cells and natural killer (NK) cells. The low affinity IL-2R contains the IL-2Rα polypeptide chain.

Since IL-2 activates Treg cells via the high affinity IL-2R and activates resting CD4+ and CD8+ T cells and natural killer (NK) cells via the intermediate affinity IL-2R, one possible approach for selectively activating CD4+ T cells, CD8+ T cells and NK cells without activating Tregs is to selectively target IL-2 to the intermediate affinity IL-2R. Given that the difference between the high affinity IL-2 receptor and the low affinity IL-2 receptor is the presence of the IL-2Rα polypeptide chain in the high affinity IL-2 receptor, IL-2 can potentially be selectively targeted to the intermediate affinity IL-2R by blocking or impairing the interaction between IL-2 and the IL-2Rα polypeptide chain.

Oncolytic viruses (OVs) are viruses that selectively or preferentially infect and kill cancer cells. Live replicating OVs have been tested in clinical trials in a variety of human cancers. OVs can induce anti-tumor immune responses, as well as direct lysis of tumor cells. OVs can occur naturally or can be constructed by modifying other viruses. Common OVs include those that are constructed based-on attenuated strains of Herpes Simplex Virus (HSV), Adenovirus (Ad), Measles Virus (MV), Coxsackie virus (CV), Vesicular Stomatitis Virus (VSV), and Vaccinia Virus (VV).

Vaccinia virus (VV) is a member of the orthopoxvirus genus of the poxvirus family. It has a linear, double-stranded DNA genome approximately 190 kb in length, which encodes about 200 genes. Vaccinia virus replicates in the cytoplasm of a host cell. The large vaccinia virus genome codes for various enzymes and proteins used for viral DNA replication. During replication, vaccinia virus produces several infectious forms which differ in their outer membranes: the intracellular mature virion (IMV), the intracellular enveloped virion (IEV), the cell-associated enveloped virion (CEV) and the extracellular enveloped virion (EEV). IMV is the most abundant infectious form and is thought to be responsible for spread between hosts; the CEV is believed to play a role in cell-to-cell spread; and the EEV is thought to be important for long range dissemination within the host organism. EEV-specific proteins are encoded by the genes A33R, A34R, A36R, A56R, B5R, and F13 L. A34, a type II transmembrane glycoprotein encoded by the A34R gene, is involved in the induction of actin tails, the release of enveloped virus from the surfaces of infected cells, and the disruption of the virus envelop after ligand binding prior to virus entry.

### SUMMARY

In some aspects, the present disclosure provides human interleukin 2 (IL-2) variants, and related fusion proteins, compositions, methods, and uses. The IL-2 variants retain the ability to bind to the intermediate-affinity dimeric IL-2 receptor complex (containing IL-2Rβ+IL-2Rγ) but have decreased or no binding to the IL-2 receptor alpha (“IL-2Rα”/CD25) as compared to wild-type human IL-2 polypeptide, or have decreased or no binding to the high-affinity trimeric IL-2 receptor complex (containing IL-2Rα+IL-2Rβ+IL-2Rγ) as compared to wild-type human IL-2 polypeptide.

The IL-2 variants provided herein have one or more amino acid substitutions as compared to the wild-type human IL-2 amino acid sequence. In some embodiments, the amino acid substitution(s) in the IL-2 variants result in one or more engineered N-glycosylation site(s) in the IL-2 variant protein.

In some embodiments, the isolated human interleukin 2 (IL-2) variant comprises at least one amino acid substitution as compared to wild-type human IL-2, wherein wild-type human IL-2 has the amino acid sequence as shown in SEQ ID NO:

1 and the IL-2 variant comprises one or more substitutions at amino acid positions selected from the group consisting of: a) K35, b) both R38 and L40, c) both T41 and K43, d) both K43 and Y45, e) both E62 and K64, and f) both L72 and Q74. Optionally, the variant comprises one or more substitutions at amino acid positions selected from the group consisting of: a) K35, wherein the K35 substitution is K35N, b) both R38 and L40, wherein the R38 substitution is R38N and the L40 substitution is L40S or L40T, c) both T41 and K43, wherein the T41 substitution is T41N and the K43 substitution is K43S or K43T, d) both K43 and Y45, wherein the K43 substitution is K43N and the Y45 substitution is Y45S or Y45T, e) both E62 and K64, wherein the E62 substitution is E62N and the K64 substitution is K64S or K64T, and f) both L72 and Q74, wherein the L72 substitution is L72N and the Q74 substitution is Q74S or Q74T. Optionally, the IL-2 variant comprises a substitution in position K35, and wherein the IL-2 variant further comprises substitutions at positions selected from the group consisting of: a) both R38 and L40, wherein the R38 substitution is R38N and the L40 substitution is L40S or L40T, b) both T41 and K43, wherein the T41 substitution is T41N and the K43 substitution is K43S or K43T, c) both K43 and Y45, wherein the K43 substitution is K43N and the Y45 substitution is Y45S or Y45T, d) both E62 and K64, wherein the E62 substitution is E62N and the K64 substitution is K64S or K64T, e) both L72 and Q74, wherein the L72 substitution is L72N and the Q74 substitution is Q74S or Q74T, and f) E62, wherein the E62 substitution is E62N, E62A, E62K, or, E62R. Optionally, the IL-2 variant comprises substitutions at positions R38 and L40, and wherein the IL-2 variant further comprises substitutions at positions selected from the group consisting of: a) both T41 and K43, wherein the T41 substitution is T41N and the K43 substitution is K43S or K43T, b) both K43 and Y45, wherein the K43 substitution is K43N and the Y45 substitution is Y45S or Y45T, c) both E62 and K64, wherein the E62 substitution is E62N and the K64 substitution is K64S or K64T, d) both L72 and Q74, wherein the L72 substitution is L72N and the Q74 substitution is Q74S or Q74T, and e) E62, wherein the E62 substitution is E62N, E62A, E62K, or, E62R. Optionally, the IL-2 variant comprises substitutions at positions T41 and K43, and wherein the IL-2 variant further comprises substitutions at positions selected from the group consisting of: a) both E62 and K64, wherein the E62 substitution is E62N and the K64 substitution is K64S or K64T, b) both L72 and Q74, wherein the L72 substitution is L72N and the Q74 substitution is Q74S or Q74T, and c) E62, wherein the E62 substitution is E62N, E62A, E62K, or, E62R. Optionally, the IL-2 variant comprises substitutions at positions K43 and Y45, and wherein the IL-2 variant further comprises substitutions at positions selected from the group consisting of: a) both E62 and K64, wherein the E62 substitution is E62N and the K64 substitution is K64S or K64T, b) both L72 and Q74, wherein the L72 substitution is L72N and the Q74 substitution is Q74S or Q74T, and c) E62, wherein the E62 substitution is E62N, E62A, E62K, or, E62R. Optionally, the IL-2 variant comprises substitutions at positions E62 and K64, and wherein the IL-2 variant further comprises substitutions at positions L72 and Q74, wherein the L72 substitution is L72N and the Q74 substitution is Q74S or Q74T.

In some embodiments, an isolated human interleukin 2 (IL-2) variant provided herein comprises at least four amino acid substitutions as compared to wild-type human IL-2, wherein wild-type human IL-2 has the amino acid sequence as shown in SEQ ID NO: 1 and the IL-2 variant comprises substitutions at amino acid positions selected from the group consisting of: a) each of R38, L40, K43, and Y45; or b) each of K43, Y45, L72, and Q74. Optionally, the IL-2 variant comprises substitutions at amino acid positions R38, L40, K43, and Y45, and the R38 substitution is R38N. Optionally, the IL-2 variant comprises substitutions at amino acid positions R38, L40, K43, and Y45, and the L40 substitution is L40T. Optionally, the IL-2 variant comprises substitutions at amino acid positions R38, L40, K43, and Y45, and the K43 substitution is K43N. Optionally, the IL-2 variant comprises substitutions at amino acid positions R38, L40, K43, and Y45, and the Y45 substitution is Y45T. Optionally, the IL-2 variant comprises substitutions at amino acid positions R38, L40, K43, and Y45, and the R38 substitution is R38N and the K43 substitution is K43N. Optionally, the IL-2 variant comprises substitutions at amino acid positions R38, L40, K43, and Y45, and the amino acid substitutions are R38N, L40T, K43N, and Y45T. Optionally, the IL-2 variant comprises substitutions at amino acid positions K43, Y45, L72, and Q74, and the K43 substitution is K43N. Optionally, the IL-2 variant comprises substitutions at amino acid positions K43, Y45, L72, and Q74, and Y45 substitution is Y45T. Optionally, the IL-2 variant comprises substitutions at amino acid positions K43, Y45, L72, and Q74, and the L72 substitution is L72N. Optionally, the IL-2 variant comprises substitutions at amino acid positions K43, Y45, L72, and Q74, and the Q74 substitution is Q74T. Optionally, the IL-2 variant comprises substitutions at amino acid positions K43, Y45, L72, and Q74, and the K43 substitution is K43N and the L72 substitution is L72N. Optionally, the IL-2 variant comprises substitutions at amino acid positions K43, Y45, L72, and Q74, and the amino acid substitutions are K43N, Y45T, L72N, and Q74T. Optionally, the IL-2 variant comprises the amino acid sequence as shown in SEQ ID NO: 31 or SEQ ID NO: 35.

In some embodiments, provided herein is an isolated human interleukin 2 (IL-2) variant comprising at least one amino acid substitution as compared to wild-type human IL-2, wherein wild-type human IL-2 has the amino acid sequence as shown in SEQ ID NO: 1 and the IL-2 variant comprises an amino acid substitution at position E62. Optionally, the E62 substitution is E62N, E62A, E62K, or, E62R.

In some embodiments, provided herein is an isolated human interleukin 2 (IL-2) variant that comprises the amino acid sequence as shown in SEQ ID NO: 31 or 35.

In some embodiments, provided herein is an isolated human interleukin 2 (IL-2) variant comprising at least four amino acid substitutions as compared to wild-type human IL-2, wherein wild-type human IL-2 has the amino acid sequence as shown in SEQ ID NO: 1 and the IL-2 variant comprises the four amino acid substitutions R38N, L40T, K43N, and Y45T.

In some embodiments, provided herein is an isolated human interleukin 2 (IL-2) variant comprising at least four amino acid substitutions as compared to wild-type human IL-2, wherein wild-type human IL-2 has the amino acid sequence as shown in SEQ ID NO: 1 and the IL-2 variant comprises the four amino acid substitutions K43N, Y45T, L72N, and Q74T.

In some embodiments, an isolated human interleukin 2 (IL-2) variant provided herein has reduced binding to human IL-2 receptor alpha (IL-2Rα) as compared to wild-type human IL-2.

In some embodiments, an isolated human interleukin 2 (IL-2) variant provided herein is glycosylated on an introduced asparagine (N) residue substitution(s).

In some embodiments, an isolated human interleukin 2 (IL-2) variant provided herein further comprises substitutions at one or both of the positions T3 and C125. Optionally, the substitutions at positions T3 and C125 are T3A or T3G and C125A or C125S.

In some embodiments, provided herein is an isolated fusion protein comprising: a) an IL-2 variant provided herein; and b) an Fc region of a human antibody, wherein the IL-2 variant is covalently linked to the Fc region.

In some embodiments, provided herein is a heterodimeric protein comprising: a) an isolated fusion protein provided herein, wherein the Fc region of the human antibody is a first Fc region; and b) a second Fc region of a human antibody, wherein the first Fc region and the second Fc region are covalently linked by at least one disulfide bond. Optionally, the first Fc region comprises at least one amino acid modification, as compared to a wild-type human IgG Fc region, to form a knob or a hole, wherein the second Fc region comprises at least one amino acid modification, as compared to a wild-type human IgG Fc region, to form a knob or a hole, and wherein one of the first and second Fc regions contains a knob and one of the first and second Fc regions contains a hole. Optionally, the Fc region comprising the knob comprises the mutations Y349C and T366W, and wherein the Fc region comprising the hole comprises the mutations S354C, T366S, L368A, and Y407V.

In some embodiments, provided herein is an isolated fusion protein comprising: a) an IL-2 variant provided herein; and b) an antibody comprising a Fc domain, wherein the Fc domain comprises a first Fc region and a second Fc region, wherein the IL-2 variant is covalently linked to a Fc region of the antibody. Optionally, the Fc domain has decreased, or no antibody dependent cellular cytotoxicity (ADCC) activity as compared to a wild-type Fc domain. In some embodiments, provided herein is an isolated fusion protein comprising: a) an IL-2 variant provided herein; and b) an antibody comprising a Fc domain, wherein the antibody comprises a first light chain and a second light chain, wherein the IL-2 variant is covalently linked to a light chain of the antibody. Optionally, the Fc domain has decreased or no ADCC activity as compared to a wild-type Fc domain. Optionally, the antibody binds to a tumor or immune cell. Optionally, the antibody is selected from the group consisting of an anti-B7H4 antibody, an anti-CTLA-4 antibody, an anti-CD3 antibody, an anti-B7H4/anti-CD3 bispecific antibody, an anti-CD28 antibody, an anti-B7H4/anti-CD28 bispecific antibody, an anti-EDB1 antibody, an anti-ULBP2 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-8 antibody, an anti-IL-7Ralpha (CD127) antibody, an anti-IL15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-MARCO antibody, an anti-CXCR4 antibodies, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7H3 antibody, an KLRG1 antibody, and an anti-GITR antibody. Optionally, the IL-2 variant is covalently linked to the Fc region or the light chain, respectively, by a polypeptide linker and/or a polypeptide tag.

In some embodiments, provided herein is an isolated nucleic acid encoding an IL-2 variant, fusion protein or heterodimeric protein described herein. For example, in an embodiment, provided herein is a polynucleotide encoding an IL-2 variant containing substitutions R38N, L40T, K43N, and Y45T, wherein the polynucleotide comprises the nucleotide sequence as shown in SEQ ID NO: 32.

In some embodiments, provided herein is a recombinant expression vector comprising a nucleic acid encoding an IL-2 variant described herein.

In some embodiments, provided herein is a pharmaceutical composition comprising an IL-2 variant, fusion protein, or heterodimeric protein as described herein, and a pharmaceutically acceptable carrier.

In some embodiments, provided herein is a method for treating disease, such as cancer, in a subject in need thereof, the method comprising administering to the subject an effective amount of an IL-2 variant, fusion protein, heterodimeric protein, or pharmaceutical composition described herein, such that one or more symptoms associated with the disease is ameliorated in the subject. Optionally, the method further comprises administering an effective amount of a second therapeutic agent, optionally wherein the administration is separate, sequential, or simultaneous.

In some embodiments, provided herein is a method of stimulating the immune system in a subject in need thereof, the method comprising administering to the subject an effective amount of an IL-2 variant, fusion protein, heterodimeric protein, or pharmaceutical composition described herein, such that the immune system is stimulated in the subject.

In some embodiments, provided herein is an IL-2 variant, fusion protein, heterodimeric protein, or pharmaceutical composition described herein, for use in the manufacture of a medicament for use in the treatment of disease in an individual in need thereof.

In some embodiments, provided herein is a method of preparing a variant of a reference protein, wherein the reference protein binds to a binding partner protein, and wherein a binding domain in the reference protein interacts with the binding partner protein, the method comprising: introducing a glycosylation site in the binding domain of the reference protein, wherein the glycosylation site comprises the amino acid sequence N-x-S, N-x-T, S-x-N, or T-x-N, wherein introducing the glycosylation site comprises introducing at least one amino acid substitution in the amino acid sequence of the binding domain of the reference protein to generate the amino acid sequence N-x-S, N-x-T, S-x-N, or T-x-N, wherein x is any amino acid except proline, and wherein at least one of the N, S, or T residue(s) in the N-x-S, N-x-T, S-x-N, or T-x-N sequence is an amino acid substitution, in order to generate a glycovariant of the reference protein, wherein the variant has reduced binding to the binding partner protein as compared to the reference protein. Optionally, the method comprises introducing at least two amino acid substitutions in the binding domain of the reference protein, wherein introducing the glycosylation site comprises introducing at least two amino acid substitutions in the amino acid sequence of the binding domain of the reference protein to generate the amino acid sequence N-x-S, N-x-T, S-x-N, or T-x-N, wherein the N, S, or T residues in the N-x-S, N-x-T, S-x-N, or T-x-N sequence is an amino acid substitution.

In some other aspects, the present disclosure provides recombinant oncolytic viruses that comprises a nucleotide sequence encoding IL-2 variant provided herein, compositions comprising said IL-2 variants or oncolytic viruses, as well as methods and uses related to the oncolytic viruses. In some embodiments, the recombinant oncolytic virus comprises a nucleotide sequence that encodes a human IL-2 variant comprising the amino acid sequence of SEQ ID NO: 29.

In some embodiments, the recombinant oncolytic virus further comprises a nucleotide sequence encoding a heterologous thymidine kinase (TK) polypeptide. in a particular embodiment, heterologous TK polypeptide is an HSV-TK variant comprising the amino acid sequence of SEQ ID NO: 28.

In some embodiments, the recombinant oncolytic virus further comprises a modification that renders the virus thymidine kinase deficient. In a particular embodiment, the modification is a deletion of at least portion of the virus J2R gene.

In some other embodiments, the recombinant oncolytic virus provided by the present disclosure further comprises a modification that enhances the spread of progeny virions. In a particular embodiment, the modification results in K151E substitution in the viral A34R gene product.

In some embodiments, the recombinant oncolytic virus is a recombinant vaccinia virus. In a particular embodiment, the present disclosure provides a replication competent, recombinant oncolytic vaccinia virus that comprises: a) a nucleotide sequence encoding an IL-2 variant of SEQ ID NO: 29; b) a nucleotide sequence that encodes an HSV-TK variant comprising the amino acid sequence of SEQ ID NO: 28; c) A34R gene that encodes an A34 protein comprising a K151E substitution relative to the wild-type A34R gene product; and d) a deletion of at least portion of the virus J2R gene, wherein the recombinant oncolytic vaccinia virus is stain Copenhagen. In a specific embodiment, the A34 protein encoded by the A34R gene of the virus comprises the amino acid sequence of SEQ ID NO: 38. In another specific embodiment, the A34R gene of the virus comprise the nucleotide sequence of SEQ ID NO;39.

In some other aspects, the present disclosure provides compositions comprising the recombinant oncolytic virus, and method of using the oncolytic virus or composition for inducing oncolysis or treating cancer in an individual having a tumor or cancer.

In some other embodiments, the present disclosure provides a method of controlling the replication of a replication-competent, recombinant oncolytic virus in the body of an individual who has been administered the virus, comprising administering to the individual and effective amount of a synthetic analogs of 2′-deoxy-guanosine, such as ganciclovir.

Examples of other aspects and embodiments are described in detail below.

### BRIEF DESCRIPTION OF DRAWINGS

FIG. 1. Schematic representation of full genomes for recombinant oncolytic viruses VV91, VV93, and VV96. Abbreviations: LITR=left inverted terminal repeat; RITR=right inverted terminal repeat; A-O=viral gene regions historically defined by HindIII digest fragments; PSEL=synthetic early late promoter; mIL2v=mouse interleukin-2 variant; *=mutation encoding substitution of lysine to glutamate at position 151 of A34 protein; PF17=promoter from the F17R gene; HSV TK.007=herpes simplex virus thymidine kinase gene with mutation encoding alanine to histidine substitution at position 168.

FIG. 2. Schematic representation of full genomes for recombinant oncolytic viruses VV94 and IGV-121. Abbreviations: LITR=left inverted terminal repeat; RITR=right inverted terminal repeat; A-O=viral gene regions historically defined by HindIII digest fragments; PSEL=synthetic early late promoter; mIL2v=mouse interleukin-2 variant; *=mutation encoding substitution of lysine to glutamate at position 151 of A34 protein; PF17=promoter from the F17R gene; HSV TK.007=herpes simplex virus thymidine kinase gene with mutation encoding alanine to histidine substitution at position 168.

FIG. 3. Schematic representation of full genomes for recombinant oncolytic viruses VV101-VV103. Abbreviations: LITR=left inverted terminal repeat; RITR=right inverted terminal repeat; A-O=viral gene regions historically defined by HindIII digest fragments; PSEL=synthetic early late promoter; hIL2v=human interleukin-2 variant; *=mutation encoding substitution of lysine to glutamate at position 151 of A34 protein; PF17=promoter from the F17R gene; HSV TK.007=herpes simplex virus thymidine kinase gene with mutation encoding alanine to histidine substitution at position 168.

FIG. 4. mIL-2v expression analysis following infection of cells with recombinant oncolytic vaccinia viruses.

FIG. 5. hIL-2v expression analysis following infection of cells with recombinant oncolytic vaccinia viruses.

FIG. 6. HSV TK.007 expression analysis following infection of cells with recombinant oncolytic vaccinia viruses.

FIG. 7A-7G. Assessment of virotherapy-induced tumor growth inhibition on C57BL/6 female mice implanted SC with MC38 tumor cells. Tumor growth trajectories are shown for individual mice in groups treated with vehicle only (A) or Copenhagen vaccinia virus containing the A34R K151E mutation armed with either a Luciferase-2A-GFP reporter (Cop.Luc-GFP.A34R-K151E; VV16) (B), mIL-2v only (Cop.mGM-CSF.A34R-K151E; VV27) (C), mIL-2v and HSV TK.007 in a forward orientation in the B16R gene locus (Cop.mIL-2v.A34R-K151E.HSV TK.007 (B16R_For); VV91) (D), mIL-2v and HSV TK.007 in a reverse orientation in the J2R gene locus (Cop.mIL-2v.A34R-K151E.HSV TK.007 (J2R_Rev); VV93) (E), or mIL-2v and HSV TK.007 in a reverse orientation in the B16R gene locus (Cop.mIL-2v.A34R-K151E.HSV TK.007 (B16R_Rev); VV96) (F). The dashed vertical line on each graph represents time point when mice received intratumoral injections of vehicle or virus. The dashed horizontal line on each graph represents the tumor volume threshold used as a criterion to remove animals from the study. Average tumor volumes (mm3)±95% confidence intervals for each treatment group are shown through day 28 post-tumor implant (G), which was the last tumor measurement time point when all animals in each group were still alive.

FIG. 8. Statistical comparison of virotherapy-induced tumor growth inhibition using ANCOVA. Values in bold font represent comparative ANCOVA results where p≤0.05.

FIG. 9. Survival of MC38 tumor-implanted C57BL/6 female mice following treatment with vehicle or virus on day 12 after implantation. The point of intersection between each group's curve and the horizontal dashed line indicates the median (50%) survival threshold for group.

FIG. 10. IL-2 levels detected in sera collected from MC38 tumor-bearing C57BL/6 female mice 24 hours (hr) and 48 hr after intratumoral injection with vehicle or recombinant Cop vaccinia viruses. Each symbol represents the calculated IL-2 serum levels for an individual mouse, while bars represent group geometric mean (N=9/group). Error bars represent 95% confidence intervals.

FIG. 11A-11F. Assessment of virotherapy-induced tumor growth inhibition on C57BL/6 female mice implanted SC with LLC tumor cells. Tumor growth trajectories are shown for individual mice in groups treated with vehicle only (A) or Copenhagen vaccinia virus containing the A34R K151E mutation and armed with either a Luciferase-2A-GFP reporter (Cop.Luc-GFP.A34R-K151E; VV16) (B), mIL-2v only (Cop.IL-2v.A34R-K151E; VV27) (C), mIL-2v and HSV TK.007 in a forward orientation in the B16R gene locus (Cop.mIL-2v.A34R-K151E.HSV TK.007 (B16R_For); VV91) (D), mIL-2v and HSV TK.007 in a reverse orientation in the J2R gene locus (Cop.mIL-2v.A34R-K151E.HSV TK.007 (J2R_Rev); VV93) (E), mIL-2v and HSV TK.007 in a reverse orientation in the B16R gene locus (Cop.mIL-2v.A34R-K151E.HSV TK.007 (B16R_Rev); VV96) (F). The dashed vertical line on each graph represents time point when mice received intratumoral injections of vehicle or virus. The dashed horizontal line on each graph represents the tumor volume threshold used as a criterion to remove animals from the study

FIG. 12. IL-2 levels detected in sera collected from LLC tumor-bearing C57BL/6 female mice 24, 48, and 72 hr after intratumoral injection with vehicle or recombinant Cop vaccinia viruses. Each symbol represents the calculated IL-2 serum levels for an individual mouse, while bars represent group geometric mean (N=5/group). Error bars represent 95% confidence intervals.

FIG. 13A-13F. Assessment of virotherapy-induced tumor growth inhibition using single (day 11) IV virus delivery on C57BL/6 female mice implanted SC with MC38 tumor cells. Tumor growth trajectories are shown for each treatment as group averages±95% confidence intervals up through day 32 post-tumor implantation until time of sacrifice (A) or for individual mice in each group until time of sacrifice or study termination (B-F). Test viruses included WR vaccinia viruses containing the A34R K151E mutation and armed with either a Luciferase-2A-GFP reporter (WR.Luc-GFP.A34R-K151E; VV17) (C), mIL-2v only (WR.mIL-2v.A34R-K151E; VV79) (D), mIL-2v with HSV TK.007 in a reverse orientation in the J2R gene locus (WR.mIL-2v.A34R-K151E.HSV TK.007 (J2R_Rev); VV94) (E), and mIL-2v and HSV TK.007 in a forward orientation in the B15R/B17R gene locus (WR.mIL-2v.A34R-K151E.HSV TK.007 (B16R_For); IGV-121) (F).

FIG. 14. Statistical comparison of virotherapy-induced tumor growth inhibition using ANCOVA for subcutaneous MC38 tumor model study. Values in bold font represent comparative ANCOVA results where p values 0.05 were observed.

FIG. 15. Survival of MC38 tumor-bearing C57BL/6 female mice following IV treatment with recombinant oncolytic vaccinia viruses on day 11 after SC tumor implantation. P values represent the statistical results of Log-rank test (Mantel-Cox) comparisons between select virus groups.

FIG. 16. IL-2 levels detected in sera collected from MC38 tumor-bearing C57BL/6 female mice 72 hr (day 14) after IV injection with 5e7 pfu recombinant WR vaccinia viruses. Each symbol represents IL-2 serum levels detected in an individual mouse, while bars represent the group geometric means (N=10/group). Error bars represent 95% confidence intervals.

FIG. 17A-17D. Assessment of virotherapy-induced tumor growth inhibition using single (day 14) IV virus delivery on C57BL/6 female mice implanted SC with LLC tumor cells. Tumor growth trajectories are shown for each treatment as group averages±95% confidence intervals up through day 27 post-tumor implantation until time of sacrifice (A) or for individual mice in each group until time of sacrifice or study termination (B-D). Test viruses included WR vaccinia viruses armed with either a Luciferase-2A-GFP reporter (WR.Luc-GFP; VV3) (C), or mIL-2v and HSV TK.007 in a forward orientation in the B15R/B17R gene locus with the A34R K151E mutation (WR.mIL-2v.A34R-K151E.HSV TK.007 (B16R_For); IGV-121)) (D).

FIG. 18. Statistical comparison of virotherapy-induced tumor growth inhibition using ANCOVA for subcutaneous LLC tumor model study. Values in bold font represent comparative ANCOVA results where p values 0.05 were observed.

FIG. 19. Survival of LLC tumor-bearing C57BL/6 female mice following IV treatment with recombinant oncolytic vaccinia viruses on day 14 after SC tumor implantation. P values represent the statistical results of Log-rank test (Mantel-Cox) comparisons between select virus groups.

FIG. 20A-20I. Assessment of virotherapy-induced tumor growth inhibition on C57BL/6 female mice implanted SC with MC38 tumor cells. Tumor growth trajectories are shown for individual mice in groups treated with vehicle only (A), Copenhagen vaccinia virus armed with either mIL-2v and HSV TK.007 in a forward orientation in the B16R gene locus (Cop.mIL-2v.A34R-K151E.HSV TK.007 (B16R_For); VV91)) at 5e7 pfu (B), hIL-2v and HSV TK.007 in a forward orientation in the B16R gene locus (Cop.hIL-2v.A34R-K151E.HSV TK.007 (B16R_For); VV102)) at 5e7 pfu (C), mGM-CSF and a LacZ reporter transgene (Cop.mGM-CSF/LacZ; (VV10) at 5e7 pfu (D), a Luciferase-2A-GFP reporter (Cop.Luc-GFP; VV7) at 2e8 pfu (E), mIL-2v and HSV TK.007 in a forward orientation in the B16R gene locus (Cop.mIL-2v.A34R-K151E.HSV TK.007 (B16R_For); VV91)) at 2e8 pfu (F), hIL-2v and HSV TK.007 in a forward orientation in the B16R gene locus (Cop.hIL-2v.A34R-K151E.HSV TK.007 (B16R_For); VV102)) at 2e8 pfu (G), and mGM-CSF and a LacZ reporter transgene (Cop.mGM-CSF/LacZ; (VV10) at 2e8 pfu (H). The dashed vertical line on each graph represents time point when mice received intratumoral injections of vehicle or virus. The dashed horizontal line on each graph represents the tumor volume threshold used as a criterion to remove animals from the study. Average tumor volumes (mm3) for each treatment group are shown through day 28 post-tumor implant (I).

FIG. 21. Statistical comparison of virotherapy-induced tumor growth inhibition using ANCOVA. Columns show the statistical results (p values) of comparisons between specific treatment group pairs. Values in bold font represent comparative ANCOVA results where p≤0.05.

FIG. 22A-B. Survival of MC38 tumor-implanted C57BL/6 female mice following treatment with vehicle or virus on day 11 after implantation. Mice were designated daily as deceased upon reaching tumor volume ≥1400 mm3. The point of intersection between each group's curve and the horizontal dashed line indicates the median (50%) survival threshold for group. (A) shows groups dosed with 5e7 pfu virus. (B) shows groups dosed with virus at 2e8 pfu.

FIG. 23. Mouse IL-2 levels detected in sera collected from MC38 tumor-bearing C57BL/6 female mice 24 hr after intratumoral injection with vehicle or recombinant Cop vaccinia viruses. Each symbol represents the calculated IL-2 serum levels for an individual mouse, while bars represent group geometric mean (N=10/group). Error bars represent 95% confidence intervals.

FIG. 24. Human IL-2 levels detected in sera collected from MC38 tumor-bearing C57BL/6 female mice 24 hr after intratumoral injection with vehicle or recombinant Cop vaccinia viruses. Each symbol represents the calculated IL-2 serum levels for an individual mouse, while bars represent group geometric mean (N=9/group). Error bars represent 95% confidence intervals.

FIG. 25. Assessment of virotherapy-induced tumor growth inhibition on Nude female mice implanted SC with HCT-116 tumor cells. Average tumor volumes (mm3) for each treatment group are shown through day 40 post-tumor implant. The dashed vertical line on each graph represents time point when mice received intratumoral injections of vehicle or virus. The dashed horizontal line on each graph represents the tumor volume threshold used as a criterion to remove animals from the study.

FIG. 26. Schematic representation of full genomes for VV97-100.

FIG. 27. Schematic representation of full genomes for VV110.

FIG. 28. Schematic representation of full genomes for VV117.

FIG. 29A-C. Assessment of STAT5 phosphorylation in murine splenocytes incubated with IL-2 variant transgenes expressed by recombinant WR vaccinia viruses. Comparison of pSTAT5 induction in subsets of murine splenocytes incubated with either hIL-2, hIL-2 variant, or hIL-2 glycovariants. IL-2 functionality was assessed using measurement of intracellular pSTAT5 levels as a readout of IL-2R-mediated signaling. Splenocytes were additionally stained with antibodies to cell surface markers (CD3, CD4, CD8, CD25, and NKp46) and an intracellular protein (FoxP3) to delineate various subsets of murine lymphocytes expressing different IL2R complexes. Graphs show changes in median fluorescence intensity (MFI) values of intracellular staining of pSTAT5 (y-axis) in response to increasing treatment concentrations of hIL-2, hIL-2 variant, or hIL-2 glycovariant protein secreted by the indicated viruses (x-axis). Abbreviations: pSTAT5=phosphorylated signal transducer and activator of transcription 5; MFI=median fluorescence intensity; Treg=CD3+CD4+CD25+ Foxp3+ T regulatory cells.

FIG. 30. Body weights of MC38 tumor-implanted C57BL/6 female mice following treatment with vehicle or virus on day 11 after implantation.

FIG. 31. IL-2 levels detected in sera collected from MC38 tumor-bearing C57BL/6 female mice 72 hr (day 14) after IV injection with 5e7 pfu recombinant WR vaccinia viruses. Statistics were performed using a One-way Anova test with a Tukey's post-hoc multiple group comparison test as compared to VV99 with *=p<0.05; **=p<0.01 and ***=p<0.001

FIG. 32. Table 3. Inflammatory cytokine levels detected in sera collected from MC38 tumor-bearing C57BL/6 female mice 72 hr (day 14) after IV injection with 5e7 pfu recombinant WR vaccinia viruses. Each column shows geometric mean cytokine levels (N=10/test group) for the designated cytokine. *=p<0.05; **=p<0.01; +=p<0.001; \hat{ }=p<0.0001

FIG. 33. Assessment of virotherapy-induced tumor growth inhibition using single (administered on day 11) IV virus delivery on C57BL/6 female mice implanted SC with MC38 tumor cells.

FIG. 34. Table 4. Statistical comparison of virotherapy-induced tumor growth inhibition using ANCOVA for subcutaneous MC38 tumor model study. Values in bold font represent comparative ANCOVA results where p values 0.05 were observed.

FIG. 35. Survival of MC38 tumor-bearing C57BL/6 female mice following IV treatment with recombinant oncolytic vaccinia viruses on day 11 after SC tumor implantation. The point of intersection between each group's curve and the horizontal dashed line indicates the median (50%) survival threshold for the group.

FIG. 36. Table 5. Statistical comparison of survival following virotherapy in the subcutaneous MC38 tumor model study. Survival data from FIG. 35 was analyzed by Log-rank test (Mantel-Cox). P values represent the statistical results of Log-rank test (Mantel-Cox) comparisons between select virus groups.

FIG. 37. Assessment of virotherapy-induced tumor growth inhibition on Nude female mice implanted SC with HCT-116 tumor cells. Average tumor volumes (mm3) for each treatment group are shown through day 43 post-tumor implant. The dashed vertical line on each graph represents time point when mice received IV injections of vehicle or virus.

FIG. 38. Table 6. Statistical comparison of virotherapy-induced tumor growth inhibition using ANCOVA on subcutaneous HCT-116 tumors in Nude mice. Values in bold font represent comparative ANCOVA results where p values 0.05 were observed.

FIG. 39. Assessment of virotherapy-induced survival on Nude female mice implanted SC with HCT-116 tumor cells. Euthanasia was performed once tumors reached 2000 mm3. The dashed vertical line on each graph represents time point when mice received IV injections of vehicle or virus (3E6 PFU). The dashed horizontal line on the graph represents 50 percent survival, or median survival.

FIG. 40. Table 7. Statistical comparison of virotherapy-induced survival in Nude female mice implanted SC with HCT-116 tumor cells. P values are listed for each group comparison.

FIG. 41. Assessment of virotherapy-induced tumor growth inhibition using single (day 16) IV virus delivery on C57BL/6 female mice implanted SC with MC38 tumor cells.

FIG. 42. Table 8. Statistical comparison of virotherapy-induced tumor growth inhibition using ANCOVA for subcutaneous MC38 tumor model study. Values in bold font represent comparative ANCOVA results where p values 0.05 were observed.

FIG. 43. Survival of MC38 tumor-bearing C57BL/6 female mice following IV treatment with recombinant oncolytic vaccinia viruses on day 16 after SC tumor implantation. P values represent the statistical results of Log-rank test (Mantel-Cox) comparisons between select virus groups.

FIG. 44. Table 9. Statistical comparison of virotherapy-induced survival. Survival was monitored and then analyzed by Log-rank test (Mantel-Cox). P values are listed for each group comparison.

FIG. 45. Assessment of virotherapy-induced tumor growth inhibition using single (day 18) IV virus delivery on C57BL/6 female mice implanted SC with B16F10 tumor cells in combination with anti-PD-1 antibody treatment.

FIG. 46. Table 10. Statistical comparison of virotherapy-induced tumor growth inhibition using ANCOVA for subcutaneous B16F10 tumor model study. Values in bold font represent comparative ANCOVA results where p values 0.05 were observed.

FIG. 47. Survival of B16F10 tumor-bearing C57BL/6 female mice following IV treatment with recombinant oncolytic vaccinia viruses on day 18 after SC tumor implantation.

FIG. 48. Table 11. Statistical comparison of virotherapy-induced survival in the B16F10 tumor model. Survival was monitored and then analyzed by Log-rank test (Mantel-Cox). P values are listed for each group comparison.

FIG. 49 depicts a schematic drawing depicting an IL-2 variant fusion protein comprising an IL-2 variant covalently linked to an Fc domain. The Fc domain contains a first Fc chain and a second Fc chain, in which the first Fc chain contains “knob” amino acid substitutions and the second Fc chain contains “hole” amino acid substitutions. The N-terminus of the IL-2 variant is covalently linked via a linker to the C-terminus of the first Fc chain.

FIG. 50A-50B depict graphs summarizing the effect of various concentrations of different IL-2 fusion proteins on pSTAT5 levels in HH cells (FIG. 50A) and iTreg cells (FIG. 50B), as determined by ELISA. The IL-2 variant fusion proteins for both FIGS. 50A and 50B are listed in FIG. 50B. The X-axis shows the IL-2 fusion protein concentration (nM) and the Y-axis shows pSTAT5 optical density (OD).

FIG. 51A-C depict graphs summarizing the effect of various concentrations of different IL-2 fusion proteins on pSTAT5 levels in CD8 T cells (FIG. 51A), NK cells (FIG. 51B), and Treg cells (FIG. 51C) as determined by flow cytometry. The IL-2 variant fusion proteins for FIGS. 51A, 51B, and 51C are listed in FIG. 51C. The X-axis shows the IL-2 fusion protein concentration (nM) and the Y-axis shows pSTAT5 mean fluorescence intensity (MFI).

FIG. 52A-C depict graphs summarizing the effect of various concentrations of different IL-2 fusion proteins on pSTAT5 levels in CD8 T cells (FIG. 52A), NK cells (FIG. 52B), and Treg cells (FIG. 52C) as determined by flow cytometry. The IL-2 variant fusion proteins for FIGS. 52A, 52B, and 52C are listed in FIG. 52C. The X-axis shows the IL-2 fusion protein concentration (nM) and the Y-axis shows pSTAT5 mean fluorescence intensity (MFI).

FIG. 53A-C depict graphs summarizing the effect of various concentrations of different IL-2 fusion proteins on pSTAT5 levels in CD8 T cells (FIG. 53A), NK cells (FIG. 53B), and Treg cells (FIG. 53C) as determined by flow cytometry. The IL-2 variant fusion proteins for FIGS. 53A, 53B, and 53C are listed in FIG. 53C. The X-axis shows the IL-2 fusion protein concentration (nM) and the Y-axis shows pSTAT5 mean fluorescence intensity (MFI).

FIG. 54A-C depict graphs summarizing the effect of various concentrations of different IL-2 fusion proteins on pSTAT5 levels in CD8 T cells (FIG. 54A), NK cells (FIG. 54B), and Treg cells (FIG. 54C) as determined by flow cytometry. The IL-2 variant fusion proteins for FIGS. 54A, 54B, and 54C are listed in FIG. 54C. The X-axis shows the IL-2 fusion protein concentration (nM) and the Y-axis shows pSTAT5 mean fluorescence intensity (MFI).

FIG. 55A-C depict graphs summarizing the effect of various concentrations of different IL-2 fusion proteins on pSTAT5 levels in CD8 T cells (FIG. 55A), NK cells (FIG. 55B), and Treg cells (FIG. 55C) as determined by flow cytometry. The IL-2 variant fusion proteins for FIGS. 55A, 55B, and 55C are listed in FIG. 55C. The X-axis shows the IL-2 fusion protein concentration (nM) and the Y-axis shows pSTAT5 mean fluorescence intensity (MFI).

FIG. 56A-C depict graphs summarizing the effect of various concentrations of different IL-2 fusion proteins on the expansion of CD8 T cells (FIG. 56A), NK cells (FIG. 56B), and Treg cells (FIG. 56C). The X-axis shows the IL-2 fusion protein (each at 3 different concentrations) and the Y-axis shows the fold-expansion of the cells.

FIG. 57A-B show the tolerability (FIG. 57A) and tumor growth inhibition activity (FIG. 57B) of different IL-2 fusion proteins in mice. In FIG. 57A, the X-axis shows days post-treatment, and the Y-axis shows percent survival of the mice. The survival data for the different proteins are depicted with lines annotated with the following symbols: solid circle: PBS (no protein); empty circle: Fc-IL2; solid triangle: Fc-IL2v; empty triangle: Fc-IL2-K43N:Y45T; solid square: Fc-IL2-R38N:L40T-K43N:Y45T; empty square: Fc-IL2-K43N:Y45T-L72N:Q74T. In FIG. 57B, the X-axis shows days post-treatment, and the Y-axis shows tumor volume (mm³). Data for the different proteins are depicted with lines annotated with the following symbols: asterisk: PBS (no protein); “X”: Fc-IL2-R38N:L40T-K43N:Y45T; “0”: Fc-IL2-K43N:Y45T-L72N:Q74T.

FIG. 58. Maximum human tumor cell killing induced by VV110 or VV12 (JX-594) at 48, 72, and 96 hours post-infection. Data are represented as mean±SD.

FIG. 59. Potency of VV110 and VV12 in human tumor cell lines induced by VV110 or VV12 (JX-594) at 48, 72, and 96 hrs post-infection. Data are represented as mean±SD.

FIG. 60. Relative potency (EC50 ratio) of VV110 and VV12 in human tumor cell lines. Data are represented as mean±SD.

FIG. 61. Infectious virus titer from spontaneous skin lesions occurring following IV administration of VV110 to cynomolgus monkeys, with or without topical acyclovir treatment. Swabs were collected from individual skin lesions on animals that received 5×10⁷ PFU VV110 IV, either without (Group 1) or with (Group 2) topical acyclovir administration.

### DETAILED DESCRIPTION

### A. Definitions

The term “antibody” refers to an immunoglobulin molecule capable of specific binding to a target antigen, such as a carbohydrate, polynucleotide, lipid, polypeptide, etc. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. A full IgG antibody molecule contains two identical heavy chains and two identical light chains. Each of the heavy chain and light chain contains a variable region and a constant region. The variable regions of the heavy and light chains each consist of four framework regions (FRs) connected by three complementarity determining regions (CDRs) also known as hypervariable regions, and contribute to the formation of the antigen binding site of antibodies. As used herein, the term “antibody” encompasses not only full polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)2, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide.

The term “degenerate variant” refers to a nucleic acid sequence that has substitutions of bases relative to a reference nucleic acid sequence but encodes the same amino acid sequence as the reference nucleic acid sequence.

The term “effective amount” refers to an amount administered to a mammal that is sufficient to cause a desired effect in the mammal.

The term “Fc region” or “Fc chain” refers to a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

The term “Fc domain” refers to the region of an antibody that comprises two Fc regions/Fc chains. For example, in standard IgG format, an antibody has two heavy chains, both of which have an Fc region/Fc chain. Collectively, the two Fc regions/Fc chains are referred to herein as an “Fc domain”.

The term “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “immune-effector-cell enhancer” or “IEC enhancer” refers to a substance capable of increasing or enhancing the number, quality, or function of one or more types of immune effector cells of a mammal. Examples of immune effector cells include cytolytic CD8 T cells, CD4 T cells, NK cells, and B cells.

The term “immune-suppressive-cell inhibitor” or “ISC inhibitor” refers to a substance capable of reducing or suppressing the number or function of immune suppressive cells of a mammal. Examples of immune suppressive cells include regulatory T cells (“Treg”), myeloid-derived suppressor cells, and tumor-associated macrophages.

The term “immune modulator” refers to a substance capable of altering (e.g., inhibiting, decreasing, increasing, enhancing, or stimulating) the immune response (as defined herein) or the working of any component of the innate, humoral or cellular immune system of a host mammal. Thus, the term “immune modulator” encompasses the “immune-effector-cell enhancer” as defined herein and the “immune-suppressive-cell inhibitor” as defined herein, as well as substance that affects other components of the immune system of a mammal.

The term “immune response” refers to any detectable response to a particular substance (such as an antigen or immunogen) by the immune system of a host mammal, such as innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines).

The term “neoplastic disorder” refers to a condition in which cells proliferate at an abnormally high and uncontrolled rate, the rate exceeding and uncoordinated with that of the surrounding normal tissues. It usually results in a solid lesion or lump known as “tumor.” This term encompasses benign and malignant neoplastic disorders. The term “malignant neoplastic disorder”, which is used interchangeably with the term “cancer” in the present disclosure, refers to a neoplastic disorder characterized by the ability of the tumor cells to spread to other locations in the body (known as “metastasis”). The term “benign neoplastic disorder” refers to a neoplastic disorder in which the tumor cells lack the ability to metastasize.

The term “oncolytic” virus refers to a virus that preferentially infects and kills cancer cells, compared to normal (non-cancerous) cells.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “preventing” or “prevent” refers to (a) keeping a disorder from occurring or (b) delaying the onset of a disorder or onset of symptoms of a disorder.

The term “replication-competent” virus refers to a virus that is capable of infecting and replicating within a particular host cell.

The term “recombinant” virus refers to a virus that has been manipulated in vitro, e.g., using recombinant nucleic acid techniques, to introduce changes to the viral genome and/or to introduce changes to the viral proteins. For example, a recombinant virus may include both wild-type, endogenous, nucleic acid sequences and mutant and/or exogenous nucleic acid sequences. A recombinant virus may also include modified protein components. A “recombinant vaccinia virus” refers to a recombinant virus that is modified or constructed based on a vaccinia virus genome backbone.

The terms “treatment,” “treating,” and the like, refer to obtaining a desired pharmacologic and/or physiologic effect, such as inhibiting a disorder, i.e., arresting its development, relieving a disorder, i.e., causing regression of the disorder, reducing the severity of a disorder, or reducing the occurrence frequency of a symptom of a disorder.

The term “therapeutically effective amount” or “efficacious amount” refers to the amount of an agent (e.g., a replication-competent, recombinant oncolytic vaccinia virus of the present disclosure), or combined amounts of two or more agents (e.g., a replication-competent, recombinant oncolytic vaccinia virus of the present disclosure and a second therapeutic agent), that, when administered to a subject for treating a disease, is sufficient to cause an intended effect, such treatment for the disease. The “therapeutically effective amount” will vary depending on the agent(s), the disease and its severity and the age, weight, etc., of the subject to be treated.

The term “Interleukin-2” or “IL-2” refer to the wild-type Interleukin-2 protein of any mammalian species, such as human, canine, feline, equine, and bovine. One exemplary wild-type human IL-2 is found as Uniprot Accession Number P60568. The amino acid sequence of full length, wild-type human IL-2 is provided in SEQ ID NO:21. Full length wild-type human IL-2 contains a signal peptide (first 20 amino acids) which is removed during the maturation of the IL-2 protein. The amino acid sequence of a mature, active form of human IL-2, which does not contain the signal peptide, is provided in SEQ ID NO:1 (APTSSSTKKTQLQLEHLLLDLQMILNGINNYKNPKLTRMLTFKFYMPKKATELKHL QCLEEELKPLEEVLNLAQSKNFHLRPRDLISNINVIVLELKGSETTFMCEYADETATI VEFLNRWITFCQSIISTLT). Unless otherwise noted, all references herein to particular amino acids in the human IL-2 sequence are to amino acids numbered according to the amino acid sequence of the mature IL-2 protein (i.e. SEQ ID NO: 1). For example, the IL-2 R38 residue refers to the 38th residue (an R) in the amino acid sequence as shown in SEQ ID NO: 1. It should be understood that the R38 residue in the amino acid sequence of SEQ ID NO:1 corresponds to R58 in the amino acid of SEQ ID NO: 21.

The term “variant IL-2,” “IL-2 variant,” or “IL-2v,” unless otherwise noted, refers to polypeptide that contains one or more amino acid substitutions relative to the amino acid sequence of a wild-type IL-2 protein and retains at least part of the activities of the wild-type IL-2 protein.

The term “IL-2 receptor alpha” refers to the alpha polypeptide chain of the IL-2 receptor. “IL-2 receptor alpha” is also known as and referred to herein as “IL-2Rα”, “IL-2R alpha”, “IL-2Rα”, and “CD25”. One exemplary wild-type human IL-2R alpha amino acid sequence is found as Uniprot Accession Number P01589.

The term “IL-2 receptor beta” refers to the beta polypeptide chain of the IL-2 receptor. “IL-2 receptor beta” is also known as and referred to herein as “IL-2Rb”, “IL-2R beta”, “IL-2Rb”, and “CD122”. One exemplary wild-type human IL-2R beta amino acid sequence is found as Uniprot Accession Number P14784.

The term “IL-2 receptor gamma” refers to the gamma polypeptide chain of the IL-2 receptor. “IL-2 receptor gamma” is also known as and referred to herein as “IL-2Ry”, “IL-2R gamma”, “IL-2Rg”, and “CD132”. One exemplary wild-type human IL-2R gamma amino acid sequence is found as Uniprot Accession Number P31785.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to “a vaccinia virus” includes a plurality of such vaccinia viruses and reference to “the variant IL-2 polypeptide” includes reference to one or more variant IL-2 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### B. IL-2 Variants and Related Aspects

B-1. IL-2 Variants

In some first aspects, the present disclosure provides IL-2 variants (e.g., human IL-2 variants) that have one or more amino acid substitutions as compared to the wild-type human IL-2 amino acid sequence. In some embodiments, the IL-2 variant comprises one or more substitutions relative to the mature human wild-type IL-2 protein sequence (SEQ ID NO: 1) at one or more of the following amino acid positions: T3, K35, R38, L40, T41, K43, Y45, E62, K64, L72, Q74, and C125.

In some embodiments, the IL-2 variant comprises amino acid substitutions at one or more of the following groups of positions: R38 and L40; T41 and K43; K43 and Y45; E62 and K64; L72 and Q74; R38, L40, K43, and Y45; K43, Y45, L72, and Q74; T3, R38, L40, K43, and Y45; T3, K43, Y45, L72, and Q74; R38, L40, K43, Y45, and C125; K43, Y45, L72, Q74, and C125; T3, R38, L40, K43, Y45, and C125; T3, K43, Y45, L72, Q74, and C125.

In some embodiments, the IL-2 variant comprises one of more of the following amino acid substitutions relative to the mature human wild-type IL-2 protein: T3A, K35N, R38N, L40S, L40T, T41N, K43S, K43T, K43N, Y45S, Y45T, E62N, E62A, E62K, E62R, K64S, K64T, L72N, Q74S, Q74T, C125A, C125S. In some particular embodiments, the IL-2 variant comprises one of more of the following amino acid substitutions relative to the mature human IL-2 protein: K35N, R38N, K43N, E62N, or L72N.

In some other embodiments, the IL-2 variants provided herein have decreased, negligible, or no binding to the IL-2Rα as compared to wild-type human IL-2 polypeptide. The IL-2 variants of the present invention further have decreased or no binding to the high-affinity IL-2 receptor tertiary complex (containing IL-2Rα+IL-2Rβ+IL-2Rγ) as compared to wild-type human IL-2 polypeptide. The IL-2 variants of the present invention retain the ability to bind to the intermediate-affinity dimeric IL-2 receptor complex (containing IL-2Rβ+IL-2Rγ).

In some embodiments, the amino acid substitutions in the IL-2 variant provided herein result in an engineered N-glycosylation site in the IL-2 variant protein. The consensus sequence for N-glycosylation is the three amino acid sequence N-x-S, N-x-T, S-x-N, or T-x-N, where N is asparagine, x is any amino acid except proline, S is serine, and T is threonine. In order to generate an engineered N-glycosylation site in the IL-2 amino acid sequence, in one embodiment, an asparagine (N) substitution is introduced into the IL-2 amino acid sequence at a position separated by one amino acid from an wild-type serine (S) or threonine (T) residue. In this situation, the amino acid sequence N-x-S, N-x-T, S-x-N, or T-x-N is generated in the IL-2 variant, wherein the N is the amino acid substitution, and the T or the S is a wild-type amino acid. In another embodiment, an S or T substitution is introduced into the IL-2 amino acid sequence at a position separated by one amino acid from a wild-type N residue. In this situation, the amino acid sequence N-x-S, N-x-T, S-x-N, or T-x-N is generated in the IL-2 variant, wherein the T or the S is the amino acid substitution, and the N is a wild-type amino acid. In another embodiment, both an N substitution and an S or T substitution is introduced into the IL-2 amino acid residue, at positions separated by one amino acid from each other. In this situation, the amino acid sequence N-x-S, N-x-T, S-x-N, or T-x-N is generated in the IL-2 variant, wherein the T or the S is an amino acid substitution, and the N is also an amino acid substitution. In some embodiments, an IL-2 variant provided herein may have multiple substitutions as compared to wild-type IL-2, in order to generate 1, 2, 3, 4, or 5 engineered N-glycosylation sites/consensus sequences in the IL-2 variant amino acid sequence.

Introduction of one or more engineered N-glycosylation sites in the IL-2 amino acid sequence results in IL-2 variants that can be glycosylated at the engineered N-glycosylation site(s). Glycosylation links an oligosaccharide moiety to the N (asparagine) residue; this oligosaccharide is also referred to as a “glycan”. In some embodiments, an IL-2 variant provided herein is referred to as a “single glycan” or “double glycan”, etc., based on the number of engineered N-glycosylation sites introduced into the IL-2 variant. For example, as used herein, a “single glycan” IL-2 variant refers to an IL-2 variant having one engineered glycosylation site and a “double glycan” IL-2 variant refers to an IL-2 variant having two engineered glycosylation sites.

In some embodiments, glycosylation of the N residue in the engineered glycosylation site(s) inhibits the interaction of the IL-2 variant with the IL-2Rα. Thus, in some embodiments, provided herein are IL-2 variants that contain one or more amino acid substitutions that result in one or more engineered N-glycosylation sites in the IL-2 sequence, that are glycosylated at the engineered glycosylation site(s), and that have reduced affinity for IL-2Rα, as compared to wild-type IL-2. Without being bound by theory, it is believed that added glycan groups on the engineered N-glycosylation sites interfere with the binding between IL-2 and IL-2Rα by sterically blocking the interaction between IL-2 and IL-2Rα. Thus, for example, when an engineered N-glycosylation site is introduced into an IL-2 variant provided herein by introducing the substitutions R38N and L40T (thereby creating the sequence N-x-T), the N residue at position 38 can be glycosylated. It is believed the glycan groups added to an engineered N residue at position 38 sterically interfere with the interaction between IL-2 and the IL-2Rα.

The strength of binding/binding affinity between a molecule of interest (e.g. IL-2 variant) and a second molecule (e.g. IL-2Rα) can be determined by methods known in the art (e.g. isothermal titration calorimetry (ITC) or surface plasmon resonance (SPR)). Typically, binding affinity is reported as the “K_{D}” value (equilibrium dissociation constant). As used herein “reduced binding” between a molecule of interest/analyte (e.g. IL-2 variant) and a ligand (e.g. IL-2Rα), as compared to that of a reference molecule/analyte (e.g. wild-type IL-2) refers to a situation where the molecule of interest binds to the ligand with lower affinity than the binding affinity between the reference molecule and the ligand. For K_{D} values, a smaller number represents stronger binding affinity (e.g. a K_{D} of 1 nM is a stronger binding affinity than a K_{D} of 5 nM). An IL-2 variant has reduced binding to IL-2Rα as compared to wild-type IL-2 when the K_{D} value for the interaction between the IL-2 variant and IL-2Rα is a larger number than the K_{D} value for the interaction between the wild-type IL-2 and IL-2Rα under the same binding conditions. In some embodiments, an IL-2 variant that has reduced binding to IL-2Rα as compared to that of wild-type IL-2 has a K_{D} value that is at least 1.5, 2, 3, 5, 10, 15, 20, 50, 100, 200, or 500 times greater than the K_{D} value of interaction between wild-type IL-2 and IL-2Rα under the same binding conditions. In situations where an IL-2 variant has reduced binding to IL-2Rα as compared to that of wild-type IL-2, in some embodiments, the ratio of a) the K_{D} value of the wild-type IL-2-IL-2Rα interaction over b) the K_{D} value of the IL-2 variant-IL-2Rα interaction [i.e. (K_{D} value of the wild-type IL-2-IL-2Rα interaction)/(K_{D} value of the IL-2 variant-IL-2Rα interaction)] is equal to or less than about 0.5, 0.25, 0.1, 0.05, 0.025, 0.01, 0.005, 0.0025, or 0.001. In some embodiments, an IL-2 variant that has reduced binding to IL-2Rα as compared to that of wild-type IL-2 has non-detectable binding to IL-2Rα, while wild-type IL-2 has detectable/measurable binding to IL-2Rα under the same binding conditions.

In some embodiments, an engineered N-glycosylation site in an IL-2 variant is generated by the amino acid substitution K35N. After the K35N substitution, the amino acid sequence for amino acid numbers 35-37 of the engineered IL-2 variant is: N35-L36-T37. Thus, a consensus N-glycosylation site (N-x-T) is generated by the K35N substitution, given the combination of the N35 substitution and the wild-type T37 amino acid residue.

In some embodiments, an engineered N-glycosylation site in an IL-2 variant is generated by the amino acid substitutions R38N and L40S or L40T. After the R38N and L40S or L40T substitutions, the amino acid sequence for amino acid numbers 38-40 of the engineered IL-2 variant is: N38-M39-S40 or N38-M39-T40. Thus, a consensus N-glycosylation site (N-x-T or N-x-S) is generated by the R38N and L40S or L40T substitutions.

In some embodiments, an engineered N-glycosylation site in an IL-2 variant is generated by the amino acid substitutions T41N and K43S or K43T. After the T41N and K43S or K43T substitutions, the amino acid sequence for amino acid numbers 41-43 of the engineered IL-2 variant is: N41-F42-S43 or N41-F42-T43. Thus, a consensus N-glycosylation site (N-x-T or N-x-S) is generated by the T41N and K43S or K43T substitutions.

In some embodiments, an engineered N-glycosylation site in an IL-2 variant is generated by the amino acid substitutions K43N and Y45S or Y45T. After the K43N and Y45S or Y45T substitutions, the amino acid sequence for amino acid numbers 43-45 of the engineered IL-2 variant is: N43-F44-S45 or N43-F44-T45. Thus, a consensus N-glycosylation site (N-x-T or N-x-S) is generated by the K43N and Y45S or Y45T substitutions.

In some embodiments, an engineered N-glycosylation site in an IL-2 variant is generated by the amino acid substitutions E62N and K64S or K64T. After the E62N and K64S or K64T substitutions, the amino acid sequence for amino acid numbers 62-64 of the engineered IL-2 variant is: N62-L63-S64 or N62-L63-T64. Thus, a consensus N-glycosylation site (N-x-T or N-x-S) is generated by the E62N and K64S or K64T substitutions.

In some embodiments, an engineered N-glycosylation site in an IL-2 variant is generated by the amino acid substitutions L72N and Q74S or Q74T. After the L72N and Q74S or Q74T substitutions, the amino acid sequence for amino acid numbers 72-74 of the engineered IL-2 variant is: N72-A73-S74 or N72-A73-T74. Thus, a consensus N-glycosylation site (N-x-T or N-x-S) is generated by the L72N and Q74S or Q74T substitutions.

In some embodiments, an amino acid substitution provided herein is not part of an engineered consensus N-glycosylation site, but the substitution also reduces the binding affinity between IL-2 and IL-2Rα. For example, the substitutions E62A, E62K, and E62R reduce binding affinity between IL-2 and IL-2Rα but are not part of a consensus N-glycosylation site. In another example, the substitution E62N can be introduced without also a substitution at position K64 (i.e. so that the E62N substitution is introduced without generating an engineered consensus N-glycosylation site). These substitutions can be combined, for example, with other amino acid substitutions provided herein that generate one or more engineered consensus N-glycosylation site(s) in the IL-2 variant.

In some embodiments, an amino acid substitution provided herein increases the homogeneity of the IL-2 variant. For example, the substitutions at positions T3 or C125 (for example, T3A, T3G, C125A, or C125S) can increase homogeneity of IL-2 protein, and can be combined, for example, with other amino acid substitutions provided herein that generate one or more engineered consensus N-glycosylation site(s) in the IL-2 variant and/or that reduce binding affinity between IL-2 and IL-2Rα.

B-2. Fusion Molecules Comprising an IL-2 Variant

In some embodiments, provided herein are IL-2 fusion proteins that comprises an IL-2 variant provided by the present disclosure linked to another protein, such as an antibody or Fc-region of an antibody. In some further embodiments, provided herein are IL-2 heterodimeric protein comprising an IL-2 variant provided by the present disclosure and two antibody Fc-regions, wherein the IL-2 variant is linked to one of the Fc regions and wherein the two Fc regions are covalently linked by a disulfide bond. The IL-2 fusion protein and heterodimeric protein are collectively referred to as IL-2 “fusion molecules.” These IL-2 fusion molecules may have improved or additional properties as compared to IL-2 variant proteins alone, such as increased stability or in vivo half-life. In another example, an IL-2 fusion molecule comprises an IL-2 variant provided herein covalently linked to an Fc region, heavy chain, or light chain of an antibody. These IL-2 variant-antibody fusion proteins can be targeted to specific cell types or tissues (e.g. tumor cells) that contain the antigen recognized by the antibody. As such, these IL-2 variant-antibody fusion proteins can deliver the IL-2 variant to a desired cell type or tissue type, while minimizing off target/peripheral exposure of the IL-2 variant and thus IL-2 related toxicities.

In some embodiments, an IL-2 fusion protein comprises a polypeptide linker (e.g., heterologous or homologous sequence) between the antibody and the IL-2 variant. The polypeptide linker can be joined or conjugated at the amino terminus, at the carboxyl terminus, or both the amino and carboxyl termini of the antibody. In some embodiments, the polypeptide linker is a glycine-serine (GS)-linker.

Antibodies useful in the IL-2 fusion proteins provided herein can be monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the antibodies have an isotype that is selected from the group consisting of IgG₁, IgG₂, IgG_{2Δa}, IgG₄, IgG_{4Δb}, IgG_{4Δc}, IgG₄ S228P, IgG_{4Δb} S228P, and IgG_{4Δc} S228P. In some embodiments, the antibodies of the IL-2 fusion proteins as described herein comprise a Fc domain, such as the Fc domain can be a human IgG1, IgG2, or IgG4.

In some embodiments, the antibodies comprise amino acid modifications at positions 223, optionally 225, and 228 (e.g., (C223E or C223R), (E225R), and (P228E or P228R)) in the hinge region and at position 409 or 368 (e.g., K409R or L368E (EU numbering scheme)) in the CH3 region of human IgG2. In some other embodiments, the antibodies comprise amino acid modifications at positions 221 and 228 (e.g., (D221R or D221E) and (P228R or P228E)) in the hinge region and at position 409 or 368 (e.g., K409R or L368E (EU numbering scheme)) in the CH3 region of human IgG1. In still other embodiments, the antibodies comprise amino acid modifications at positions 349, 354, 366, 368, and/or 407 (EU numbering scheme) in the CH3 region of the human IgG1, for example, Y349C, S354C, T366W, T366S, L368A, and/or Y407V. In some other embodiments, the antibodies comprise amino acid modifications at positions 228 (e.g., (S228D, S228E, S228R, or S228K)) in the hinge region and at position 409 or 368 (e.g., R409K, R409, or L368E (EU numbering scheme)) in the CH3 region of human IgG4. In some other embodiments, the antibodies comprise amino acid modifications at one or more of positions 265 (e.g., D265A), 330 (e.g., A330S), and 331 (e.g., P331S) of the human IgG2; or one or more positions 234 (e.g., L234A), 235 (e.g., L235A), and 237 (e.g., G237A) of the human IgG1. In some other embodiments, the antibodies comprise amino acid modifications E233P/F234V/L235A (IgG_{4Δc}) of the human IgG4. In yet another embodiment, the amino acid modifications are E233P/F234V/L235A with deletion G236 (IgG_{4Δb}) of human IgG₄.

In some embodiments, antibodies in the IL-2 fusion protein provided herein comprise a modified constant region that has increased or decreased binding affinity to a human Fc gamma receptor, are immunologically inert or partially inert, e.g., do not trigger complement mediated lysis, do not stimulate antibody-dependent cell mediated cytotoxicity (ADCC), or do not activate microglia; or have reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

In some embodiments, an antibody constant region can be modified to avoid interaction with Fc gamma receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In still other embodiments, the antibody constant region is aglycosylated for N-linked glycosylation. In some embodiments, the constant region is aglycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Examples of other antibodies used in the IL-2 fusion proteins provided herein include anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-MARCO antibody, an anti-CXCR4 antibodies, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, an anti-BTN1A1 antibody, an anti-UL16 binding protein 2 (ULBP2) antibody, and an anti-GITR antibody.

The IL-2 variants and fusion molecules provided herein may be linked to a labeling agent such as a fluorescent molecule, a radioactive molecule or any other labels known in the art. Labels are known in the art which generally provide (either directly or indirectly) a signal.

IL-2 variants and fusion molecules provided herein can be constructed by methods known in the art, for example, synthetically or recombinantly. Typically, the fusion proteins of this invention are made by preparing and expressing a polynucleotide encoding them using recombinant methods described herein, although they may also be prepared by other means known in the art, including, for example, chemical synthesis.

B-3. Polynucleotides, Vectors, and Host Cells

The present disclosure also provides polynucleotides encoding any of the IL-2 variant proteins, IL-2 variant fusion proteins, and other polypeptides as described herein. In a particular embodiment, provided herein is a polynucleotide encoding an IL-2 variant containing substitutions R38N, L40T, K43N, and Y45T, wherein the polynucleotide comprises the nucleotide sequence:

(SEQ ID NO: 32)
GCCCCTACCAGCTCCTCCACCAAGAAGACCCAGCTGCAGCTGGAGCAT
TTACTGCTGGATTTACAGATGATTTTAAACGGCATCAACAACTACAAG
AACCCCAAGCTGACTAATATGACCACCTTCAACTTCACTATGCCCAAG
AAGGCCACCGAGCTGAAGCACCTCCAGTGTTTAGAGGAGGAGCTGAAG
CCTTTAGAGGAGGTGCTGAATTTAGCCCAGAGCAAGAATTTCCATTTA
AGGCCTCGTGATTTAATCAGCAACATCAACGTGATCGTGCTGGAGCTG
AAAGGCTCCGAGACCACCTTCATGTGCGAGTACGCCGACGAGACCGCC
ACCATCGTGGAGTTTTTAAATCGTTGGATCACCTTCTGCCAGAGCATC
ATCAGCACTTTAACC.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials. It will be appreciated by those of ordinary skill in the art that, as a result of the degeneracy of the genetic code, there are many nucleotide sequences that encode a polypeptide as described herein. Different nucleotide sequences that encode the same polypeptide sequence are also referred to as “degenerate variants.”

In some other embodiments, the present disclosure provides vectors, such as expression vectors, which comprises a nucleotide sequence encoding a IL-2 variant provided herein. Examples of expression vectors include to plasmids, viral vectors (such as vector derived from adenoviruses, adeno-associated viruses, retroviruses), cosmids, and expression vector(s) disclosed in PCT Publication No. WO 87/04462. Vector components generally include one or more of the following components: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons. An expression vector can be used to direct expression of an IL-2 variant or an IL-variant fusion protein in a subject. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventricle, or pericardium

The invention also provides host cells comprising any of the polynucleotides or vectors described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

B-4. Compositions and Method of Preventing or Treating Conditions

In another aspect, the present disclosure provides pharmaceutical compositions comprising an effective amount of an IL-2 variant or an IL-2 variant fusion molecule as described herein. In some embodiments, the composition comprises an IL-2 variant fusion protein comprising an anti-ULBP2 antibody and a human IL-2 variant, wherein the human IL-2 variant is covalently linked to the Fc domain of the antibody. In some embodiments, the pharmaceutical composition further comprises a pharmaceutically acceptable carrier. Examples of suitable pharmaceutically acceptable carriers for use in a pharmaceutical composition comprising an IL-2 variant or fusion molecule include those that are suitable for pharmaceutical compositions comprising a recombinant oncolytic virus as described herein below.

In another aspect, the present disclosure provides a method of treating a cancer or tumor, method of inhibiting tumor growth or progression, or a method of inhibiting metastasis of cancer cells in a subject, comprising administering to the subject in need thereof an effective amount of a composition (e.g., pharmaceutical composition) comprising an IL-2 variant or IL-2 variant fusion molecule as described herein.

A cancer can be a liquid cancer or solid cancer. Examples of liquid cancers include multiple myeloma, Hodgkin's lymphoma, B-cell lymphoma, acute myeloid leukemia, and other hematopoietic cells related cancer. Examples of other tumors or cancers that can be treated with the method provided herein include those that can be treated with the recombinant oncolytic virus provided by the present disclosure as described below.

The IL-2 variants or IL-2 variant fusion molecules as described herein can be administered to a subject via any suitable route, such as intravenous, intramuscular, intraperitoneal, intracerebrospinal, transdermal, subcutaneous, intra-articular, sublingually, intrasynovial, via insufflation, intrathecal, oral, inhalation, or topical routes.

In some embodiments, the IL-2 variant or the IL-2 variant fusion molecules is administered in combination with one or more additional therapeutic agents. Examples of additional therapeutic agents include biotherapeutic agents, chemotherapeutic agents, vaccines, CAR-T cell-based therapy, radiotherapy, another cytokine therapy (e.g., immunostimulatory cytokines including various signaling proteins that stimulate immune response, such as interferons, interleukins, and hematopoietic growth factors), an inhibitor of other immunosuppressive pathways, an inhibitors of angiogenesis, a T cell activator, an inhibitor of a metabolic pathway, an mTOR (mechanistic target of rapamycin) inhibitor (e.g., rapamycin, rapamycin derivatives, sirolimus, temsirolimus, everolimus, and deforolimus), an inhibitor of an adenosine pathway, a tyrosine kinase inhibitor, such as inlyta, ALK (anaplastic lymphoma kinase) inhibitors (e.g., crizotinib, ceritinib, alectinib, and sunitinib), a BRAF inhibitor (e.g., vemurafenib and dabrafenib), an epigenetic modifier, an inhibitors or depletor of Treg cells and/or of myeloid-derived suppressor cells, a JAK (Janus Kinase) inhibitor (e.g., ruxolitinib and tofacitinb, varicitinib, filgotinib, gandotinib, lestaurtinib, momelotinib, pacritinib, and upadacitinib), a STAT (Signal Transducers and Activators of Transcription) inhibitor (e.g., STAT1, STAT3, and STAT5 inhibitors such as fludarabine), cyclin-dependent kinase inhibitors, immunogenic agents (for example, attenuated cancerous cells, tumor antigens, antigen presenting cells such as dendritic cells pulsed with tumor derived antigen or nucleic acids, a MEK inhibitor (e.g., trametinib, cobimetinib, binimetinib, and selumetinib), a GLS1 inhibitor, a PAP inhibitor, an oncolytic virus, an IDO (Indoleamine-pyrrole 2,3-dioxygenase) inhibitor, a PRR (Pattern Recognition Receptors) agonist, and cells transfected with genes encoding immune stimulating cytokines such as but not limited to GM-CSF).

In some embodiments, an IL-2 variant or an IL-2 variant fusion molecule is used in conjunction with, for example, an anti-PD-L1 antagonist antibody; an anti-PD-antagonist antibody such as nivolumab (OPDIVO®), pembrolizumab (KEYTRUDA®), and sasanlimab; an anti-CTLA-4 antagonist antibody such as for example ipilimumab (YERVOY®); an anti-LAG-3 antagonist antibody such as BMS-986016 and IMP701; an anti-TIM-3 antagonist antibody; an anti-B7-H3 antagonist antibody such as for example MGA271; an-anti-VISTA antagonist antibody; an anti-TIGIT antagonist antibody; an anti-CD28 antagonist antibody; an anti-CD80 antibody; an anti-CD86 antibody; an anti-B7-H4 antagonist antibody; an anti-ICOS agonist antibody; an anti-CD28 agonist antibody; an innate immune response modulator (e.g., TLRs, KIR, NKG2A); an IDO inhibitor; a 4-1BB (CD137) agonist such as PF-05082566 or urelumab (BMS-663513); an OX40 agonist (such as an anti-OX-40 agonist antibody); a GITR agonist (such as TRX518); and a cytokine (pegylated or non-pegylated) therapy such as IL-10, IL-12, IL-7, IL-15, IL-21, IL-33, CSF-1, MCSF-1, etc.

B-5. Examples of Non-Limiting Embodiments of the Disclosure

Examples of other embodiments of inventions relating to the IL-2 variants provided by the present disclosure are described in the clauses below.

Clause 1. An isolated human interleukin 2 (IL-2) variant comprising at least one amino acid substitution as compared to wild-type human IL-2, wherein wild-type human IL-2 has the amino acid sequence as shown in SEQ ID NO: 1 and the IL-2 variant comprises one or more substitutions at amino acid positions selected from the group consisting of:

a) K35,

b) both R38 and L40,

c) both T41 and K43,

d) both K43 and Y45,

e) both E62 and K64, and

f) both L72 and Q74.

Clause 2. The IL-2 variant of clause 1, wherein the variant comprises one or more substitutions at amino acid positions selected from the group consisting of:

a) K35, wherein the K35 substitution is K35N,

b) both R38 and L40, wherein the R38 substitution is R38N and the L40 substitution is L40S or L40T,

c) both T41 and K43, wherein the T41 substitution is T41N and the K43 substitution is K43S or K43T,

d) both K43 and Y45, wherein the K43 substitution is K43N and the Y45 substitution is Y45S or Y45T,

e) both E62 and K64, wherein the E62 substitution is E62N and the K64 substitution is K64S or K64T, and

f) both L72 and Q74, wherein the L72 substitution is L72N and the Q74 substitution is Q74S or Q74T.

Clause 3. The IL-2 variant of clause 1 or 2, wherein the IL-2 variant comprises a substitution in position K35, and wherein the IL-2 variant further comprises substitutions at positions selected from the group consisting of:

a) both R38 and L40, wherein the R38 substitution is R38N and the L40 substitution is L40S or L40T,

b) both T41 and K43, wherein the T41 substitution is T41N and the K43 substitution is K43S or K43T,

c) both K43 and Y45, wherein the K43 substitution is K43N and the Y45 substitution is Y45S or Y45T,

d) both E62 and K64, wherein the E62 substitution is E62N and the K64 substitution is K64S or K64T,

e) both L72 and Q74, wherein the L72 substitution is L72N and the Q74 substitution is Q74S or Q74T, and

f) E62, wherein the E62 substitution is E62N, E62A, E62K, or, E62R.

Clause 4. The IL-2 variant of clause 1 or 2, wherein the IL-2 variant comprises substitutions at positions R38 and L40, and wherein the IL-2 variant further comprises substitutions at positions selected from the group consisting of:

a) both T41 and K43, wherein the T41 substitution is T41N and the K43 substitution is K43S or K43T,

b) both K43 and Y45, wherein the K43 substitution is K43N and the Y45 substitution is Y45S or Y45T,

c) both E62 and K64, wherein the E62 substitution is E62N and the K64 substitution is K64S or K64T,

d) both L72 and Q74, wherein the L72 substitution is L72N and the Q74 substitution is Q74S or Q74T, and

e) E62, wherein the E62 substitution is E62N, E62A, E62K, or, E62R.

Clause 5. The IL-2 variant of clause 1 or 2, wherein the IL-2 variant comprises substitutions at positions T41 and K43, and wherein the IL-2 variant further comprises substitutions at positions selected from the group consisting of:

a) both E62 and K64, wherein the E62 substitution is E62N and the K64 substitution is K64S or K64T,

b) both L72 and Q74, wherein the L72 substitution is L72N and the Q74 substitution is Q74S or Q74T, and

c) E62, wherein the E62 substitution is E62N, E62A, E62K, or, E62R.

Clause 6. The IL-2 variant of clause 1 or 2, wherein the IL-2 variant comprises substitutions at positions K43 and Y45, and wherein the IL-2 variant further comprises substitutions at positions selected from the group consisting of:

a) both E62 and K64, wherein the E62 substitution is E62N and the K64 substitution is K64S or K64T,

b) both L72 and Q74, wherein the L72 substitution is L72N and the Q74 substitution is Q74S or Q74T, and

c) E62, wherein the E62 substitution is E62N, E62A, E62K, or, E62R.

Clause 7. The IL-2 variant of clause 1 or 2, wherein the IL-2 variant comprises substitutions at positions E62 and K64, and wherein the IL-2 variant further comprises substitutions at positions L72 and Q74, wherein the L72 substitution is L72N and the Q74 substitution is Q74S or Q74T.

Clause 8. An isolated human interleukin 2 (IL-2) variant comprising at least four amino acid substitutions as compared to wild-type human IL-2, wherein wild-type human IL-2 has the amino acid sequence as shown in SEQ ID NO: 1 and the IL-2 variant comprises substitutions at amino acid positions selected from the group consisting of:

a) each of R38, L40, K43, and Y45; or

b) each of K43, Y45, L72, and Q74.

Clause 9. The IL-2 variant of clause 8, wherein the IL-2 variant comprises substitutions at amino acid positions R38, L40, K43, and Y45, and wherein the R38 substitution is R38N.

Clause 10. The IL-2 variant of any one of clauses 8 or 9, wherein the IL-2 variant comprises substitutions at amino acid positions R38, L40, K43, and Y45, and wherein the L40 substitution is L40T.

Clause 11. The IL-2 variant of any one of clauses 8-10, wherein the K43 substitution is K43N.

Clause 12. The IL-2 variant of any one of clauses 8-11, wherein the Y45 substitution is Y45T

Clause 13. The IL-2 variant of clause 8, wherein the IL-2 variant comprises substitutions at amino acid positions K43, Y45, L72, and Q74, and wherein the L72 substitution is L72N.

Clause 14. The IL-2 variant of any one of clauses 8 or 13, wherein the IL-2 variant comprises substitutions at amino acid positions K43, Y45, L72, and Q74, and wherein the Q74 substitution is Q74T.

Clause 15. The IL-2 variant of any one of clauses 8-12, wherein the R38 substitution is R38N and the K43 substitution is K43N.

Clause 16. The IL-2 variant of any one of clauses 8 or 11-14, wherein the K43 substitution is K43N and the L72 substitution is L72N.

Clause 17. The IL-2 variant of any one of clauses 8-12, wherein the IL-2 variant comprises the amino acid substitutions R38N, L40T, K43N, and Y45T.

Clause 18. The IL-2 variant of clause 17, wherein the IL-2 variant comprises the amino acid sequence as shown in SEQ ID NO: 31

Clause 19. The IL-2 variant of any one of clauses 8, 11-14, or 16, wherein the IL-2 variant comprises the amino acid substitutions K43N, Y45T, L72N, and Q74T.

Clause 20. The IL-2 variant of clause 19, wherein the IL-2 variant comprises the amino acid sequence as shown in SEQ ID NO: 35.

Clause 21. An isolated human interleukin 2 (IL-2) variant that comprises the amino acid sequence as shown in SEQ ID NO: 31 or 35.

Clause 22. An isolated human interleukin 2 (IL-2) variant comprising at least four amino acid substitution as compared to wild-type human IL-2, wherein wild-type human IL-2 has the amino acid sequence as shown in SEQ ID NO: 1 and the IL-2 variant comprises the four amino acid substitutions R38N, L40T, K43N, and Y45T.

Clause 23. An isolated human interleukin 2 (IL-2) variant comprising at least four amino acid substitution as compared to wild-type human IL-2, wherein wild-type human IL-2 has the amino acid sequence as shown in SEQ ID NO: 1 and the IL-2 variant comprises the four amino acid substitutions K43N, Y45T, L72N, and Q74T.

Clause 24. The IL-2 variant of any one of clauses 1-23, wherein the IL-2 variant has reduced binding to human IL-2 receptor alpha (IL-2Rα) as compared to wild-type human IL-2.

Clause 25. The IL-2 variant of any one of clauses 1-24, wherein the IL-2 variant is glycosylated on an introduced asparagine (N) residue substitution(s).

Clause 26. The IL-2 variant of any one of clauses 1-25, wherein the IL-2 variant further comprises substitutions at one or both of the positions T3 and C125.

Clause 27. The IL-2 variant of clause 26, wherein the T3 and C125 substitutions are selected from the group consisting of: T3A, T3G, C125A, and C125S.

Clause 28. An isolated fusion protein comprising: a) an IL-2 variant of any one of clauses 1-27; and b) an Fc region of a human antibody, wherein the IL-2 variant is covalently linked to the Fc region.

Clause 29. A heterodimeric protein comprising: a) the isolated fusion protein of clause 28, wherein the Fc region of the human antibody is a first Fc region; and b) a second Fc region of a human antibody, wherein the first Fc region and the second Fc region are covalently linked by at least one disulfide bond.

Clause 30. The heterodimeric protein of clause 29, wherein the first Fc region comprises at least one amino acid modification, as compared to a wild-type human IgG Fc region, to form a knob or a hole, wherein the second Fc region comprises at least one amino acid modification, as compared to a wild-type human IgG Fc region, to form a knob or a hole, and wherein one of the first and second Fc regions contains a knob and one of the first and second Fc regions contains a hole.

Clause 31. The heterodimeric protein of clause 30, wherein the Fc region comprising the knob comprises the mutations Y349C and T366W, and wherein the Fc region comprising the hole comprises the mutations S354C, T366S, L368A, and Y407V.

Clause 32. An isolated fusion protein comprising: a) an IL-2 variant of any of clauses 1-27; and b) an antibody comprising a Fc domain, wherein the Fc domain comprises a first Fc region and a second Fc region, wherein the IL-2 variant is covalently linked to a Fc region of the antibody.

Clause 33. The isolated fusion protein of clause 32, wherein the Fc domain has decreased or no antibody dependent cellular cytotoxicity (ADCC) activity as compared to a wild-type Fc domain.

Clause 34. An isolated fusion protein comprising: a) an IL-2 variant of any of clauses 1-27; and b) an antibody comprising a Fc domain, wherein the antibody comprises a first light chain and a second light chain, wherein the IL-2 variant is covalently linked to a light chain of the antibody.

Clause 35. The isolated fusion protein of clause 34, wherein the Fc domain has decreased or no antibody dependent cellular cytotoxicity (ADCC) activity as compared to a wild-type Fc domain.

Clause 36. The fusion protein of any one of clauses 32-35, wherein the antibody binds to a tumor or immune cell.

Clause 37. The fusion protein of any one of clauses 32-36, wherein the antibody is selected from the group consisting of an anti-B7H4 antibody, an anti-CTLA-4 antibody, an anti-CD3 antibody, an anti-B7H4/anti-CD3 bispecific antibody, an anti-CD28 antibody, an anti-B7H4/anti-CD28 bispecific antibody, an anti-EDB1 antibody, an anti-ULBP2 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-8 antibody, an anti-IL-7Ralpha (CD127) antibody, an anti-IL15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-MARCO antibody, an anti-CXCR4 antibodies, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7H3 antibody, an KLRG1 antibody, and an anti-GITR antibody.

Clause 38. The isolated fusion protein or heterodimeric protein of any one of clauses 22-37, wherein the IL-2 variant is covalently linked to the Fc region or the light chain, respectively, by a polypeptide linker and/or a polypeptide tag.

Clause 39. A cell line that produces the IL-2 variant, fusion protein or heterodimeric protein of any one of clauses 1-38.

Clause 40. An isolated nucleic acid encoding the IL-2 variant, fusion protein or heterodimeric protein of any one of clauses 1-38.

Clause 41. A recombinant expression vector comprising the nucleic acid of clause 40.

Clause 42. A host cell comprising the isolated nucleic acid of clause 40 or the expression vector of clause 41.

Clause 43. A method of producing the IL-2 variant, fusion protein or heterodimeric protein of any one of clauses 1-38, the method comprising culturing the host cell of clause 42 under conditions suitable for the expression of the IL-2 variant, fusion protein, or heterodimeric protein.

Clause 44. An IL-2 variant, fusion protein, or heterodimeric protein produced according to the method of clause 43.

Clause 45. A pharmaceutical composition comprising the IL-2 variant, fusion protein, or heterodimeric protein of any one of clauses 1-38, and a pharmaceutically acceptable carrier.

Clause 46. A kit for the treatment of cancer comprising the pharmaceutical composition of clause 45, and instructions for administration of the composition to a subject in need thereof.

Clause 47. A method for treating disease in a subject in need thereof, the method comprising administering to the subject an effective amount of the IL-2 variant, fusion protein, heterodimeric protein, or pharmaceutical composition of any one of clauses 1-38 or 45, such that one or more symptoms associated with the disease is ameliorated in the subject.

Clause 48. The method of clause 47, wherein the disease is cancer.

Clause 49. The method of clause 48, wherein the disease is a solid cancer.

Clause 50. The method of clause 48, wherein the disease is a liquid cancer.

Clause 51. The method of any one of clauses 47-50, wherein the cancer is relapsed, refractory, or metastatic.

Clause 52. The method of any one of clauses 47-51, wherein the method further comprises administering an effective amount of a second therapeutic agent, optionally wherein the administration is separate, sequential, or simultaneous.

Clause 53. The method of clause 52, wherein the second therapeutic agent is an antibody selected from the group consisting of an anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, and an anti-GITR antibody.

Clause 54. A method of stimulating the immune system in a subject in need thereof, the method comprising administering to the subject an effective amount of the IL-2 variant, fusion protein, heterodimeric protein, or pharmaceutical composition of any one of clauses 1-38 or 45, such that the immune system is stimulated in the subject.

Clause 55. The IL-2 variant, fusion protein, heterodimeric protein, or pharmaceutical composition of any one of clauses 1-38 or 45, for use in the treatment of disease in an individual in need thereof.

Clause 56. The IL-2 variant, fusion protein, heterodimeric protein, or pharmaceutical composition for use of clause 55 wherein the disease is cancer, optionally wherein the cancer is a solid cancer or a liquid cancer and/or the cancer is relapsed, refractory, or metastatic.

Clause 57. The IL-2 variant, fusion protein, heterodimeric protein, or pharmaceutical composition for use of any one of clauses 55 or 56, wherein the use is in combination with a second therapeutic agent, optionally wherein the combination is for administration simultaneously, concurrently, or simultaneously.

Clause 58. The IL-2 variant, fusion protein, heterodimeric protein, or pharmaceutical composition of any one of clauses 1-38 or 45, for use in the manufacture of a medicament for use in the treatment of disease in an individual in need thereof.

### C. Recombinant Oncolytic Virus and Related Aspects

C-1. Recombinant Oncolytic Viruses

In some other aspects, the present disclosure provides a recombinant oncolytic virus comprising an inserted nucleotide sequence (transgene) encoding an IL-2 variant described herein above, such as human IL-2 variants IL-2gv1 or IL-2gv2. The virus can be constructed from a variety of oncolytic viruses known in the art, including adenoviruses, type 1 herpes simplex viruses, type 2 herpes simplex viruses, pox viruses, retroviruses, rhabdoviruses, paramyxoviruses or reoviruses, vesicular stomatitis viruses, Newcastle disease viruses, vaccinia viruses, and any species or strain within these larger groups. In some embodiments, the recombinant oncolytic virus is replication-competent. In some embodiments, the recombinant oncolytic virus is replication-incompetent. In some embodiments, the recombinant oncolytic virus is a vaccinia virus. In a particular embodiment, the recombinant oncolytic virus is a recombinant vaccinia virus Copenhagen strain.

In some embodiments, the recombinant oncolytic virus comprising an inserted nucleotide sequence encoding an IL-2 variant further comprises one or modifications or mutations to the virus genome, protein, or other components of the virus, which increases or enhances one or more desirable anti-tumor properties of the virus, such as increased or improved tumor selectivity, enhanced production of extracellular enveloped virus (EEV), enhanced spread of progeny virions, improved safety and PET-CT imaging, or enhanced anti-tumor immune response.

Examples of specific modifications or mutations to the virus genome are described in detail herein below.

C-1A. IL-2 Variants

As described above, the recombinant OV provided by the present disclosure, such as a recombinant VV, comprises an inserted nucleotide sequence encoding an IL-2 variant described herein above.

In some embodiments, the recombinant OV comprises a nucleotide sequence encoding a wild-type IL-2 polypeptide, such as human IL-2 polypeptide or murine IL-2 polypeptide, or a variant thereof. The amino acid sequence of the mature form of a wild-type human IL-2 (hIL-2) polypeptide is set forth in SEQ ID NO:1. The amino acid sequence of the full length, precursor form of the wild-type hIL-2 polypeptide is set forth in SEQ ID NO:21. The precursor form of the wild-type hIL-2 polypeptide includes a signal peptide (e.g., MYRMQLLSCIALSLALVTNS (SEQ ID NO:22)). The amino acid sequence of the mature form of a wild-type mouse IL-2 (mIL-2) polypeptide is set forth in SEQ ID NO:23. The amino acid sequence of the precursor form of the mouse wild-type IL-2 polypeptide is set forth in SEQ ID NO:24.

In some embodiments, the variant interleukin-2 (IL-2v) polypeptide has decreased binding to the IL-2 receptor alpha (“IL-2Ra”/CD25), or decreased binding to the high-affinity trimeric IL-2 receptor complex (containing IL-2Ra+IL-2Rb+IL-2Rg), as compared to wild-type human IL-2 polypeptide, but retains the ability to bind to the intermediate-affinity dimeric IL-2 receptor complex (containing IL-2Rb+IL-2Rg).

In some other embodiments, the IL-2v polypeptide, when expressed in a subject being administered the recombinant OV, has reduced toxicity, reduced stimulation of immunosuppressive T-regulatory cells (T-reg cells), or otherwise reduced immunosuppressive activities.

The IL-2v polypeptide-encoding nucleotide sequence is present in the genome of the recombinant OV and may be referred to as a “transgene.” The IL-2v polypeptide-encoding nucleotide sequence is not naturally present in wild-type vaccinia virus and is thus heterologous to wild-type vaccinia virus. Thus, the IL-2v polypeptide-encoding nucleotide sequence can be referred to as a “heterologous nucleotide sequence” or “inserted nucleotide sequence” encoding a variant IL-2 polypeptide.”

In some cases, a IL-2v polypeptide encoded by a recombinant OV of the present disclosure provides reduced undesirable biological activity when compared to wild-type IL-2. In some cases, said reduced undesirable biological activity is determined by measuring potency at inducing increased pSTAT5 levels in CD25+CD4+ Treg cells when compared to wild-type IL-2. In some cases, an IL-2v polypeptide provides reduced concentration potency when compared to wild-type IL-2 at inducing increased pSTAT5 levels in CD25+CD4+ Treg cells. In some cases, an IL-2v polypeptide provides reduced concentration potency of at least 1, at least 2 or at least 3 logs when compared to wild-type IL-2 at inducing increased pSTAT5 levels in CD25+CD4+ Treg cells. In some cases, an IL-2v polypeptide provides reduced concentration potency of about 1, about 2 or about 3 logs when compared to wild-type IL-2 at inducing increased pSTAT5 levels in CD25+CD4+ Treg cells. In some cases, said reduced undesirable biological activity is determined by measuring the proinflammatory cytokine levels after treatment with an IL-2v polypeptide encoded by the recombinant vaccinia virus when compared to wild-type IL-2, as disclosed at Example 9. In some cases, an IL-2v polypeptide provides reduced proinflammatory cytokine levels when compared to wild-type IL-2 (e.g. using the test disclosed at Example 9). In some cases, an IL-2v polypeptide provides reduced proinflammatory cytokine levels by at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, or at least 100%, when compared to wild type IL-2.

In some cases, a recombinant vaccinia virus of the present disclosure comprises a nucleotide sequence encoding an IL-2v polypeptide that includes a signal peptide (e.g., MYRMQLLSCIALSLALVTNS (SEQ ID NO:22). Thus, e.g., in some cases, a replication-competent, recombinant oncolytic vaccinia virus of the present disclosure comprises a nucleotide sequence encoding an IL-2v polypeptide having at least 95% (e.g., at least 95%, at least 98%, at least 99%, or 100%) amino acid sequence identity to the IL-2 amino acid sequence depicted in SEQ ID NO:21 (MYRMQLLSCIALSLALVTNSAPTSSSTKKTQLQLEHLLLDLQMILNGINNYKNPKLT RMLTFKFYMPKKATELKHLQCLEEELKPLEEVLNLAQSKNFHLRPRDLISNINVIVLE LKGSETTFMCEYADETATIVEFLNRWITFCQSIISTLT), and comprising a substitution of one or more of F62, Y65, and L92 of the IL-2 based on the amino acid numbering of the amino acid sequence depicted in SEQ ID NO:21. As will be appreciated, F62, Y65, and L92 of the IL-2 amino acid sequence depicted in SEQ ID NO:21 correspond to F42, Y45, and L72 of the amino acid sequence depicted in SEQ ID NO:1.

Other suitable IL-2v polypeptides include, e.g., a mouse IL-2v polypeptide comprising an amino acid sequence having at least 95% (e.g., at least 95%, at least 98%, at least 99%, or 100%) amino acid sequence identity to the amino acid sequence of SEQ ID NO:3 and comprising F76A, Y79A, and L106G substitutions (i.e., comprising Ala-76, Ala-79, and Gly-106). A nucleotide sequence encoding the IL-2v polypeptide of SEQ ID NO:3 is set forth in SEQ ID NO:2.

In some cases, a nucleotide sequence encoding a mouse IL-2v polypeptide is codon optimized for vaccinia virus. An example of a nucleotide sequence encoding a mouse IL-2v polypeptide that codon optimized for vaccinia virus is set forth in SEQ ID NO:19.

Other suitable IL-2v polypeptides include, e.g., a human IL-2v polypeptide comprising an amino acid sequence having at least 95% (e.g., at least 95%, at least 98%, at least 99%, or 100%) amino acid sequence identity to the amino acid sequence of SEQ ID NO;14 and comprising F62A, Y65A, and L92G substitutions (i.e., comprising Ala-62, Ala-65, and Gly-92).

Examples of Suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the nucleotide sequence of SEQ ID NO:12, where the encoded IL-2v polypeptide comprises F62A, Y65A, and L92G substitutions (i.e., comprises Ala-62, Ala-65, and Gly-92). Other examples of suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the nucleotide sequence of SEQ ID NO:13, where the encoded IL-2v polypeptide comprises F62A, Y65A, and L92G substitutions (i.e., comprises Ala-62, Ala-65, and Gly-92).

Other suitable IL-2v polypeptides include, e.g., a human IL-2v polypeptide comprising an amino acid sequence having at least 95% (e.g., at least 95%, at least 98%, at least 99%, or 100%) amino acid sequence identity to the amino acid sequence of SEQ ID NO:9 and comprising F42A, Y45A, and L72G substitutions (i.e., comprising Ala-42, Ala-45, and Gly-72).

Examples of suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the nucleotide sequence of SEQ ID NO:10, where the encoded IL-2v polypeptide comprises F42A, Y45A, and L72G substitutions (i.e., comprises Ala-42, Ala-45, and Gly-72). Other examples of suitable nucleotide sequences encoding an IL-2v polypeptide include, e.g., a nucleotide sequence encoding a human IL-2v polypeptide and having at least 80%, at least 85%, at least 90%, at least 95%, at least 98%, at least 99%, or 100%, nucleotide sequence identity to the nucleotide sequence of SEQ ID NO:11, where the encoded IL-2v polypeptide comprises F42A, Y45A, and L72G substitutions (i.e., comprises Ala-42, Ala-45, and Gly-72).

In some embodiments, a recombinant OV of the present disclosure comprises an inserted nucleotide sequence that encodes a human mature form IL-2v. polypeptide, wherein the a human IL-2v polypeptide comprises one or more amino acid substitutions selected from the group consisting of: K35, R38, L40, T41, F42, K43, Y45, Y45, E62, K64, L72, and Q74, based on the amino acid numbering of the IL-2 amino acid sequence depicted in SEQ ID NO:1.

In some embodiments, the recombinant OV provided by the present disclosure comprises an inserted nucleotide sequence that encodes a human IL-2v polypeptide comprising one or more amino acid substitutions relative to the human IL-2 protein sequence of SEQ ID NO: 1 at the following positions: T3, K35, R38, L40, T41, F42, K43, Y45, E62, K64, Y65, L72, Q74, and C125. In some other embodiments, the IL-2v comprises amino acid substitutions at one or more of the following groups of positions: R38 and L40; T41 and K43; K43 and Y45; E62 and K64; L72 and Q74; R38, L40, K43, and Y45; K43, Y45, L72, and Q74; T3, R38, L40, K43, and Y45; T3, K43, Y45, L72, and Q74; R38, L40, K43, Y45, and C125; K43, Y45, L72, Q74, and C125; T3, R38, L40, K43, Y45, and C125; T3, K43, Y45, L72, Q74, and C125. Examples of substitutions at a given amino acid position include T3A, K35N, R38N, L40S, L40T, T41N, K43S, K43T, K43N, Y45S, Y45T, E62N, E62A, E62K, E62R, K64S, K64T, L72N, Q74S, Q74T, C125A, and C125S.

In some particular embodiments, an IL-2v polypeptide encoded by a recombinant OV comprises at least one amino acid substitution as compared to wild-type human IL-2, wherein wild-type human IL-2 has the amino acid sequence as shown in SEQ ID NO: 1 and the IL-2 variant comprises substitutions selected from the group consisting of:

a) K35, wherein the K35 substitution is K35N

b) R38 and L40, wherein the R38 substitution is R38N and the L40 substitution is L40S or L40T,

c) T41 and K43, wherein the T41 substitution is T41N and the K43 substitution is K43S or K43T,

d) K43 and Y45, wherein the K43 substitution is K43N and the Y45 substitution is Y45S or Y45T,

e) E62 and K64, wherein the E62 substitution is E62N and the K64 substitution is K64S or K64T, and

f) L72 and Q74, wherein the L72 substitution is L72N and the Q74 substitution is Q74S or Q74T, wherein the numbering is based on the amino acid sequence of SEQ ID NO:1.

In some other particular embodiments, the IL-2v comprises a substitution at position K35, and further comprises substitutions at positions selected from the group consisting of:

a) R38 and L40, wherein the R38 substitution is R38N and the L40 substitution is L40S or L40T,

b) T41 and K43, wherein the T41 substitution is T41N and the K43 substitution is K43S or K43T,

c) K43 and Y45, wherein the K43 substitution is K43N and the Y45 substitution is Y45S or Y45T,

d) E62 and K64, wherein the E62 substitution is E62N and the K64 substitution is K64S or K64T,

e) L72 and Q74, wherein the L72 substitution is L72N and the Q74 substitution is Q74S or Q74T, and

f) E62, wherein the E62 substitution is E62N, E62A, E62K, or, E62R.

In still other particular embodiments, the IL-2 variant comprises substitutions at positions T41 and K43, and further comprises substitutions at positions selected from the group consisting of:

a) E62 and K64, wherein the E62 substitution is E62N and the K64 substitution is K64S or K64T,

b) L72 and Q74, wherein the L72 substitution is L72N and the Q74 substitution is Q74S or Q74T, and

c) E62, wherein the E62 substitution is E62N, E62A, E62K, or, E62R

In some further particular embodiments, the IL-2 variant comprises K43N and Y45T, and further comprises substitutions selected from the group consisting of:

a) E62N and K64S or K64T,

b) L72N and Q74S or Q74T,

c) E62N, E62A, E62K, or E62R;

e) R38N and L40T; and

f) L72N and Q74T.

In some particular embodiments, the recombinant OV comprises an inserted nucleotide sequence that encodes an IL-2v polypeptide comprising an amino acid sequence selected from the group consisting of:

a) an amino acid sequence having at least 95% (e.g., at least 95%, at least 98%, at least 99%, or 100%) amino acid sequence identity to the amino acid sequence of SEQ ID NO:29 (MYRMQLLSCIALSLALVTNSAPTSSSTKKTQLQLEHLLLDLQMILNGINNYKNPKLT NMTTFNFTMPKKATELKHLQCLEEELKPLEEVLNLAQSKNFHLRPRDLISNINVIVLE LKGSETTFMCEYADETATIVEFLNRWITFCQSIISTLT) and comprising substitutions R58N, L60T, K63N, and Y65T; and

b) an amino acid sequence having at least 95% (e.g., at least 95%, at least 98%, at least 99%, or 100%) amino acid sequence identity to the amino acid sequence of SEQ ID NO:31 (APTSSSTKKTQLQLEHLLLDLQMILNGINNYKNPKLTNMTTFNFTMPKKATELKHL QCLEEELKPLEEVLNLAQSKNFHLRPRDLISNINVIVLELKGSETTFMCEYADETATI VEFLNRWITFCQSIISTLT), and comprising substitutions R38N, L40T, K43N, and Y45T.

In a particular embodiment, the inserted nucleotide sequence encodes an IL-2v polypeptide comprising the amino acid sequence of SEQ ID NO:29 or SEQ ID NO:31.

In some other particular embodiments, the recombinant OV comprises an inserted nucleotide sequence that encode an IL-2v polypeptide comprising an amino acid sequence selected from the group consisting of:

a) an amino acid sequence having at least 95% (e.g., at least 95%, at least 98%, at least 99%, or 100%) amino acid sequence identity to the amino acid sequence of SEQ ID NO:33 (MYRMQLLSCIALSLALVTNSAPTSSSTKKTQLQLEHLLLDLQMILNGINNYKNPKLT RMLTFNFTMPKKATELKHLQCLEEELKPLEEVLNNATSKNFHLRPRDLISNINVIVLE LKGSETTFMCEYADETATIVEFLNRWITFCQSIISTLT) and comprising substitutions K63N, Y65T, L92N, and Q94T; and

b) an amino acid sequence having at least 95% (e.g., at least 95%, at least 98%, at least 99%, or 100%) amino acid sequence identity to the amino acid sequence of SEQ ID NO:35 (APTSSSTKKTQLQLEHLLLDLQMILNGINNYKNPKLTRMLTFNFTMPKKATELKHL QCLEEELKPLEEVLNNATSKNFHLRPRDLISNINVIVLELKGSETTFMCEYADETATI VEFLNRWITFCQSIISTLT) and comprising substitutions K43N, Y45T, L72N, and Q74T.

In a particular embodiment, the inserted nucleic acid encoding the IL-2v polypeptide comprises the nucleotide sequence of SEQ ID NO:30 (ATGTATCGTATGCAGCTGCTGAGCTGCATCGCTTTATCTTTAGCTTTAGTGACC AACAGCGCCCCTACCAGCTCCTCCACCAAGAAGACCCAGCTGCAGCTGGAGC ATTTACTGCTGGATTTACAGATGATTTTAAACGGCATCAACAACTACAAGAACC CCAAGCTGACTAATATGACCACCTTCAACTTCACTATGCCCAAGAAGGCCACC GAGCTGAAGCACCTCCAGTGTTTAGAGGAGGAGCTGAAGCCTTTAGAGGAGG TGCTGAATTTAGCCCAGAGCAAGAATTTCCATTTAAGGCCTCGTGATTTAATCA GCAACATCAACGTGATCGTGCTGGAGCTGAAAGGCTCCGAGACCACCTTCATG TGCGAGTACGCCGACGAGACCGCCACCATCGTGGAGTTTTTAAATCGTTGGAT CACCTTCTGCCAGAGCATCATCAGCACTTTAACC) or SEQ ID N0:32 (GCCCCTACCAGCTCCTCCACCAAGAAGACCCAGCTGCAGCTGGAGCATTTAC TGCTGGATTTACAGATGATTTTAAACGGCATCAACAACTACAAGAACCCCAAGC TGACTAATATGACCACCTTCAACTTCACTATGCCCAAGAAGGCCACCGAGCTG AAGCACCTCCAGTGTTTAGAGGAGGAGCTGAAGCCTTTAGAGGAGGTGCTGAA TTTAGCCCAGAGCAAGAATTTCCATTTAAGGCCTCGTGATTTAATCAGCAACAT CAACGTGATCGTGCTGGAGCTGAAAGGCTCCGAGACCACCTTCATGTGCGAGT ACGCCGACGAGACCGCCACCATCGTGGAGTTTTTAAATCGTTGGATCACCTTC TGCCAGAGCATCATCAGCACTTTAACC), or a degenerate variant of the nucleotide sequence of SEQ ID NO:30 or SEQ ID NO:32.

In another particular embodiment, the inserted nucleic acid encoding the IL-2v polypeptide comprises the nucleotide sequence of SEQ ID NO:34 (ATGTATCGTATGCAGCTGCTGAGCTGCATCGCTTTATCTTTAGCTTTAGTGACC AACAGCGCCCCTACCAGCTCCTCCACCAAGAAGACCCAGCTGCAGCTGGAGC ATTTACTGCTGGATTTACAGATGATTTTAAACGGCATCAACAACTACAAGAACC CCAAGCTGACTCGTATGCTGACCTTCAACTTCACTATGCCCAAGAAGGCCACC GAGCTGAAGCACCTCCAGTGTTTAGAGGAGGAGCTGAAGCCTTTAGAGGAGG TGCTGAATAACGCCACCAGCAAGAATTTCCATTTAAGGCCTCGTGATTTAATCA GCAACATCAACGTGATCGTGCTGGAGCTGAAAGGCTCCGAGACCACCTTCATG TGCGAGTACGCCGACGAGACCGCCACCATCGTGGAGTTTTTAAATCGTTGGAT CACCTTCTGCCAGAGCATCATCAGCACTTTAACC) or SEQ ID N0:36 (GCCCCTACCAGCTCCTCCACCAAGAAGACCCAGCTGCAGCTGGAGCATTTAC TGCTGGATTTACAGATGATTTTAAACGGCATCAACAACTACAAGAACCCCAAGC TGACTCGTATGCTGACCTTCAACTTCACTATGCCCAAGAAGGCCACCGAGCTG AAGCACCTCCAGTGTTTAGAGGAGGAGCTGAAGCCTTTAGAGGAGGTGCTGAA TAACGCCACCAGCAAGAATTTCCATTTAAGGCCTCGTGATTTAATCAGCAACAT CAACGTGATCGTGCTGGAGCTGAAAGGCTCCGAGACCACCTTCATGTGCGAGT ACGCCGACGAGACCGCCACCATCGTGGAGTTTTTAAATCGTTGGATCACCTTC TGCCAGAGCATCATCAGCACTTTAACC), or a degenerate variant of the nucleotide sequence of SEQ ID NO:34 or SEQ ID NO:36.

In some cases, a replication-competent, recombinant oncolytic vaccinia virus of the present disclosure comprises a homologous recombination donor fragment encoding an IL-2v polypeptide, where the homologous recombination donor fragment comprises a nucleotide sequence having at least 80%, at least 85%, at least 90%, at least 95%, at least 98%, at least 99%, or 100%, nucleotide sequence identity to the nucleotide sequence set forth in any one of SEQ ID NO:4 (VV27/VV38 homologous recombination donor fragment), SEQ ID NO:5 (VV39 homologous recombination donor fragment), SEQ ID NO:15 (VV75 homologous recombination donor fragment containing hIL-2v (human codon optimized)), SEQ ID NO:16 (Copenhagen J2R homologous recombination plasmid containing hIL-2v (human codon optimized)), SEQ ID NO:17 (homologous recombination donor fragment containing hIL-2v (vaccinia virus codon optimized)), SEQ ID NO:18 (Copenhagen J2R homologous recombination plasmid containing hIL-2v (vaccinia virus codon optimized)), and SEQ ID NO:20 (mouse IL-2 variant (vaccinia virus codon optimized) homologous recombination donor fragment).

In some cases, a replication-competent, recombinant oncolytic vaccinia virus of the present disclosure comprises a nucleotide sequence having at least 80%, at least 85%, at least 90%, at least 95%, at least 98%, at least 99%, or 100%, nucleotide sequence identity to the nucleotide sequence set forth in SEQ ID NO:6 (Copenhagen J2R homologous recombination plasmid) and comprises a nucleotide sequence encoding an IL-2v polypeptide.

In some cases, a replication-competent, recombinant oncolytic vaccinia virus of the present disclosure comprises a nucleotide sequence having at least 80%, at least 85%, at least 90%, at least 95%, at least 98%, at least 99%, or 100%, nucleotide sequence identity to the nucleotide sequence set forth in SEQ ID NO:7 (Copenhagen J2R homologous recombination plasmid containing mouse IL-2 variant (mIL-2v) polypeptide).

In some cases, a recombinant oncolytic vaccinia virus of the present disclosure comprises a nucleotide sequence having at least 80%, at least 85%, at least 90%, at least 95%, at least 98%, at least 99%, or 100%, nucleotide sequence identity to the nucleotide sequence set forth in SEQ ID NO:8 (Western Reserve J2R homologous recombination plasmid containing mIL-2v).

In some specific cases, a recombinant VV of the present disclosure is VV27, (Copenhagen vaccinia containing A34R-K151E and mIL-2v transgene). In some cases, the recombinant VV comprises, in place of the mIL-2v polypeptide, a human IL-2 variant (hIL-2v) polypeptide, as described above.

In some specific cases, a recombinant OV of the present disclosure is VV38, (Copenhagen vaccinia containing mIL-2v transgene). In some cases, the recombinant VV comprises, in place of the mIL-2v polypeptide, a human IL-2 variant (hIL-2v) polypeptide, as described above.

In some specific cases, a recombinant OV of the present disclosure is VV39, (Western Reserve vaccinia containing mIL-2v transgene). In some cases, the recombinant VV comprises, in place of the mIL-2v polypeptide, a human IL-2 variant (hIL-2v) polypeptide, as described above.

In some other specific embodiments, a recombinant OV of the present disclosure is VV97, VV98, VV110, or VV117 as described in the Examples.

C-1B. Heterologous Thymidine Kinase (TK) Polypeptide

In some embodiments, the a replication-competent, recombinant oncolytic vaccinia virus that comprises an inserted nucleotide sequence encoding an IL-2 variant as described herein above further comprises an inserted nucleotide sequence encoding a heterologous thymidine kinase (TK) polypeptide. In some embodiments, the heterologous TK polypeptide is a variant of a herpes simplex virus TK (HSV-TK) polypeptide. A variant of wild-type HSV-TK is also referred to herein as an “HSV-TKv.” The HSV-TKv is in some cases a type I TK polypeptide, i.e., a TK polypeptide that can catalyze phosphorylation of deoxyguanosine (dG) to generate dG monophosphate, respectively.

In some instances, the heterologous TK-encoding nucleotide sequence, such as the nucleotide sequence encoding HSV-TKv, replaces all or a part of the vaccinia virus TK-encoding nucleotide sequence. In wild-type vaccinia virus, the J2R region encodes vaccinia virus TK. For example, in some cases, the heterologous TK polypeptide-encoding nucleotide sequence replaces at least 10%, at least 15%, at least 20%, at least 25%, at least 30%, at least 40%, at least 50%, at least 75%, or 100%, of the J2R region of vaccinia virus. In some cases, replication-competent, recombinant oncolytic vaccinia virus of the present disclosure comprises a modification such that transcription of the endogenous (vaccinia virus-encoded) TK-encoding gene is reduced or eliminated. For example, in some cases, transcription of the endogenous (vaccinia virus-encoded) TK-encoding gene is reduced by at least 50%, at least 60%, at least 70%, at least 80%, at least 90%, or more than 90%, compared to the transcription of the endogenous (vaccinia virus-encoded) TK-encoding gene without the modification.

In some cases, the replication of the replication-competent, recombinant oncolytic vaccinia virus is inhibited with ganciclovir at a lower concentration than the concentration at which replication of a replication-competent, recombinant oncolytic vaccinia virus encoding a wild-type HSV-TK polypeptide is inhibited. For example, the ganciclovir inhibitory concentration at which replication of a replication-competent, recombinant oncolytic vaccinia virus of the present disclosure that encodes a variant of wild-type HSV-TK is inhibited by 50% of maximum (IC50) is at least 10%, at least 15%, at least 20%, at least 25%, at least 30%, at least 40%, at least 50%, at least 60%, at least 70%, or at least 80% lower than the ganciclovir IC50 for inhibition of replication of a replication-competent, recombinant oncolytic vaccinia virus encoding a wild-type HSV-TK polypeptide.

In some embodiments, the heterologous TK polypeptide encoded by a nucleotide sequence present in a replication-competent, recombinant oncolytic vaccinia virus is a variant of wild-type HSV-TK, where the TKv polypeptide comprises one or more amino acid substitutions relative to wild-type HSV-TK (SEQ ID NO:25). In some embodiments, the HSV-TKv polypeptide encoded by a nucleotide sequence present in a replication-competent, recombinant oncolytic vaccinia virus of the present disclosure comprises from 1 to 40 amino acid substitutions relative to wild-type HSV-TK. For example, a TKv polypeptide encoded by a nucleotide sequence present in a replication-competent, recombinant oncolytic vaccinia virus of the present disclosure comprises from 1 to 5, from 5 to 10, from 10 to 15, from 15 to 20, from 20 to 25, from 25 to 30, from 30 to 35, or from 35 to 40, amino acid substitutions relative to wild-type HSV-TK (SEQ ID NO:25).

In some particular embodiments, a heterologous TK polypeptide present in a recombinant vaccinia virus of the present disclosure comprises an amino acid sequence having at least 80%, at least 85%, at least 90%, at least 95%, at least 98%, or at least 99%, amino acid sequence identity to the wild-type HSV-TK amino acid sequence of SEQ ID NO:25
(MASYPGHQHASAFDQAARSRGHSNRRTALRPRRQQEATEVRPEQKMPT LLRVYIDGPHGMGKTTTTQLLVALGSRDDIVYVPEPMTYWRVLGASETIANI YTTQHRLDQGEISAGDAAVVMTSAQITMGMPYAVTDAVLAPHIGGEAGSSH APPPALTLIFDRHPIAALLCYPAARYLMGSMTPQAVLAFVALIPPTLPGTNIVL GALPEDRHIDRLAKRQRPGERLDLAMLAAIRRVYGLLANTVRYLQGGGSW REDWGQLSGTAVPPQGAEPQSNAGPRPH IGDTLFTLFRAPELLAPNGDLY NVFAWALDVLAKRLRPMHVFILDYDQSPAGCRDALLQLTSGMIQTHVTTPG SIPTICDLARTFAREMGEAN) and comprises one or more amino acid substitutions relative to SEQ ID NO:25.

In some cases, the heterologous TK polypeptide comprises one or more amino acid substitutions relative to the wild-type HSV-TK amino acid sequence (set forth in SEQ ID NO:25. For example, in some cases, the heterologous TK polypeptide comprises a substitution of one or more of L159, 1160, F161, A168, and L169.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, but has a substitution at L159, i.e., amino acid 159 is other than Leu. For example, amino acid 159 is Gly, Ala, Val, Ile, Pro, Phe, Tyr, Trp, Ser, Thr, Cys, Met, Gin, Asn, Lys, Arg, His, Asp, or Glu. In some cases, the substitution is an L159I substitution. In some cases, the substitution is an L159A substitution. In some cases, the substitution is an L159V substitution.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, but has a substitution at 1160, i.e., amino acid 160 is other than Ile. For example, amino acid 160 is Gly, Ala, Val, Leu, Pro, Phe, Tyr,

Trp, Ser, Thr, Cys, Met, Gin, Asn, Lys, Arg, His, Asp, or Glu. In some cases, the substitution is an I160L substitution. In some cases, the substitution is an I160V substitution. In some cases, the substitution is an I160A substitution. In some cases, the substitution is an I160F substitution. In some cases, the substitution is an I160Y substitution. In some cases, the substitution is an I160W substitution.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, but has a substitution at F161, i.e., amino acid 161 is other than Phe. For example, amino acid 161 is Gly, Ala, Val, Leu, Ile, Pro, Tyr, Trp, Ser, Thr, Cys, Met, Gin, Asn, Lys, Arg, His, Asp, or Glu. In some cases, the substitution is an F161A substitution. In some cases, the substitution is an F161L substitution. In some cases, the substitution is an F161V substitution. In some cases, the substitution is an F161I substitution.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, but has a substitution at A168, i.e., amino acid 168 is other than Ala. For example, amino acid 168 is Gly, Val, Leu, Ile, Pro, Phe, Tyr, Trp, Ser, Thr, Cys, Met, Gln, Asn, Lys, Arg, His, Asp, or Glu. In some cases, the substitution is A168H. In some cases, the substitution is A168R. In some cases, the substitution is A168K. In some cases, the substitution is A168Y. In some cases, the substitution is A168F. In some cases, the substitution is A168W. In some cases, the TKv polypeptide does not include any other substitutions other than a substitution of A168.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, but has a substitution at L169, i.e., amino acid 169 is other than Leu. For example, amino acid 169 is Gly, Ala, Val, Ile, Pro, Phe, Tyr, Trp, Ser, Thr, Cys, Met, Gln, Asn, Lys, Arg, His, Asp, or Glu. In some cases, the substitution is L169F. In some cases, the substitution is L169M. In some cases, the substitution is L169Y. In some cases, the substitution is L169W.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, where: i) amino acid 159 is other than Leu; ii) amino acid 160 is other than Ile; iii) amino acid 161 is other than Phe; iv) amino acid 168 is other than Ala; and v) amino acid 169 is other than Leu. In some cases, the heterologous TK polypeptide comprises an amino acid sequence having at least 80%, at least 85%, at least 90%, at least 95%, at least 98%, at least 99%, or 100%, amino acid sequence identity to the following amino acid sequence:

(“dm30”; SEQ ID NO: 26)
MASYPGHQHASAFDQAARSRGHSNRRTALRPRRQQEATEVRPEQKMPT
LLRVYIDGPHGMGKTTTTQLLVALGSRDDIVYVPEPMTYWRVLGASET
IANIYTTQHRLDQGEISAGDAAVVMTSAQITMGMPYAVTDAVLAPHIG
GEAGSSHVPPPALTILADRHPIAYFLCYPAARYLMGSMTPQAVLAFVA
LIPPTLPGTNIVLGALPEDRHIDRLAKRQRPGERLDLAMLAAIRRVYG
LLANTVRYLQGGGSWREDWGQLSGTAVPPQGAEPQSNAGPRPHIGDTL
FTLFRAPELLAPNGDLYNVFAWALDVLAKRLRPMHVFILDYDQSPAGC
RDALLQLTSGMIQTHVTTPGSIPTICDLARTFAREMGEAN,

where amino acid 159 is Ile, amino acid 160 is Leu, amino acid 161 is Ala, amino acid 168 is Tyr, and amino acid 169 is Phe.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, where: i) amino acid 159 is other than Leu; ii) amino acid 160 is other than Ile; iii) amino acid 161 is other than Phe; iv) amino acid 168 is other than Ala; and v) amino acid 169 is other than Leu. In some cases, the heterologous TK polypeptide comprises an amino acid sequence having at least 80%, at least 85%, at least 90%, at least 95%, at least 98%, at least 99%, or 100%, amino acid sequence identity to the following amino acid sequence:
MASYPGHQHASAFDQAARSRGHSNRRTALRPRRQQEATEVRPEQKMPTL LRVYIDGPHGMGKTTTTQLLVALGSRDDIVYVPEPMTYWRVLGASETIANIY TTQHRLDQGEISAGDAAVVMTSAQITMGMPYAVTDAVLAPHIGGEAGSSHA PPPALTIFLDRHPIAFMLCYPAARYLMGSMTPQAVLAFVALIPPTLPGTNIVL GALPEDRHIDRLAKRQRPGERLDLAMLAAIRRVYGLLANTVRYLQGGGSW REDWGQLSGTAVPPQGAEPQSNAGPRPHIGDTLFTLFRAPELLAPNGDLY NVFAWALDVLAKRLRPMHVFILDYDQSPAGCRDALLQLTSGMIQTHVTTPG SIPTICDLARTFAREMGEAN (“SR39”; SEQ ID NO:27), where amino acid 159 is Ile, amino acid 160 is Phe, amino acid 161 is Leu, amino acid 168 is Phe, and amino acid 169 is Met.

In some cases, the heterologous TK polypeptide comprises an amino acid sequence having at least 80%, at least 85%, at least 90%, at least 95%, at least 98%, or at least 99%, amino acid sequence identity to the wild-type HSV-TK amino acid sequence set forth in SEQ ID NO:25, where amino acid 168 is other than Ala, e.g., where amino acid 168 is Gly, Val, Ile, Leu, Pro, Phe, Tyr, Trp, Ser, Thr, Cys, Met, Gln, Asn, Lys, Arg, His, Asp, or Glu. In some cases, amino acid 168 is His. In some cases, amino acid 168 is Arg. In some cases, amino acid 168 is Lys. In some cases, the heterologous TK polypeptide comprises an amino acid sequence having at least 80%, at least 85%, at least 90%, at least 95%, at least 98%, at least 99%, or 100%, amino acid sequence identity to the following amino acid sequence:
MASYPGHQHASAFDQAARSRGHSNRRTALRPRRQQEATEVRPEQKMPTL LRVYIDGPHGMGKTTTTQLLVALGSRDDIVYVPEPMTYWRVLGASETIANIY TTQHRLDQGEISAGDAAVVMTSAQITMGMPYAVTDAVLAPHIGGEAGSSHA PPPALTLIFDRHPIAHLLCYPAARYLMGSMTPQAVLAFVALIPPTLPGTNIVLG ALPEDRHIDRLAKRQRPGERLDLAMLAAIRRVYGLLANTVRYLQGGGSWRE DWGQLSGTAVPPQGAEPQSNAGPRPHIGDTLFTLFRAPELLAPNGDLYNV FAWALDVLAKRLRPMHVFILDYDQSPAGCRDALLQLTSGMIQTHVTTPGSIP TICDLARTFAREMGEAN (“TK.007”; SEQ ID NO:28), where amino acid 168 is His.
   The heterologous TK polypeptide of SEQ ID NO:28 where amino acid 168 is His is also referred to as “TK.007” or HSV-TK.007″ in the present disclosure.

C-1C. Other Insertions, Deletions, or Mutations

In addition to an inserted nucleotide sequence encoding an IL-2v polypeptide and an inserted nucleotide sequence encoding a heterologous TK as described herein above, a recombinant vaccinia virus provided by the present disclosure may comprise further modifications that increase or enhance its desirable properties as an oncolytic virus, such as modifications to render deficient the function of a specific protein, to suppress or enhance the expression of a specific gene or protein, or to express an exogenous protein.

In some embodiments, the recombinant vaccinia virus provided by the present disclosure further comprises one or more modifications that increase the tumor-selectivity of the oncolytic vaccinia viruses. As used herein, “tumor selective” means toxicity to tumor cells (for example, oncolytic) higher than that to normal cells (for example, non-tumor cell). Examples of such modifications include: (1) modification that renders the virus deficient in the function of vaccinia growth factor (VGF) (McCart et al. (2001) Cancer Research 61:8751); (2) modification to the vaccinia virus TK gene, the hemagglutinin (HA) gene, or F3 gene or an interrupted F3 locus (WO 2005/047458); (3) modification that renders the vaccinia virus deficient in the function of VGF and O1L (WO 2015/076422); (4) insertion of a micro RNA whose expression is decreased in cancer cells into the 3′ noncoding region of the B5R gene (WO 2011/125469); (5) modifications that render the vaccinia virus deficient in the function of B18R (Kim et al. (2007) PLoS Medicine 4:e353), ribonucleotide reductase (Gammon et al. (2010) PLoS Pathogens 6:e1000984), serine protease inhibitor (e.g., SPI-1, SPI-2) (Guo et al. (2005) Cancer Research 65:9991), SPI-1 and SPI-2 (Yang et al. (2007) Gene Therapy 14:638), ribonucleotide reductase genes F4L or I4L (Child et al. (1990) Virology 174:625; Potts et al. (2017) EMBO Mol. Med. 9:638), B18R (B19R in Copenhagen strain) (Symons et al. (1995) Cell 81:551), A48R (Hughes et al. (1991) J. Biol. Chem. 266:20103); B8R (Verardi et al. (2001) J. Virol. 75:11), B15R (B16R in Copenhagen strain) (Spriggs et al. (1992) Cell 71:145), A41R (Ng et al. (2001) Journal of General Virology 82:2095), A52R (Bowie et al. (2000) Proc. Natl. Acad. Sci. USA 97:10162), F1L (Gerlic et al. (2013) Proc. Natl. Acad. Sci. USA 110:7808), E3L (Chang et al. (1992) Proc. Natl. Acad. Sci. USA 89:4825), A44R-A46R (Bowie et al. (2000) Proc. Natl. Acad. Sci. USA 97:10162), K1L (Bravo Cruz et al. (2017) Journal of Virology 91:e00524), A48R, B18R, C11R, and TK (Mejías-Pérez et al. (2017) Molecular Therapy: Oncolytics 8:27), E3L and K3L regions (WO 2005/007824), or O1L (Schweneker et al. (2012) J. Virol. 86:2323). Moreover, a recombinant vaccinia virus may comprise a modification that renders the vaccinia virus deficient in the extracellular region of B5R (Bell et al. (2004) Virology 325:425), deficient in the A34R region (Thirunavukarasu et al. (2013) Molecular Therapy 21:1024), or deficient in interleukin-1μ (IL-1μ) receptor (WO 2005/030971). Moreover, vaccinia virus having a combination of two or more of such genetic modifications may be used in a replication-competent, recombinant oncolytic vaccinia virus of the present disclosure. Such insertion of a foreign gene or deletion or mutation of a gene on the vaccinia virus genome can be made, for example, by a known homologous recombination or site-directed mutagenesis.

As used herein, the term “deficient” or “deficiency” means that the gene region or protein specified by this term has reduced or no function. A recombinant oncolytic vaccinia virus of the present disclosure that comprises a modification such that the recombinant oncolytic vaccinia virus is rendered “deficient” in a given vaccinia virus gene exhibits reduced production and/or activity of a gene product (e.g., mRNA gene product; polypeptide gene product); for example, the amount and/or activity of the gene product is less than 75%, less than 60%, less than 50%, less than 40%, less than 30%, less than 25%, less than 20%, less than 15%, less than 10%, less than 5%, or less than 1% of the amount and/or activity of the same gene product produced by wild-type vaccinia virus, or by a control vaccinia virus that does not comprise the genetic alteration.

Modifications that can render a gene or protein deficient include, but is not limited to: i) mutation (e.g., substitution, inversion, etc.) and/or truncation and/or deletion of the gene region specified by this term; ii) mutation and/or truncation and/or deletion of a promoter region controlling expression of the gene region; and iii) mutation and/or truncation and/or deletion of a polyadenylation sequence such that translation of a polypeptide encoded by the gene region is reduced or eliminated. Examples of such modifications includes: deletion in a region consisting of the specified gene region or the deletion in a neighboring gene region comprising the specified gene region; a mutation and/or truncation and/or deletion of a promoter region that reduces transcription of a gene region can result in deficiency; incorporation of a transcriptional termination element such that translation of a polypeptide encoded by the gene region is reduced or eliminated; through use of a gene-editing enzyme or a gene-editing complex (e.g., a CRISPR/Cas effector polypeptide complexed with a guide RNA) to reduce or eliminate transcription of the gene region; through use of competitive reverse promoter/polymerase occupancy to reduce or eliminate transcription of the gene region; and insertion of a nucleic acid into the gene region, thereby knocking out the gene region.

In some specific embodiments, a recombinant virus of the present disclosure, such as a vaccinia virus, that comprises an inserted nucleotide sequence encoding an IL-2v polypeptide provided herein above, wherein the virus lacks the virus's endogenous thymidine kinase (TK) activity. As used herein, the term “endogenous” refers to any materials, such as polynucleotide, polypeptide, or protein, that is naturally present or naturally expressed within an organism, such as a virus, or a cell thereof. The vaccinia virus TK is encoded by the TK gene and open-reading frame (ORF) J2R on the vaccinia virus genome. A virus that lacks endogenous TK activity may be referred to as being “thymidine kinase deficient” or “TK deficient.” In some cases, a recombinant vaccinia virus of the present disclosure comprises a deletion of all or a portion of the vaccinia virus TK coding region, such that the vaccinia virus is TK deficient. For example, in some cases, a replication-competent, recombinant oncolytic vaccinia virus of the present disclosure comprises a J2R deletion. See, e.g., Mejia-Perez et al. (2018) Mol. Ther. Oncolytics 8:27. In some cases, a replication-competent, recombinant oncolytic vaccinia virus of the present disclosure comprises an insertion into the J2R region, thereby resulting in reduced or no vaccinia virus TK activity. In some other embodiments, the recombinant oncolytic virus is both virus TK gene deficient and B16R gene deficient.

In some other embodiments, the replication-competent, recombinant oncolytic vaccinia virus provided by the present disclosure further comprises a modification that enhances the spread of progeny virions. In a particular embodiment, the present disclosure provides a replication-competent, recombinant oncolytic vaccinia virus that comprises an inserted nucleotide sequence encoding an IL-2v polypeptide provided herein above, wherein the A34R gene of the virus comprises a K151E substitution (i.e., comprising a modification that provides for a K151E substitution in the encoded polypeptide). See, e.g., Blasco et al. (1993) J. Virol. 67(6):3319-3325; and Thirunavukarasu et al. (2013) Mol. Ther. 21:1024. The A34R gene encodes vaccinia virus gp22-24 (also known as Protein A34). The A34R gene encodes a viral coat protein (A34 protein). The amino acid sequence of an A34 protein of the vaccinia virus strain Copenhagen is available at UniProt (UniProtKB-P21057 (Q34_VACCC)), which consists of 168 amino acids. The amino acid sequence of a A34 protein comprising K151E substitution is set forth in SEQ ID NO:38 (MKSLNRQTVSMFKKLSVPAAIMMILSTIISGIGTFLHYKEELMPSACANGWIQYDKH CYLDTNIKMSTDNAVYQCRKLRARLPRPDTRHLRVLFSIFYKDYVVVSLKKTNNKWL DINNDKDIDISKLTNFKQLNSTTDAEACYIYKSGKLVETVCKSTQSVLCVKKFYK). A nucleotide sequence of the A34R gene that encodes the A34 protein comprising K151E mutation is set forth in SEQ ID NO:39 (ATGAAATCGCTTAATAGACAAACTGTAAGTATGTTTAAGAAGTTGTCGGTGCCG GCCGCTATAATGATGATACTCTCAACCATTATTAGTGGCATAGGAACATTTCTG CATTACAAAGAAGAACTGATGCCTAGTGCTTGCGCCAATGGATGGATACAATAC GATAAACATTGTTATCTAGATACCAACATTAAAATGTCCACAGATAATGCGGTTT ATCAGTGTCGTAAATTACGAGCTAGATTGCCTAGACCTGATACTAGACATCTGA GAGTATTGTTTAGTATTTTTTATAAAGATTATTGGGTAAGTTTAAAAAAGACCAAT AATAAATGGTTAGATATTAATAATGATAAAGATATAGATATTAGTAAATTAACAAA TTTTAAACAACTAAACAGTACGACGGATGCTGAAGCGTGTTATATATACAAGTC TGGAAAACTGGTTGAAACAGTATGTAAAAGTACTCAATCTGTACTATGTGTTAAA AAATTCTACAAGTGA) (which contains A415G mutation relative to the wild-type gene sequence).

In some other embodiments, the recombinant oncolytic vaccinia virus provided by the present disclosure comprises: (1) an inserted nucleotide sequence encoding an IL-2v polypeptide; (2) an inserted nucleotide sequence encoding a heterologous TK polypeptide; and (3) an K151E substitution in the A34R gene, wherein the recombinant vaccinia virus is TK deficient. In some particular embodiments, the IL-2v polypeptide encoded by the recombinant vaccinia virus comprises an amino acid sequence having at least 95% (e.g., at least 95%, at least 98%, at least 99%, or 100%) identity to the amino acid sequence of in SEQ ID NO:29 and comprises an amino acid substitutions R58N, L60T, K63N, and Y65T, wherein the amino acid numbering is based on the amino acid sequence of SEQ ID NO:29. In some further particular embodiments, the heterologous TK polypeptide comprises an amino acid sequence having at least 95% (e.g., at least 95%, at least 98%, at least 99%, or 100%) identity to the amino acid sequence of in SEQ ID NO:28 where amino acid 168 is His. In a particular embodiment, a recombinant vaccinia virus provided by the present disclosure comprises: (1) an inserted nucleotide sequence encoding an IL-2v polypeptide; (2) an inserted nucleotide sequence encoding a heterologous TK polypeptide; and (3) an K151E substitution in the A34R gene, wherein the recombinant vaccinia virus is Strain Copenhagen and is TK deficient, wherein the IL-2v polypeptide comprises the amino acid sequence SEQ ID NO:29, and wherein the heterologous TK polypeptide comprises an amino acid sequence of SEQ ID NO:28.

C-2. Construction of Recombinant Oncolytic Virus

A replication-competent, recombinant oncolytic virus provided by the present disclosure can be constructed by method known in the art. Specifically, the oncolytic vaccinia virus of the present disclosure can be constructed from any of a variety of strains of vaccinia virus, either known now or discovered in the future. Strains of the vaccinia virus suitable for use include, but not limited to, the strains Lister, New York City Board of Health (NYBH), Wyeth, Copenhagen, Western Reserve (WR), Modified Vaccinia Ankara (MVA), EM63, Ikeda, Dalian, LIVP, Tian Tan, IHD-J, Tashkent, Bern, Paris, Dairen, and derivatives the like. In some cases, a replication-competent, recombinant oncolytic vaccinia virus of the present disclosure is a Copenhagen strain vaccinia virus. In some cases, a replication-competent, recombinant oncolytic vaccinia virus of the present disclosure is a WR strain vaccinia virus.

The nucleotide sequences of the genomes of vaccinia viruses of various strains are known in the art. See, e.g., Goebel et al. (1990) Virology 179:247; Goebel et al. (1990) Virology 179:517. The nucleotide sequence of the Copenhagen strain vaccinia virus is known; see, e.g., GenBank Accession No. M35027. The nucleotide sequence of the WR strain vaccinia virus is known; see, e.g., GenBank Accession No. AY243312; and GenBank Accession No. NC_006998. The WR strain of vaccinia virus is available from the American Type Culture Collection (ATCC); ATCC VR-1354

A replication-competent, recombinant oncolytic virus, such as a vaccinia virus, of the present disclosure exhibits oncolytic activity. The oncolytic activity of a virus can be evaluated by any suitable method known in the art. Examples of methods for evaluating whether a given virus exhibits oncolytic activity include in vitro methods for evaluating decrease of the survival rate of cancer cells by the addition of the virus. Examples of cancer cells or cell lines that may be used include the malignant melanoma cell RPMI-7951 (for example, ATCC HTB-66), the lung adenocarcinoma HCC4006 (for example, ATCC CRL-2871), the lung carcinoma A549 (for example, ATCC CCL-185), the lung carcinoma HOP-62 (for example, DCTD Tumor Repository), the lung carcinoma EKVX (for example, DCTD Tumor Repository), the small cell lung cancer cell DMS 53 (for example, ATCC CRL-2062), the lung squamous cell carcinoma NCI-H226 (for example, ATCC CRL-5826), the kidney cancer cell Caki-1 (for example, ATCC HTB-46), the bladder cancer cell 647-V (for example, DSMZ ACC 414), the head and neck cancer cell Detroit 562 (for example, ATCC CCL-138), the breast cancer cell JIMT-1 (for example, DSMZ ACC 589), the breast cancer cell MDA-MB-231 (for example, ATCC HTB-26), the breast cancer cell MCF7 (for example, ATCC HTB-22), the breast cancer HS-578T (for example, ATCC HTB-126), the breast ductal carcinoma T-47D (for example, ATCC HTB-133), the esophageal cancer cell OE33 (for example, ECACC 96070808), the glioblastoma U-87MG (for example, ECACC 89081402), the neuroblastoma GOTO (for example, JCRB JCRB0612), the myeloma RPMI 8226 (for example, ATCC CCL-155), the ovarian cancer cell SK-OV-3 (for example, ATCC HTB-77), the ovarian cancer cell OVMANA (for example, JCRB JCRB1045), the cervical cancer HeLa (for example, ATCC CCL-2), the colon cancer cell RKO (for example, ATCC CRL-2577), the colon cancer cell HT-29 (for example, ATCC HTB-38), the colon cancer Colo 205 (for example, ATCC CCL-222), the colon cancer SW620 (for example, ATCC CCL-227), the colorectal carcinoma HCT 116 (for example, ATCC CCL-247), the pancreatic cancer cell BxPC-3 (for example, ATCC CRL-1687), the bone osteosarcoma U-2 OS (for example, ATCC HTB-96), the prostate cancer cell LNCaP clone FGC (for example, ATCC CRL-1740), the hepatocellular carcinoma JHH-4 (for example, JCRB JCRB0435), the mesothelioma NCI-H28 (for example, ATCC CRL-5820), the cervical cancer cell SiHa (for example, ATCC HTB-35), and the gastric cancer cell Kato III (for example, RIKEN BRC RCB2088).

A nucleic acid comprising a nucleotide sequence encoding an IL-2 variant polypeptide or heterologous TK polypeptide can be introduced into a vaccinia virus using established techniques, including reactivation with helper virus and homologous recombination. For example, a plasmid (also referred to as transfer vector plasmid DNA) in which a nucleic acid comprising a nucleotide sequence encoding an IL-2 variant polypeptide is inserted can be generated, generating a recombinant transfer vector; the recombinant transfer vector can be introduced into cells infected with vaccinia virus. The nucleic acid comprising a nucleotide sequence encoding the IL-2v polypeptide is then introduced into the vaccinia virus from the recombinant transfer vector via homologous recombination.

Similarly, a plasmid (also referred to as transfer vector plasmid DNA) in which a nucleotide sequence encoding a heterologous TK polypeptide is inserted can be generated, generating a recombinant transfer vector; the recombinant transfer vector can be introduced into cells transfected with digested genomic DNA from Vaccinia virus and infected with a helper virus. The nucleotide sequence encoding the TKv polypeptide is then introduced into the vaccinia virus from the recombinant transfer vector via homologous recombination. The region in which a nucleotide sequence encoding a TKv polypeptide is introduced can be the endogenous vaccinia virus TK-encoding gene, e.g., J2R. The nucleic acid encoding a TKv polypeptide can replace all or a portion of vaccinia virus J2R.

In some case, the nucleotide sequence encoding the IL-2v polypeptide or heterologous TK polypeptide is operably linked to a transcriptional control element, e.g., a promoter. In some cases, the promoter provides for expression of the polypeptide in tumor cells. Suitable promoters include, but are not limited to, a pSEL promoter, a PSFJ1-10 promoter, a PSFJ2-16 promoter, a pHyb promoter, a Late-Early optimized promoter, a p7.5K promoter, a p11K promoter, a T7.10 promoter, a CPX promoter, a modified H5 promoter, an H4 promoter, a HF promoter, an H6 promoter, and a T7 hybrid promoter.

In some cases, the nucleotide sequence encoding the IL-2v polypeptide or heterologous TK polypeptide is operably linked to a regulatable promoter. In some cases, the regulatable promoter is a reversible promoter. In some cases, the nucleotide sequence encoding the IL-2v polypeptide or heterologous TK polypeptide is operably linked to a tetracycline-regulated promoter, (e.g., a promoter system such as TetActivators, TetON, TetOFF, Tet-On Advanced, Tet-On 3G, etc.). In some cases, the nucleotide sequence encoding the IL-2v polypeptide or heterologous TK polypeptide is operably linked to a repressible promoter. In some cases, the nucleotide sequence encoding the IL-2v polypeptide or heterologous TK polypeptide is operably linked to a promoter that is tetracycline repressible, e.g., the promoter is repressed in the presence of tetracycline or a tetracycline analog or derivative. In some cases, the nucleotide sequence encoding the IL-2v polypeptide or heterologous TK polypeptide is operably linked to a TetOFF promoter system. Bujard and Gossen (1992) Proc. Natl. Acad. Sci. USA 89:5547. For example, a TetOFF promoter system is repressed (inactive) in the presence of tetracycline (or suitable analog or derivative, such as doxycycline); once tetracycline is removed, the promoter is active and drives expression of the polypeptide. In some cases, the nucleotide sequence encoding the IL-2v polypeptide or heterologous TK polypeptide is operably linked to a promoter that is tetracycline activatable, e.g., the promoter is activated in the presence of tetracycline or a tetracycline analog or derivative.

The region in which a nucleic acid comprising a nucleotide sequence encoding an IL-2v polypeptide is introduced can be a gene region that is inessential for the life cycle of vaccinia virus. For example, the region in which a nucleic acid comprising a nucleotide sequence encoding an IL-2v polypeptide is introduced can be a region within the VGF gene in vaccinia virus deficient in the VGF function, a region within the O1L gene in vaccinia virus deficient in the O1L function, or a region or regions within either or both of the VGF and O1L genes in vaccinia virus deficient in both VGF and O1L functions. In the above, the foreign gene(s) can be introduced so as to be transcribed in the direction same as or opposite to that of the VGF and O1L genes. As another example, the region in which a nucleic acid comprising a nucleotide sequence encoding an IL-2v polypeptide is introduced can be a region within the B18 gene (B19 in Copenhagen) in vaccinia virus deficient in B18 (B19) function. In a particular embodiment, the inserted nucleotide sequence encoding an IL-2 variant polypeptide is located in the region of the endogenous vaccinia virus TK-encoding gene, e.g., J2R. The nucleotide sequence encoding a IL-2 variant polypeptide can replace entire or a portion of virus J2R gene. In another particular embodiment, the inserted nucleotide sequence encoding the heterologous tk, such as the HSV-tk.007 polypeptide is located in the region of the virus B16R gene (which is called B15R gene in other vaccinia virus strains like Western Reserve) and may replace the entire or portion of the B16R gene.

C-3. Compositions Comprising a Recombinant Oncolytic Virus

In another aspect, the present disclosure provides a composition comprising a recombinant oncolytic virus, such as a vaccinia virus, provided by the present disclosure. The composition can be in any form suitable for the particular active ingredient included, such as solution or suspension. In some cases, the composition is a pharmaceutical composition suitable for administration to a human.

In some embodiments, the pharmaceutical composition further comprises a pharmaceutically acceptable carrier. As used herein, the term “pharmacologically acceptable carrier” refers to any substance or material that, when combined with the active ingredient, allows the active ingredient to retain biological activity and has no significant long-term or permanent detrimental effect when administered to a subject, and encompasses terms such as pharmacologically acceptable “vehicle,” “stabilizer,” “diluent,” “auxiliary” or “excipient.” Such a carrier generally is mixed with the active ingredient (e.g., an IL-2 variant, or a fusion molecule, or a recombinant oncolytic vaccinia virus of the present disclosure) and can be a solid, semi-solid, or liquid agent. Any of a variety of pharmaceutically acceptable carriers can be used including, without limitation, buffers, preservatives, tonicity adjusters, salts, antioxidants, bulking agents, emulsifying agents, wetting agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a subject pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a subject pharmaceutical composition.

A pharmaceutical composition comprising a recombinant oncolytic virus of the present disclosure may contain the virus in an amount of from about 10² plaque-forming units (pfu) per ml (pfu/ml) to about 10⁴ pfu/ml, from about 10⁴ pfu/ml to about 10⁵ pfu/ml, from about 10⁵ pfu/ml to about 10⁶ pfu/ml, from about 10⁶ pfu/ml to about 10⁷ pfu/ml, from about 10⁷ pfu/ml to about 10⁸ pfu/ml, from about 10⁸ pfu/ml to about 10⁹ pfu/ml, from about 10⁹ pfu/ml to about 10¹⁰ pfu/ml, from about 10¹⁰ pfu/ml to about 10¹¹ pfu/ml, or from about 10¹¹ pfu/ml to about 10¹² pfu/ml.

C-4. Uses of the Recombinant Oncolytic Viruses

C-4A. Uses and Administration

In another aspect, the present disclosure provides uses of, as well as method of using, the recombinant oncolytic viruses and compositions comprising the recombinant oncolytic virus. The uses or methods includes those for inducing oncolysis, or treating cancer, in an individual having a tumor, the methods comprising administering to the individual in need thereof an effective amount of a replication-competent, recombinant oncolytic vaccinia virus of the present disclosure or a composition of the present disclosure. Administration of an virus of the present disclosure is also referred to herein as “virotherapy.”

In some cases, an “effective amount” of a replication-competent, recombinant oncolytic virus of the present disclosure is an amount that, when administered in one or more doses to an individual in need thereof, reduces the number of cancer cells or tumor mass in the individual. For example, in some cases, an “effective amount” of a replication-competent, recombinant oncolytic vaccinia virus is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the recombinant virus, or in the absence of administration with the recombinant vaccinia virus. In some cases, an “effective amount” of a recombinant virus is an amount that, when administered in one or more doses to an individual in need thereof, reduces the number of cancer cells in the individual to undetectable levels. In some cases, an “effective amount” of a recombinant vaccinia virus of the present disclosure is an amount that, when administered in one or more doses to an individual in need thereof, reduces the tumor mass in the individual. For example, in some cases, an “effective amount” of a recombinant vaccinia virus of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the recombinant virus, or in the absence of administration with the replication-competent, recombinant oncolytic virus.

In some cases, an “effective amount” of a recombinant virus of the present disclosure is an amount that, when administered in one or more doses to an individual in need thereof, increases survival time of the individual. For example, in some cases, an “effective amount” of a recombinant virus of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the recombinant oncolytic virus.

In some cases, an “effective amount” of a recombinant oncolytic virus of the present disclosure is an amount that, when administered in one or more doses to an individual in need thereof, provides for an increase in the number of IFN-γ-producing T cells. For example, in some cases, an “effective amount” of a recombinant virus of the present disclosure is an amount that, when administered in one or more doses to an individual in need thereof, provides for an increase in the number of IFN-γ-producing T cells in the individual of at least 10%, at least 25%, at least 50%, at least 2-fold, at least 5-fold, or at least 10-fold, compared to the number of IFN-γ-producing T cells in the individual before administration of the replication-competent, recombinant oncolytic virus, or in the absence of administration with the replication-competent, recombinant oncolytic virus.

In some cases, an “effective amount” of a recombinant virus of the present disclosure is an amount that, when administered in one or more doses to an individual in need thereof, provides for an increase in the circulating level of IL-2 or IL-2v in the individual. For example, in some cases, an “effective amount” of a recombinant virus is an amount that, when administered in one or more doses to an individual in need thereof, provides for an increase in the circulating level of IL-2 or IL-2v in the individual at least 10%, at least 25%, at least 50%, at least 2-fold, at least 5-fold, or at least 10-fold, compared to the circulating level of IL-2 or IL-2v in the individual before administration of the oncolytic virus, or in the absence of administration with the oncolytic vaccinia virus.

In some cases, an “effective amount” of a recombinant oncolytic virus of the present disclosure is an amount that, when administered in one or more doses to an individual in need thereof, provides for an increase in the circulating level of IL-2v polypeptide in the individual. For example, in some cases, an “effective amount” of a recombinant virus of the present disclosure is an amount that, when administered in one or more doses to an individual in need thereof, provides for an increase in the circulating level of IL-2v polypeptide in the individual at least 10%, at least 25%, at least 50%, at least 2-fold, at least 5-fold, or at least 10-fold, compared to the circulating level of IL-2v polypeptide in the individual before administration of the oncolytic vaccinia virus, or in the absence of administration with the oncolytic vaccinia virus.

In some cases, an “effective amount” of a recombinant oncolytic virus of the present disclosure is an amount that, when administered in one or more doses to an individual in need thereof, provides for an increase in the number of CD8⁺ tumor-infiltrating lymphocytes (TILs). For example, in some cases, an “effective amount” of the virus in amount that, when administered in one or more doses to an individual in need thereof, provides for an increase in the number of CD8⁺ TILs of at least 10%, at least 25%, at least 50%, at least 2-fold, at least 5-fold, or at least 10-fold, compared to the number of CD8⁺ TILs in the individual before administration of the virus, or in the absence of administration with the virus.

In some cases, an “effective amount” of a recombinant oncolytic virus of the present disclosure is an amount that, when administered in one or more doses to an individual in need thereof, induces a durable anti-tumor immune response, e.g., an anti-tumor immune response that provides for reduction in tumor cell number and/or tumor mass and/or tumor growth for at least 1 month, at least 2 months, at least 6 months, or at least 1 year.

A suitable dosage can be determined by an attending physician or other qualified medical personnel, based on various clinical factors. As is well known in the medical arts, dosages for any one patient depend upon many factors, including the patient's size, body surface area, age, tumor burden, and other relevant factors.

A recombinant virus of the present disclosure can be administered in an amount of from about 10² plaque-forming units (pfu) to about 10⁴ pfu, from about 10⁴ pfu to about 10⁵ pfu, from about 10⁵ pfu to about 10⁶ pfu, from about 10⁶ pfu to about 10⁷ pfu, from about 10⁷ pfu to about 10⁸ pfu, from about 10⁸ pfu to about 10⁹ pfu, from about 10⁹ pfu to about 10¹⁰ pfu, or from about 10¹⁰ pfu to about 10″ pfu, per dose.

In some cases, a recombinant virus of the present disclosure is administered in a total amount of from about 1×10⁹ pfu to 5×10¹¹ pfu. In some cases, a recombinant vaccinia virus of the present disclosure is administered in a total amount of from about 1×10⁹ pfu to about 5×10⁹ pfu, from about 5×10⁹ pfu to about 10¹⁰ pfu, from about 10¹⁰ pfu to about 5×10¹⁰ pfu, from about 5×10¹⁰ pfu to about 10¹¹ pfu, or from about 10¹¹ pfu to about 5×10¹¹ pfu. In some cases, a recombinant vaccinia virus of the present disclosure is administered in a total amount of about 2×10¹⁰ pfu.

In some cases, a recombinant virus of the present disclosure is administered in an amount of from about 1×10⁸ pfu/kg patient weight to about 5×10⁹ pfu/kg patient weight. In some cases, a recombinant vaccinia virus of the present disclosure is administered in an amount of from about 1×10⁸ pfu/kg patient weight to about 5×10⁸ pfu/kg patient weight, from about 5×10⁸ pfu/kg patient weight to about 10⁹ pfu/kg patient weight, or from about 10⁹ pfu/kg patient weight to about 5×10⁹ pfu/kg patient weight. In some cases, a recombinant virus of the present disclosure is administered in an amount of 1×10⁸ pfu/kg patient weight. In some cases, a recombinant vaccinia virus of the present disclosure is administered in an amount of 2×10⁸ pfu/kg patient weight. In some cases, a recombinant vaccinia virus of the present disclosure is administered in an amount of 3×10⁸ pfu/kg patient weight. In some cases, a recombinant virus of the present disclosure is administered in an amount of 4×10⁸ pfu/kg patient weight. In some cases, a recombinant virus of the present disclosure is administered in an amount of 5×10⁸ pfu/kg patient weight.

In some cases, multiple doses of a recombinant virus of the present disclosure are administered. The frequency of administration of a recombinant virus of the present disclosure can vary depending on any of a variety of factors, e.g., severity of the symptoms, etc. For example, in some embodiments, a recombinant vaccinia virus of the present disclosure is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid).

The duration of administration of a recombinant virus of the present disclosure can vary, depending on any of a variety of factors, e.g., patient response, etc. For example, a recombinant virus of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A recombinant oncolytic virus of the present disclosure is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intrathecal, intracranial, subcutaneous, intradermal, topical application, intravenous, intraarterial, intraperitoneal, intrabladder, rectal, nasal, oral, and other enteral and parenteral routes of administration. Routes of administration may be combined, if desired, or adjusted depending upon the recombinant vaccinia virus and/or the desired effect. A recombinant vaccinia virus of the present disclosure can be administered in a single dose or in multiple doses.

In some cases, a recombinant oncolytic virus of the present disclosure is administered intravenously, intramuscularly, locally, intratumorally, peritumorally, intracranially, subcutaneously, intra-arterially, intraperitoneally, via an intrabladder route of administration, or intrathecally.

C-4B. Combinations

In some cases, a recombinant oncolytic virus of the present disclosure is administered in combination with another therapy or agent. For example, the recombinant virus may be administered as an adjuvant therapy to a standard cancer therapy, administered in combination with another cancer therapy, or administered in combination with an agent that enhances the anti-tumor effect of the recombinant vaccinia virus. Standard cancer therapies include surgery (e.g., surgical removal of cancerous tissue), radiation therapy, bone marrow transplantation, chemotherapeutic treatment, antibody treatment, biological response modifier treatment, immunotherapy treatment, and certain combinations of the foregoing. Thus, in an embodiment, the present disclosure provide a method of treating cancer in an individual, comprising administering to the individual in need thereof: a) a recombinant vaccinia virus of the present disclosure, or a composition comprising same; and b) a second cancer therapy. In some cases, the second cancer therapy is selected from chemotherapy, biological therapy (such as therapies with antibodies), radiotherapy, immunotherapy, hormone therapy, anti-vascular therapy, cryotherapy, toxin therapy, oncolytic virus therapy (e.g., an oncolytic virus other than a recombinant vaccinia virus of the present disclosure), a cell therapy, and surgery.

Radiation therapy includes, but is not limited to, x-rays or gamma rays that are delivered from either an externally applied source such as a beam, or by implantation of small radioactive sources.

Examples of suitable antibodies for use in cancer treatment include trastuzumab (Herceptin), bevacizumab (Avastin™), cetuximab (Erbitux™) panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), ⁹⁰Y-labelled ibritumomab tiuxetan (Zevalin™), ¹³¹I-labelled tositumoma (Bexxar™), etc. Suitable antibodies for use in cancer treatment include, but are not limited to, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MEDI4736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin_5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and hematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 8106 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

In some cases, a method of the present disclosure comprises administering: a) an effective amount of a recombinant oncolytic virus, such as a vaccinia virus, of the present disclosure; and b) an anti-PD-1 antibody. In some cases, a method of the present disclosure comprises administering: a) an effective amount of a recombinant oncolytic virus of the present disclosure; and b) an anti-PD-L1 antibody. Suitable anti-PD-1 antibodies include, but are not limited to, pembrolizumab (Keytruda®; MK-3475), Nivolumab (Opdivo®; BMS-926558; MDX1106), Pidilizumab (CT-011), AMP-224, AMP-514 (MEDI-0680), PDR001, and PF-06801591. Suitable anti-PD-L1 antibodies include, but are not limited to, BMS-936559 (MDX1105), durvalumab (MEDI4736; Imfinzi), Atezolizumab (MPDL33280A; Tecentriq). See, e.g., Sunshine and Taube (2015) Curr. Opin. Pharmacol. 23:32; and Heery et al. (2017) The Lancet Oncology 18:587; Iwai et al. (2017) J. Biomed. Sci. 24:26; Hu-Lieskovan et al. (2017) Annals of Oncology 28: issue Suppl. 5, mdx376.048; and U.S. Patent Publication No. 2016/0159905.

In some cases, a suitable antibody is a bispecific antibody, e.g., a bispecific monoclonal antibody. Catumaxomab, blinatumomab, solitomab, pasotuxizumab, and flotetuzumab are non-limiting examples of bispecific antibodies suitable for use in cancer therapy. See, e.g., Chames and Baty (2009) MAbs 1:539; and Sedykh et al. (2018) Drug Des. Devel. Ther. 12:195.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α; (7) interferon-γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOLμ, TAXOTEREμ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotereμ docetaxel, as noted above) and paclitaxel conjugates (e.g., paclitaxel-PEG, paclitaxel-dextran, or paclitaxel-xylose).

Cell therapy includes chimeric antigen receptor (CAR) T cell therapy (CAR-T therapy); natural killer (NK) cell therapy; dendritic cell (DC) therapy (e.g., DC-based vaccine); T cell receptor (TCR) engineered T cell-based therapy; and the like.

C-4C. Cancers and Tumors

Cancer cells that may be treated by methods and compositions of the present disclosure include cancer cells from or in any organ or tissue, such as bladder, blood, bone, bone marrow, brain, breast, colon, esophagus, gastrointestine, gum, head, kidney, liver, lung, nasopharynx, neck, ovary, prostate, skin, stomach, spinal cord, testis, tongue, or uterus. In addition, the cancer may be of any histological type, for example: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; pancreatic cancer; rectal cancer; and hairy cell leukemia.

Tumors that can be treated using a method of the present disclosure include, e.g., a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

In some cases, the tumor is a colorectal adenocarcinoma. In some cases, the tumor is non-small cell lung carcinoma. In some cases, the tumor is a triple-negative breast cancer. In some cases, the tumor is a solid tumor. In some cases, the tumor is a liquid tumor. In some cases, the tumor is recurrent. In some cases, the tumor is a primary tumor. In some cases, the tumor is metastatic.

A variety of subjects are suitable for treatment with a subject method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent other than a an oncolytic vaccinia virus of the present disclosure for the cancer and failed to respond to such treatment, or who has been treated with an agent other than an oncolytic vaccinia virus of the present disclosure for the cancer but relapsed after initial response to such treatment.

C-5. Oncolytic Virus Immunogenic Compositions

In another aspect, the recombinant oncolytic virus provided by the present disclosure further comprises, in its genome, a nucleotide sequence encoding a cancer antigen, such as a tumor-associated antigen and neoantigen. The term “cancer-associated antigen” (also known as tumor-associated antigen) is protein that is expressed more by cancer cells than by normal cells. The term “neoantigen” refers to a protein that is expressed in cancer cells but not normal cells. In some embodiments, the recombinant vaccinia virus comprises, in its genome: i) a nucleotide sequence encoding an IL-2v polypeptide described herein above; ii) an nucleotide sequence encoding a heterologous TK polypeptide; and iii) a nucleotide sequence encoding a cancer antigen. Such recombinant vaccinia viruses, when administered to an individual in need thereof (e.g., an individual having a cancer), can induce or enhance an immune response in the individual to the encoded cancer antigen. The immune response can reduce the number of cancer cells in the individual. Suitable IL-2v polypeptides and heterologous TK polypeptides are as described above.

Examples of cancer-associated antigens include α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2) (See, e.g., Vigneron et al. (2013) Cancer Immunity 13:15; and Vigneron (2015) BioMed Res. Int'l Article ID 948501; and epidermal growth factor receptor (EGFR) vIII polypeptide (see, e.g., Wong et al. (1992) Proc. Natl. Acad. Sci. USA 89:2965; and Miao et al. (2014) PLoSOne 9:e94281); a MUC1 polypeptide; a human papillomavirus (HPV) E6 polypeptide; an LMP2 polypeptide; an HPV E7 polypeptide; an epidermal growth factor receptor (EGFR) vIII polypeptide; a HER-2/neu polypeptide; a melanoma antigen family A, 3 (MAGE A3) polypeptide; a p53 polypeptide; a mutant p53 polypeptide; an NY-ESO-1 polypeptide; a folate hydrolase (prostate-specific membrane antigen; PSMA) polypeptide; a carcinoembryonic antigen (CEA) polypeptide; a melanoma antigen recognized by T-cells (melanA/MART1) polypeptide; a Ras polypeptide; a gp100 polypeptide; a proteinase3 (PR1) polypeptide; a bcr-abl polypeptide; a tyrosinase polypeptide; a survivin polypeptide; a prostate specific antigen (PSA) polypeptide; an hTERT polypeptide; a sarcoma translocation breakpoints polypeptide; a synovial sarcoma X (SSX) breakpoint polypeptide; an EphA2 polypeptide; a prostate acid phosphatase (PAP) polypeptide; a melanoma inhibitor of apoptosis (ML-IAP) polypeptide; an alpha-fetoprotein (AFP) polypeptide; an epithelial cell adhesion molecule (EpCAM) polypeptide; an ERG (TMPRSS2 ETS fusion) polypeptide; a NA17 polypeptide, a paired-box-3 (PAX3) polypeptide; an anaplastic lymphoma kinase (ALK) polypeptide; an androgen receptor polypeptide; a cyclin B1 polypeptide; an N-myc proto-oncogene (MYCN) polypeptide; a Ras homolog gene family member C (RhoC) polypeptide; a tyrosinase-related protein-2 (TRP-2) polypeptide; a mesothelin polypeptide; a prostate stem cell antigen (PSCA) polypeptide; a melanoma associated antigen-1 (MAGE A1) polypeptide; a cytochrome P450 1B1 (CYP1B1) polypeptide; a placenta-specific protein 1 (PLAC1) polypeptide; a BORIS polypeptide (also known as CCCTC-binding factor or CTCF); an ETV6-AML polypeptide; a breast cancer antigen NY-BR-1 polypeptide (also referred to as ankyrin repeat domain-containing protein 30A); a regulator of G-protein signaling (RGS5) polypeptide; a squamous cell carcinoma antigen recognized by T-cells (SART3) polypeptide; a carbonic anhydrase IX polypeptide; a paired box-5 (PAX5) polypeptide; an OY-TES1 (testis antigen; also known as acrosin binding protein) polypeptide; a sperm protein 17 polypeptide; a lymphocyte cell-specific protein-tyrosine kinase (LCK) polypeptide; a high molecular weight melanoma associated antigen (HMW-MAA); an A-kinase anchoring protein-4 (AKAP-4); a synovial sarcoma X breakpoint 2 (SSX2) polypeptide; an X antigen family member 1 (XAGE1) polypeptide; a B7 homolog 3 (B7H3; also known as CD276) polypeptide; a legumain polypeptide (LGMN1; also known as asparaginyl endopeptidase); a tyrosine kinase with Ig and EGF homology domains-2 (Tie-2; also known as angiopoietin-1 receptor) polypeptide; a P antigen family member 4 (PAGE4) polypeptide; a vascular endothelial growth factor receptor 2 (VEGF2) polypeptide; a MAD-CT-1 polypeptide; a fibroblast activation protein (FAP) polypeptide; a platelet derived growth factor receptor beta (PDGFβ) polypeptide; a MAD-CT-2 polypeptide; a Fos-related antigen-1 (FOSL) polypeptide; and a Wilms tumor-1 (WT-1) polypeptide.

Amino acid sequences of cancer-associated antigens are known in the art; see, e.g., MUC1 (GenBank CAA56734); LMP2 (GenBank CAA47024); HPV E6 (GenBank AAD33252); HPV E7 (GenBank AHG99480); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); AFP (GenBank NP_001125); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CAO99273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFβ (GenBank NP_002600); MAD-CT-2 (GenBank NP_001138574); FOSL (GenBank NP_005429); and WT-1 (GenBank NP_000369). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; and Zhang et al. (1996) Nature 383:168.

In some cases, a recombinant oncolytic virus, such as a vaccinia virus, of the present disclosure is replication incompetent. In some cases, the recombinant virus comprises a modification of a virus gene that results in inability of the virus to replicate. One or more virus genes encoding gene products required for replication can be modified such that the virus is unable to replicate. For example, a recombinant virus can be modified to reduce the levels and/or activity of an intermediate transcription factor (e.g., A8R and/or A23R) (see, e.g., Wyatt et al. (2017) mBio 8:e00790; and Warren et al. (2012) J. Virol. 86:9514) and/or a late transcription factor (e.g., one or more of G8R, A1L, and A2L) (see, e.g., Yang et al. (2013) Virology 447:213). Reducing the levels and/or activity of an intermediate transcription factor and/or a late transcription factor can result in a modified vaccinia virus that can express polypeptide(s) encoded by a nucleotide sequence(s) that is operably linked to an early viral promoter; however, the virus will be unable to replicate. Modifications include, e.g., deletion of all or part of the gene; insertion into the gene; and the like. For example, all or a portion of the A8R gene can be deleted. As another example, all or a portion of the A23R gene can be deleted. As another example, all or a portion of the G8R gene can be deleted. As another example, all or a portion of the A1L gene can be deleted. As another example, all or a portion of the A2L gene can be deleted.

As noted above, in some cases, a recombinant vaccinia virus of the present disclosure is non-oncolytic.

C-6. Administration of Analogs of 2′-Deoxyguanosine

In another aspect, the present disclosure provides administration of a recombinant oncolytic virus comprising a heterologous TK polypeptide described herein in combination with a synthetic analog of 2′-deoxyguanosine.

Oncolytic viruses may cause adverse side effects in a subject who received administration of the virus. Examples of the side effects include skin lesions, such vesicular lesions or “vesicular rash.” In some embodiments, the present disclosure provides a method of treating cancer in an individual, comprising administering to the individual: b) an effective amount of a replication-competent, recombinant oncolytic vaccinia virus of the present disclosure; and b) an effective amount of a synthetic analog of 2′-deoxy-guanosine, wherein the oncolytic vaccinia virus comprises a heterologous TK polypeptide. In some other embodiments, the present disclosure provides a method of treating, reducing, or managing a side effect of the recombinant oncolytic vaccinia virus of the present disclosure, which comprises administering an effective amount of a synthetic analog of 2′-deoxy-guanosine to a subject who has received administration of the recombinant oncolytic vaccinia virus, wherein the wherein the oncolytic vaccinia virus comprises a heterologous TK polypeptide.

An “effective amount” of a synthetic analog of 2′-deoxy-guanosine is an amount that is effective to reduce an adverse side effect caused by the replication-competent, recombinant oncolytic vaccinia virus administered. For example, where the adverse side effect is skin lesions, an effective amount of a synthetic analog of 2′-deoxy-guanosine is an amount that, when administered to an individual in one or more doses, is effective to reduce the number and/or severity and/or duration of vaccinia virus-induced skin lesions in the individual. For example, an effective amount of a synthetic analog of 2′-deoxy-guanosine can be an amount that, when administered to an individual in one or more doses, is effective to reduce the number and/or severity and/or duration of vaccinia virus-induced skin lesions in the individual by at least 10%, at least 15%, at least 20%, at least 25%, at least 30%, at least 40%, at least 50%, at least 60%, at least 75%, or more than 75%, compared with the number and/or severity and/or duration of vaccinia virus-induced skin lesions in the individual prior to administration of the synthetic analog of 2′-deoxy-guanosine or in the absence of administration of the synthetic analog of 2′-deoxy-guanosine. In some cases, an effective amount of a synthetic analog of 2′-deoxy-guanosine is an amount that, when administered to an individual in one or more doses, is effective to reduce shedding of virus from vaccinia virus-induced skin lesions. For example, in some cases, an effective amount of a synthetic analog of 2′-deoxy-guanosine is an amount that, when administered to an individual in one or more doses, is effective to reduce shedding of virus from vaccinia virus-induced skin lesions by at least 10%, at least 15%, at least 20%, at least 25%, at least 30%, at least 40%, at least 50%, at least 60%, at least 75%, or more than 75%, compared with the level or degree of virus shedding from vaccinia virus-induced skin lesions in the individual prior to administration of the synthetic analog of 2′-deoxy-guanosine or in the absence of administration of the synthetic analog of 2′-deoxy-guanosine. Where the adverse side effect is a skin lesion, in some cases, the synthetic analog of 2′-deoxy-guanosine can be administered by any convenient route of administration (e.g., topically, orally, intravenously, etc.). For example, where the adverse side effect is a skin lesion, in some cases, the synthetic analog of 2′-deoxy-guanosine can be administered topically. For reducing skin lesions, the synthetic analog of 2′-deoxy-guanosine is typically administered topically, for example, by application of the 2′-deoxy-guanosine analog to the lesion area of the skin.

Administration of a synthetic analog of 2′-deoxy-guanosine reduces replication of a replication-competent, recombinant oncolytic vaccinia virus that comprises a heterologous TK polypeptide. Such reduction in replication of the replication-competent, recombinant oncolytic vaccinia virus of the present disclosure may be desirable, e.g., to control the level of replication-competent, recombinant oncolytic vaccinia virus in an individual, to control the effect of the replication-competent, recombinant oncolytic vaccinia virus, and the like. Thus, in some other embodiments, the preset disclosure provides a method of treating cancer in an individual, comprising: a) administering an effective amount of a replication-competent, recombinant oncolytic vaccinia virus of the present disclosure; and b) administering an effective amount of a synthetic analog of 2′-deoxy-guanosine. In some cases, an effective amount of a synthetic analog of 2′-deoxy-guanosine is an amount that, when administered to an individual in one or more doses, is effective to reduce replication of a replication-competent, recombinant oncolytic vaccinia virus of the present disclosure in an individual by at least 10%, at least 15%, at least 20%, at least 25%, at least 30%, at least 40%, at least 50%, at least 60%, at least 75%, or more than 75%, compared with the level of replication of the replication-competent, recombinant oncolytic vaccinia virus in the individual prior to administration of the synthetic analog of 2′-deoxy-guanosine or in the absence of administration of the synthetic analog of 2′-deoxy-guanosine.

A synthetic analog of 2′-deoxy-guanosine can be administered after administration of a replication-competent, recombinant oncolytic vaccinia virus of the present disclosure. For example, a synthetic analog of 2′-deoxy-guanosine can be administered 1 day to 7 days, from 7 days to 2 weeks, from 2 weeks to 1 month, from 1 month to 3 months, or more than 3 months, after administration of the replication-competent, recombinant oncolytic vaccinia virus.

In some cases, administration of a synthetic analog of 2′-deoxy-guanosine to an individual to whom a replication-competent, recombinant oncolytic vaccinia virus of the present disclosure has been administered, induces rapid, systemic, tumor lysis (lysis of cancer cells) in the individual. For example, a synthetic analog of 2′-deoxy-guanosine can be administered to an individual once oncolytic vaccinia virus-induced slowing of tumor growth has occurred and/or once viral replication is at or just after its peak and/or once circulating antibody to vaccinia virus proteins are at or just after their peak. Whether slowing of tumor growth has occurred, following administration of a replication-competent, recombinant oncolytic vaccinia virus of the present disclosure, can be determined using any of a variety of established methods to measure tumor growth and/or cancer cell number. Whether replication of a replication-competent, recombinant oncolytic vaccinia virus of the present disclosure in an individual is at its peak or just after its peak can be determined by detecting and/or measuring levels of TKv polypeptide in the individual, as described herein, where a non-limiting example of a suitable method is PET. Whether circulating antibody to a replication-competent, recombinant oncolytic vaccinia virus of the present disclosure is at or just after its peak can be measured using standard methods for measuring the levels of an antibody, where such methods include, e.g., enzyme-linked immunosorbent assay (ELISA), radioimmunoassay (RIA), and the like.

As an example, a method of the present disclosure can comprise: a) administering to an individual in need thereof an effective amount of a recombinant oncolytic virus of the present disclosure; b) measuring: i) tumor size and/or cancer cell number in the individual; and/or ii) levels of TKv polypeptide in the individual; and/or iii) levels of antibody to the recombinant oncolytic virus in the individual; and c) where the measuring step indicates that: i) tumor growth has slowed and/or the number of cancer cells has decreased, compared to the tumor growth and/or the number of cancer cells before administration of the recombinant oncolytic virus; and/or ii) the level of TKv polypeptide in the individual is at or just past its peak; and/or iii) the level of circulating antibody to the recombinant oncolytic virus in the individual is at or just past its peak, administering a synthetic analog of 2′-deoxy-guanosine. For example, a method of the present disclosure can comprise: a) administering to an individual in need thereof an effective amount of a replication-competent, recombinant oncolytic virus of the present disclosure; and b) administering to the individual an effective amount of a synthetic analog of 2′-deoxy-guanosine, where the administration step (b) is carried out from 5 days to 20 days (e.g., 5 days, 6 days, 7 days, 8 days, 9 days, 10 days, 11 days, 12 days, 13 days, 14 days, 15 days, 16 days, 17 days, 18 days, 19 days, or 20 days) after step (a).

Suitable synthetic analogs of 2′-deoxy-guanosine include, e.g., acyclovir (acycloguanosine), 5′-iododeoxyuridine (also referred to as “idoxuridine”), ganciclovir, valganciclovir, famciclovir, valaciclovir, 2′-fluoro-2′-deoxy-5-iodo-1-beta-d-arabinofuranosyluracil (FIAU), and the like. The structures of some of the suitable synthetic analogs of 2′-deoxy-guanosine are shown below.

ganciclovir:

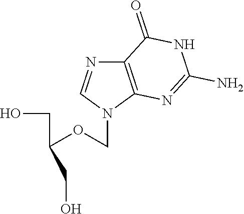

valganciclovir:

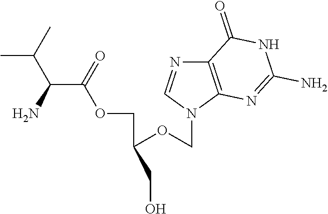 [SMILES: CC(C)[C@H](N)C(=O)OC[C@H](CO)OCn1cnc2c(=O)[nH]c(N)nc21]

valaciclovir:

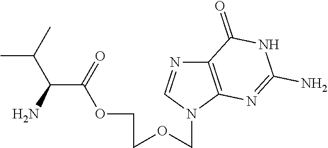 [SMILES: CC(C)[C@H](N)C(=O)OCCOCn1cnc2c(=O)[nH]c(N)nc21]

famciclovir:

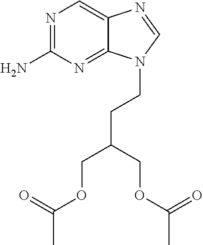 [SMILES: CC(=O)OCC(CCn1cnc2cnc(N)nc21)COC(C)=O]

In some particular embodiments, the synthetic analog of 2′-deoxy-guanosine is ganciclovir or acyclovir.

A synthetic analog of 2′-deoxy-guanosine may be administered in a dose of less than 4000 mg per day orally. In some cases, a suitable oral dose of a synthetic analog of 2′-deoxy-guanosine is in the range of from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day. In some cases, a suitable oral dose of a synthetic analog of 2′-deoxy-guanosine is in the range of from about 2500 mg per day to about 3000 mg per day, from about 3000 mg per day to about 3500 mg per day, or from about 3500 mg per day to about 4000 mg per day.

As one non-limiting example, ganciclovir can be administered in a dose of 1000 mg 3 times per day, for a total daily dose of 3000 mg. Ganciclovir can be administered in a total daily dose of less than 3000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, ganciclovir is administered via oral administration.

As another non-limiting example, acyclovir can be administered in a total daily dose of from 1000 mg to 4000 mg. Acyclovir can be administered in a total daily dose of less than 4000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, acyclovir is administered via oral administration.

As another example valganciclovir is administered in a total daily dose of from about 900 mg to about 1800 mg. Valganciclovir can be administered in a total daily dose of less than 1800 mg (e.g., from about 500 mg/day to about 600 mg/day, from about 600 mg/day to about 700 mg/day, from about 700 mg/day to about 800 mg/day, from about 800 mg/day to about 900 mg/day, from about 900 mg/day to about 1000 mg/day, from about 1000 mg/day to about 1200 mg/day, from about 1200 mg/day to about 1400 mg/day, or from about 1400 mg/day to about 1600 mg/day). In some cases, valganciclovir is administered via oral administration.

As another example, famciclovir is administered in a total daily dose of from about 2000 mg/day to about 4000 mg/day. Famciclovir can be administered in a total daily dose of less than 4000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, famciclovir is administered via oral administration.

As another example valacyclovir is administered in a total daily dose of from about 2000 mg to about 4000 mg. Valacyclovir can be administered in a total daily dose of less than 4000 mg (e.g., from about 50 mg per day to about 2500 mg per day, e.g., from about 50 mg per day to about 100 mg per day, from about 100 mg per day to about 200 mg per day, from about 200 mg per day to about 300 mg per day, from about 300 mg per day to about 400 mg per day, from about 400 mg per day to about 500 mg per day, from about 500 mg per day to about 600 mg per day, from about 600 mg per day to about 700 mg per day, from about 700 mg per day to about 800 mg per day, from about 800 mg per day to about 900 mg per day, from about 900 mg per day to about 1000 mg per day, from about 1000 mg per day to about 1250 mg per day, from about 1250 mg per day to about 1500 mg per day, from about 1500 mg per day to about 1750 mg per day, from about 1750 mg per day to about 2000 mg per day, from about 2000 mg per day to about 2250 mg per day, or from about 2250 mg per day to about 2500 mg per day). In some cases, valacyclovir is administered via oral administration.

As another example, ganciclovir is administered in a total daily dose of about 10 mg/kg. Ganciclovir can be administered in a total daily dose of less than 10 mg/kg (e.g., from about 1 mg/kg to about 2 mg/kg, from about 2 mg/kg to about 3 mg/kg, from about 3 mg/kg to about 4 mg/kg, from about 4 mg/kg to about 5 mg/kg, from about 5 mg/kg to about 6 mg/kg, from about 6 mg/kg to about 7 mg/kg, from about 7 mg/kg to about 8 mg/kg, or from about 8 mg/kg to about 9 mg/kg). In some cases, ganciclovir is administered via injection (e.g., intramuscular injection, intravenous injection, or subcutaneous injection).

As another example, acyclovir is administered in a total daily dose of from about 15 mg/kg to about 30 mg/kg, or from about 30 mg/kg to about 45 mg/kg. Acyclovir can be administered in a total daily dose of less than 45 mg/kg (e.g., from about 5 mg/kg to about 7.5 mg/kg, from about 7.5 mg/kg to about 10 mg/kg, from about 10 mg/kg to about 12.5 mg/kg, from about 12.5 mg/kg to about 15 mg/kg, from about 15 mg/kg to about 20 mg/kg, from about 20 mg/kg to about 25 mg/kg, from about 25 mg/kg to about 30 mg/kg, or from about 30 mg/kg to about 35 mg/kg. In some cases, acyclovir is administered via injection (e.g., intramuscular injection, intravenous injection, or subcutaneous injection).

As another example, valganciclovir is administered in a total daily dose of about 10 mg/kg. Valganciclovir can be administered in a total daily dose of less than 10 mg/kg (e.g., from about 1 mg/kg to about 2 mg/kg, from about 2 mg/kg to about 3 mg/kg, from about 3 mg/kg to about 4 mg/kg, from about 4 mg/kg to about 5 mg/kg, from about 5 mg/kg to about 6 mg/kg, from about 6 mg/kg to about 7 mg/kg, from about 7 mg/kg to about 8 mg/kg, or from about 8 mg/kg to about 9 mg/kg). In some cases, valganciclovir is administered via injection (e.g., intramuscular injection, intravenous injection, or subcutaneous injection).

In some cases, a synthetic analog of 2′-deoxy-guanosine is administered topically. Formulations suitable for topical administration include, e.g., dermal formulations (e.g., liquids, creams, gels, and the like) and ophthalmic formulations (e.g., creams, liquids, gels, and the like). Topical doses of ganciclovir can be, e.g., 1 drop of a 0.15% formulation 5 times per day, e.g., for ophthalmic indications. Topical doses of acyclovir can be, e.g., application 6 times per day of a 5% formulation in an amount sufficient to cover a skin lesion. Topical doses of idoxuridine can be, e.g., application every 4 hours of 1 drop of a 0.5% ointment or a 0.1% cream.

In some cases, a synthetic analog of 2′-deoxy-guanosine is administered in a dose less than 10 mg/kg body weight intravenously. In some cases, a suitable intravenous dose of a synthetic analog of 2′-deoxy-guanosine is in the range of from about 1 mg/kg body weight to about 2.5 mg/kg body weight, from about 2.5 mg/kg body weight to about 5 mg/kg body weight, from about 5 mg/kg body weight to about 7.5 mg/kg body weight, or from about 7.5 mg/kg body weight to about 10 mg/kg body weight.

C-7. Examples of Non-Limiting Aspects of the Disclosure

Aspects, including embodiments, of the oncolytic virus-related subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A recombinant oncolytic virus (OV) comprising, in its genome: (1) a nucleotide sequence encoding a variant interleukin-2 (IL-2) polypeptide, wherein the variant IL-2 polypeptide has reduced undesirable properties as compared to the wild-type IL-2; and (2) a nucleotide sequence encoding a heterologous thymidine kinase (TK) polypeptide.

Aspect 2. The OV of aspect 1, wherein the OV further comprises a modification that renders the vaccinia thymidine kinase deficient.

Aspect 3. The OV of aspect 2, wherein the modification results in a lack of J2R expression and/or function.

Aspect 4. The OV of any one of aspects 1-3, wherein the virus is a Copenhagen strain vaccinia virus.

Aspect 5. The OV of any one of aspects 1-3, wherein the virus is a WR strain vaccinia virus.

Aspect 6. The OV of any one of aspects 1-5, wherein the virus comprises an A34R gene comprising a K151E substitution.

Aspect 7. The OV of any one of aspects 1-6, wherein the variant IL-2 polypeptide comprises substitutions of one or more of F42, Y45, and L72, based on the amino acid numbering of the IL-2 amino acid sequence depicted in SEQ ID NO:1.

Aspect 8. The OV of any one of aspects 1-7, wherein IL-2v polypeptide comprises an F42L, F42A, F42G, F42S, F42T, F42Q, F42E, F42D, F42R, or F42K substitution, based on the amino acid numbering of the IL-2 amino acid sequence depicted in SEQ ID NO:1.

Aspect 9. The OV of any one of aspects 1-8, wherein IL-2v polypeptide comprises a Y45A, Y45G, Y45S, Y45T, Y45Q, Y45E, Y45N, Y45D, Y45R, or Y45K substitution, based on the amino acid numbering of the IL-2 amino acid sequence depicted in SEQ ID NO:1.

Aspect 10. The OV of any one of aspects 1-9, wherein IL-2v polypeptide comprises CD25 is an L72G, L72A, L725, L72T, L72Q, L72E, L72N, L72R, or L72K substitution, based on the amino acid numbering of the IL-2 amino acid sequence depicted in SEQ ID NO:1.

Aspect 11. The OV of any one of aspects 1-10, wherein the IL-2v polypeptide comprises F42A, Y45A, and L72G substitutions, based on the amino acid numbering of the IL-2 amino acid sequence depicted in SEQ ID NO:1.

Aspect 12. The OV of any one of aspects 1-11, wherein the IL-2v polypeptide-encoding nucleotide sequence is operably linked to a regulatable promoter.

Aspect 13. The OV of aspect 12, wherein the regulatable promoter is regulated by tetracycline or a tetracycline analog or derivative.

Aspect 14. A composition comprising: a) the OV of any one of aspects 1-13; and b) a pharmaceutically acceptable excipient.

Aspect 15. A method of inducing oncolysis in an individual having a tumor, the method comprising administering to the individual an effective amount of the OV of any one of aspects 1-13, or the composition of aspect 14.

Aspect 16. The method of aspect 15, wherein said administering comprises administering a single dose of the virus or the composition.

Aspect 17. The method of aspect 16, wherein the single dose comprises at least 10⁶ plaque forming units (pfu) of the virus.

Aspect 18. The method of aspect 16, wherein the single dose comprises from 10⁹ to 10¹² pfu of the virus.

Aspect 19. The method of aspect 15, wherein said administering comprises administering multiple doses of the virus or the composition.

Aspect 20. The method of aspect 19, wherein the virus or the composition is administered every other day.

Aspect 21. The method of any one of aspects 15-20, wherein the virus or the composition is administered once per week.

Aspect 22. The method of any one of aspects 15-20, wherein the virus or the composition is administered every other week.

Aspect 23. The method of any one of aspects 15-21, wherein the tumor is a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

Aspect 24. The method of any one of aspects 15-22, wherein the tumor is a colorectal adenocarcinoma.

Aspect 25. The method of any one of aspects 15-22, wherein the tumor is non-small cell lung carcinoma.

Aspect 26. The method of any one of aspects 15-22, wherein the tumor is a triple-negative breast cancer.

Aspect 27. The method of any one of aspects 15-22, wherein the tumor is a solid tumor.

Aspect 28. The method of any one of aspects 15-22, wherein the tumor is a liquid tumor.

Aspect 29. The method of any one of aspects 15-28, wherein the tumor is recurrent.

Aspect 30. The method of any one of aspects 15-28, wherein the tumor is a primary tumor.

Aspect 31. The method of any one of aspects 15-28, wherein the tumor is metastatic.

Aspect 32. The method of any one of aspects 15-31, further comprising administering to the individual a second cancer therapy.

Aspect 33. The method of aspect 32, wherein the second cancer therapy is selected from chemotherapy, biological therapy, radiotherapy, immunotherapy, hormone therapy, anti-vascular therapy, cryotherapy, toxin therapy, oncolytic virus therapy, a cell therapy, and surgery.

Aspect 34. The method of aspect 32, wherein the second cancer therapy comprises an anti-PD1 antibody or an anti-PD-L1 antibody.

Aspect 35. The method of any one of aspects 15-34, wherein the individual is immunocompromised.

Aspect 36. The method of any one of aspects 15-35, wherein said administering of the vaccinia virus or the composition is intratumoral.

Aspect 37. The method of any one of aspects 15-35, wherein said administering of the vaccinia virus or the composition is peritumoral.

Aspect 38. The method of any one of aspects 15-35, wherein said administering of the vaccinia virus or the composition is intravenous.

Aspect 39. The method of any one of aspects 15-35, wherein said administering of the vaccinia virus or the composition is intra-arterial.

Aspect 40. The method of any one of aspects 15-35, wherein said administering of the vaccinia virus or the composition is intrabladder.

Aspect 41. The method of any one of aspects 15-35, wherein said administering of the vaccinia virus or the composition is intrathecal.

Aspect 42. A recombinant OV comprising, in its genome, a nucleotide sequence encoding a variant interleukin-2 (IL-2v) polypeptide, wherein the IL-2v polypeptide comprises one or more amino acid substitutions that provides for reduced binding to CD25, compared to wild-type IL-2.

Aspect 43. A recombinant OV comprising, in its genome, a nucleotide sequence encoding a variant interleukin-2 (IL-2v) polypeptide comprising SEQ ID NO: 9, wherein the vaccinia virus is a Copenhagen strain vaccinia virus, is vaccinia thymidine kinase deficient, and comprises an A34R gene comprising a K151E substitution.

Aspect 44. The virus of aspect 43, further comprising a signal peptide.

Aspect 45. The virus of aspect 44, wherein the signal peptide comprises SEQ ID NO:22.

Aspect 46. A recombinant OV comprising, in its genome, a variant interleukin-2 (IL-2v) nucleotide sequence comprising SEQ ID NO:10, wherein the vaccinia virus is a Copenhagen strain vaccinia virus, is vaccinia thymidine kinase deficient, and comprises an A34R gene comprising a K151E substitution.

Aspect 47. A recombinant OV comprising, in its genome, a variant interleukin-2 (IL-2v) nucleotide sequence comprising SEQ ID NO:12, wherein the vaccinia virus is a Copenhagen strain vaccinia virus, is vaccinia thymidine kinase deficient, and comprises an A34R gene comprising a K151E substitution.

Aspect 48. A composition comprising: (i) the virus of any one of aspects 42-47 and (ii) a pharmaceutically acceptable carrier.

Aspect 49. A recombinant OV comprising, in its genome, a nucleotide sequence encoding a variant interleukin-2 (IL-2v) polypeptide, wherein the IL-2v polypeptide provides reduced undesirable biological activity when compared to wild-type IL-2.

Aspect 50. A recombinant OV comprising in its genome, a nucleotide sequence encoding a human variant IL-2, wherein the variant IL-2 comprises one or more substitutions relative to the human IL-2 protein sequence of SEQ ID NO: 1 at positions selected from T3, R38, L40, K43, Y45, E62, Y65, L72, Q74, and C125.

Aspect 51. The recombinant OV of Aspect 50, wherein the variant IL-2 comprises amino acid substitutions at one or more of the following groups of positions: R38 and L40; T41 and K43; K43 and Y45; E62 and K64; L72 and Q74; R38, L40, K43, and Y45; K43, Y45, L72, and Q74; T3, R38, L40, K43, and Y45; T3, K43, Y45, L72, and Q74; R38, L40, K43, Y45, and C125; K43, Y45, L72, Q74, and C125; T3, R38, L40, K43, Y45, and C125; T3, K43, Y45, L72, Q74, and C125.

Aspect 52. The recombinant OV of Aspect 50, wherein the variant IL-2 comprises one or more amino acid substitutions selected from the groups consisting of T3A, K35N, R38N, L40S, L40T, T41N, K43S, K43T, K43N, Y45S, Y45T, E62N, E62A, E62K, E62R, K64S, K64T, L72N, Q74S, Q74T, C125A, and C125S.

Aspect 53. The recombinant OV of Aspect 50, wherein the variant IL-2 polypeptide comprises R38N, L40T, K43N, and Y45T substitutions, based on the amino acid number of the IL-2 amino acid sequence depicted in SEQ ID NO:1.

Aspect 54. The recombinant OV referenced in any one of Aspect 1-53, which is recombinant oncolytic vaccinia virus.

### D. Description of Sequences Disclosed in Application

| SEQ | Description |
|---|---|
| ID NO | (AA: amino acid sequence; NT: nucleotide sequence) |
| 1 | AA - human mature form wild-type IL-2 (without signal peptide) |
| 2 | NT - encoding mouse IL-2 variant comprising F76A, Y79A, and |
| | L106G (of SEQ ID NO: 3) |
| 3 | AA - mouse IL-2 variant polypeptide comprising F76A, Y79A, |
| | and L106G (mIL-2v) |
| 4 | NT - VV27/VV38 homologous recombination donor fragment |
| 5 | NT - VV39 homologous recombination donor fragment |
| 6 | NT - Copenhagen J2R homologous recombination plasmid |
| 7 | NT - Copenhagen J2R homologous recombination plasmid |
| | containing mouse IL-2 variant (mIL-2v) polypeptide |
| 8 | NT - Western Reserve J2R homologous recombination plasmid |
| | containing mIL-2v |
| 9 | AA - human IL-2 variant comprising F42A, Y45A, and L72G |
| | (without signal peptide) |
| 10 | NT - encoding human IL-2 variant comprising F42A, Y45A, and |
| | L72G (codon-optimized?) |
| 11 | NT - codon-optimized, encoding human IL-2 variant comprising |
| | F42A, Y45A, and L72G |
| 12 | NT - encoding human precursor form IL-2 variant comprising |
| | F62A, Y65A, and L92G (of SEQ ID NO: 14) |
| 13 | NT - codon-optimized, encoding human precursor form IL-2 |
| | variant comprising F62A, Y65A, and L92G (of SEQ ID NO: |
| | 14?) |
| 14 | AA - human precursor form IL-2 variant comprising F62A, |
| | Y65A, and L92G (with signal peptide) |
| 15 | NT - VV75 homologous recombination donor fragment |
| | containing hIL-2v (human codon optimized) |
| 16 | NT- Copenhagen J2R homologous recombination plasmid |
| | containing hIL-2v (human codon optimized) |
| 17 | NT - homologous recombination donor fragment containing |
| | hIL-2v (vaccinia virus codon optimized) |
| 18 | Copenhagen J2R homologous recombination plasmid |
| | containing hIL-2v (vaccinia virus codon optimized) |
| 19 | NT - codon optimized, encoding a mouse IL-2 variant of |
| | SEQ ID NO: 3 |
| 20 | mouse IL-2 variant (vaccinia virus codon optimized) |
| | homologous recombination donor fragment) |
| 21 | AA - human Precursor form (full-length) wild-type IL-2 |
| | polypeptide (hIL-2) |
| 22 | AA - signal peptide of human IL-2 |
| 23 | AA -mouse mature form wild-type IL-2 polypeptide (mIL-2) |
| 24 | AA - mouse precursor form wild-type IL-2 polypeptide |
| 25 | AA - wild-type HSV-TK |
| 26 | AA - HSV-TK variant comprising 159Ile, 160 Leu, 161Ala, |
| | 168 Tyr, and 169 Phe |
| 27 | AA - HSV-TK variant comprising 159Ile, 160Phe, 161Leu, |
| | 168Phe, and 169 Met |
| 28 | AA - HSV-TK variant (i.e., HSV-TK.007) comprising 168H |
| 29 | AA - human IL-2 variant comprising R58N/L60T/K63N/Y65T |
| | (IL-2gv1; with signal peptide) |
| 30 | NT - encoding human IL-2 gv1 of SEQ ID NO: 29 (with |
| | signal peptide) |
| 31 | AA - human IL-2 gv1 without signal peptide (comprising |
| | R38N/L40T/K43N/Y45T) |
| 32 | NT - encoding human IL-2 gv1 of SEQ ID NO: 31 (without |
| | signal peptide) |
| 33 | AA - human IL-2 variant comprising K63N/Y65T/L92N/Q94T |
| | (IL-2 gv2; with signal peptide) |
| 34 | NT - encoding human IL-2 gv2 of SEQ ID NO: 33 |
| | (K63N/Y65T/L92N/Q94T; with signal peptide) |
| 35 | AA - human IL-2 gv2 (comprising K43N/Y45T/L72N/Q74T; |
| | without signal peptide) |
| 36 | NT - encoding human IL-2 gv2 of SEQ ID NO: 35 |
| | (comprising K43N/Y45T/L72N/Q74T; without signal peptide) |
| 37 | AA -Linker peptide |
| 38 | AA - A34 protein comprising K151E substitution |
| 39 | NT - A34R gene encoding the amino acid sequence of SEQ ID |
| | NO: 38 (A34 protein comprising K151E) |

### E. Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); i.v., intravenous(ly); i.t., intratumoral(ly); and the like. For clarity, a description of certain transgenes referenced in the Examples is provided Table 1:

TABLE 1
Description of Certain Transgenes Referenced in the Examples

| | | SEQ |
|---|---|---|
| Transgene | Description | ID NO |
| hIL-2 | Human full length wild-type IL-2 polypeptide | 21 |
| hIL-2v | human full-length IL-2 variant comprising F62A/ | 14 |
| | Y65A/L92G | |
| hIL-2gv or | human full-length IL-2 variant comprising | 29 |
| hIL-2gv1 | R58N/L60T/K63N/Y65T | |
| hIL-2gv2 | human full-length IL-2 variant comprising | 33 |
| | K63N/Y65T/L92N/Q94T | |
| mIL-2v | Mouse full-length IL-2 variant comprising F76A, | 3 |
| | Y79A, and L106G | |
| HSV- | HSV TK variant comprising 168H | 28 |
| TK.007 | | |

### Example 1: Generation of Recombinant Vaccinia Virus Constructs

Certain features of exemplary recombinant vaccinia virus constructs generated in connection with the examples provided below are summarized in Table 2, below. Each virus in Table 2 has a deletion of the J2R gene except VV10 and VV18 which have an insertional inactivation of the J2R gene. VV27, VV79, VV91-VV96 and IGV-121 have the gene encoding a mouse IL-2 variant (with F76A, Y79A, L106G substitutions; SEQ ID NO:3) which was codon optimized for expression in mouse cells. VV75 and VV100-VV103 have the gene encoding a human IL-2 variant (with F62A, Y65A, and L92G substitutions; SEQ ID NO:14) which was codon optimized for expression in human cells. VV97, VV110, and VV117 have the gene encoding a human IL-2 glycovariant (also referred to as “IL-2gv” or “IL-2gv1;” with R58N, L60T, K63N, and Y65T substitutions; SEQ ID NO:29) which was codon optimized for expression in human cells. VV98 has the gene encoding a human IL-2 glycovariant 2 (also referred to as “IL-2gv2;” with K63N, Y65T, L92N, and Q94T substitutions; SEQ ID NO:33) which was codon optimized for expression in human cells. VV99 has the gene encoding a human IL-2 (wildtype) which was codon optimized for expression in human cells.

TABLE 2
Features of Recombinant Vaccinia Virus Constructs

| | Description | | | | | |
|---|---|---|---|---|---|---|
| Virus | | | Transgene 1 | | Transgene 2 | |
| Construct | | | location/ | | location/ | |
| ID# | Strain | Transgene 1 | orientation | Transgene 2 | orientation | A34R |
| VV7 | Cop | Luc-GFP | J2R/forward | | | WT |
| VV10 | Cop | mGM-CSF | J2R/reverse | LacZ | J2R/forward | WT |
| VV16 | Cop | Luc-GFP | J2R/forward | | | K151E |
| VV18 | Cop | mGM-CSF | J2R/reverse | LacZ | J2R/forward | K151E |
| VV27 | Cop | mIL-2v | J2R/forward | | | K151E |
| VV75 | Cop | hIL-2v | J2R/forward | | | K151E |
| VV90 | Cop | | | | | K151E |
| VV91 | Cop | mIL-2v | J2R/forward | HSVTK.007 | B16R partial/ | K151E |
| | | | | | forward | |
| VV92 | Cop | mIL-2v | J2R/forward | HSVTK.007 | B16R partial/ | K151E |
| | | | | | reverse | |
| VV93 | Cop | mIL-2v | J2R/forward | HSVTK.007 | J2R/reverse | K151E |
| VV95 | Cop | mIL-2v | J2R/forward | HSVTK.007 | B16R/forward | K151E |
| VV96 | Cop | mIL-2v | J2R/forward | HSVTK.007 | B16R/reverse | K151E |
| VV101 | Cop | hIL-2v | J2R/forward | HSVTK.007 | J2R/reverse | K151E |
| VV102 | Cop | hIL-2v | J2R/forward | HSVTK.007 | B16R partial/ | K151E |
| | | | | | forward | |
| VV103 | Cop | hIL-2v | J2R/forward | HSVTK.007 | B16R/reverse | K151E |
| VV110 | Cop | hIL-2gv | J2R/forward | HSVTK.007 | B16R partial/ | K151E |
| | | | | | forward | |
| VV3 | WR | Luc-GFP | J2R/forward | | | WT |
| VV17 | WR | Luc-GFP | J2R/forward | | | K151E |
| VV79 | WR | mIL-2v | J2R/forward | | | K151E |
| VV94 | WR | mIL-2v | J2R/forward | HSVTK.007 | J2R/reverse | K151E |
| VV97 | WR | hIL-2gv | J2R/forward | | | WT |
| VV98 | WR | hIL-2gv2 | J2R/forward | | | WT |
| VV99 | WR | hIL-2 | J2R/forward | | | WT |
| VV100 | WR | hIL-2v | J2R/forward | | | WT |
| VV117 | WR | hIL-2gv | J2R/forward | HSVTK.007 | B15R-B17L/ | K151E |
| | | | | | forward | |
| IGV-121 | WR | mIL-2v | J2R/forward | HSVTK.007 | B15R-B17L/ | K151E |
| | | | | | forward | |

VV27 Construction

The virus is based on the Copenhagen strain of vaccinia and carries the gene encoding the mouse IL-2 variant under the control of a synthetic early late promoter and operator. The virus was engineered for enhanced extracellular enveloped virus (EEV) production by incorporation of a K151E substitution in the A34R gene. VV27 was constructed using a helper virus-mediated, restriction enzyme-guided, homologous recombination repair and rescue technique. First, the gene encoding mouse IL-2v (F76A, Y79A, L106G) was codon optimized for expression in mouse cells and synthesized by GeneWiz (South Plainfield, N.J.). The DNA was digested with BgIII/AsiSI and inserted into the Copenhagen J2R homologous recombination plasmid also digested with BgIII/AsiSI. The mouse IL-2v gene and flanking left and right vaccinia homology regions were amplified by PCR to generate the homologous recombination donor fragment. BSC-40 cells were infected with Shope Fibroma Virus (SFV), a helper virus, for one hour and subsequently transfected with a mixture of the donor amplicon and purified vaccinia genomic DNA previously restriction digested within the J2R region. The parent genomic DNA originated from a Copenhagen strain vaccinia virus carrying firefly luciferase and GFP in place of the native J2R gene and a K151E mutation (substitution) within the A34R gene for enhanced EEV production. Transfected cells were incubated until significant cytopathic effects were observed and total cell lysate was harvested by 3 rounds of freezing/thawing and sonication. Lysates were serially diluted, plated on BSC-40 monolayers, and covered by agar overlay. GFP negative plaques were isolated under a fluorescent microscope over a total of three rounds of plaque purification. One plaque (KR144) was selected for intermediate amplification in BSC-40 cells in a T225 flask, prior to large scale amplification in HeLa cells in a 20-layer cell factory. The virus was purified by sucrose gradient ultracentrifugation and thoroughly characterized in quality control assays, including full genome next generation sequencing.

VV38 Construction

The virus is based on the Copenhagen strain of vaccinia and carries the gene encoding the mouse IL-2 variant under the control of a synthetic early late promoter and operator. The virus is identical to VV27 except that it carries a wildtype A34R gene and is not engineered for enhanced EEV production. VV38 was constructed using a helper virus-mediated, restriction enzyme-guided, homologous recombination repair and rescue technique. BSC-40 cells were infected with SFV helper virus for 1-2 hours and subsequently transfected with a mixture of the donor amplicon and purified vaccinia genomic DNA previously digested with AsiSI in the J2R region. The parent genomic DNA originated from a Copenhagen strain vaccinia virus carrying firefly luciferase and GFP in place of the native J2R gene. Transfected cells were incubated until significant cytopathic effects were observed and total cell lysate was harvested by 3 rounds of freezing/thawing and sonication. Lysates were serially diluted, plated on BSC-40 monolayers, and covered by agar overlay. GFP negative plaques were isolated under a fluorescent microscope for a total of three rounds of plaque purification. One plaque (LW226) was selected for intermediate amplification in BSC-40 cells in a T225 flask, prior to large scale amplification in HeLa cells in a 20-layer cell factory. The virus was purified by sucrose gradient ultracentrifugation and thoroughly characterized in quality control assays, including full genome next generation sequencing.

VV39 Construction

The virus is based on the Western Reserve (WR) strain of vaccinia and carries the gene encoding the mouse IL-2 variant under the control of a synthetic early late promoter and operator. VV39 was constructed using a helper virus-mediated, restriction enzyme-guided, homologous recombination repair and rescue technique. BSC-40 cells were infected with SFV helper virus for 1-2 hours and subsequently transfected with a mixture of the donor amplicon and purified vaccinia genomic DNA previously digested with AsiSI in the J2R region. The parent genomic DNA originated from a WR strain vaccinia virus carrying a luciferase-2A-GFP reporter gene cassette in place of the native J2R gene and a wild-type A34R, which is not engineered for enhanced EEV production. Transfected cells were incubated until significant cytopathic effects were observed and total cell lysate was harvested by 3 rounds of freezing/thawing and sonication. Lysates were serially diluted, plated on BSC-40 monolayers, and covered by agar overlay. GFP negative plaques were isolated under a fluorescent microscope for a total of three rounds of plaque purification. One plaque (LW228) was selected for intermediate amplification in BSC-40 cells in a T225 flask, prior to large scale amplification in HeLa cells in a 20-layer cell factory. The virus (lot #180330) was purified by sucrose gradient ultracentrifugation and thoroughly characterized in quality control assays, including full genome next generation sequencing.

VV79 Construction

VV79, the WR strain equivalent of Copenhagen VV27, is identical to VV39 except for the addition of the A34R K151E substitution. It was constructed using helper virus mediated, homologous recombination repair and rescue techniques to insert the K151E mutation into the VV39 parental virus backbone.

VV101 Construction

VV101 is an armed oncolytic virus based upon the Copenhagen (Cop) strain of vaccinia virus. It differs from the parental Copenhagen smallpox vaccine strain by four genetic modifications, including 1) deletion of the native vaccinia J2R (thymidine kinase) gene, 2) insertion of a human IL-2 variant (hIL-2v) expression cassette controlled by a synthetic early-late promoter within the J2R locus, 3) insertion of a herpes simplex virus (HSV) thymidine kinase variant (TK.007) expression cassette controlled by an F17 promoter within the J2R locus in the opposite orientation as the hIL-2v cassette, and 4) mutation within the viral A34R gene that introduces a lysine to glutamate substitution at position 151 of the A34 protein (K151E). VV101 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the J2R region of vaccinia Copenhagen. Second, vaccinia nucleic acids were extracted from purified VV27 and transfected into Shope Fibroma Virus infected BSC-40 cells along with the HSV TK.007/J2R homologous recombination plasmid. Following a 3-day incubation, lysates were harvested by repeated freezing and thawing. Viruses were carried through 4 rounds of plaque purification and screened for the presence of HSV-TK.007 by PCR. The virus, labelled VV93, was expanded in HeLa cells, purified by sucrose gradient centrifugation, and characterized in quality control assays, including full genome next generation sequencing. Finally, VV101 was constructed from VV93 by replacing the gene encoding mouse IL-2 variant (mIL-2v) with a gene encoding hIL-2v, optimized for expression in human, using helper virus mediated, homologous recombination repair and rescue techniques as described above. Following recombination, plaque purification and screening, VV101 was expanded in HeLa cells, purified by sucrose gradient centrifugation, and characterized in quality control assays, including full genome next generation sequencing.

VV102 Construction

VV102 is an armed oncolytic virus based upon the Copenhagen strain of vaccinia virus. It differs from the parental Copenhagen smallpox vaccine strain by four genetic modifications, including 1) deletion of the native vaccinia J2R gene, 2) insertion of a hIL-2v expression cassette controlled by a synthetic early-late promoter within the J2R locus, 3) insertion of an HSV thymidine kinase variant (HSV TK.007; SEQ ID NO:28) expression cassette controlled by an F17 promoter within the B16R locus, replacing 157 bases of the native B16R gene, and 4) mutation within the viral A34R gene that introduces a lysine to glutamate substitution at position 151 of the A34 protein (K151E). VV102 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the B16R region of vaccinia Copenhagen. Second, vaccinia nucleic acids were extracted from purified VV27 (described in IGNT-001) and transfected into Shope Fibroma Virus infected BSC-40 cells along with the HSV TK.007/B16 homologous recombination plasmid. Following a 3-day incubation, lysates were harvested by repeated freezing and thawing. Viruses were carried through 4 rounds of plaque purification and screened for the presence of HSV TK.007 by PCR. The virus, labelled VV91, was expanded in HeLa cells, purified by sucrose gradient centrifugation, and characterized in quality control assays, including full genome next generation sequencing. Finally, VV102 was constructed from VV91 by replacing the gene encoding mIL-2v with a gene encoding hIL-2v, optimized for expression in human, using helper virus mediated, homologous recombination repair and rescue techniques as described above. Following recombination, plaque purification and screening, VV102 was expanded in HeLa cells, purified by sucrose gradient centrifugation, and characterized in quality control assays, including full genome next generation sequencing.

VV103 Construction

VV103 is an armed oncolytic virus based upon the Copenhagen strain of vaccinia virus. It differs from the parental Copenhagen smallpox vaccine strain by four genetic modifications, including 1) deletion of the native vaccinia J2R gene, 2) insertion of a hIL-2v expression cassette controlled by a synthetic early-late promoter within the J2R locus, 3) insertion of an HSV thymidine kinase variant (TK.007) expression cassette controlled by an F17 promoter within the B16R locus, replacing the entire native B16R gene, and 4) mutation within the viral A34R gene that introduces a lysine to glutamate substitution at position 151 of the A34 protein (K151E). VV103 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the B16R region of vaccinia Copenhagen. Second, vaccinia nucleic acids were extracted from purified VV27 (described in IGNT-001) and transfected into Shope Fibroma Virus infected BSC-40 cells along with the HSV TK.007/B16 homologous recombination plasmid. Following a 3-day incubation, lysates were harvested by repeated freezing and thawing. Viruses were carried through 4 rounds of plaque purification and screened for the presence of HSV TK.007 by PCR. The virus, labelled VV96, was expanded in HeLa cells, purified by sucrose gradient centrifugation, and characterized in quality control assays, including full genome next generation sequencing. Finally, VV103 was constructed from VV96 by replacing the gene encoding mIL-2v with a gene encoding hIL-2v, optimized for expression in human, using helper virus mediated, homologous recombination repair and rescue techniques as described above. Following recombination, plaque purification and screening, VV103 was expanded in HeLa cells, purified by sucrose gradient centrifugation, and characterized in quality control assays, including full genome next generation sequencing.

VV94 Construction

VV94 is an armed oncolytic virus based upon the mouse-adapted Western Reserve (WR) strain of vaccinia virus. It differs from the parental WR strain by four genetic modifications, including 1) deletion of the native vaccinia J2R gene, 2) insertion of a mIL-2v expression cassette controlled by a synthetic early-late promoter within the J2R locus in the forward orientation, 3) insertion of an HSV thymidine kinase variant (TK.007) expression cassette controlled by an F17 promoter within the J2R locus in the reverse orientation, and 4) mutation within the viral A34R gene that introduces a lysine to glutamate substitution at position 151 of the A34 protein (K151E). VV94 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the WR J2R region. Second, vaccinia nucleic acids were extracted from purified VV79 and transfected into Shope Fibroma Virus infected BSC-40 cells along with the HSVTK.007/J2R homologous recombination amplicon. Following a 3-day incubation, lysates were harvested by repeated freezing and thawing. Viruses were carried through 4 rounds of plaque purification and screened for the presence of HSV-TK.007 by PCR. The virus, labelled VV94, was expanded first in BSC-40 cells, then in HeLa cells, purified by sucrose gradient centrifugation, and characterized in quality control assays, including full genome next generation sequencing.

VV110 Construction

VV110 is an armed oncolytic virus based upon the Copenhagen strain of vaccinia virus. It differs from the parental Copenhagen smallpox vaccine strain by four genetic modifications, including 1) deletion of the native vaccinia J2R gene, 2) insertion of a human IL-2 glycovariant (R58N, L60T, K63N, and Y65T; SEQ ID NO:29) expression cassette controlled by a synthetic early-late promoter within the J2R locus, 3) insertion of an HSV thymidine kinase variant (TK.007) expression cassette controlled by an F17 promoter within the B16R locus, replacing 157 bases of the native B16R gene, and 4) mutation within the viral A34R gene that introduces a lysine to glutamate substitution at position 151 of the A34 protein (K151E). VV110 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the B16R region of vaccinia Copenhagen. Second, vaccinia nucleic acids were extracted from purified VV27 (described in IGNT-001) and transfected into Shope Fibroma Virus infected BSC-40 cells along with the HSV TK.007/B16 homologous recombination plasmid. Following a 3-day incubation, lysates were harvested by repeated freezing and thawing. Viruses were carried through 4 rounds of plaque purification and screened for the presence of HSV TK.007 by PCR. The virus, labelled VV91, was expanded in HeLa cells, purified by sucrose gradient centrifugation, and characterized in quality control assays, including full genome next generation sequencing. Finally, VV110 was constructed from VV91 by replacing the gene encoding mouse IL-2v with a gene encoding human IL-2 glycovariant, optimized for expression in human, using helper virus mediated, homologous recombination repair and rescue techniques as described above. Following recombination, plaque purification and screening, VV110 was expanded in HeLa cells, purified by sucrose gradient centrifugation, and characterized in quality control assays, including full genome next generation sequencing.

IGV-121 Construction

IGV-121 is an armed oncolytic virus based upon the mouse-adapted WR strain of vaccinia virus. It differs from the parental WR strain by four genetic modifications, including 1) deletion of the native vaccinia J2R gene, 2) insertion of a mIL-2v variant expression cassette controlled by a synthetic early-late promoter within the J2R locus, 3) insertion of an HSV thymidine kinase variant (TK.007) expression cassette controlled by an F17 promoter in the intergenic region between B15R (also known as WR197) and B17L (WR198), and 4) mutation within the viral A34R gene that introduces a lysine to glutamate substitution at position 151 of the A34 protein (K151E). IGV-121 was constructed using helper virus mediated, homologous recombination repair and rescue techniques. First, the gene encoding HSV TK.007 was codon optimized for expression by vaccinia virus and synthesized by Genscript. The gene was cloned downstream of an F17 promoter (P_{F17}) in a homologous recombination vector targeting the intergenic region between B15R and B17L of vaccinia WR strain. Second, vaccinia nucleic acids were extracted from purified VV79 (WR strain with J2R replaced by mouse IL-2v and A34R K151E mutation) and transfected into Shope Fibroma Virus infected Vero-B4 cells along with the HSV TK.007/B15R-B17L homologous recombination plasmid. Following a 2-day incubation, lysates were harvested by repeated freezing, thawing, and sonication. Viruses were carried through 3 rounds of plaque purification on BSC-40 cells. The virus, labelled IGV-121, was expanded in HeLa S3 cells, purified by sucrose gradient centrifugation, and characterized in quality control assays, including full genome next generation sequencing.

VV117 Construction

VV117 is an armed oncolytic virus based upon the mouse-adapted WR strain of vaccinia virus. It differs from the parental WR strain by four genetic modifications, including 1) deletion of the native vaccinia J2R gene, 2) insertion of a human IL-2 glycovariant (R58N, L60T, K63N, and Y65T; SEQ ID NO:29) expression cassette controlled by a synthetic early-late promoter within the J2R locus, 3) insertion of an HSV thymidine kinase variant (TK.007) expression cassette controlled by an F17 promoter in the intergenic region between B15R (also known as WR197) and B17L (WR198), and 4) mutation within the viral A34R gene that introduces a lysine to glutamate substitution at position 151 of the A34 protein (K151E). VV117 was constructed using helper virus mediated, homologous recombination repair and rescue techniques described previously. Vaccinia nucleic acids were extracted from purified IGV-121 (WR strain with J2R replaced by mouse IL-2v, A34R K151E mutation, and HSV TK.007 inserted into the intergenic region between B15R and B17R) and transfected into Shope Fibroma Virus infected BSC-40 cells along with the human IL-2 glycovariant homologous recombination plasmid. Following a 3-day incubation, lysates were harvested by repeated freezing, thawing, and sonication. Viruses were carried through 3 rounds of plaque purification on BSC-40 cells. The virus, labelled VV117, was expanded in HeLa cells, purified by sucrose gradient centrifugation, and characterized in quality control assays, including full genome next generation sequencing.

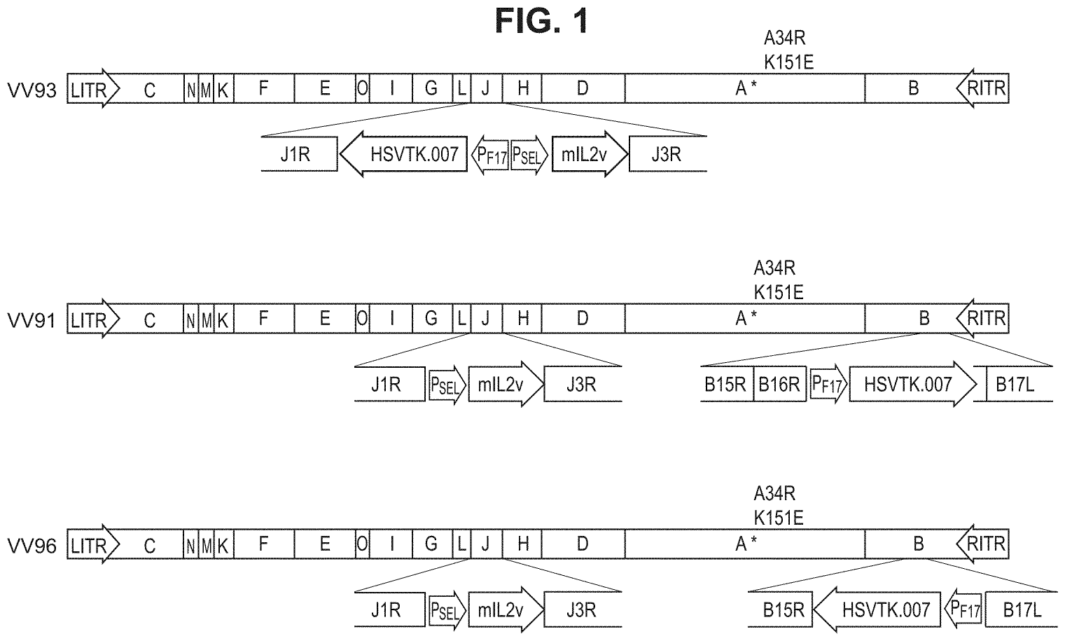

FIG. 1 is a schematic representation of full genomes for VV91, VV93, and VV96.

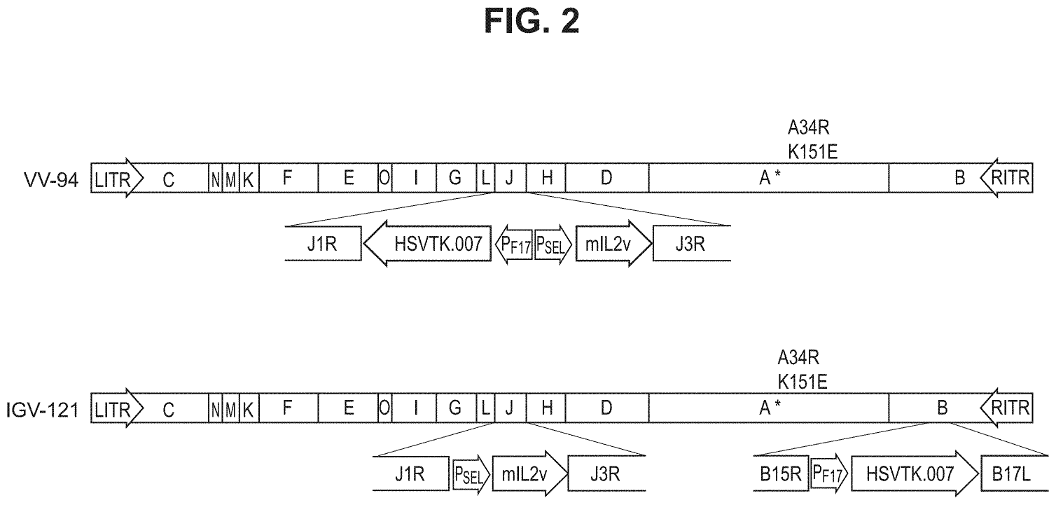

FIG. 2 is a schematic representation of full genomes for VV94 and IGV-121.

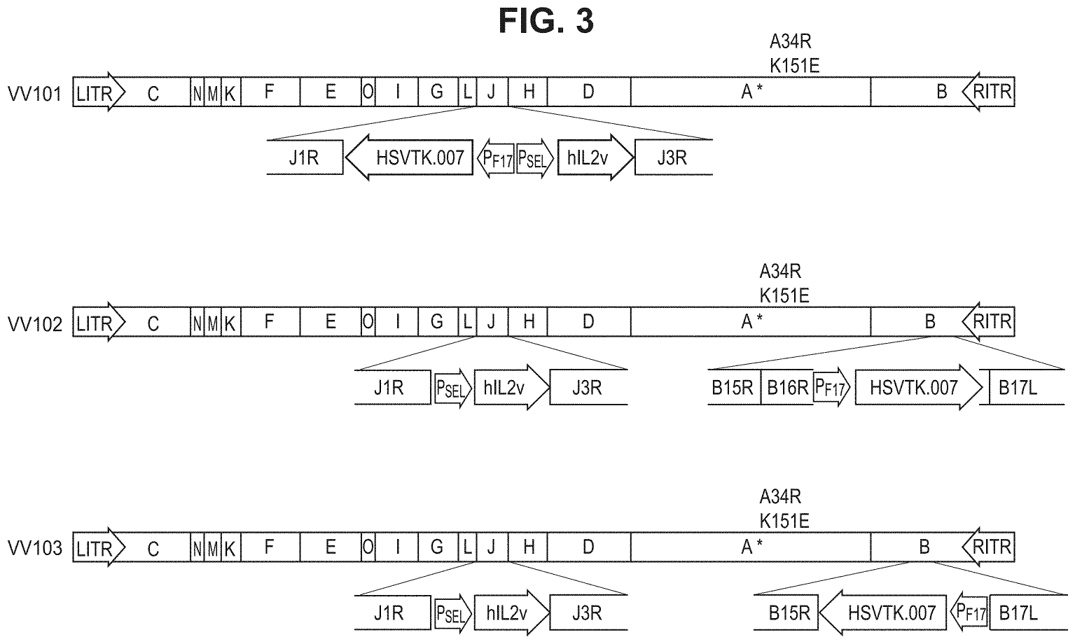

FIG. 3 is a schematic representation of full genomes for VV101-VV103.

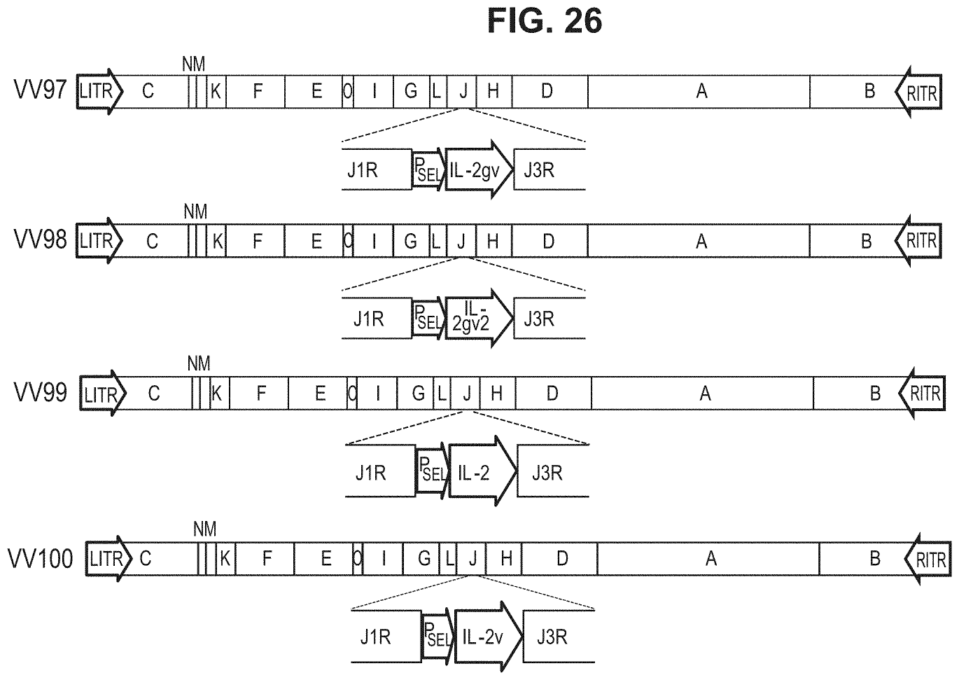

FIG. 26 is a schematic representation of full genomes for VV97-100. Abbreviations: LITR=left inverted terminal repeat; RITR=right inverted terminal repeat; A-O=viral gene regions historically defined by HindIII digest fragments; P_{SEL}=synthetic early late promoter; IL-2gv=human interleukin-2 glycovariant (R58N:L60T, K63N:Y65T); IL-2gv2=human interleukin-2 glycovariant 2 (K63N:Y65T, L92N:Q94T); IL-2=human interleukin-2 (wildtype); IL-2v=human interleukin-2 variant (F62A, Y65A, L92G)

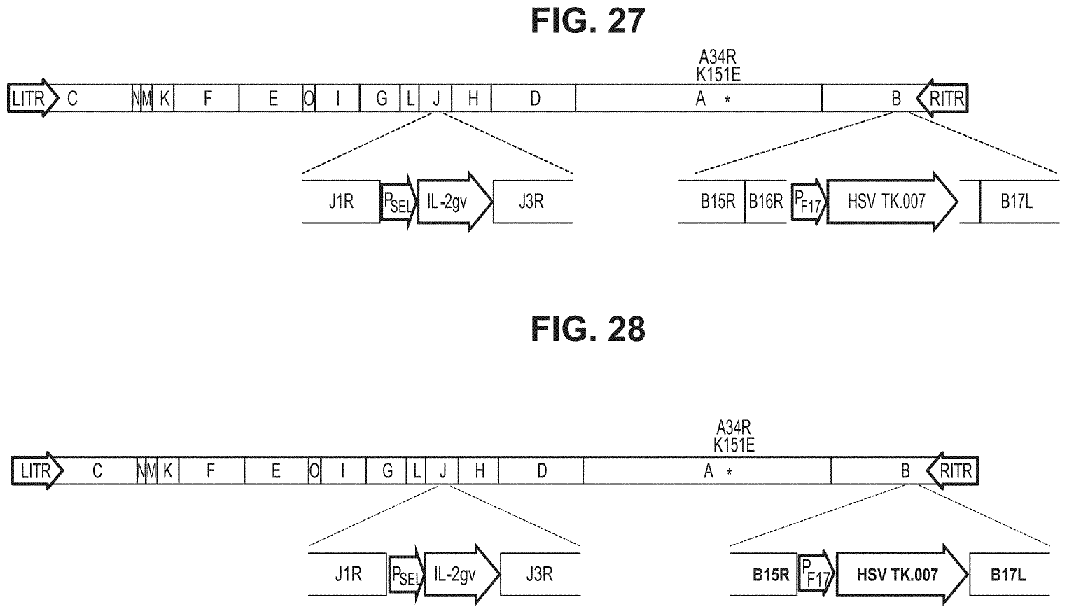

FIG. 27 is a schematic representation of full genomes for VV110. Abbreviations: LITR=left inverted terminal repeat; RITR=right inverted terminal repeat; A-O=viral gene regions historically defined by HindIII digest fragments; P_{SEL}=synthetic early late promoter; IL-2gv=human interleukin-2 glycovariant; *=mutation encoding substitution of lysine to glutamate at position 151 of A34 protein; P_{F17}=promoter from the F17R gene; HSV TK.007=herpes simplex virus thymidine kinase gene with mutation encoding alanine to histidine substitution at position 168

FIG. 28 is a schematic representation of full genomes for VV117. Abbreviations: LITR=left inverted terminal repeat; RITR=right inverted terminal repeat; A-O=viral gene regions historically defined by HindIII digest fragments; P_{SEL}=synthetic early late promoter; IL2gv=human interleukin-2 glycovariant; *=mutation encoding substitution of lysine to glutamate at position 151 of A34 protein; P_{F17}=promoter from the F17R gene; HSV TK.007=herpes simplex virus thymidine kinase gene with mutation encoding alanine to histidine substitution at position 168

### Example 2: IL-2v Expression from Recombinant Vaccinia Viruses in Virus-Infected Cells by Western Blot

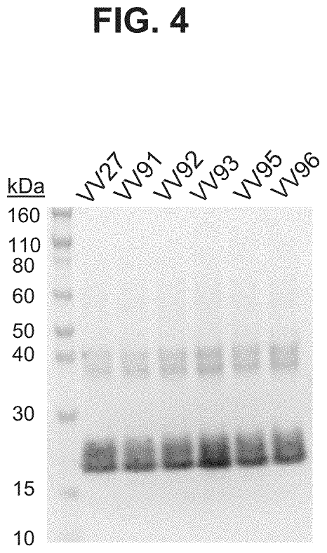

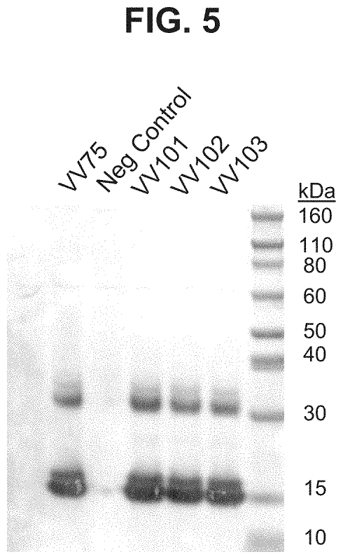

HeLa cells were plated at 6e5 cells/well in 2 mL of culture media in 6-well plates and after approx. 24 hr in culture infected with virus at MOI=3 for 24 hr. Cells from each well were subsequently harvested and lysed in 200 μL Laemmli buffer then diluted 1:1 with milliQ water. 12 μL of sample was prepared to a final volume of 20 μL in Tris-buffered saline (TBS) containing Reducing Agent and 1× NuPage LDS sample buffer prior to incubation at 95° C. for 5 min and loading on a NuPage 4-12% Bis-Tris gel. Gel electrophoresis with 1×MES running buffer was performed at 200V for 30 min. Proteins were transferred to a PVDF membrane using an iBlot device and Western Blot was performed using an iBlot device. For detection of mIL-2v the following antibodies were used—anti-IL-2 primary antibody (Abcam, ab11510) at 1:2000 dilution, goat anti-rat IgG-HRP secondary antibody (Invitrogen, #629526) at 1:1000 dilution. For detection of hIL-2v the following antibodies were used—anti-IL-2 primary antibody (Novus Biologicals, NBP2-16948) at 1:500 dilution, mouse anti-rabbit IgG-HRP secondary antibody (Pierce, #31460) at 1:2000 dilution. TMB substrate was subsequently added to the membrane to visualize bands. Membrane was rinsed with water, dried and scanned. Results are shown in FIG. 4 (mIL-2v expression analysis following infection of cells with recombinant oncolytic vaccinia viruses) and FIG. 5 (hIL-2v expression analysis following infection of cells with recombinant oncolytic vaccinia viruses).

### Example 3: HSV TK.007 Expression from Recombinant Vaccinia Viruses in Virus-Infected Cells by RT-qPCR

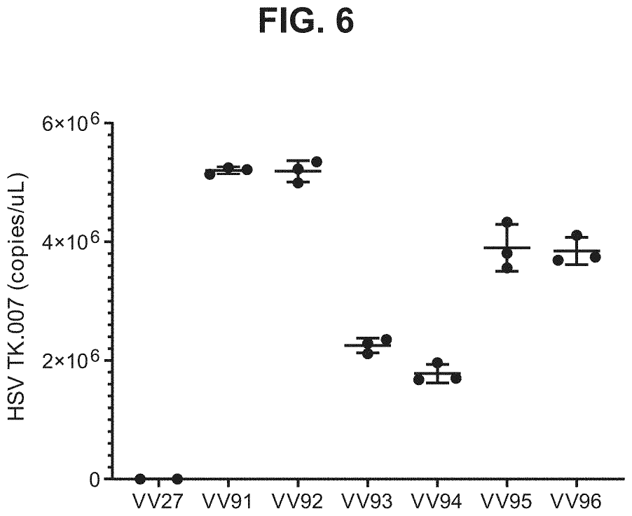

HeLa cells were plated at 7e4 cells/well in 2 mL of culture media in 6-well plates and after approximately 72 hr in culture infected with virus at MOI=3 for 18 hr. Cells from each well were subsequently harvested and processed for RNA extraction using the Rneasy Plus Universal Mini Kit (Qiagen, #73404). 500 ng total RNA was reverse transcribed using the High Capacity cDNA Reverse Transcription Kit (applied Biosystems, #4368814). cDNA was diluted 1:10 prior to use in qPCR to analyze HSV TK.007 mRNA expression levels using primers and probes specific for the HSV TK.007 transgene encoded in the recombinant viruses and PrimeTime Gene Expression Master Mix (IDT, #1055772). PCR was conducted on a ViiA7 instrument (Applied Biosystems). Plasmid DNA containing the HSV TK.007 cDNA sequence was used as a standard and copies/μL in each test sample determined from the standard curve. Results are shown in FIG. 6 (HSV TK.007 expression analysis following infection of cells with recombinant oncolytic vaccinia viruses).

### Example 4: Recombinant Oncolytic Vaccinia Virus Activity in MC38 Tumor-Bearing C57BL/6 Mice (Cop Viruses Expressing mIL-2v)

Female C57BL/6 mice (8-10 weeks old) were implanted subcutaneously (SC) on the right upper rear flank with 5e5 MC38 tumor cells. MC38 is a murine colon adenocarcinoma cell line. See, e.g., Cancer Research (1975) vol. 35, pp. 2434-2439. Eleven days after tumor cell implantation, mice were randomized based on tumor volume into separate treatment groups (average tumor volume per group ˜50 mm³; N=18/group). On day 12 post-implantation, tumors were directly injected with 60 μL vehicle (30 mM Tris, 10% sucrose, pH 8.0) or 60 μL vehicle containing 5e7 plaque forming units (pfu) of recombinant Copenhagen (Cop) vaccinia virus variant. Tumor-bearing mice were observed daily, and both tumor volumes and body weights measured bi-weekly until mice were humanely sacrificed either due to i) tumor volume surpassing 1400 mm³, ii) 20% body weight loss, or iii) severely diminished health status. Groups of mice were treated as follows:

Group i) vehicle only;

Group ii) VV16: Cop vaccinia virus carrying the A34R-K151E mutation (amino acid substitution) and armed with a Luciferase and green fluorescent protein (Luc-2A-GFP) dual reporter cassette;

Group iii) VV27: Cop vaccinia virus carrying the A34R-K151E substitution and armed with a mIL-2v transgene;

Group iv) VV91: Cop vaccinia virus carrying the A34R-K151E substitution, armed with a mIL-2v transgene, and encoding HSV TK.007 (B16R insertion, forward orientation);

Group v) VV93: Cop vaccinia virus carrying the A34R-K151E substitution, armed with a murine mIL-2v) transgene, and encoding HSV TK.007 (J2R insertion, reverse orientation); or

Group vi) VV96: Cop vaccinia virus carrying the A34R-K151E substitution, armed with a mIL-2v transgene, and encoding HSV TK.007 (B16R insertion, reverse orientation).

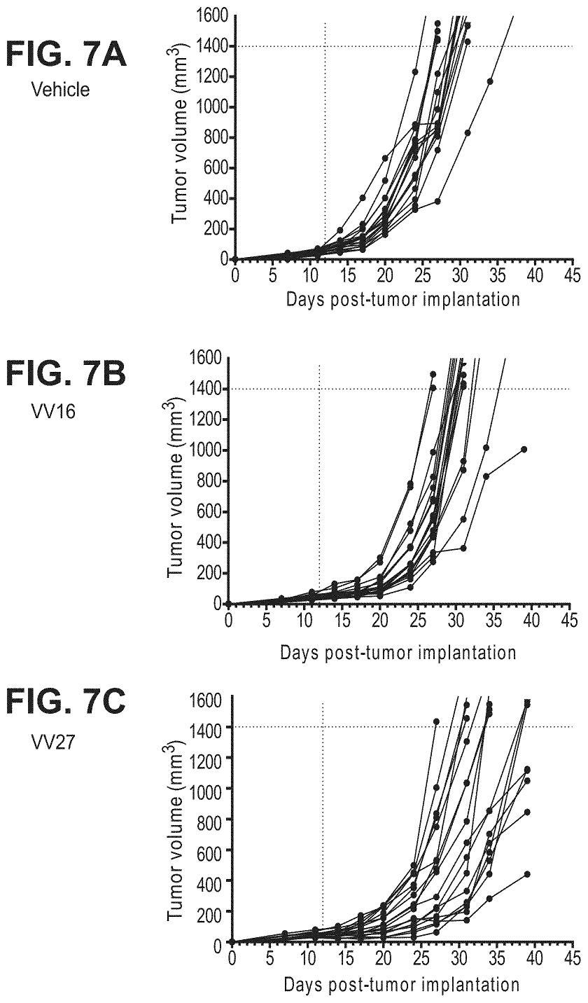

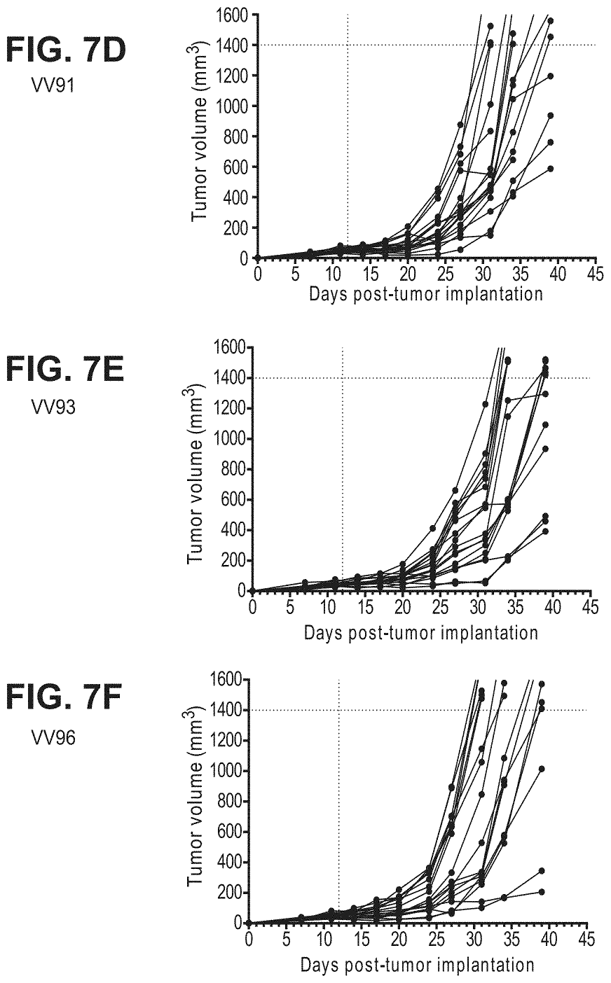

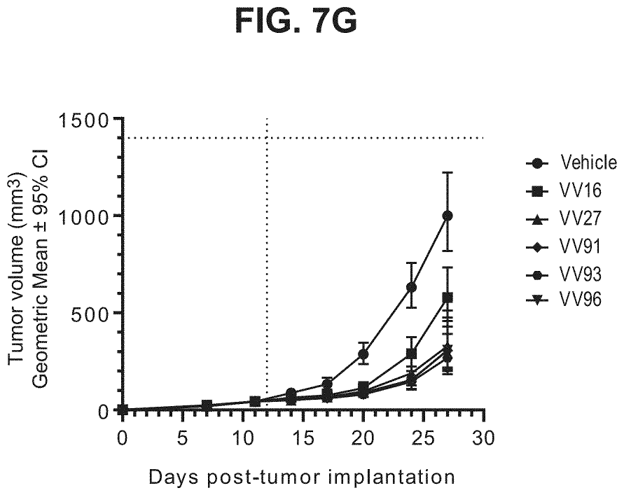

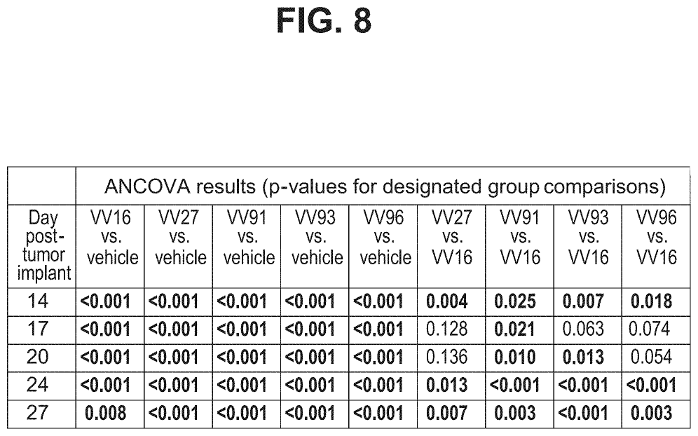

Comparisons between the tumor growth profiles of groups (i)-(vi) (FIG. 7) revealed that all test viruses produced a statistically significant inhibitory effect on tumor growth over multiple consecutive days, all of the mIL-2v-armed Cop vaccinia viruses (VV27, VV91, VV93, and VV96) produced a statistically significant inhibitory effect on tumor growth over multiple consecutive days compared to control virus (VV16) (FIG. 8, ANCOVA results), and that there were no statistically significant differences observed when comparing VV27 (mIL-2v only) to either VV91, VV93, or VV96 (each with mIL-2v and HSV TK.007).

FIG. 7A-7G show results of assessment of virotherapy-induced tumor growth inhibition on C57BL/6 female mice implanted SC with MC38 tumor cells. Tumor growth trajectories are shown for individual mice in groups treated with vehicle only (A) or Copenhagen vaccinia virus containing the A34R K151E mutation armed with either a Luciferase-2A-GFP reporter (Cop.Luc-GFP.A34R-K151E; VV16) (B), mIL-2v only (Cop.mGM-CSF.A34R-K151E; VV27) (C), mIL-2v and HSV TK.007 in a forward orientation in the B16R gene locus (Cop.mIL-2v.A34R-K151E.HSV TK.007 (B16R_For); VV91) (D), mIL-2v and HSV TK.007 in a reverse orientation in the J2R gene locus (Cop.mIL-2v.A34R-K151E.HSV TK.007 (J2R_Rev); VV93) (E), or mIL-2v and HSV TK.007 in a reverse orientation in the B16R gene locus (Cop.mIL-2v.A34R-K151E.HSV TK.007 (B16R_Rev); VV96) (F). The dashed vertical line on each graph represents time point when mice received intratumoral injections of vehicle or virus. The dashed horizontal line on each graph represents the tumor volume threshold used as a criterion to remove animals from the study. Average tumor volumes (mm³)±95% confidence intervals for each treatment group are shown through day 28 post-tumor implant (G), which was the last tumor measurement time point when all animals in each group were still alive.

FIG. 8 shows results of statistical comparison of virotherapy-induced tumor growth inhibition using ANCOVA. Tumor volumes for individual mice in each group after vehicle/virus treatment (day 14 to day 27 post-tumor implantation) were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p≤0.05.

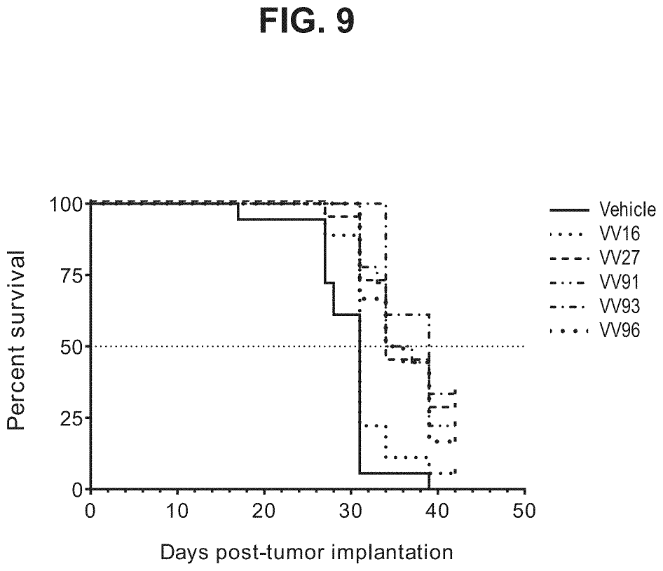

Survival of animals in each treatment group (N=18/group) was also assessed up through day 41 post-tumor implantation (FIG. 9). The unarmed vaccinia control (VV16) did not significantly improve survival over vehicle control (Log rank/Mantel-Cox test, p=0.133). However, mice treated with armed vaccinia viral variants VV27, VV91, VV93, and VV96 showed a statistically significant mean survival advantage over the reporter transgene-armed vaccinia control (VV16) treatment group (Log rank/Mantel-Cox test, p=0.009, 0.006, <0.0001, and 0.013 respectively).

FIG. 9 shows results of survival of MC38 tumor-implanted C57BL/6 female mice following treatment with vehicle or virus on day 12 after implantation. Mice were designated daily as deceased upon reaching tumor volume 1400 mm³. The point of intersection between each group's curve and the horizontal dashed line indicates the median (50%) survival threshold for group.

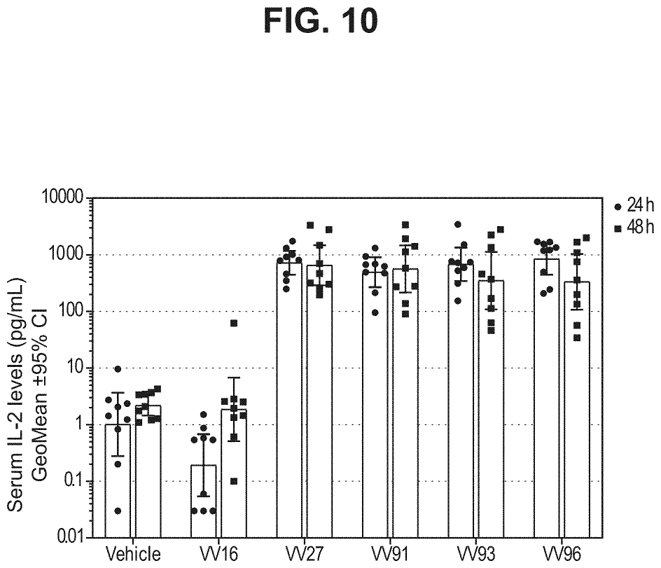

In addition to monitoring tumor growth inhibition and survival, sera were collected from tumor-bearing mice 24 hr and 48 hr after injection with vehicle or recombinant Cop vaccinia virus to assess circulating IL-2 levels. Circulating IL-2 levels in sera collected from each treatment group 24 hr and 48 hr after receiving intratumoral injections were quantified by ELISA (FIG. 10). Measurable levels of IL-2 were detected in the serum from most animals treated with the mIL-2v-armed Cop vaccinia virus variants (VV27, VV91, VV93, and VV96), while background levels of IL-2 were seen in any animal from the vehicle or other Cop vaccinia virus (VV16) groups. This latter result indicates that intratumoral injection of Cop vaccinia viruses lacking the mIL-2v transgene, at least at the tested dose levels, was insufficient to induce increased circulating IL-2 levels in the sera of treated animals. Thus, elevated levels seen in the sera of mice treated with the mIL-2v-armed Cop vaccinia virus should be indicative of transgene-mediated expression following intratumoral injection.

FIG. 10 shows results of IL-2 levels detected in sera collected from MC38 tumor-bearing C57BL/6 female mice 24 hr and 48 hr after intratumoral injection with vehicle or recombinant Cop vaccinia viruses. Each symbol represents the calculated IL-2 serum levels for an individual mouse, while bars represent group geometric mean (N=9/group). Error bars represent 95% confidence intervals.

### Example 5: mIL-2v-Armed Vaccinia Virus Activity in Lewis Lung Carcinoma (LLC) Tumor-Bearing C57BL/6 Mice (Cop Viruses Expressing mIL-2v)

Female C57BL/6 mice (8-10 weeks old) were implanted subcutaneously (SC) on the left upper rear flank with 1e5 LLC tumor cells. Twelve days after tumor cell implantation, mice were randomized based on tumor volume into separate treatment groups (average tumor volume per group ˜50 mm³; N=20/group). On day 13 post-implantation, tumors were directly injected with 60 μL vehicle (30 mM Tris, 10% sucrose, pH 8.0) or 60 μL vehicle containing 5e7 plaque forming units (pfu) of recombinant Copenhagen (Cop) vaccinia virus variant. Tumor-bearing mice were observed daily, and both tumor volumes and body weights measured bi-weekly until mice were humanely sacrificed either due to i) tumor volume surpassing 1400 mm³, ii) ≥20% body weight loss, or iii) severely diminished health status. Groups of mice were treated as follows:

Group i) vehicle only;

Group ii) VV16: Cop vaccinia virus carrying the A34R-K151E mutation (amino acid substitution) and armed with a Luciferase and green fluorescent protein (Luc-2A-GFP) dual reporter cassette;

Group iii) VV27: Cop vaccinia virus carrying the A34R-K151E substitution and armed with a mIL-2v transgene;

Group iv) VV91: Cop vaccinia virus carrying the A34R-K151E substitution, armed with a mIL-2v transgene, and encoding HSV TK.007 (B16R insertion, forward orientation);

Group v) VV93: Cop vaccinia virus carrying the A34R-K151E substitution, armed with a mIL-2v transgene, and encoding HSV TK.007 (J2R insertion, reverse orientation); or

Group vi) VV96: Cop vaccinia virus carrying the A34R-K151E substitution, armed with a mIL-2v transgene, and encoding HSV TK.007 (B16R insertion, reverse orientation).

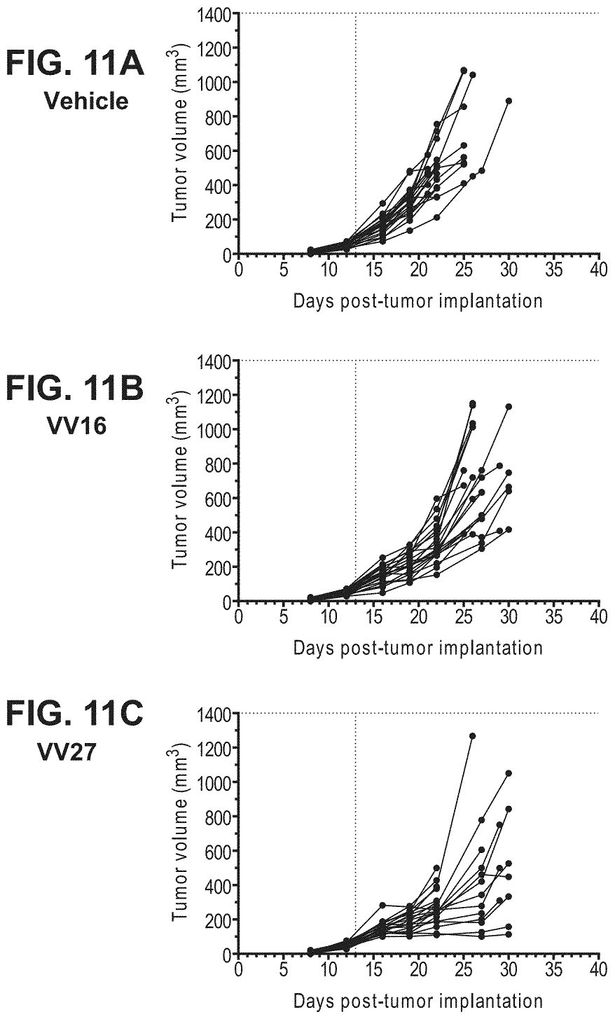

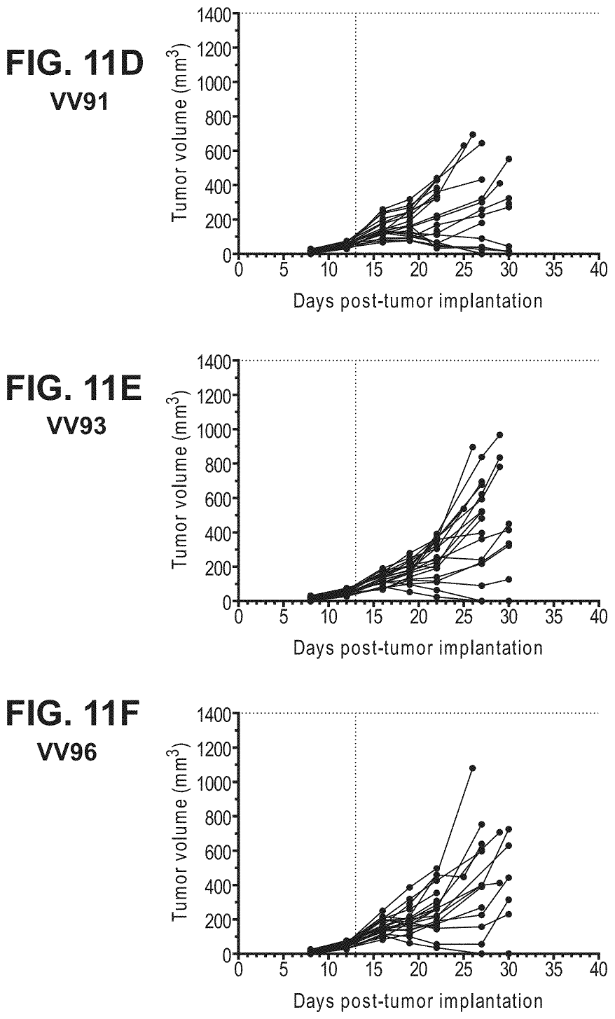

Comparisons between the tumor growth profiles of groups (i)-(vi) (FIG. 11) revealed that all test viruses produced an inhibitory effect on tumor growth, with the mIL-2v-armed Cop vaccinia viruses (VV27, VV91, VV93, and VV96) produced a more striking inhibitory effect on tumor growth compared to control virus (VV16). Many animals on study were humanely sacrificed due to tumor ulcerations and associated diminished health status limiting statistical analyses of tumor growth inhibition associated with different virus variants. However, analysis of individual animals demonstrated that 7/20, 2/20, and 1/20 tumors were either <50 mm³ or completely regressed by day 30 post-implant following treatment with VV91, VV93, or VV96, respectively, with no small tumors or complete regressions observed in other treatment groups.

FIG. 11A-11F show results of assessment of virotherapy-induced tumor growth inhibition on C57BL/6 female mice implanted SC with LLC tumor cells. Tumor growth trajectories are shown for individual mice in groups treated with vehicle only (A) or Copenhagen vaccinia virus containing the A34R K151E mutation and armed with either a Luciferase-2A-GFP reporter (Cop.Luc-GFP.A34R-K151E; VV16) (B), mIL-2v only (Cop.IL-2v.A34R-K151E; VV27) (C), mIL-2v and HSV TK.007 in a forward orientation in the B16R gene locus (Cop.mIL-2v.A34R-K151E.HSV TK.007 (B16R_For); VV91) (D), mIL-2v and HSV TK.007 in a reverse orientation in the J2R gene locus (Cop.mIL-2v.A34R-K151E.HSV TK.007 (J2R_Rev); VV93) (E), mIL-2v and HSV TK.007 in a reverse orientation in the B16R gene locus (Cop.mIL-2v.A34R-K151E.HSV TK.007 (B16R_Rev); VV96) (F). The dashed vertical line on each graph represents time point when mice received intratumoral injections of vehicle or virus. The dashed horizontal line on each graph represents the tumor volume threshold used as a criterion to remove animals from the study.

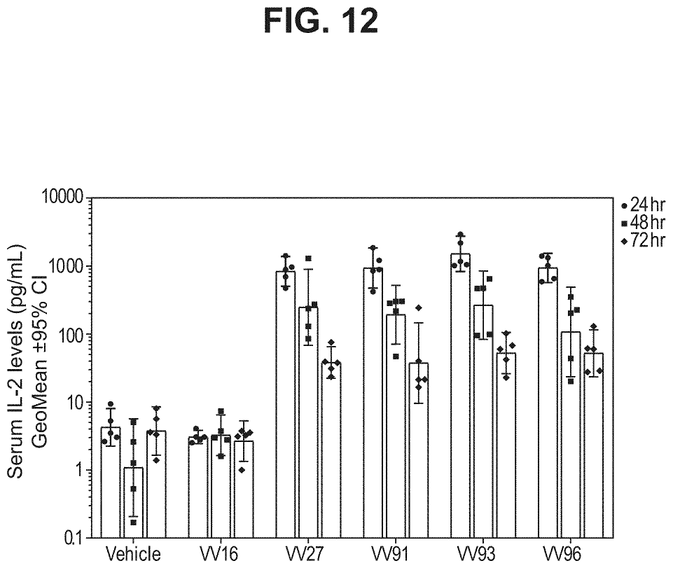

In addition to monitoring tumor growth inhibition and survival, sera were collected from tumor-bearing mice 24, 48, and 72 hr after injection with vehicle or recombinant Cop vaccinia virus to assess circulating IL-2 levels. Circulating IL-2 levels in sera collected from each treatment group at these timepoints after receiving intratumoral injections were quantified by ELISA (FIG. 12). Measurable levels of IL-2 were detected in the serum from most animals treated with the mIL-2v-armed Cop vaccinia virus variants (VV27, VV91, VV93, and VV96), while background levels of IL-2 were seen in any animal from the vehicle or other Cop vaccinia virus (VV16) groups. This latter result indicates that intratumoral injection of Cop vaccinia viruses lacking the mIL-2v transgene, at least at the tested dose levels, was insufficient to induce increased circulating IL-2 levels in the sera of treated animals. Thus, elevated levels seen in the sera of mice treated with the mIL-2v-armed Cop vaccinia virus should be indicative of transgene-mediated expression following intratumoral injection.

FIG. 12 shows results of IL-2 levels detected in sera collected from LLC tumor-bearing C57BL/6 female mice 24, 48, and 72 hr after intratumoral injection with vehicle or recombinant Cop vaccinia viruses. Each symbol represents the calculated IL-2 serum levels for an individual mouse, while bars represent group geometric mean (N=5/group). Error bars represent 95% confidence intervals.

### Example 6: Single IV Virotherapy Using Recombinant Oncolytic Vaccinia Virus in MC38 Tumor-Bearing C57BL/6 Mice (WR Viruses Expressing mIL-2v)

C57BL/6 female mice were implanted SC on the left flank with 5e5 MC38 tumor cells. Ten days after tumor cell implantation, mice were randomized based on tumor volume into separate treatment groups (average tumor volume per group ˜50 mm³; N=15/group). On day 11 post-tumor cell implantation, mice were injected IV with 100 μL of vehicle (30 mM Tris, 10% sucrose, pH8.0) or 100 μL of vehicle containing 5e7 pfu recombinant WR vaccinia virus. Tumor-bearing mice were observed daily, and both tumor volume and body weight were measured bi-weekly until mice were humanely sacrificed either due to i) tumor volume surpassing 1400 mm³, ii) 20% body weight loss, iii) severely diminished health status or iv) study termination.

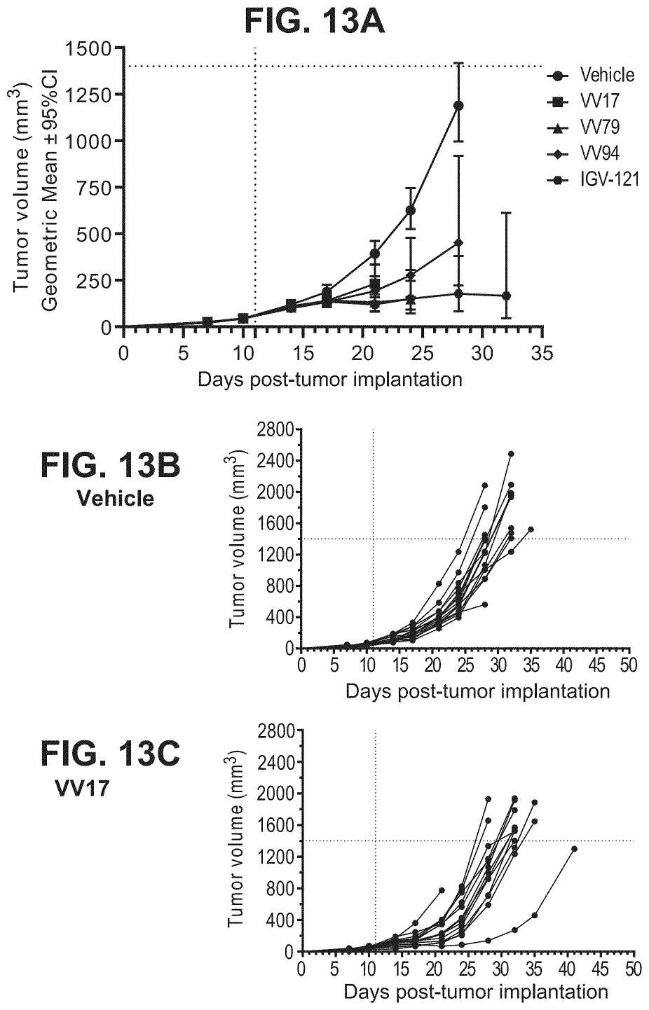

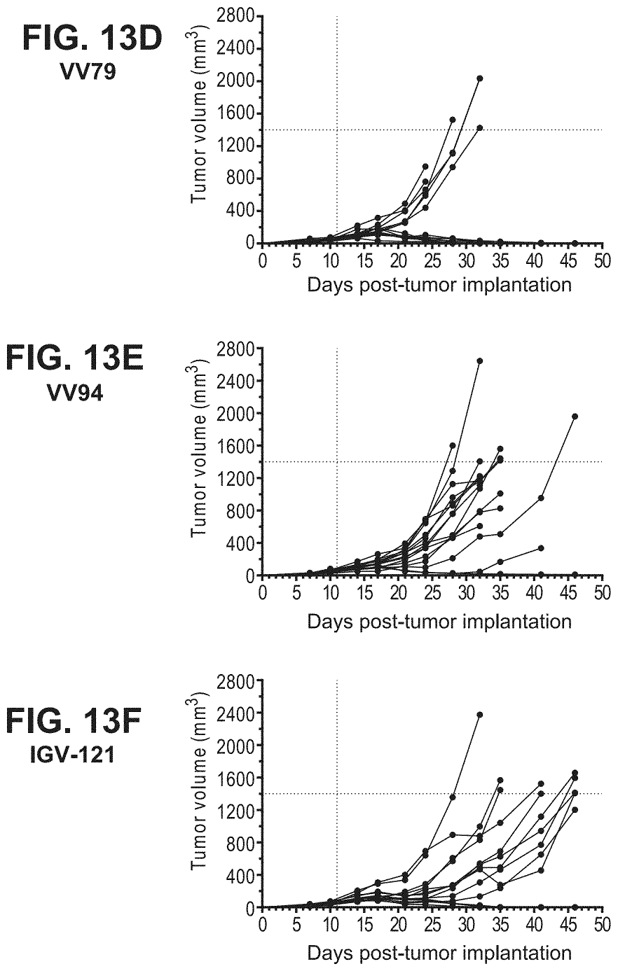

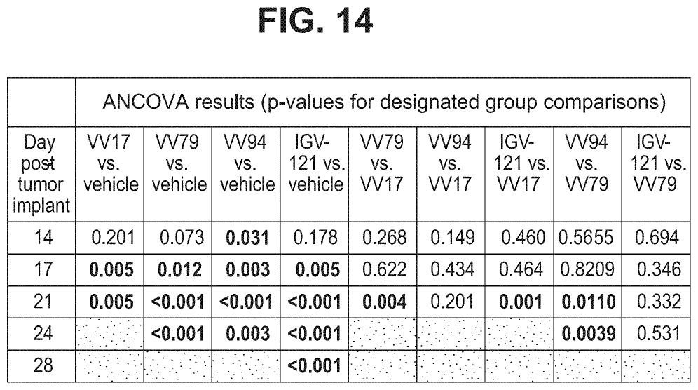

Analysis of tumor growth profiles, shown as group averages for each test virus (FIG. 13A) or as individual mice within each test group (FIG. 13B-13F), revealed an important finding. IV administration of mIL-2v transgene-armed WR viruses (encoding mIL-2v alone or with HSV TK.007) led to statistically significant inhibition of MC38 tumor growth compared to vehicle and reporter transgene-armed WR virus (VV17) treatment. There was no statistically significant difference between tumor growth inhibition induced by VV79 and IGV-121, however there was a statistically significant difference detected between VV79 and VV94 (FIG. 14, ANCOVA results).

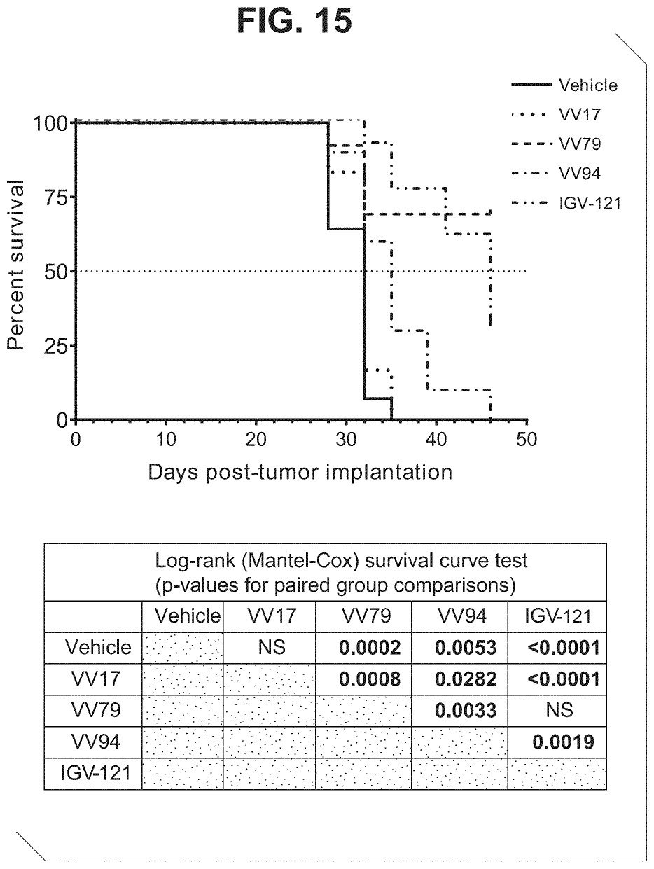

Survival results for the same test viruses showed very similar outcomes as those reported above for tumor growth inhibition. This included statistically superior group survival associated with mIL-2v transgene-armed WR viruses in the presence or absence of the HSV TK.007 compared to the corresponding Luc-GFP reporter-armed WR virus (FIG. 15). Overall, IV delivery of mIL-2v transgene-armed WR virus variants proved to be an effective anti-tumor therapy in the MC38 SC tumor model and demonstrated the potency of a single therapeutic administration of virus.

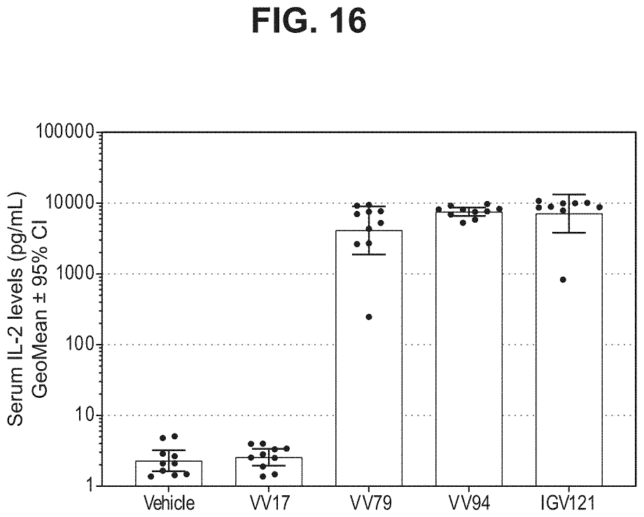

Sera were also collected from MC38 tumor-bearing mice in each test group at 72 hr (day 14) after the IV virus dose for assessment of circulating IL-2 levels. Consistent with other studies where mIL-2v transgene-armed viruses were tested, elevated and statistically significant serum levels of IL-2 were detected in all test groups where mIL-2v transgene-armed WR virus was administered (FIG. 16).

FIG. 13A-13F show results of assessment of virotherapy-induced tumor growth inhibition using single (day 11) IV virus delivery on C57BL/6 female mice implanted SC with MC38 tumor cells. Tumor growth trajectories are shown for each treatment as group averages±95% confidence intervals up through day 32 post-tumor implantation until time of sacrifice (A) or for individual mice in each group until time of sacrifice or study termination (B-F). Test viruses included WR vaccinia viruses containing the A34R K151E mutation and armed with either a Luciferase-2A-GFP reporter (WR.Luc-GFP.A34R-K151E; VV17) (C), mIL-2v only (WR.mIL-2v.A34R-K151E; VV79) (D), mIL-2v with HSV TK.007 in a reverse orientation in the J2R gene locus (WR.mIL-2v.A34R-K151E.HSV TK.007 (J2R_Rev); VV94) (E), and mIL-2v and HSV TK.007 in a forward orientation in the B15R/B17R gene locus (WR.mIL-2v.A34R-K151E.HSV TK.007 (B16R_For); IGV-121) (F). Dashed vertical lines on each graph represent time points when mice received IV injections of virus. The dashed horizontal line on each graph represents the tumor volume threshold used as a criterion to remove animals from the study.

FIG. 14. Statistical comparison of virotherapy-induced tumor growth inhibition using ANCOVA for subcutaneous MC38 tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values 0.05 were observed.

FIG. 15 shows results of survival of MC38 tumor-bearing C57BL/6 female mice following IV treatment with recombinant oncolytic vaccinia viruses on day 11 after SC tumor implantation. Mice were designated daily as deceased upon reaching tumor volume ≥1400 mm³. The point of intersection between each group's curve and the horizontal dashed line indicates the median (50%) survival threshold for the group. P values represent the statistical results of Log-rank test (Mantel-Cox) comparisons between select virus groups.

FIG. 16 shows results of IL-2 levels detected in sera collected from MC38 tumor-bearing C57BL/6 female mice 72 hr (day 14) after IV injection with 5e7 pfu recombinant WR vaccinia viruses. Each symbol represents IL-2 serum levels detected in an individual mouse, while bars represent the group geometric means (N=10/group). Error bars represent 95% confidence intervals.

### Example 7: Single IV Virotherapy Using Recombinant Oncolytic Vaccinia Virus in LLC Tumor-Bearing C57BL/6 Mice (WR Viruses Expressing mIL-2v)

In this set of experiments, C57BL/6 female mice were implanted SC on the right flank with 1e5 LLC tumor cells. Twelve days after tumor cell implantation, mice were randomized based on tumor volume into separate treatment groups (average tumor volume per group ˜50 mm³; N=20/group). On day 14 mice were injected IV with 100 μL of vehicle (30 mM Tris, 10% sucrose, pH8.0) or 100 μL of vehicle containing 5e7 pfu recombinant WR vaccinia virus variants. Tumor-bearing mice were observed daily, and both tumor volume and body weight were measured bi-weekly until mice were humanely sacrificed either due to i) tumor volume surpassing 2000 mm³, ii) ≥20% body weight loss, iii) severely diminished health status or iv) study termination.

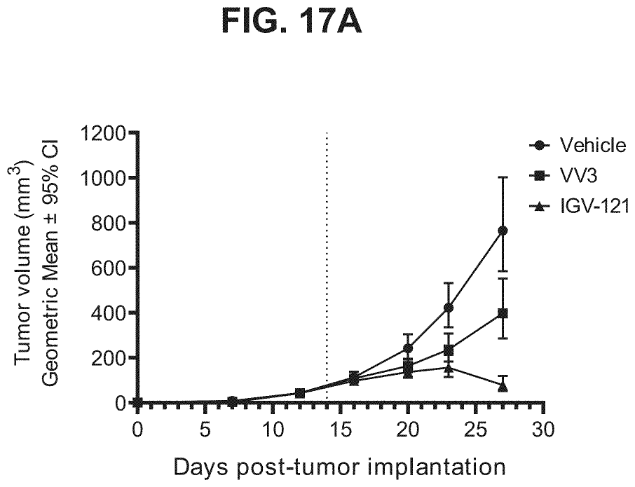

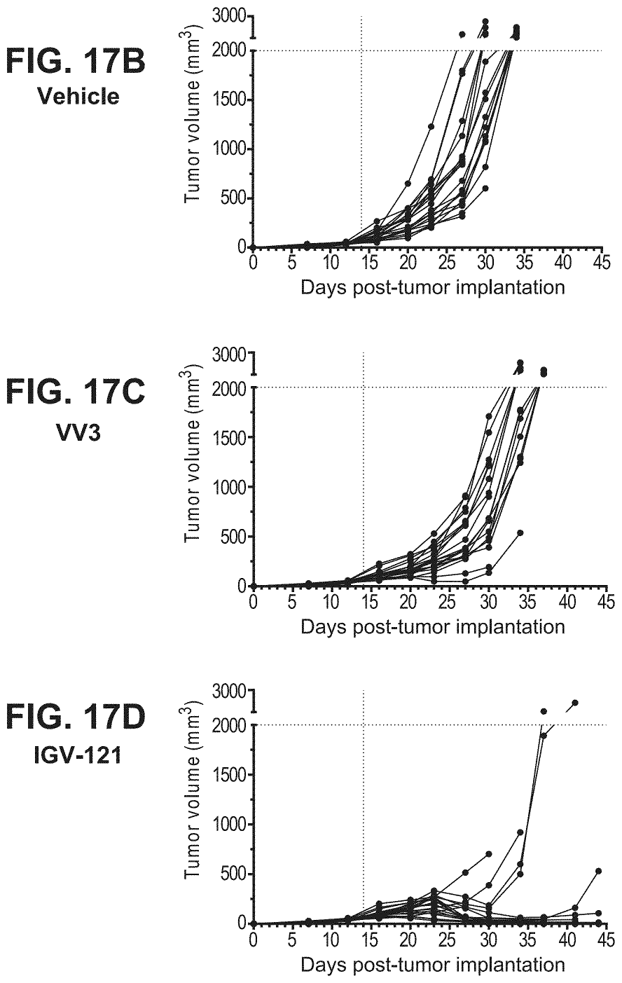

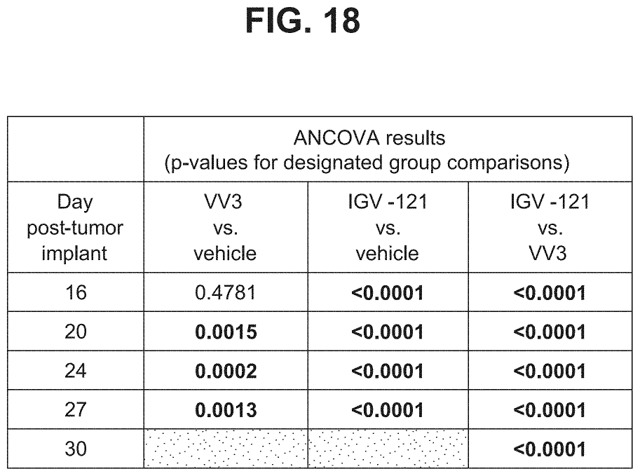

Analysis of tumor growth profiles, shown as group averages for each test virus (FIG. 17A) or as individual mice within each test group (FIG. 17B-17D) demonstrated that IV administration of the mIL-2v transgene-armed WR viruses encoding HSV TK.007 and the A34R-K151E mutation (IGV-121) led to statistically significant inhibition of LLC tumor growth compared to reporter transgene-armed WR virus treatment (FIG. 18, ANCOVA results).

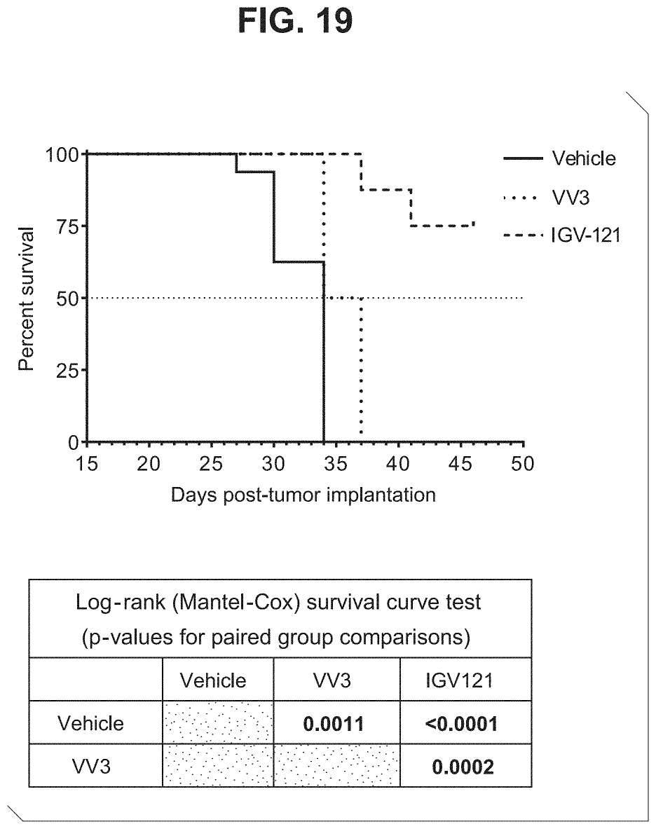

Survival results for the same test viruses showed very similar outcomes as those reported above for tumor growth inhibition. This included statistically superior group survival associated with mIL-2v and HSV TK.007 transgene-armed WR viruses compared to the corresponding Luc-GFP reporter-armed WR viruses (FIG. 19). Overall, IV delivery of the mIL-2v transgene-armed WR virus variant proved to be an effective anti-tumor therapy in the LLC SC tumor model and demonstrated the potency of a single therapeutic administration of virus.

FIG. 17A-17D show results of assessment of virotherapy-induced tumor growth inhibition using single (day 14) IV virus delivery on C57BL/6 female mice implanted SC with LLC tumor cells. Tumor growth trajectories are shown for each treatment as group averages±95% confidence intervals up through day 27 post-tumor implantation until time of sacrifice (A) or for individual mice in each group until time of sacrifice or study termination (B-D). Test viruses included WR vaccinia viruses armed with either a Luciferase-2A-GFP reporter (WR.Luc-GFP; VV3) (C), or mIL-2v and HSV TK.007 in a forward orientation in the B15R/B17R gene locus with the A34R K151E mutation (WR.mIL-2v.A34R-K151E.HSV TK.007 (B16R_For); IGV-121)) (D). Dashed vertical lines on each graph represent time points when mice received IV injections of virus. The dashed horizontal line on each graph represents the tumor volume threshold used as a criterion to remove animals from the study.

FIG. 18 shows results of statistical comparison of virotherapy-induced tumor growth inhibition using ANCOVA for subcutaneous LLC tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values 0.05 were observed.

FIG. 19 shows results of survival of LLC tumor-bearing C57BL/6 female mice following IV treatment with recombinant oncolytic vaccinia viruses on day 14 after SC tumor implantation. Mice were designated daily as deceased upon reaching tumor volume 2000 mm³. The point of intersection between each group's curve and the horizontal dashed line indicates the median (50%) survival threshold for the group. P values represent the statistical results of Log-rank test (Mantel-Cox) comparisons between select virus groups.

### Example 8: Recombinant Oncolytic Vaccinia Virus Activity in MC38 Tumor-Bearing C57BL/6 Mice (Cop Viruses Expressing mIL-2v or hIL-2v)

Female C57BL/6 mice (8-10 weeks old) were implanted subcutaneously (SC) on the right upper rear flank with 5e5 MC38 tumor cells. Ten days after tumor cell implantation, mice were randomized based on tumor volume into separate treatment groups (average tumor volume per group ˜50 mm³; N=20/group). On day 11 post-implantation, tumors were directly injected with 60 μL vehicle (30 mM Tris, 10% sucrose, pH 8.0) or 60 μL vehicle containing either 5e7 or 2e8 plaque forming units (pfu) of recombinant Copenhagen (Cop) vaccinia virus variant. Tumor-bearing mice were observed daily, and both tumor volumes and body weights measured bi-weekly until mice were humanely sacrificed either due to i) tumor volume surpassing 1400 mm³, ii) ≥20% body weight loss, or iii) severely diminished health status. Groups of mice were treated as follows:

Group i) vehicle only;

Group ii) VV7 at 2e8 pfu dose level: Cop vaccinia virus armed with a Luciferase and green fluorescent protein (Luc-2A-GFP) dual reporter cassette;

Group iii) VV91 at 5e7 pfu dose level: Cop vaccinia virus carrying the A34R-K151E substitution, armed with a murine interleukin 2 variant (mIL-2v) transgene, and encoding HSV TK.007 (B16R insertion, forward orientation);

Group iv) VV91 at 2e8 pfu dose level: Cop vaccinia virus carrying the A34R-K151E substitution, armed with a murine interleukin 2 variant (mIL-2v) transgene, and encoding HSV TK.007 (B16R insertion, forward orientation);

Group v) VV102: at 5e7 pfu dose level: Cop vaccinia virus carrying the A34R-K151E substitution, armed with a human interleukin 2 variant (hIL-2v) transgene, and encoding HSV TK.007 (B16R insertion, forward orientation);

Group vi) VV102 at 2e8 pfu dose level: Cop vaccinia virus carrying the A34R-K151E substitution, armed with a human interleukin 2 variant (hIL-2v) transgene, and encoding HSV TK.007 (B16R insertion, forward orientation);

Group vii) VV10 at 5e7 pfu dose level: Cop vaccinia virus armed with mouse GM-CSF and LacZ reporter transgenes; or

Group viii) VV10 at 2e8 pfu dose level: Cop vaccinia virus armed with mouse GM-CSF and LacZ reporter transgenes;

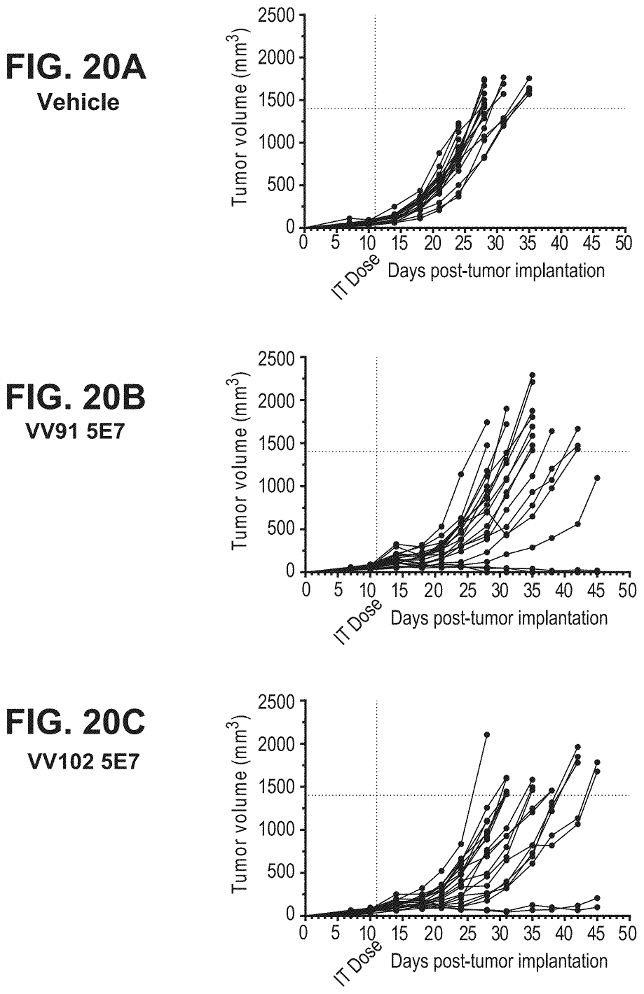

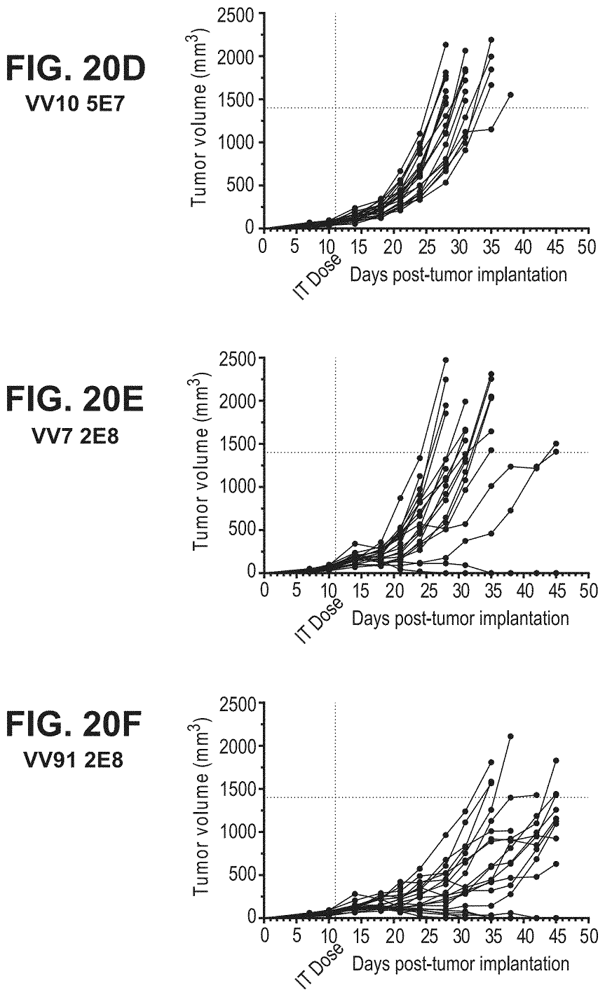

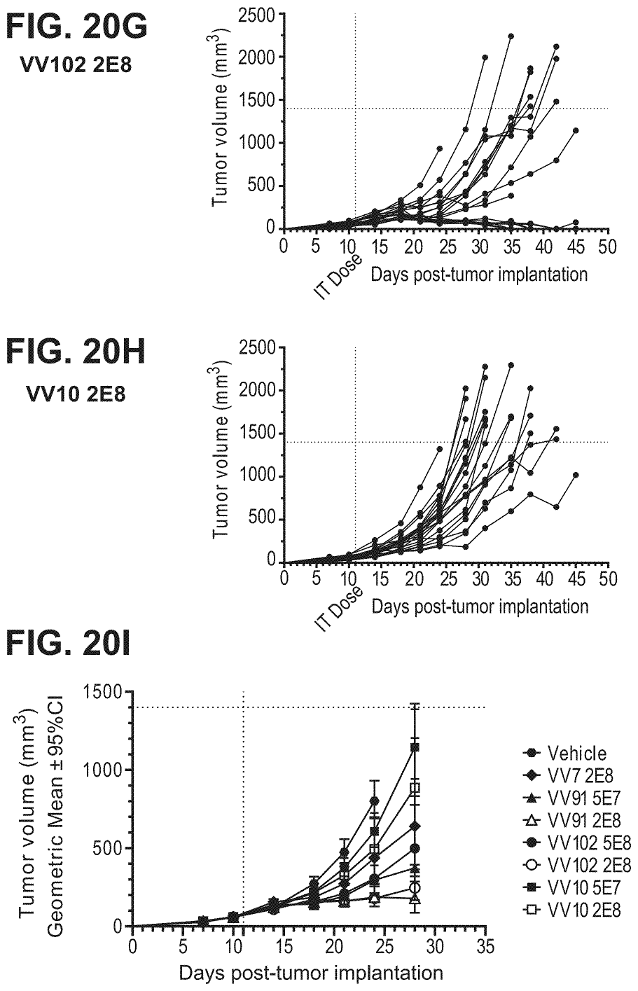

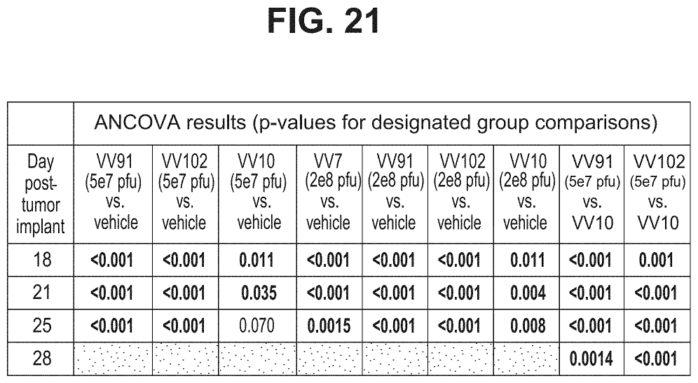

Comparisons between the tumor growth profiles of groups (i)-(viii) (FIG. 20) revealed that all test viruses produced a statistically significant inhibitory effect on tumor growth over multiple consecutive days, and that the mouse and human IL-2v-armed Cop vaccinia viruses (VV91 and VV102, respectively) produced a statistically significant inhibitory effect on tumor growth over multiple consecutive days compared to mouse GM-CSF-armed Cop vaccinia virus (VV10) (FIG. 21, ANCOVA results). There were no statistically significant differences observed when comparing tumor growth inhibition effects induced by VV91 (mIL-2v and HSV TK.007) to VV102, (hIL-2v and HSV TK.007).

FIG. 20A-20I show results of assessment of virotherapy-induced tumor growth inhibition on C57BL/6 female mice implanted SC with MC38 tumor cells. Tumor growth trajectories are shown for individual mice in groups treated with vehicle only (A), Copenhagen vaccinia virus armed with either mIL-2v and HSV TK.007 in a forward orientation in the B16R gene locus (Cop.mIL-2v.A34R-K151E.HSV TK.007 (B16R_For); VV91)) at 5e7 pfu (B), hIL-2v and HSV TK.007 in a forward orientation in the B16R gene locus (Cop.hIL-2v.A34R-K151E.HSV TK.007 (B16R_For); VV102)) at 5e7 pfu (C), mGM-CSF and a LacZ reporter transgene (Cop.mGM-CSF/LacZ; (VV10) at 5e7 pfu (D), a Luciferase-2A-GFP reporter (Cop.Luc-GFP; VV7) at 2e8 pfu (E), mIL-2v and HSV TK.007 in a forward orientation in the B16R gene locus (Cop.mIL-2v.A34R-K151E.HSV TK.007 (B16R_For); VV91)) at 2e8 pfu (F), hIL-2v and HSV TK.007 in a forward orientation in the B16R gene locus (Cop.hIL-2v.A34R-K151E.HSV TK.007 (B16R_For); VV102)) at 2e8 pfu (G), and mGM-CSF and a LacZ reporter transgene (Cop.mGM-CSF/LacZ; (VV10) at 2e8 pfu (H). The dashed vertical line on each graph represents time point when mice received intratumoral injections of vehicle or virus. The dashed horizontal line on each graph represents the tumor volume threshold used as a criterion to remove animals from the study. Average tumor volumes (mm³) for each treatment group are shown through day 28 post-tumor implant (I).

FIG. 21 shows results of statistical comparison of virotherapy-induced tumor growth inhibition using ANCOVA. Tumor volumes for individual mice in each group after vehicle/virus treatment (day 14 to day 28 post-tumor implantation) were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p≤0.05.

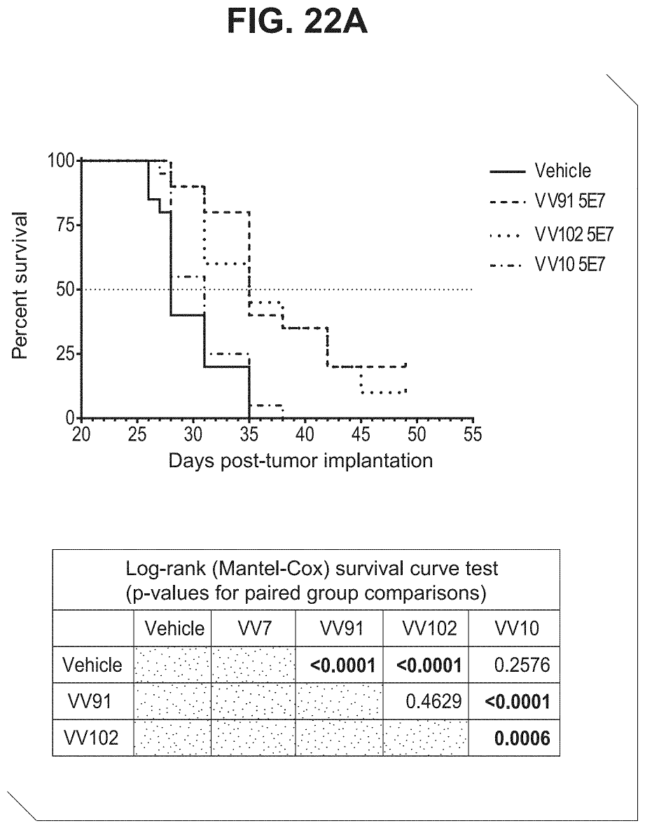

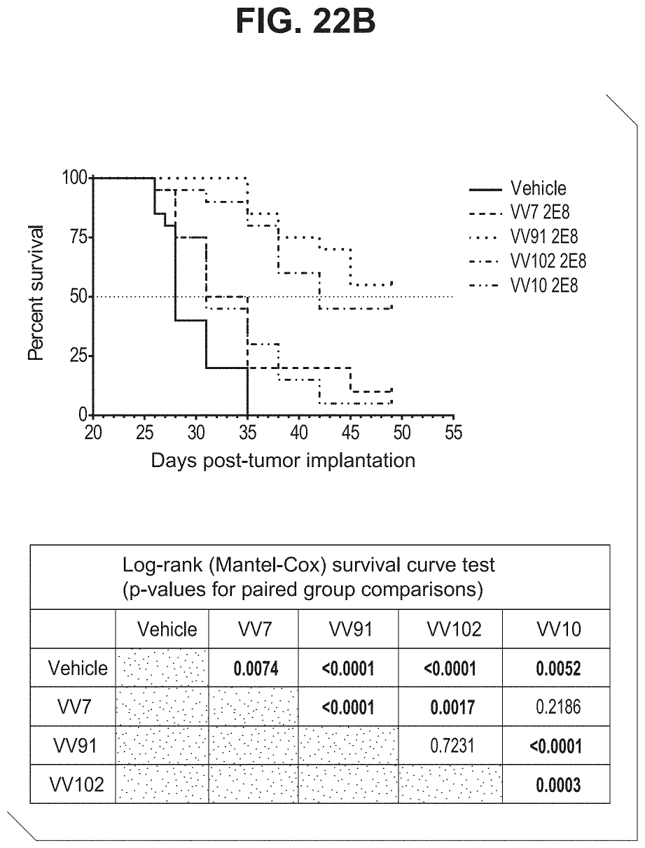

Survival of animals in each treatment group (N=20/group) was also assessed up through day 42 post-tumor implantation (FIG. 22). In this case, mice treated with both VV91 and VV102 showed a statistically significant mean survival advantage over vehicle, VV7, and VV10 treatment groups (see table in FIG. 22 for P values from Log rank/Mantel-Cox test).

FIG. 22A-22B show results of survival of MC38 tumor-implanted C57BL/6 female mice following treatment with vehicle or virus on day 11 after implantation. Mice were designated daily as deceased upon reaching tumor volume 1400 mm³. The point of intersection between each group's curve and the horizontal dashed line indicates the median (50%) survival threshold for group. (A) shows groups dosed with 5e7 pfu virus. (B) shows groups dosed with virus at 2e8 pfu.

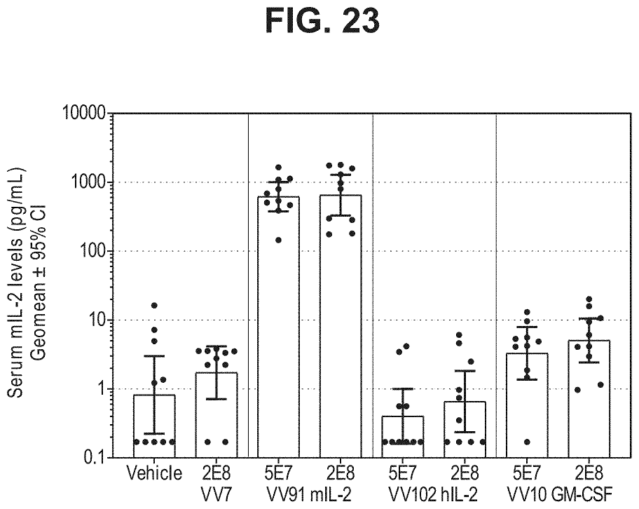

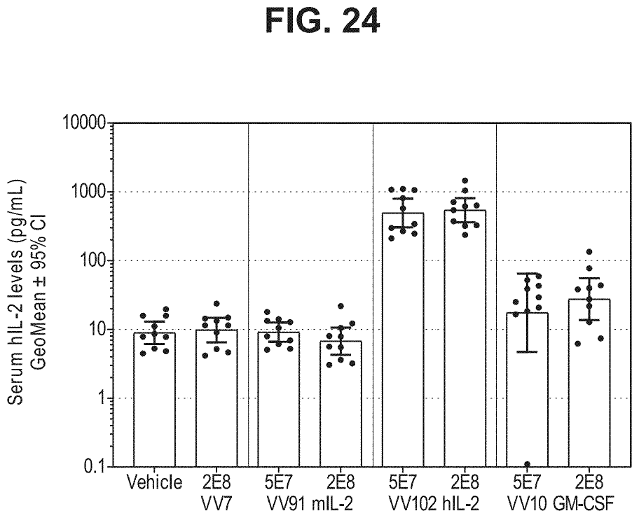

In addition to monitoring tumor growth inhibition and survival, sera were collected from tumor-bearing mice 24 hr after injection with vehicle or recombinant Cop vaccinia virus to assess circulating IL-2 levels. Circulating mouse IL-2 and human IL-2 levels in sera collected from each treatment group 24 hr after receiving intratumoral injections were quantified by ELISA (FIG. 23 and FIG. 24, respectively). Measurable levels of IL-2 were detected in the serum from most animals treated with the IL-2v-armed Cop vaccinia virus variants (VV91, and VV102), while background levels of IL-2 were seen in any animal from the vehicle or other Cop vaccinia virus (VV7 and, VV10) groups. Notably, significantly elevated levels of mouse IL-2 ere only detected in serum of mice receiving mIL-2v expressing virus (VV91) and significantly elevated levels of human IL-2 were only detected in serum of mice receiving hIL-2v expressing virus (VV102). Thus, elevated levels seen in the sera of mice treated with the IL-2v-armed Cop vaccinia virus should be indicative of transgene-mediated expression following intratumoral injection.

FIG. 23 shows results of mouse IL-2 levels detected in sera collected from MC38 tumor-bearing C57BL/6 female mice 24 hr after intratumoral injection with vehicle or recombinant Cop vaccinia viruses. Each symbol represents the calculated IL-2 serum levels for an individual mouse, while bars represent group geometric mean (N=10/group). Error bars represent 95% confidence intervals.

FIG. 24 shows results of human IL-2 levels detected in sera collected from MC38 tumor-bearing C57BL/6 female mice 24 hr after intratumoral injection with vehicle or recombinant Cop vaccinia viruses. Each symbol represents the calculated IL-2 serum levels for an individual mouse, while bars represent group geometric mean (N=9/group). Error bars represent 95% confidence intervals.

### Example 9: Recombinant Oncolytic Vaccinia Virus Activity in HCT-116 Tumor-Bearing Nude Mice (Cop Viruses Expressing hIL-2v)

Nude female mice were implanted SC on the right flank with 5e6 HCT-116 tumor cells. Eight days after tumor cell implantation, mice were randomized based on tumor volume into separate treatment groups (average tumor volume per group ˜50 mm³; N=20/group). On day 9 post-tumor cell implantation, mice were injected IV with 100 μL of vehicle only or vehicle containing a suboptimal dose (3e5 pfu) of recombinant oncolytic Cop vaccinia virus. Tumor-bearing mice were observed daily, and both tumor volume and body weight were measured bi-weekly until mice were humanely sacrificed either due to i) tumor volume surpassing 1400 mm³, ii) 20% body weight loss, iii) severely diminished health status, or iv) study termination. Groups of mice were treated as follows:

Group i) vehicle only;

Group ii) VV90: Cop vaccinia virus carrying the A34R-K151E mutation (amino acid substitution) with no transgene inserted into the deleted J2R gene region;

Group iii) VV27: Cop vaccinia virus carrying the A34R-K151E substitution and armed with a murine interleukin 2 variant (mIL-2v) transgene (VV27);

Group iv) VV91: Cop vaccinia virus carrying the A34R-K151E substitution, armed with a murine interleukin 2 variant (mIL-2v) transgene, and encoding HSV TK.007 (B16R insertion, forward orientation);

Group v) VV93: Cop vaccinia virus carrying the A34R-K151E substitution, armed with a murine interleukin 2 variant (mIL-2v) transgene, and encoding HSV TK.007 (J2R insertion, reverse orientation); or

Group vi) VV96: Cop vaccinia virus carrying the A34R-K151E substitution, armed with a murine interleukin 2 variant (mIL-2v) transgene, and encoding HSV

TK.007 (B16R insertion, reverse orientation).

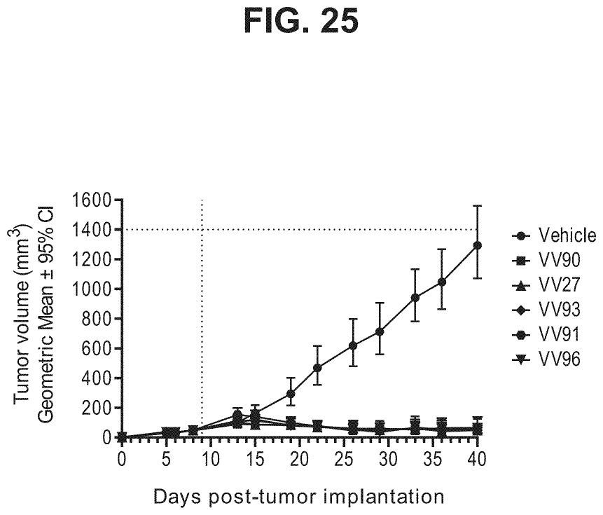

Comparisons between the tumor growth profiles of groups (i)-(vi) (FIG. 25) revealed that all test viruses produced a statistically significant inhibitory effect on tumor growth over multiple consecutive days in the human xenograft tumors.

FIG. 25 shows results of assessment of virotherapy-induced tumor growth inhibition on Nude female mice implanted SC with HCT-116 tumor cells. Average tumor volumes (mm³) for each treatment group are shown through day 40 post-tumor implant. The dashed vertical line on each graph represents time point when mice received intratumoral injections of vehicle or virus. The dashed horizontal line on each graph represents the tumor volume threshold used as a criterion to remove animals from the study.

### Example 10: Functional Assessment of hIL-2gv and hIL-2v Protein Produced from Cells Infected with Transgene Armed WR Vaccinia Virus

IL-2 binding to the IL-2 receptor complex results in phosphorylation of the signaling molecule, STAT5. Thus, phosphorylation of STAT5 can be used to measure IL-2 receptor signaling. To collect transgene produced by the vaccinia viruses, HeLa cells were infected with the indicated virus at a MOI=3 in a T-150 flask for 24 hours. After the incubation, the supernatants were collected and concentrated, and the IL-2 level in the concentrated supernatant was determined by MSD assay and normalized in the pSTAT5 assay. To assess IL2 transgene bioactivity, splenocytes from naïve C57BL/6 female mice were isolated, plated at 1e6 cells/well in a round bottom 96-well plate, and incubated with virus isolated IL-2, IL-2 glycovariant, or IL-2 variant for 15 minutes. The cells were fixed and permeabilized, stained with anti-CD3, anti-CD4, anti-CD8, anti-CD25, anti-Foxp3, anti-NKp46, and anti-pSTAT5 antibodies and acquired on an LSR Fortessa flow cytometer. The median fluorescence intensity of pSTAT5 was analyzed in specific cell populations using FlowJo software. The IL-2 glycovariants (i.e., IL-2gv1 and IL-2gv2) encoded by the recombinant vaccinia virus showed reduced activity on Treg cells (CD3+CD4+CD25+ Foxp3+) when compared to wild-type IL-2 as indicated by reduced concentration potency at inducing pSTAT5. In contrast, the IL-2 variant (IL-2v) and IL-2 glycovariants demonstrated similar signaling concentration potency as wild-type IL-2 in both CD8+ T cells and NK cells. Taken together, these data are consistent with the expected ability of hIL-2 glycovariant and hIL-2 variant produced in human cells to be comparable to wild-type hIL-2 at stimulating cells expressing the intermediate-affinity IL-2R, but only weakly active on cells expressing the high-affinity IL-2Rα (aka CD25).

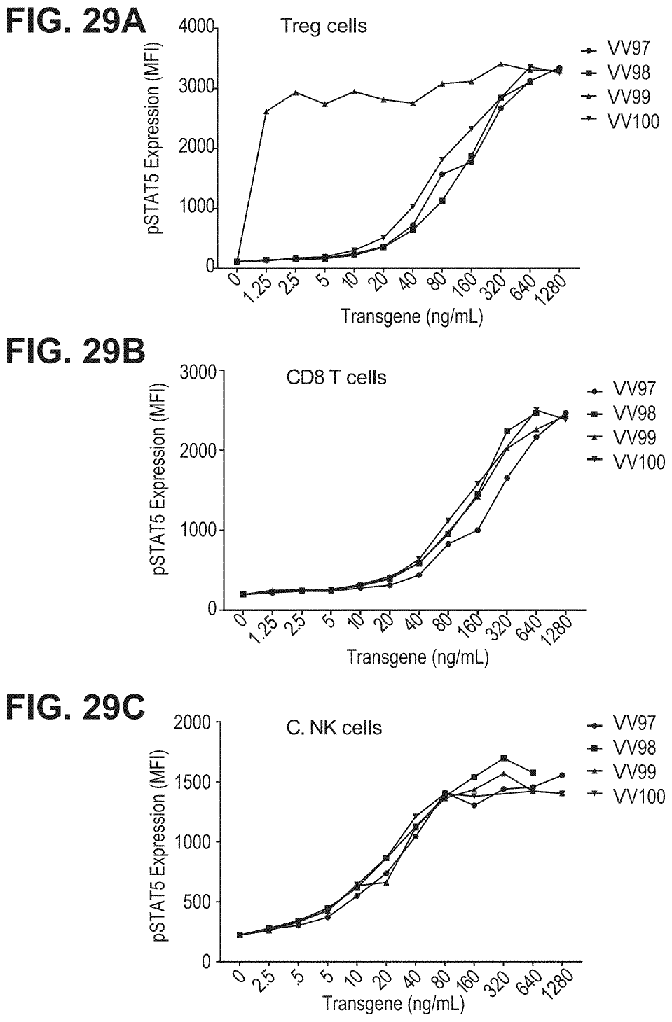

FIG. 29A-29C show results of assessment of STAT5 phosphorylation in murine splenocytes incubated with IL-2 variant transgenes expressed by recombinant WR vaccinia viruses. Comparison of pSTAT5 induction in subsets of murine splenocytes incubated with either hIL-2, hIL-2 variant, or hIL-2 glycovariants. IL-2 functionality was assessed using measurement of intracellular pSTAT5 levels as a readout of IL-2R-mediated signaling. Splenocytes were additionally stained with antibodies to cell surface markers (CD3, CD4, CD8, CD25, and NKp46) and an intracellular protein (FoxP3) to delineate various subsets of murine lymphocytes expressing different IL2R complexes. Graphs show changes in median fluorescence intensity (MFI) values of intracellular staining of pSTAT5 (y-axis) in response to increasing treatment concentrations of hIL-2, hIL-2 variant, or hIL-2 glycovariant protein secreted by the indicated viruses (x-axis). Abbreviations: pSTAT5=phosphorylated signal transducer and activator of transcription 5; MFI=median fluorescence intensity; Treg=CD3+CD4+CD25+Foxp3+T regulatory cells.

### Example 11: Recombinant Oncolytic Vaccinia Virus Activity in MC38 Tumor-Bearing C57BL/6 Mice Following IV Administration (WR Viruses Expressing hIL-2, hIL-2v, hIL-2gv1, hIL-2gv2)

C57BL/6 female mice were implanted SC on the left flank with 5e5 MC38 tumor cells. Ten days after tumor cell implantation, mice were randomized based on tumor volume into separate treatment groups (average tumor volume per group ˜60 mm³; N=20/group). On day 11 post-tumor cell implantation, mice were injected IV with 100 μL of vehicle (30 mM Tris, 10% sucrose, pH8.0) or 100 μL of vehicle containing 5e7 pfu recombinant WR vaccinia virus. Tumor-bearing mice were observed daily, and both tumor volume and body weight were measured bi-weekly until mice were humanely sacrificed either due to i) tumor volume surpassing 1400 mm³, ii) 20% body weight loss, iii) severely diminished health status or iv) study termination.

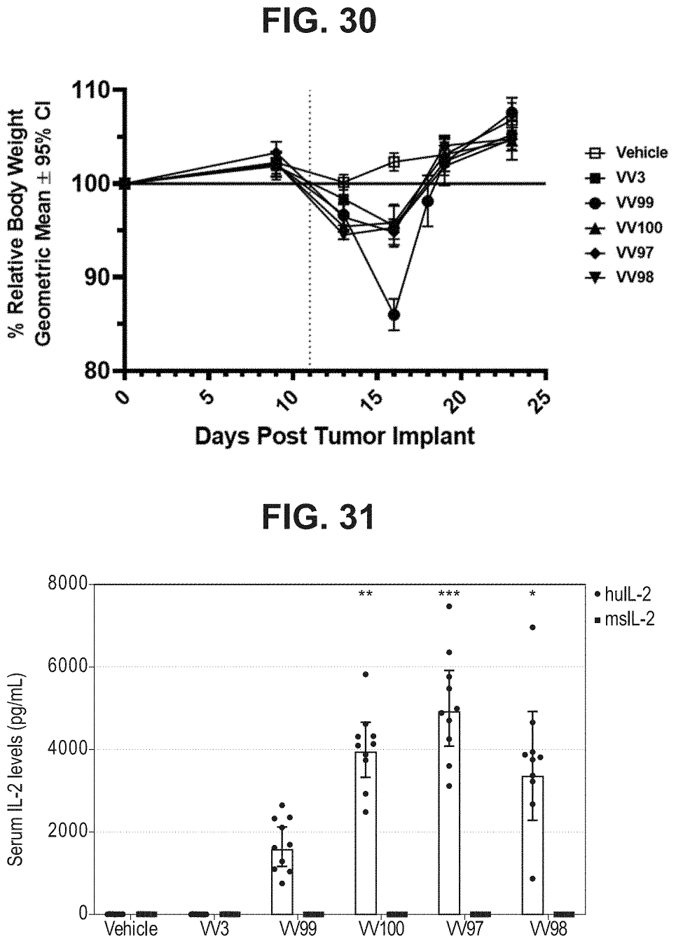

Body weight analysis indicated that wild-type IL-2 transgene-armed WR virus was associated with greater loss of body weight compared to animals that received any other variant (FIG. 30.)

FIG. 30 shows results of body weights of MC38 tumor-implanted C57BL/6 female mice following treatment with vehicle or virus on day 11 after implantation. Body weights are shown in % based on the individual body weights at treatment start. Each treatment is shown as group geometric means±95% confidence intervals up through day 24 post-tumor implantation. Animals with more than 20% body weight loss in comparison to their initial body weight were euthanized for humane reasons. Test viruses included WR vaccinia viruses armed with either a Luciferase-2A-GFP reporter (WR.Luc-GFP (VV3) WT IL-2, IL-2v, IL-2gv1 or IL-2gv2. Dashed vertical lines on each graph represent time points when mice received IV injections of virus. The horizontal line on the graph represents the 100% body weight baseline representing the initial body weight of each mouse.

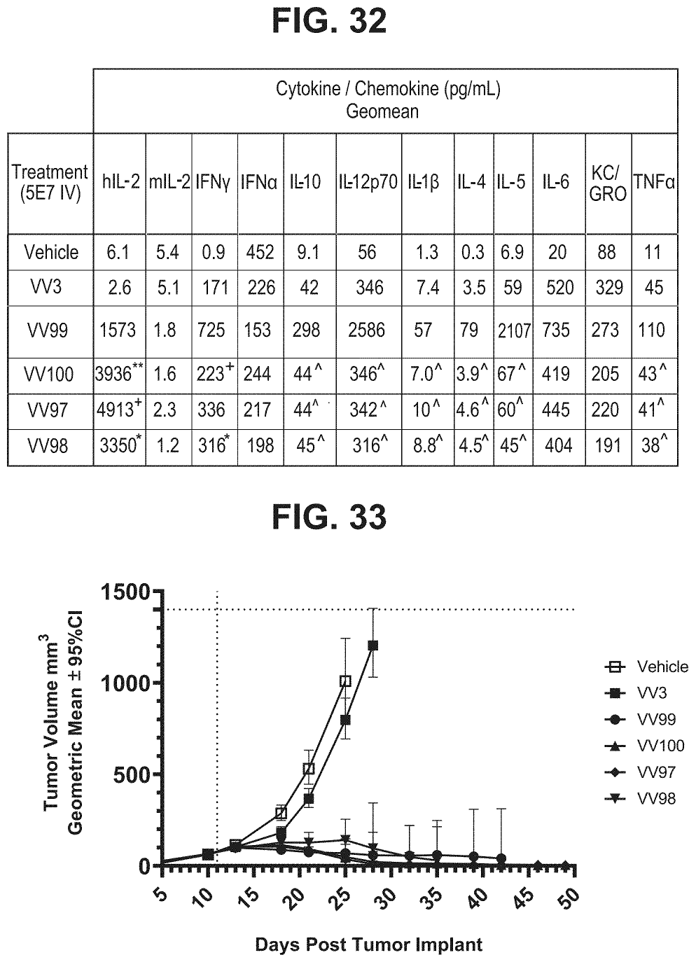

Sera were also collected from MC38 tumor-bearing mice in each test group at 72 hr (day 14 post tumor implant) after the IV virus dose for assessment of circulating IL-2 and inflammatory cytokine levels. Consistent with other studies where IL-2 transgene-armed viruses were tested, elevated and statistically significant serum levels of IL-2 were detected in all test groups where IL-2 transgene-armed WR virus was administered (FIG. 31). Furthermore, mice receiving hIL-2gv armed oncolytic viruses had statistically significant elevated serum levels of IL-2 compared to animals receiving wildtype hIL-2 armed oncolytic virus. Analysis of inflammatory cytokines revealed that IV administration of hIL-2, but not hIL-2gv transgene armed WR vaccinia virus caused a significant elevation in several pro-inflammatory cytokines, including IFNγ, IL-12p70, IL-1β, TNFα, IL-4, IL-5, and IL-10. (FIG. 32. TABLE 3)

FIG. 31 shows results of IL-2 levels detected in sera collected from MC38 tumor-bearing C57BL/6 female mice 72 hr (day 14) after IV injection with 5e7 pfu recombinant WR vaccinia viruses. Each symbol represents IL-2 serum levels detected in an individual mouse, while bars represent the group geometric means (N=10/group). Error bars represent 95% confidence intervals. Statistics were performed using a One-way Anova test with a Tukey's post-hoc multiple group comparison test as compared to VV99 with *=p<0.05; **=p<0.01 and ***=p<0.001

FIG. 32 (Table 3) shows show results of inflammatory cytokine levels detected in sera collected from MC38 tumor-bearing C57BL/6 female mice 72 hr (day 14) after IV injection with 5e7 pfu recombinant WR vaccinia viruses. Serum cytokine levels were measured 72 hr following intravenous administration of MC38 tumor-bearing C57BL/6 mice. Statistical comparison between cytokine levels detected as compared to VV99-treated animals were performed using a one-way ANOVA with a Tukey's post-hoc multiple group comparison test. Each column shows geometric mean cytokine levels (N=10/test group) for the designated cytokine. *=p<0.05; **=p<0.01; +=p<0.001; \hat{ }=p<0.0001

Analysis of tumor growth profiles, shown as group averages for each test virus (FIG. 33) revealed an important finding. IV administration of all IL-2 transgene-armed WR viruses led to statistically significant inhibition of MC38 tumor growth compared to vehicle and reporter transgene-armed WR virus (VV3) treatment. All variants significantly reduced tumor growth compared to vehicle control or VV3. Some time points also revealed statistically significant differences between IL-2 variant and glycovariant containing viruses compared to wild-type IL-2 however the most striking finding was that all viral variants that contained any form of IL-2 resulted in transgene-mediated reductions in tumor growth. (Table 4, ANCOVA results).

FIG. 33 shows results of assessment of virotherapy-induced tumor growth inhibition using single (administered on day 11) IV virus delivery on C57BL/6 female mice implanted SC with MC38 tumor cells. Tumor growth curves are shown for each treatment as group geometric means±95% confidence intervals up through day 49 post-tumor implantation at which time the study was terminated. Once 15% of animals were euthanized due to tumor burden reaching 1400 mm3, that group no longer reported geometric mean data. Test viruses included WR vaccinia viruses armed with either a Luciferase-2A-GFP reporter (WR.Luc-GFP (VV3), WT IL-2, IL-2v, IL-2gv1 or IL-2gv2. Dashed vertical lines on each graph represent time points when mice received IV injections of virus. The dashed horizontal line on each graph represents the tumor volume threshold used as a criterion to remove animals from the study.

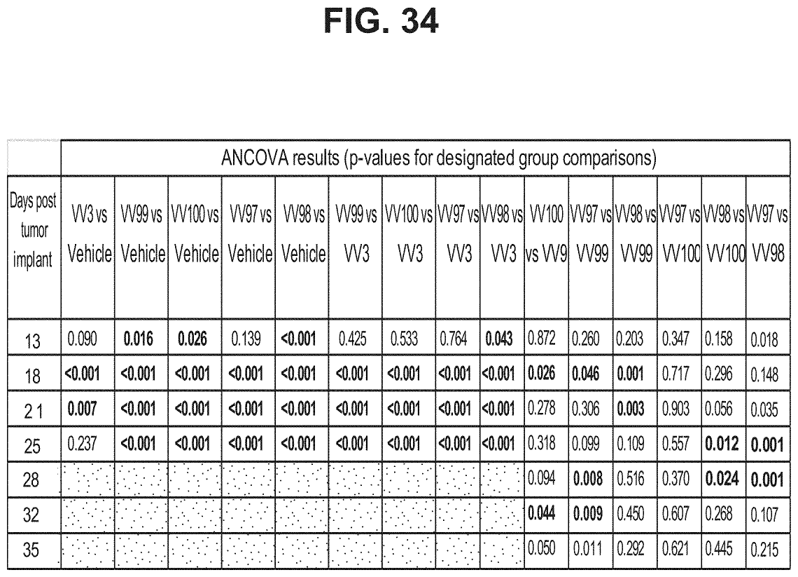

FIG. 34 (Table 4) shows results of statistical comparison of virotherapy-induced tumor growth inhibition using ANCOVA for subcutaneous MC38 tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values 0.05 were observed.

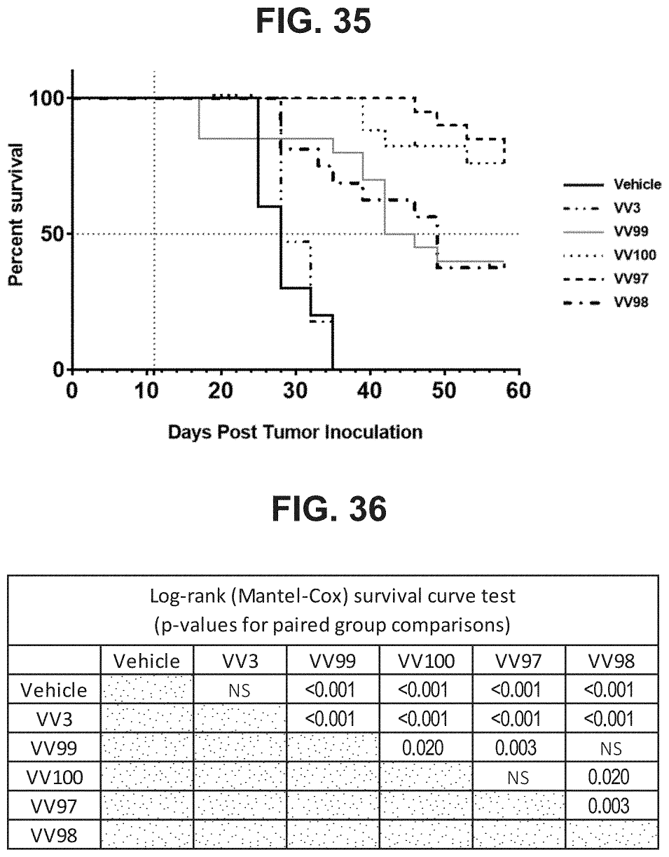

Survival results for the same test viruses showed WT IL-2 had a lower threshold of tolerability than variants as a greater number of animals expired due to morbidities unrelated to tumor burden and experienced a shorter median survival compared to variants bearing the IL-2 variant or glycovariant (FIG. 35). This included statistically superior group survival associated with IL-2v/gv transgene-armed WR viruses compared to the corresponding Luc-GFP reporter-armed WR virus (FIG. 36, Table 5). Overall, IV delivery of IL-2v or IL-2gv transgene-armed WR virus variants proved to be an effective anti-tumor therapy in the MC38 SC tumor model and demonstrated the potency of a single therapeutic administration of virus and less toxicity than wild type IL-2.

FIG. 35 show results of survival of MC38 tumor-bearing C57BL/6 female mice following IV treatment with recombinant oncolytic vaccinia viruses on day 11 after SC tumor implantation. Mice were monitored daily and were designated as deceased upon reaching tumor volume 1400 mm³, if the animal lost >20% body weight, or was determined to be moribund based on clinical observations. The point of intersection between each group's curve and the horizontal dashed line indicates the median (50%) survival threshold for the group.

FIG. 36 (Table 5) shows results of statistical comparison of survival following virotherapy in the subcutaneous MC38 tumor model study. Survival data from FIG. 35 was analyzed by Log-rank test (Mantel-Cox). P values represent the statistical results of Log-rank test (Mantel-Cox) comparisons between select virus groups.

### Example 12: Recombinant Oncolytic Vaccinia Virus Activity in HCT-116 Tumor-Bearing Nude Mice (Cop Viruses Expressing hIL-2gv, hIL-2v and Wyeth Viruses Expressing hGM-CSF/LacZ)

Nude female mice were implanted SC on the right flank with 5e6 HCT-116 tumor cells. Twelve days after tumor cell implantation, mice were randomized based on tumor volume into separate treatment groups (average tumor volume per group ˜150 mm³; N=16/group). On day 13 post-tumor cell implantation, mice were injected IV with 100 μL of vehicle only or vehicle containing a 3e6 pfu of recombinant oncolytic Cop vaccinia virus. Tumor-bearing mice were observed daily, and both tumor volume and body weight were measured bi-weekly until mice were humanely sacrificed either due to i) tumor volume surpassing 1400 mm³, ii) 20% body weight loss, iii) severely diminished health status, or iv) study termination. Groups of mice were treated as follows: Group i) vehicle only; Group ii) VV7: Cop vaccinia virus carrying luc-2A-GFP transgene inserted into the deleted J2R gene region; Group iii) VV102: Cop vaccinia virus carrying hIL2v transgene inserted into the deleted J2R gene region, with K151E mutation and HSV-TK.007; Group iv) VV75: Cop vaccinia virus carrying hIL2v transgene inserted into the deleted J2R gene region, with K151E mutation; Group v) VV08: Wyeth vaccinia virus carrying luc-2A-GFP transgene inserted into the deleted J2R gene region; Group vi) VV12: Wyeth vaccinia virus carrying hGM-CSF transgene inserted into the deleted J2R gene region; or Group vii) VV110: Cop vaccinia virus carrying hIL2gv transgene inserted into the deleted J2R gene region, with K151E mutation and HSV-TK.007.

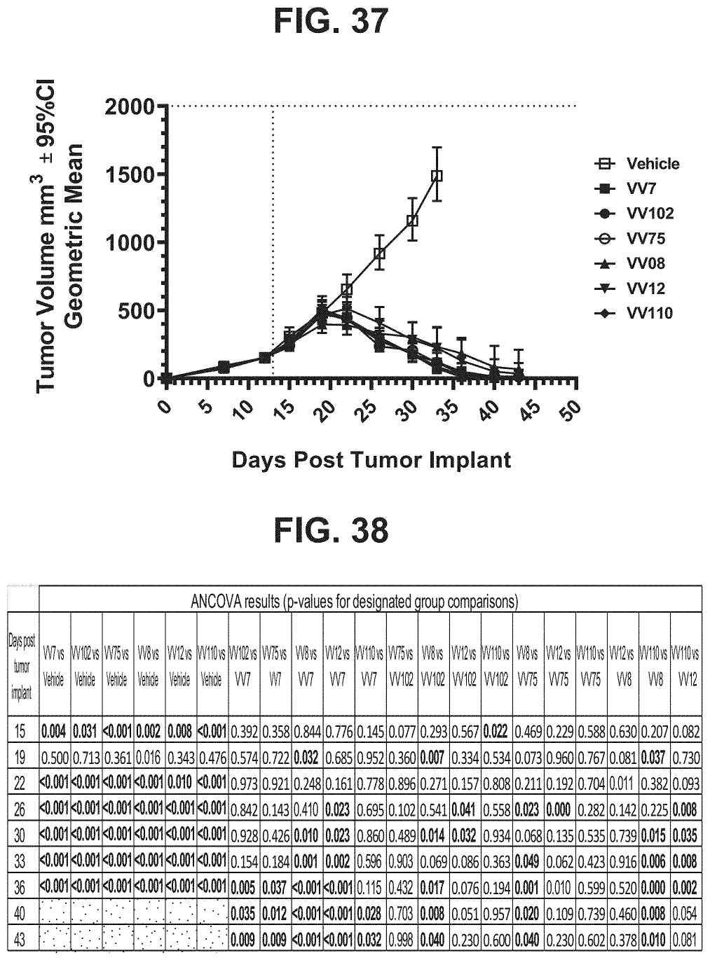

Comparisons between the tumor growth profiles of groups (i)-(vii) (FIG. 37) revealed that all test viruses had an produced an inhibitory effect on tumor growth over multiple consecutive days in the HCT-116 human xenograft model. Statistical significance was achieved for different comparisons as shown in FIG. 38, Table 6.

FIG. 37 shows results of assessment of virotherapy-induced tumor growth inhibition on Nude female mice implanted SC with HCT-116 tumor cells. Average tumor volumes (mm³) for each treatment group are shown through day 43 post-tumor implant. The dashed vertical line on each graph represents time point when mice received IV injections of vehicle or virus. The dashed horizontal line on the graph represents the tumor volume threshold used as a criterion to remove animals from the study.

FIG. 38 (Table 6) show results of statistical comparison of virotherapy-induced tumor growth inhibition using ANCOVA on subcutaneous HCT-116 tumors in Nude mice. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values 0.05 were observed.

Nude mice bearing HCT-116 tumors and treated IV with viruses as described above were monitored for survival. Tumor reaching 2000 mm3 was defined as euthanasia criteria and animals were monitored daily for 45 days.

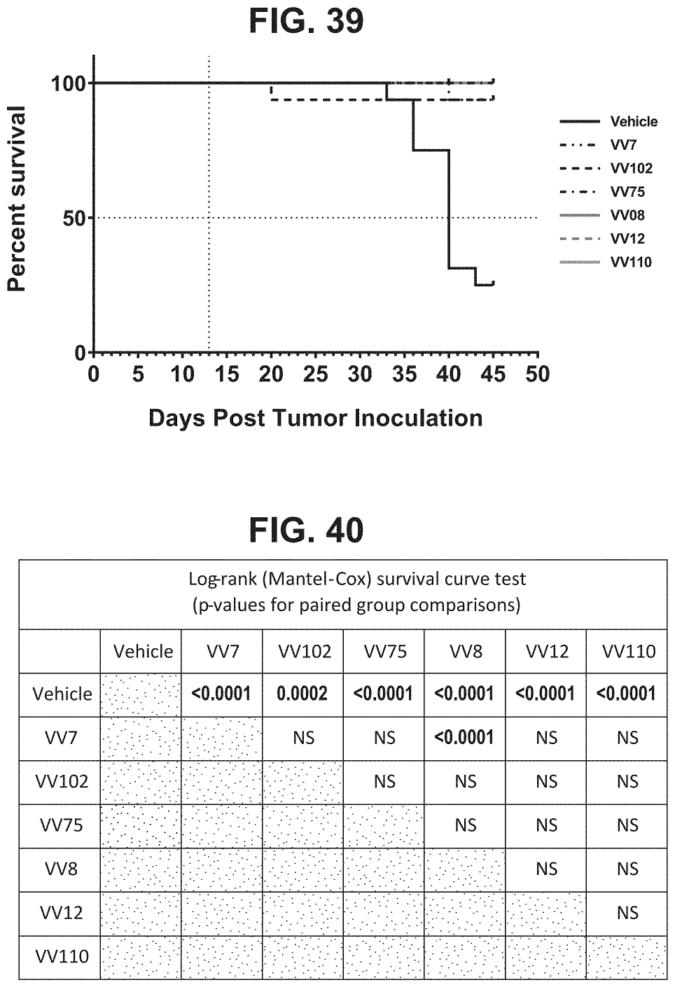

FIG. 39 shows results of assessment of virotherapy-induced survival on Nude female mice implanted SC with HCT-116 tumor cells. Euthanasia was performed once tumors reached 2000 mm3. The dashed vertical line on each graph represents time point when mice received IV injections of vehicle or virus (3E6 PFU). The dashed horizontal line on the graph represents 50 percent survival, or median survival.

FIG. 40 (Table 7) show results of statistical comparison of virotherapy-induced survival in Nude female mice implanted SC with HCT-116 tumor cells. Survival was monitored and then analyzed by Log-rank test (Mantel-Cox). P values are listed for each group comparison.

### Example 13: Recombinant Oncolytic Vaccinia Virus Activity in MC38 Tumor-Bearing C57BL/6 Mice Following IV Administration (WR Viruses Expressing hIL-2v, hIL-2gv1, mIL-2v)

C57BL/6 female mice were implanted SC on the left flank with 5e5 MC38 tumor cells. Fifteen days after tumor cell implantation, mice were randomized based on tumor volume into separate treatment groups (average tumor volume per group ˜100 mm³; N=20/group). On day 16 post-tumor cell implantation, mice were injected IV with 100 μL of vehicle (30 mM Tris, 10% sucrose, pH8.0) or 100 μL of vehicle containing 5e7 pfu recombinant WR vaccinia virus. Tumor-bearing mice were observed daily, and both tumor volume and body weight were measured bi-weekly until mice were humanely sacrificed either due to i) tumor volume surpassing 1400 mm³, ii) 20% body weight loss, iii) severely diminished health status or iv) study termination.

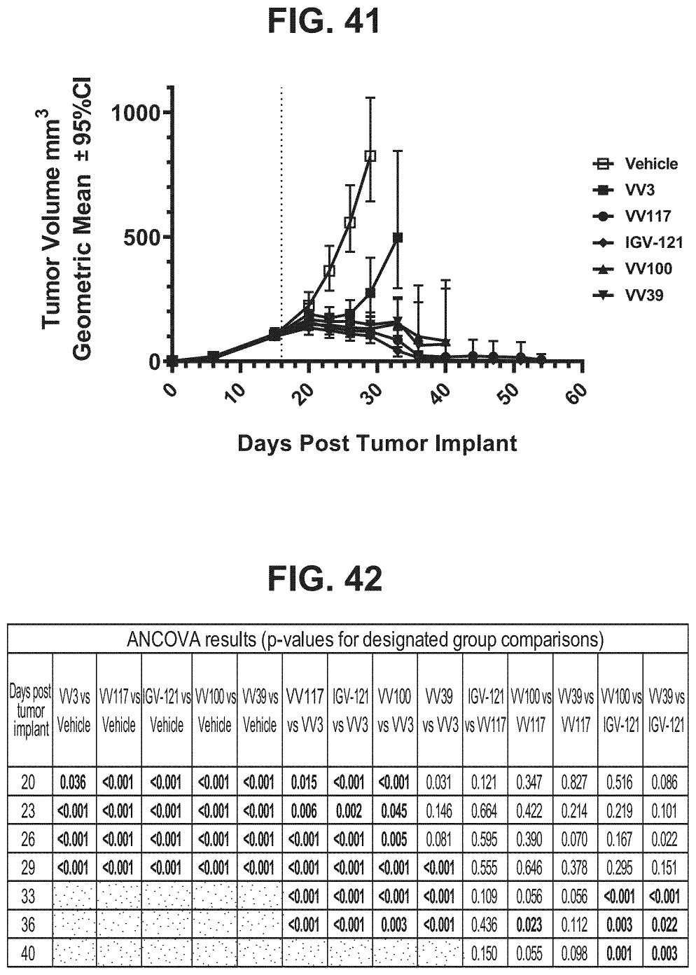

Analysis of tumor growth profiles, shown as group averages for each test virus (FIG. 41) revealed an important finding. IV administration of all IL-2 transgene-armed WR viruses led to statistically significant inhibition of MC38 tumor growth compared to vehicle and reporter transgene-armed WR virus (VV3) treatment. Addition of the K151E mutation & HSV TK.007 transgene further improved tumor growth inhibition. There was no statistically significant difference between tumor growth inhibition induced by VV117 and IGV-121, however there was a statistically significant difference detected between VV117 and VV100 and between IGV-121 and VV39 (FIG. 42, Table 8, ANCOVA results).

FIG. 41 shows results of assessment of virotherapy-induced tumor growth inhibition using single (day 16) IV virus delivery on C57BL/6 female mice implanted SC with MC38 tumor cells. Tumor growth trajectories are shown for each treatment as group averages±95% confidence intervals up through day 55 post-tumor implantation until time of sacrifice. Test viruses included WR vaccinia viruses armed with either a Luciferase-2A-GFP reporter (WR.Luc-GFP (VV3)), hIL-2gv1 (WR.hIL-2gv1.HSV TK.007.A34K151E (VV117, IGV-121), hIL-2v (VV100) or mIL-2v (VV3)). Dashed vertical lines on each graph represent time points when mice received IV injections of virus. The dashed horizontal line on each graph represents the tumor volume threshold used as a criterion to remove animals from the study.

FIG. 42 (Table 8) show results of statistical comparison of virotherapy-induced tumor growth inhibition using ANCOVA for subcutaneous MC38 tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values ≤0.05 were observed.

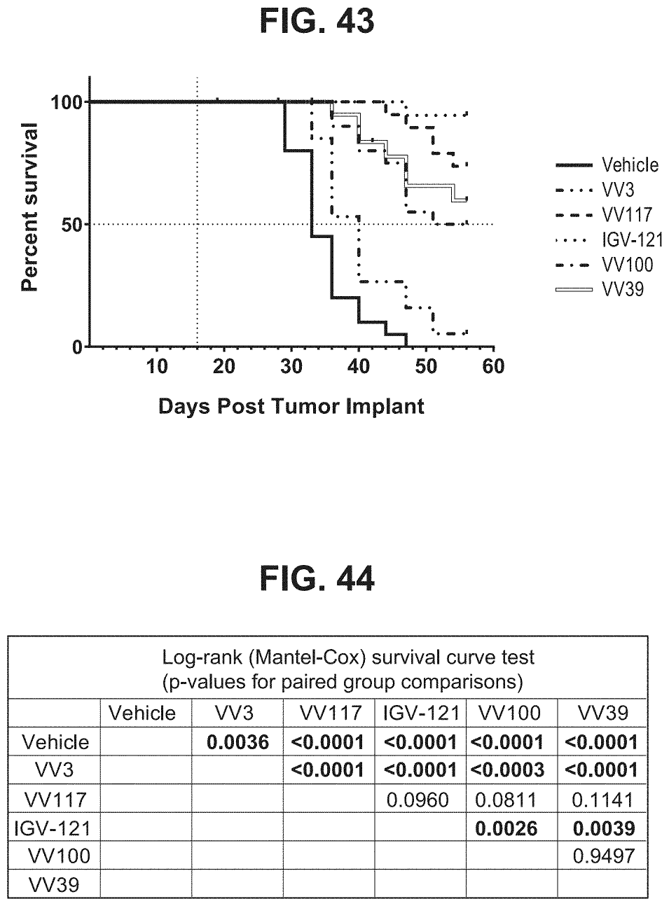

Survival results for the same test viruses showed very similar outcomes as those reported above for tumor growth inhibition (FIG. 43). This included statistically superior group survival associated with IL-2v/gv transgene-armed WR viruses compared to the corresponding Luc-GFP reporter-armed WR virus (FIG. 44, Table 9). Overall, IV delivery of IL-2v transgene-armed WR virus variants proved to be an effective anti-tumor therapy in the MC38 SC tumor model and demonstrated the potency of a single therapeutic administration of virus.

FIG. 43 shows results of survival of MC38 tumor-bearing C57BL/6 female mice following IV treatment with recombinant oncolytic vaccinia viruses on day 16 after SC tumor implantation. Mice were designated daily as deceased upon reaching tumor volume ≥1400 mm³. The point of intersection between each group's curve and the horizontal dashed line indicates the median (50%) survival threshold for the group. P values represent the statistical results of Log-rank test (Mantel-Cox) comparisons between select virus groups.

FIG. 44 (Table 9) show results of statistical comparison of virotherapy-induced survival. Survival was monitored and then analyzed by Log-rank test (Mantel-Cox). P values are listed for each group comparison.

### Example 14: Recombinant Oncolytic Vaccinia Virus Activity in B16 Tumor-Bearing C57BL/6 Mice Following IV Administration (WR Viruses Expressing hIL-2gv1)

C57BL/6 female mice were implanted SC on the right flank with 2.5e5 B16F10 tumor cells. Seventeen days after tumor cell implantation, mice were randomized based on tumor volume into separate treatment groups (average tumor volume per group ˜100 mm³; N=20/group). On day 18 post-tumor cell implantation, mice were injected IV with 100 μL of vehicle (30 mM Tris, 10% sucrose, pH8.0) or 100 μL of vehicle containing 5e7 pfu recombinant WR vaccinia virus. On days 21, 24, 27, 31, 34 and 38 post-tumor cell implantation, mice were injected SC with 100 uL of antibody formulation (2 mg/mL, anti PD-1 or IgG1 Isotype). Tumor-bearing mice were observed daily, and both tumor volume and body weight were measured bi-weekly until mice were humanely sacrificed either due to i) tumor volume surpassing 1400 mm³, ii) ≥20% body weight loss, iii) severely diminished health status or iv) study termination.

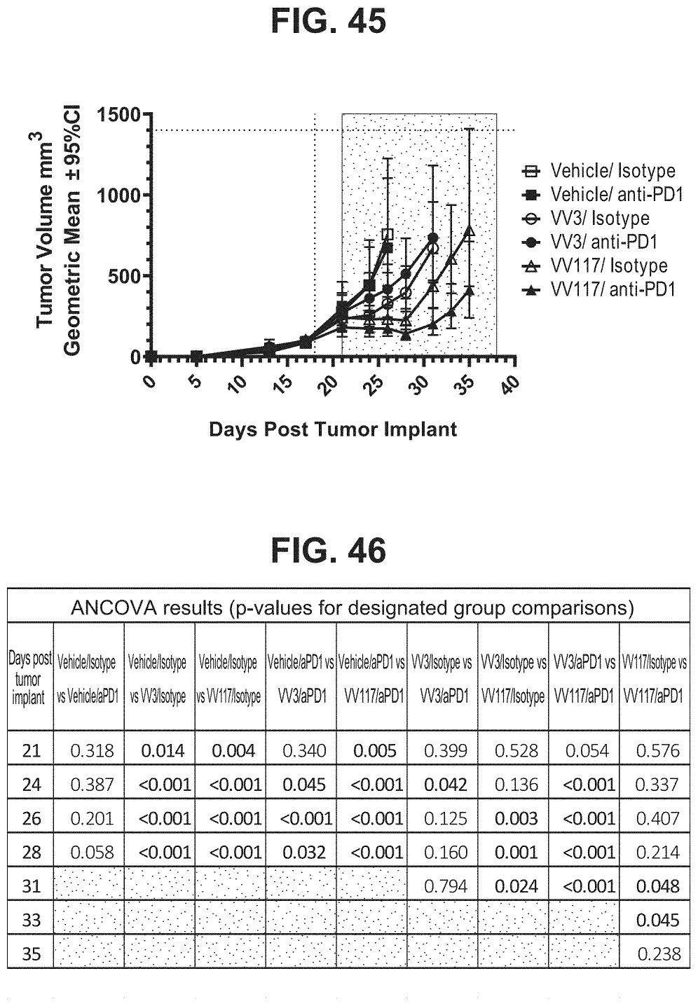

Analysis of tumor growth profiles, shown as group averages for each test virus (FIG. 45) revealed an important finding. IV administration of IL-2gv transgene-armed WR viruses led to statistically significant inhibition of MC38 tumor growth compared to vehicle and reporter transgene-armed WR virus (VV3) treatment. There was no statistically significant difference between tumor growth inhibition induced by anti PD-1 or IgG1 Isotype antibody treatment on vehicle treated tumors. For VV3 and VV117, however there was a statistically significant difference detected between anti PD-1 or IgG1 isotype antibody treatment. (FIG. 46, Table 10, ANCOVA results).

FIG. 45 shows results of assessment of virotherapy-induced tumor growth inhibition using single (day 18) IV virus delivery on C57BL/6 female mice implanted SC with B16F10 tumor cells in combination with anti-PD-1 antibody treatment. Tumor growth trajectories are shown for each treatment as group averages±95% confidence intervals up through day 35 post-tumor implantation until time of sacrifice. Test viruses included WR vaccinia viruses armed with either a Luciferase-2A-GFP reporter (WR.Luc-GFP (VV3)) hIL-2gv1 (WR.hIL-2gv1.HSV TK.007.A34K151E (VV117). Dashed vertical lines on each graph represent time points when mice received IV injections of virus. The grey block indicates the time window for bi-weekly SC anti-PD1 antibody treatment (day 21 to day 38). The dashed horizontal line on each graph represents the tumor volume threshold used as a criterion to remove animals from the study.

FIG. 46 (Table 10) show results of statistical comparison of virotherapy-induced tumor growth inhibition using ANCOVA for subcutaneous B16F10 tumor model study. Tumor volumes for individual mice in each group on multiple days after treatment were analyzed by ANCOVA to determine statistically significant inhibitory effects on tumor growth across various treatment groups. Columns show the statistical results (p values) of comparisons between specific treatment group pairs. Values in bold font represent comparative ANCOVA results where p values 0.05 were observed.

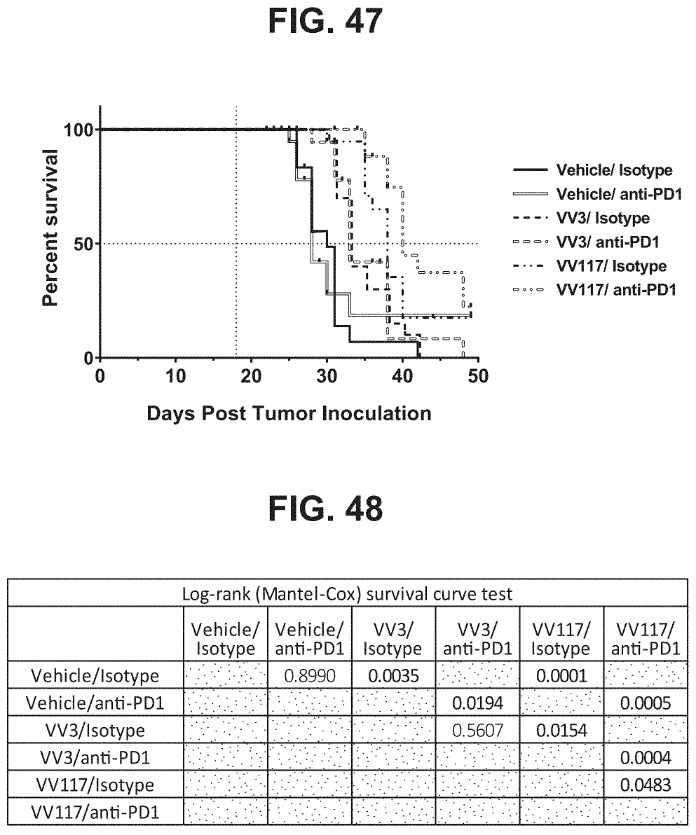

Survival results for the same test viruses showed very similar outcomes as those reported above for tumor growth inhibition. This included statistically superior group survival associated with IL-2gv transgene-armed WR viruses compared to the corresponding Luc-GFP reporter-armed WR virus (FIG. 47). No survival benefit was observed for vehicle treated tumors treated with anti PD-1 antibodies in comparison to isotype treated tumors. For VV3 and VV117, however there was a statistically significant difference detected between anti PD-1 and IgG1 isotype antibody treatment (FIG. 48, Table 11). Overall, IV delivery of IL-2gv transgene-armed WR virus variants proved to be an effective anti-tumor therapy in the B16F10 SC tumor model and demonstrated the potency of a single therapeutic administration of virus.

FIG. 47 show results of survival of B16F10 tumor-bearing C57BL/6 female mice following IV treatment with recombinant oncolytic vaccinia viruses on day 18 after SC tumor implantation. Mice were designated daily as deceased upon reaching tumor volume ≥1400 mm³. The point of intersection between each group's curve and the horizontal dashed line indicates the median (50%) survival threshold for the group.

FIG. 48 (Table 11) show results of statistical comparison of virotherapy-induced survival in the B16F10 tumor model. Survival was monitored and then analyzed by Log-rank test (Mantel-Cox). P values are listed for each group comparison.

### Example 15

In this example, various potential single and double glycan human IL-2 variants were expressed and assayed for glycosylation at the introduced potential glycosylation sites.

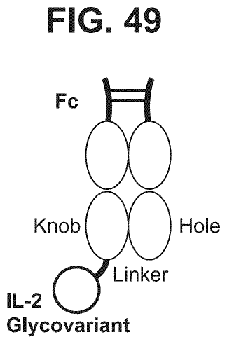

Each of the IL-2 variants were expressed as fusion proteins, where the IL-2 variant was linked to covalently linked to a human IgG1 Fc domain via a linker having the amino acid sequence: GGGGSGGGGS (SEQ ID NO:37). The Fc domain contains a first Fc chain and a second Fc chain, in which the first Fc chain contains “knob” amino acid substitutions and the second Fc chain contains “hole” amino acid substitutions, in order to promote heterodimer formation between the Fc chains. The N-terminus of the IL-2 variant was covalently linked via the linker to the C-terminus of the first Fc chain. A schematic of the Fc-IL-2 molecule is depicted in FIG. 49.

To prepare the genes encoding the fusion proteins, gene syntheses were performed using endogenous codons of human IL-2 (Refseq: NM_000586.3, CCDS: CCDS3726.1, UniProtKB: P60568) fused to the C-terminus of human IgG1 Fc (fragment crystallizable UniProtKB: P01857). Fc fragments started at the upper hinge residue D221 (position 221-447 EU numbering) and included effector function inactivating mutations L234A, L235A, and G237A. A knob-in-hole heavy chain pair was utilized to fuse a single IL-2 variant to the C-terminus of the knob chain using a GGGGSGGGGS linker (SEQ ID NO: 37). Knob chain pairing mutations were made at Y349C and T366W, and hole chain mutations at S354C, T366S, L368A, and Y407V. Constant regions also contained D356E and L358M allotype mutations from G1m to nG1m1. Genes were subcloned into the mammalian expression vector pCEP4 (Invitrogen) by ATUM (Newark, Calif.).

Fusion proteins were expressed by transient transfection using either Expi293 or ExpiCHO expression systems (ThermoFisher Scientific) following supplier's instructions. Fc-IL2 fusion proteins were purified by tandem Protein A affinity chromatography using a 5 mL HiTrap MabSelect SuRe column (GE Heathcare) and size exclusion chromatography using a HiLoad 16/600 Superdex 200 pg column (GE Healthcare) on an AKTA Avant 25 chromatography system (GE Healthcare). Purified fusion proteins were filter sterilized and stored at −80° C. before use.

The purity and homogeneity of the Fc-IL2 fusion proteins were evaluated by analytical size exclusion chromatography with an Agilent 1260 HPLC on a TSKgel SuperSW mAb HR column (Tosoh Bioscience, microfluidic electrophoretic separation using a LabChip GXII Touch (PerkinElmer), and mass spectrometry. The intact mass of the purified fusion proteins was confirmed by Xevo G2-XS QTof Quadrupole Time-of-Flight Mass Spectrometry (Waters) coupled to an Acquity UPLC Protein BEH C4 300 Å 1.7 μm column (Agilent).

Purified Fc-IL2 variant molecules were subjected to PNGase F treatment to detect whether the molecule was glycosylated, and if so, if at one or both (if applicable) introduced potential glycosylation sites. Specifically, Fc-IL2 fusion proteins were deglycosylated first in non-reducing and reducing conditions using rapid PNGase F enzyme (New England Biolabs, P0710S and P0711S) to determine mass of the intact proteins (non-reduced) and reduced proteins. Characterization of N-linked glycans from Fc-IL2-fusion proteins was carried out using ‘GlycoWorks™ RapiFluor-MS™ N-Glycan Kit’ (Waters) following supplier's protocols. Proteins were processed with RapiGest solution and denatured. Rapid PNGase F was added to release the N-linked glycans as glycosylamines. Following digestion, the amino group of the released glycosylamines were labeled with RFMS-labels according to the manufacturer's instructions. Labelled N-glycans were purified using a Waters hydrophilic interaction liquid chromatography (HILIC) pElution plate in an ammonium formate and acetonitrile solution were then directly analyzed by LC-MS (Waters).

Table A lists the potential single and double glycan IL-2 variant molecules that were expressed and assayed for glycosylation. As a control, an Fc-IL2 (wild type) molecule was also expressed and tested. In the IL-2 proteins listed below, “Site 1” is the first listed potential glycosylation site in the protein name, and “Site 2” is the second listed potential glycosylation site in the protein name (if applicable). For example, in the protein “Fc-IL2-R38N140T-T41N:K43T”, Site 1 is R38N:L40T and Site 2 is T41N:K43T. Additionally, glycosylation was also confirmed by mass spectrometry through detection of aspartic acid formation after PNGase F treatment. The total number of glycan modification are shown that include the Asn297 site on the Fc domain as well as those specifically attributed to the fused IL-2 cytokine. (Each molecule has 2 Asn297 glycans, so each molecule has at least 2 total N-glycans).

TABLE A
Potential single and double glycan IL-2 variant molecules
that were expressed and assayed for glycosylation

| | Microfluidic | | | |
|---|---|---|---|---|
| | Electrophoresis | | Mass Spectrometry | |
| | Site 1 | Site 2 | Total N- | IL-2 N- |
| Protein | (%) | (%)¹ | Glycan | Glycan |
| Fc-IL2 | — | — | 2 | 0 |
| Fc-IL2-K35N | 31 | — | 2 or 3 | 0 or 1 |
| Fc-IL2-R38N:K40T | >99 | — | 3 | 1 |
| Fc-IL2-T41N:K43T | >99 | — | 3 | 1 |
| Fc-IL2-F42N:F44T | 0 | — | 2 | 0 |
| Fc-IL2-K43N:Y45T | >99 | — | 3 | 1 |
| Fc-IL2-E62N:K64T | 0 | — | 2 | 0 |
| Fc-IL2-E68N:L70T | 0 | — | 2 | 0 |
| Fc-IL2-L72N:Q74T | 94 | — | 3 | 1 |
| Fc-IL2-K35N-T41N:K43T | 38 | >99 | 3 or 4 | 1 or 2 |
| Fc-IL2-R38N:L40T- | >99 | — | 3 | 1 |
| T41N:K43T | | | | |
| Fc-IL2-R38N:L40T- | >99 | >99 | 4 | 2 |
| K43N:Y45T | | | | |
| Fc-IL2-R38N:L40T- | >99 | 0 | 3 | 1 |
| E62N:K64T | | | | |
| Fc-IL2-R38N:L40T- | >99 | >99 | 4 | 2 |
| L72N:Q74T | | | | |
| Fc-IL2-T41N:K43T- | >99 | 0 | 3 | 1 |
| E62N:K64T | | | | |
| Fc-IL2-T41N:K43T- | >99 | >99 | 4 | 2 |
| L72N:Q74T | | | | |
| Fc-IL2-K43N:Y45T- | >99 | 0 | 3 | 1 |
| E62N:K64T | | | | |
| Fc-IL2-K43N:Y45T- | >99 | >99 | 4 | 2 |
| L72N:Q74T | | | | |

¹Partial occupancy of dual glycokines were assigned based on single site occupancy results. Occupancy of Fc-IL2-R38N:L40T-T41N:K43T was arbitrarily assigned in the absence of peptide mapping.

As shown in Table A, the introduced glycosylation sites R38N:K40T, T41N:K43T, K43N:Y45T, and L72N:Q74T were all highly glycosylated (over 90% of molecules with most exceeding >99%) on the relevant asparagine. Partial glycosylation was observed for the potential glycosylation site K35N, where the introduced asparagine was moderately glycosylated (about a third of the molecules). In contrast, the introduced potential glycosylation site F42N:F44T, E62N:K64T, and E68N:L70T were not glycosylated.

### Example 16

In this example, various double glycan IL-2 variants described above were assayed for binding affinity to human IL-2Rα and human IL-2Rβ.

All experiments were performed on a Biacore 8K Surface Plasmon Resonance based biosensor (GE Healthcare). Purified soluble ligands were covalently coupled onto a CM5 sensor chip using an Amine Coupling Kit (GE Healthcare, Product #BR100050) following the manufacturer's recommendations. HBS-EP+ running buffer (10 mM HEPES pH 7.4, 0.15 M NaCl, 3 mM EDTA, 0.005% P-20), ranging in concentration was injected on all flow cells for 7 minutes at 20 μL/min. CD25 and CD122 was captured to surface densities of ˜20 and ˜500 RU, respectively. A non-derivatized flow cell was used as a reference surface. All flow cells were blocked with 100 mM ethylenediamine in 200 mM borate buffer pH 8.5 for 7 minutes at 10 μL/min.

Protein interaction experiments were performed using HBS-EP+(pH 7.4) at 25° C. on each spot. Following capture of antigens, analyte (1.23, 3.7, 11.1, 33.3, 100, 300, and 900 nM concentrations of IL-2 variants) was injected at a flow rate of 50 μL/min in all flow cells for 50 seconds. After each analyte injection, dissociation was monitored for 5 minutes, followed by regeneration of all flow cells with a 20 second injection of 10 mM glycine (pH 2.1). Buffer cycles were collected for each sample for double-referencing purposes (double-referencing as described in Myszka, D. G., Improving biosensor analysis. J. Mol. Recognit. 12, 279-284 (1999)). For kinetic analysis, the double-referenced sensorgrams were fit globally to a simple 1:1 Langmuir with mass transport binding model using Biacore 8K Evaluation Software version 1.1.1.7442. For steady-state affinity analysis, the double-referenced equilibrium binding responses were fit with a 1:1 Langmuir steady-state model using Biacore 8K Evaluation Software version 1.1.1.7442.

The kinetics and affinity parameters for tested IL-2 variants are shown in Table B below:

TABLE B
kinetics and affinity parameters for tested IL-2 variants

| | hIL2Rα (CD25) kinetics | | | hILR2β (CD122) kinetics | | |
|---|---|---|---|---|---|---|
| | kₐ | k_{d} | K_{D} | kₐ | k_{d} | K_{D} |
| Molecule | (1/Ms) | (1/s) | (nM) | (1/Ms) | (1/s) | (nM) |
| Fc-IL2 (wt) | 2.27E+06 | 8.59E−02 | 37.85 ± | 1.31E+05 | 2.79E−01 | 2123.02 ± |
| | | | 0.08 | | | 66.87 |
| Fc-IL2- | Non- | Non- | N/A | 8.26E+04 | 2.92E−01 | 3542.87 ± |
| R38N:L40T- | detectable | detectable | | | | 110.64 |
| K43N:Y45T | binding | binding | | | | |
| Fc-IL2- | Non- | Non- | N/A | 8.99E+04 | 2.35E−01 | 2615.05 ± |
| K43N:Y45T- | detectable | detectable | | | | 55.23 |
| L72N:Q74T | binding | binding | | | | |

As shown in Table B, the Fc-IL2-R38N:L40T-K43N:Y45T and Fc-IL2-K43N:Y45T-L72N:Q74T variants retain similar binding affinity to human IL-2Rβ as the wild-type IL-2 Fc fusion. Wild-type Fc-IL-2 fusion□demonstrated much higher binding affinity to IL-2Rα than to IL-2Rβ. In contrast, the Fc-IL2-R38N:L40T-K43N:Y45T and Fc-IL2-K43N:Y45T-L72N:Q74T variants do not have measurable binding to IL-2Rα.

### Example 17

In this example, various single and double glycan IL-2 variants described above were assayed for activation of lymphocytes containing either the IL-2 CD122/CD132 (β/γ) receptor complex (HH cells) or the IL-2 CD25/CD122/CD132 (α/β/γ) receptor complex (induced Tregs (or “iTregs”)).

Various Fc-linked single and double glycan IL-2 variants as described in Example 15 were tested. The tested variants were: Fc-IL2-R38N:L40 T; Fc-IL2-T41N:K43T; Fc-IL2-K43N:Y45T; Fc-IL2-E62N:K64T; Fc-IL2-L72N:Q74T; Fc-IL2-R38N: L40T-K43N:Y45T; Fc-IL2-K43N:Y45T-E62N:K64T; and Fc-IL2-K43N:Y45T-L72N:Q74T. In addition, Fc-linked wild-type human IL-2 (“Fc-IL2”) and Fc-linked IL2v (“Fc-IL2v”) were also tested. “IL2v” is a variant of human IL-2 that has mutations to abolish IL-2Rα binding (Klein, C, et al, Oncoimmunology, Vol. 6, No. 3, 2017). IL2v has the following mutations to eliminate interaction between IL-2 and IL-2Rα: F42A, Y45A, and L72G. In addition, IL2v has the mutations T3A and C125A.

The ability of the IL-2 variants to activate HH cells and iTregs was measured by monitoring relative changes in phosphorylated STAT5 (pSTAT5) in response to treatment of the cells with the IL-2 variants. pSTAT5 is known to be a downstream consequence of IL-2 signaling. HH T cells (ATCC CRL-2105) are a line of T cells that lack the alpha chain of the IL-2 receptor complex, but that contain the beta and gamma chains of the IL-2 receptor complex. iTreg cells were prepared from Fresh Leuko Paks obtained from STEMCELL Technologies (CAT #70500.1, Donor #D001003551).

For IL-2 activation, the HH cells and iTreg cells were plated at 2*10e6 cells/well in 50 ul serum-free RPMI 1640 media (Gibco), and allowed to rest at 37° C. After resting, cells were treated the IL-2 molecules listed above, and cells were then pelleted by centrifugation.

Following treatment with the IL-2 molecules, cell induction status was evaluated by the InstantOne ELISA pSTAT5 detection kit (Invitrogen).

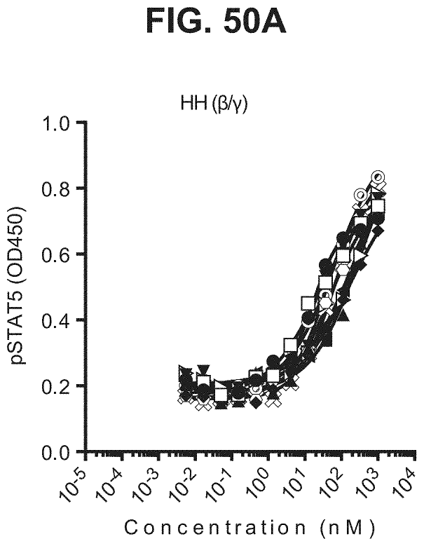

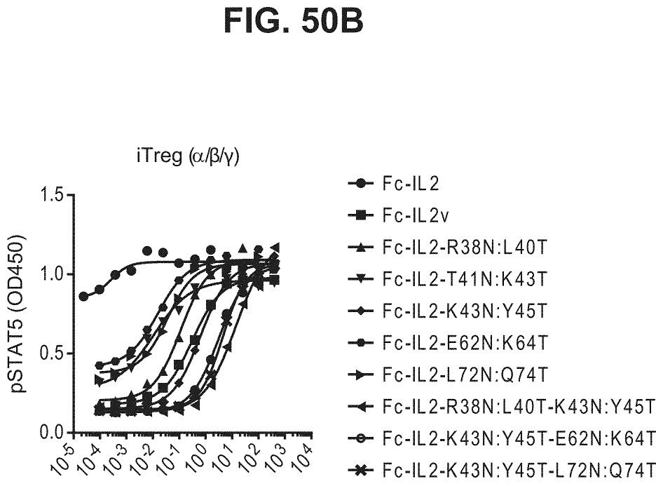

FIGS. 50A and 50B depict the effect of various concentrations of the listed IL-2 variants on the activation of HH cells and iTregs, respectively, as measured by increase in pSTAT5 induction. As shown in FIG. 50A, all of the tested IL-2 variants have similar effectiveness at activating HH cells. Specifically, for each tested concentration of Fc-IL2-R38N:L40T; Fc-IL2-T41N:K43T; Fc-IL2-K43N:Y45T; Fc-IL2-E62N:K64T; Fc-IL2-L72N:Q74T; Fc-IL2-R38N:L40T-K43N:Y45T; Fc-IL2-K43N:Y45T-E62N:K64T; and Fc-IL2-K43N:Y45T-L72N:Q74T proteins, these molecules result in a similar increase in pSTAT5 optical density (OD) in HH cells as is generated by treatment of the cells with corresponding concentrations of Fc-IL2 (wild-type). In contrast, as shown in FIG. 50B, each of the tested IL-2 variants has reduced activation of iTreg cells as compared to wild-type IL-2.

Based the data as shown in FIGS. 50A-50B, EC50 values were calculated for each of the tested Fc-IL-2 molecules by calculating the concentration of the respective Fc-IL-2 variant that resulted in a pSTAT5 level 50% of the maximum value using GraphaPad Prism 8. The EC50 values for the different Fc-IL-2 molecules and cell types are provided in Table C below.

TABLE C
The EC50 values for the different Fc-IL-2 molecules and cell types

| | HH T cell | iTreg cell | Selectivity |
|---|---|---|---|
| Protein | (nM) | (nM) | HH/iTreg |
| Fc-IL2 (wild- | 16.9 | <2 × 10⁻⁴ | <1.18 × 10⁻⁵ |
| type) | | | |
| Fc-IL2v | 24.9 | 0.31 | 1.24 × 10⁻² |
| Fc-IL2- | 163 | 0.11 | 6.75 × 10⁻⁴ |
| R38N:L40T | | | |
| Fc-IL2- | 37.2 | 0.01 | 2.69 × 10⁻⁴ |
| T41N:K43T | | | |
| Fc-IL2- | 166 | 0.64 | 3.86 × 10⁻³ |
| K43N:Y45T | | | |
| Fc-IL2- | 52.1 | 0.02 | 3.84 × 10⁻⁴ |
| E62N:K64T | | | |
| Fc-IL2- | 47.6 | 0.04 | 8.4 × 10⁻⁴ |
| L72N:Q74T | | | |
| Fc-IL2- | 218 | 14.1 | 6.47 × 10⁻² |
| R38N:L40T- | | | |
| K43N:Y45T | | | |
| Fc-IL2- | 66.2 | 3.07 | 4.64 × 10⁻² |
| K43N:Y45T- | | | |
| E62N:K64T | | | |
| Fc-IL2- | 65.2 | 4.49 | 6.89 × 10⁻² |
| K43N:Y45T- | | | |
| L72N:Q74T | | | |

As shown in FIGS. 50A, 50B, and Table C, most of the different IL-2 variant fusions have a similar efficacy (i.e. within 10×/an order of magnitude) as wild-type IL-2 fusion in activating HH cells (FIG. 50A and Table C). In contrast, the IL-2 variants have significantly reduced efficacy (i.e. reduced more than 100-fold/2 orders of magnitude) as compared to wild-type IL-2 in activating iTregs (FIG. 50B and Table C). Table C also provides a determination for the selectivity of each molecule for activation of HH cells vs iTreg cells (EC50 HH cells/EC50 iTreg cells), where a higher value indicates greater relative selectivity for HH cells vs. iTreg cells. As shown in Table C, the IL-2 variants Fc-IL2-R38N:L40T-K43N:Y45T, Fc-IL2-K43N:Y45T-E62N:K64T, and Fc-IL2-K43N:Y45T-L72N:Q74T have the greatest relative selectivity for HH cells vs iTreg cells of the tested molecules.

### Example 18

In this example, various single and double glycan IL-2 variants described above were tested for their activation of STAT5 signaling in CD8 T cells, NK cells, and Treg cells in human peripheral blood mononuclear cells (hPBMCs).

IL-2 Variants—Single Glycan Variants

In this experiment, the ability of the IL-2 variants to activate CD8 T cells, NK cells, and Treg cells was measured by monitoring relative changes in pSTAT5 in response to treatment of the cells with the IL-2 variants. The IL-2 variants tested in this experiment were each fused to a human IgG Fc domain as described in Example 15. The tested variants were: Fc-IL2-K35N; Fc-IL2-R38N:L40T; Fc-IL2-T41N:K43T; Fc-IL2-K43N:Y45T; Fc-IL2-E62N:K64T; Fc-IL2-L72N:Q74T. In addition, Fc-linked wild-type human IL-2 (“Fc-IL2”) and Fc-linked IL2v (“Fc-IL2v”) as described in Example 3 were also tested.

Blood from healthy volunteers was taken and hPBMCs were isolated using a ficoll-paque (GE Healthcare) gradient, washed with PBS to remove platelets, and cleared of red blood cells using ACK lysis buffer (Gibco). Cells were then plated at 1*10e6 cells/well in 90 uL serum-free RPMI 1640 media (Gibco) and allowed to rest for 2-4 hours at 37° C. After resting, cells were treated the IL-2 molecules listed above (10 uL), at the indicated concentrations for 20 minutes at 37° C., and 25 uL 20% PFA was immediately added with gentle pipetting. Cells were then pelleted by centrifugation and aspirated (400 RCF, 7 minutes).

Phosflow Perm Buffer III (BD Biosciences) was added (200 uL), and the cells were mixed gently by pipetting up and down once to prevent clumping. Cells were then washed twice with 200 uL FACS buffer followed by pelleting by centrifugation (400 RCF, 7 minutes). Cells were in resuspended in 200 uL FACS buffer and incubated with the antibodies in Table D and Table E per standard procedure then suspended in 200 uL FACS buffer for FACS analysis. Data was analyzed using FlowJo v10 software.

TABLE D

| Human CD4 | | | | |
|---|---|---|---|---|
| Panel 1 | | | | |
| Marker | Fluor | Clone | Vendor | Catalog# |
| CD3 | AF488 | UCHT1 | BioLegend | 300415 |
| CD4 | Bv605 | RPA-T4 | BioLegend | 300556 |
| CD25 | Bv421 | 2A3 | BD | 564033 |
| FoxP3 | PE | 236A/E7 | Invitrogen | 12-477 |
| pSTAT5 | AF647 | 47/Stat5 | BD | 562076 |
| | | pY694 | | |
| CD8 | APC-Cy7 | RPA-T8 | BD | 557760 |
| CD56 | BV711 | HCD56 | BioLegend | 318336 |

TABLE E

| Human CD4 | | | | |
|---|---|---|---|---|
| Panel 2 | | | | |
| Marker | Fluor | Clone | Vendor | Catalog# |
| CD3 | BV711 | UCHT1 | BioLegend | 300415 |
| CD4 | Bv605 | RPA-T4 | BioLegend | 300556 |
| CD25 | Bv421 | 2A3 | BD | 564033 |
| FoxP3 | PE | 259D/C7 | BD | 560046 |
| pSTAT5 | AF647 | 47/Stat5 | BD | 562076 |
| | | pY694 | | |
| CD8 | APC-Cy7 | RPA-T8 | BD | 557760 |
| CD56 | FITC | HCD56 | BioLegend | 318336 |

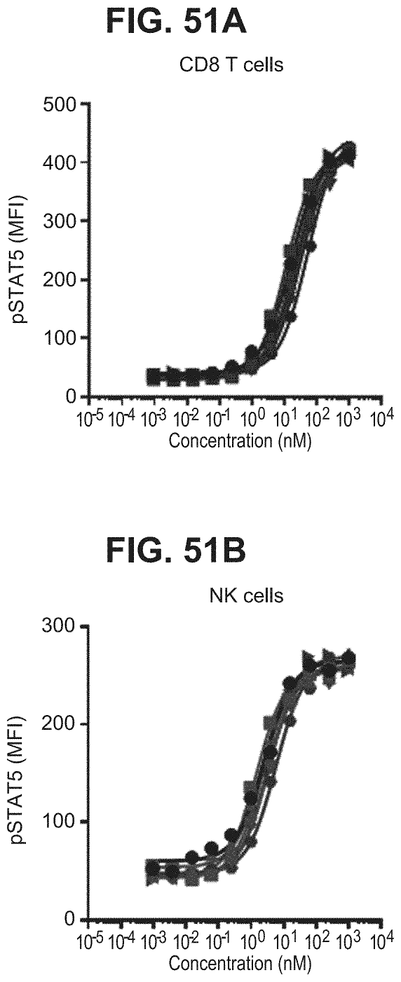

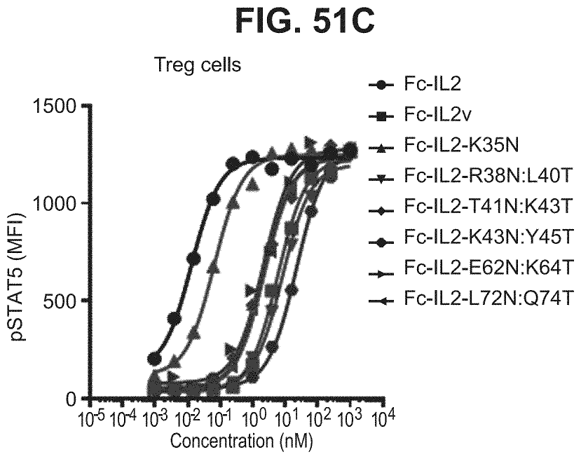

FIGS. 51A, 51B, and 51C depict the effect of various concentrations of the listed IL-2 variants on the activation of CD8 T cells, NK cells, and Treg cells, respectively, as measured by increase in pSTAT5 in the cells. As shown in FIGS. 51A and 51B, all of the tested IL-2 variants have similar effectiveness at activating CD8 T cells and NK cells. Specifically, for each tested concentration of Fc-IL2-K35N; Fc-IL2-R38N:L40 T; Fc-IL2-T41N:K43T; Fc-IL2-K43N:Y45T; Fc-IL2-E62N:K64T; and Fc-IL2-L72N:Q74T molecules, these molecules result in a similar increase in pSTAT5 mean fluorescence intensity (MFI) in CD8 T cells and NK cells as is generated by treatment of the cells with corresponding concentrations of Fc-IL2 (wild-type). In contrast, as shown in FIG. 51C, each of the tested Fc-IL-2 variants has reduced activation of Treg cells as compared to wild-type Fc-IL-2.

Based the data as shown in FIGS. 51A-51C, EC50 values were calculated for each of the tested Fc-IL-2 molecules as described above. The EC50 values are provided in Table F below.

TABLE F

| | CD8 T | NK cell | Treg cell | Selectivity | Selectivity |
|---|---|---|---|---|---|
| Protein | cell (nM) | (nM) | (nM) | CD8/Treg | NK/Treg |
| Fc-IL2 (wild- | 15.7 | 2.52 | 0.01 | 0.000637 | 0.004 |
| type) | | | | | |
| Fc-IL2v | 11.3 | 1.53 | 5.88 | 0.52 | 3.84 |
| FC-IL2-K35N | 24.6 | 2.54 | 0.07 | 0.00284 | 0.0276 |
| Fc-IL2- | 26.5 | 3.06 | 7.89 | 0.3 | 2.58 |
| R38N:L40T | | | | | |
| Fc-IL2- | 31.2 | 3.05 | 2.13 | 0.07 | 0.7 |
| T41N:K43T | | | | | |
| Fc-IL2- | 57.6 | 6.37 | 24.2 | 0.42 | 3.8 |
| K43N:Y45T | | | | | |
| Fc-IL2- | 25.5 | 3.65 | 2.26 | 0.09 | 0.62 |
| E62N:K64T | | | | | |
| Fc-IL2- | 22.1 | 2.33 | 2.29 | 0.1 | 0.98 |
| L72N:Q74T | | | | | |

Also provided in Table F is a value for selectivity of the respective IL-2 variant for CD8 T cells vs Treg cells or NK cells vs Treg cells, where larger numbers indicate greater selectivity for CD8 T cells or NK cells over Treg cells. As shown in Table F, the various IL-2 variants activate CD8 T cells and NK cells at a similar EC50 to Fc-IL2 (wild-type), but activate Treg cells far less than the wild-type fusion protein. Similarly, the Fc-IL2 variants have greater selectivity for CD8 T cells and NK cells vs Treg cells, as compared to Fc-IL2 (wild-type).

IL-2 Variants—Double Glycan Variants

Next, various double glycan IL-2 variants were tested. The IL-2 variants tested in this experiment were each covalently linked to a human IgG Fc domain as described in Example 15. The tested double glycan IL-2 variant molecules were: Fc-IL2-R38N: L40T-K43N:Y45T; Fc-IL2-R38N: L40T-E62N: K64T; Fc-IL2-R38N: L40T-L72N:Q74T; Fc-IL2-T41N:K43T-E62N:K64T; Fc-IL2-T41N:K43T-L72N:Q74T; Fc-IL2-K43N:Y45T-E62N:K64T; and Fc-IL2-K43N:Y45T-L72N:Q74T. In addition, the single glycan IL-2 variant molecules Fc-IL2-R38N:L40T; Fc-IL2-T41N:K43T; and Fc-IL2-K43N:Y45T, and Fc-IL2 (wild-type) and Fc-IL2v were also tested for comparison.

hPBMCs were prepared as above for the single glycan variants. The cells were then treated with the IL-2 double glycan variants and related control IL-2 molecules listed immediately above, and then prepared for flow cytometry as described above for the single glycan variants.

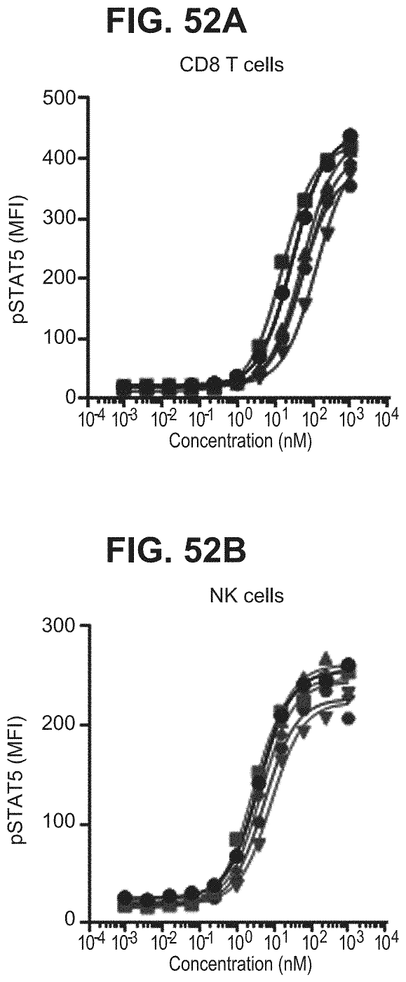

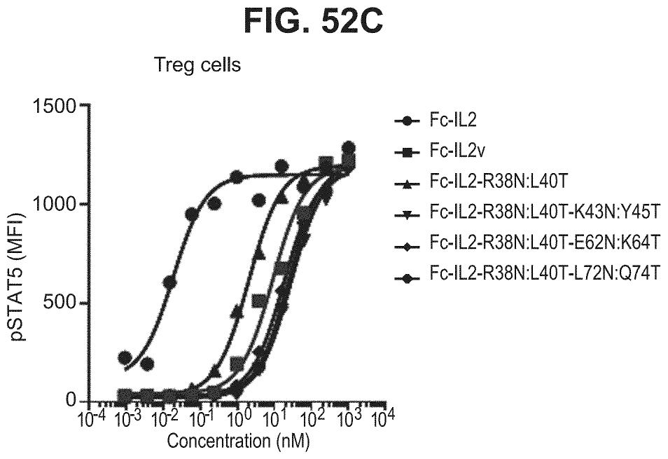

FIGS. 52A, 52B, and 52C depict the effect of various concentrations of R38N:L40T-containing double glycan IL-2 variants on the activation of CD8 T cells, NK cells, and Treg cells, respectively, as measured by increase in pSTAT5 in the cells. As shown in FIGS. 52A and 52B, all of the tested IL-2 variants have similar effectiveness at activating CD8 T cells and NK cells. In contrast, as shown in FIG. 52C, each of the tested R38N:L40T-containing double glycan IL-2 variants have substantially reduced activation of Treg cells as compared to the wild-type IL-2 molecule, and also reduced activation of Treg cells as compared to the R38N:L40T single glycan IL-2 variant molecule.

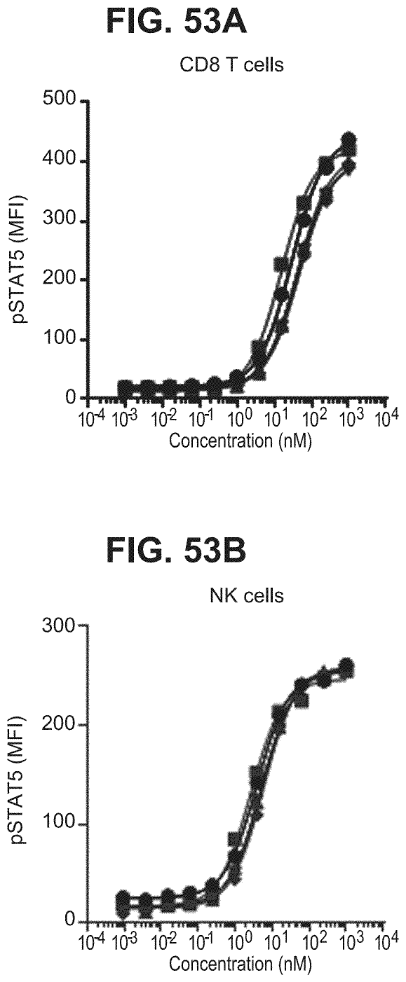

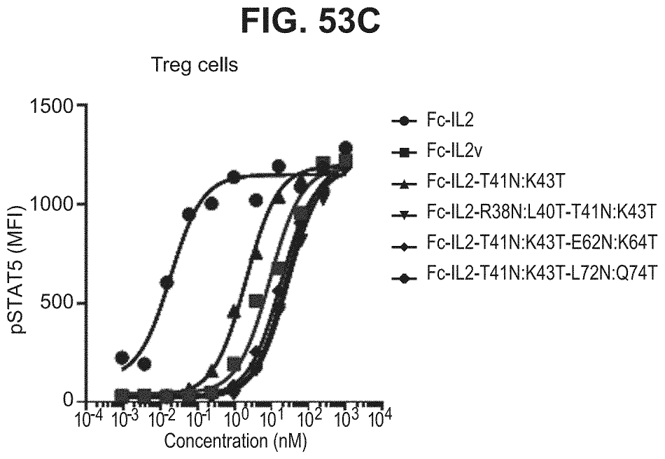

FIGS. 53A, 53B, and 53C depict the effect of various concentrations of T41N:K43T-containing double glycan IL-2 variants on the activation of CD8 T cells, NK cells, and Treg cells, respectively, as measured by increase in pSTAT5 in the cells. As shown in FIGS. 53A and 53B, all of the tested IL-2 variants have similar effectiveness at activating CD8 T cells and NK cells. In contrast, as shown in FIG. 53C, each of the tested T41N:K43T-containing double glycan IL-2 variants has substantially reduced activation of Treg cells as compared to the wild-type IL-2 molecule, and also reduced activation of Treg cells as compared to the T41N:K43T single glycan IL-2 variant molecule.

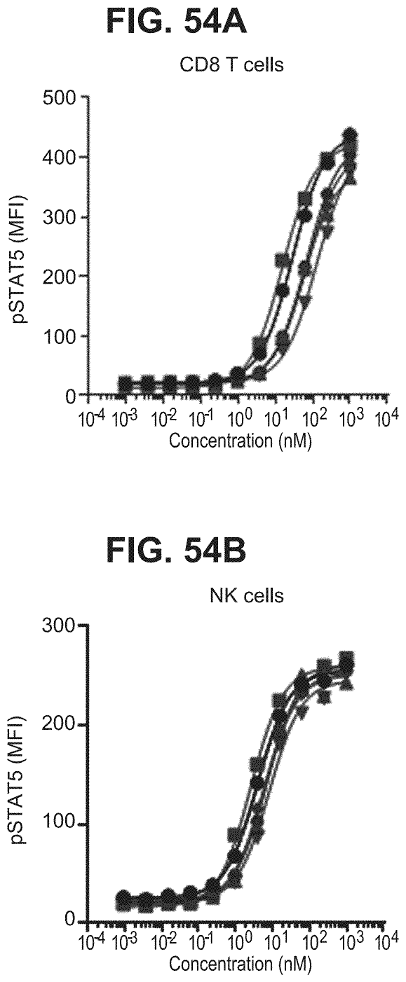

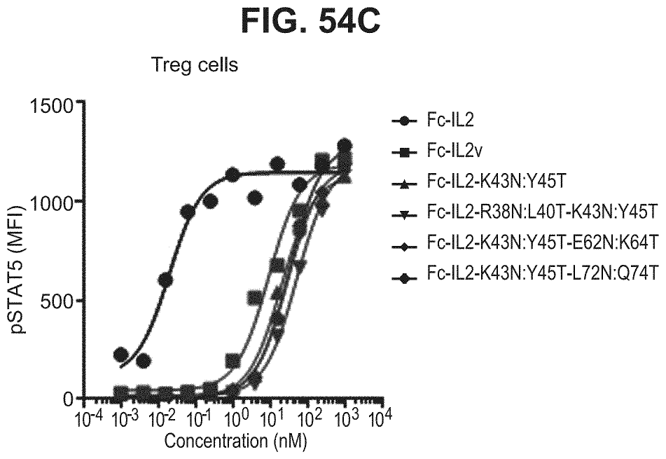

FIGS. 54A, 54B, and 54C depict the effect of various concentrations of K43N-Y45T-containing double glycan IL-2 variants on the activation of CD8 T cells, NK cells, and Treg cells, respectively, as measured by increase in pSTAT5 in the cells. As shown in FIGS. 54A and 54B, all of the tested IL-2 variants have similar effectiveness at activating CD8 T cells and NK cells. In contrast, as shown in FIG. 54C, each of the tested K43N-Y45T-containing double glycan IL-2 variants has substantially reduced activation of Treg cells as compared to the wild-type IL-2 molecule.

### Example 19

In this example, various IL-2 variants containing a single introduced glycosylation site (R38N:L40T) and a substitution at amino acid position 62 were tested for their activation of CD8 T cells, NK cells, and Treg cells. The tested variants were: Fc-IL2-R38N:L40T-E62A; Fc-IL2-R38N:L40T-E62N; Fc-IL2-R38N:L40T-E62K; and Fc-IL2-R38N:L40T-E62R. In addition, the double glycan variant Fc-IL2-R38N:L40T-E62N:K64T, Fc-linked wild-type human IL-2 (“Fc-IL2”) and Fc-linked IL2v (“Fc-IL2v”) were also tested as controls. The ability of the IL-2 variants to activate CD8 T cells, NK cells, and Treg cells was measured by monitoring relative changes in phosphorylated STAT5 (pSTAT5) in response to treatment of the cells with the IL-2 variants. Cell activation/pSTAT5 assays were performed as described in Example 18.

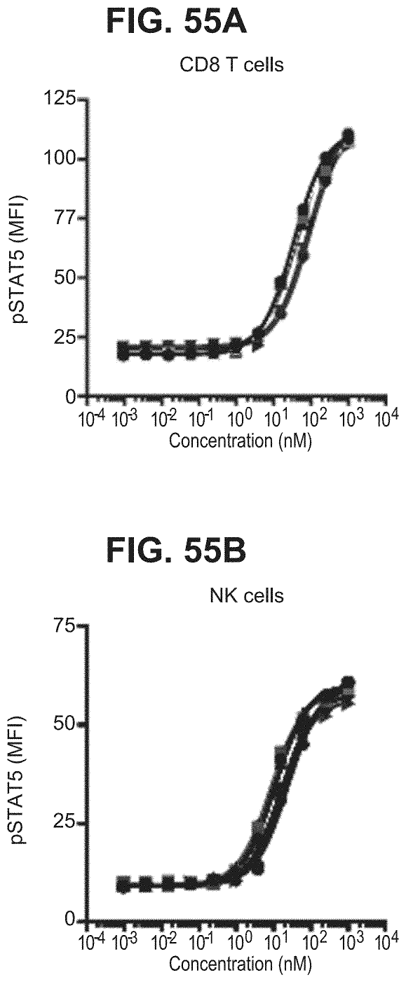

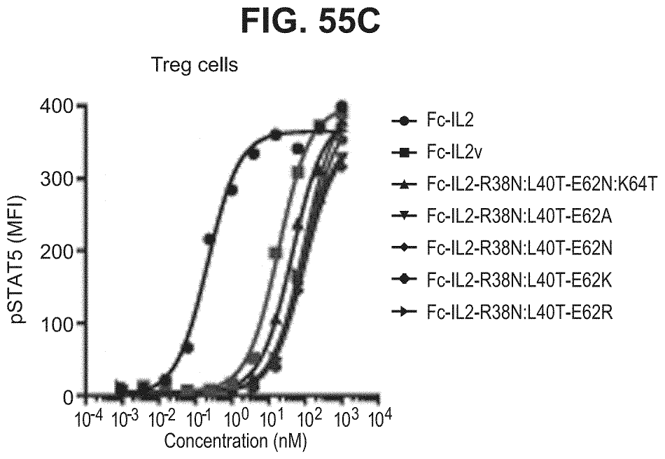

FIGS. 55A, 55B, and 55C depict the effect of various concentrations of the IL-2 variant fusion proteins on the activation of CD8 T cells, NK cells, and Treg cells, respectively, as measured by increase in pSTAT5 in the cells. As shown in FIGS. 55A and 55B, all of the tested IL-2 variants have similar effectiveness at activating CD8 T cells and NK cells. In contrast, as shown in FIG. 55C, each of the tested IL-2 variants has substantially reduced activation of Treg cells as compared to the wild-type IL-2 molecule.

### Example 20

In this example, various single and double glycan IL-2 variants described above were tested for their effect on expansion of CD8 T cells, NK cells, and Treg cells in vivo. The tested variants were: Fc-IL2-K43N:Y45T; Fc-IL2-R38N:L40T-K43N:Y45T; Fc-IL2-K43N:Y45T-L72N:Q74T. In addition, Fc-linked wild-type human IL-2 (“Fc-IL2”) and Fc-linked IL2v (“Fc-IL2v”) were also tested as controls.

Mice were randomized into groups to receive one of the molecules listed above or PBS control. The treatment groups were as follows: PBS; Fc-IL2; Fc-IL2v; Fc-IL2-K43N:Y45T; Fc-IL2-R38N:L40T-K43N:Y45T; Fc-IL2-K43N:Y45T-L72N:Q74T. Fc-IL2 fusion molecules were dosed at 0.5, 1, or 2 mg/kg daily for 4 consecutive days by subcutaneous injection with the respective control or IL-2 variant at the concentration thereof for their assigned group. Immunophenotyping was conducted on day 3 after the first treatment (day 0) by collecting spleens from each group.

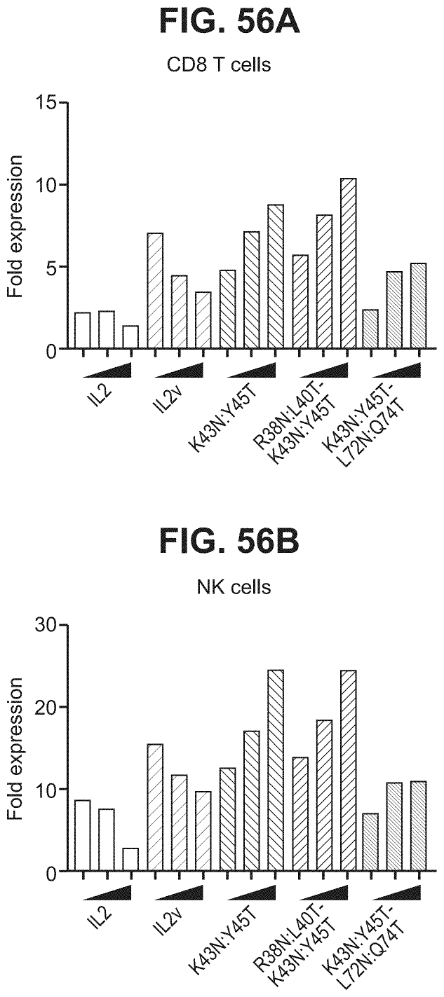

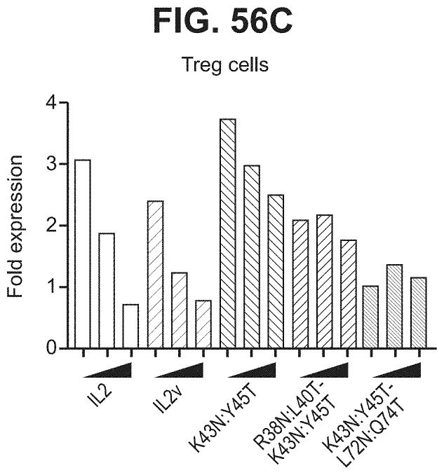

FIGS. 56A, 56B, and 56C depict the effect of various concentrations of the listed single and double glycan IL-2 variants on the expansion of CD8 T cells, NK cells, and Treg cells, respectively, as measured by fold-expansion of the cells. In the wild-type Fc-IL2 groups, 2/3 mice in the 1 mg/kg group and 1/3 mice in the 2 mg/kg group did not survive treatments. As shown in FIGS. 56A and 56B, each of Fc-IL2-K43N:Y45T; Fc-IL2-R38N:L40T-K43N:Y45T; and Fc-IL2-K43N:Y45T-L72N:Q74T promoted the expansion of CD8 T cells and NK cells, and greater concentrations of these molecules increased the expansion of CD8 T cells and NK cells. In contrast, Fc-IL2 and Fc-IL2v did not increase the expansion of CD8 T cells and NK cells; in fact, increasing concentrations of these molecules decreased the expansion of CD8 T cells and NK cells due to systemic toxicity. As shown in FIG. 56C, increasing concentrations of each of Fc-IL2; Fc-IL2v; Fc-IL2-K43N:Y45T demonstrated a modest increase in Treg proliferation with an inverse dose-response, whereas Fc-IL2-R38N:L40T-K43N:Y45T and Fc-IL2-K43N:Y45T-L72N:Q74T had minimal effects on the proliferation of Treg cells at all doses.

### Example 21

In this example, various single and double glycan IL-2 variants described above were tested for tolerability and tumor growth inhibition in mice. The tested variants were: Fc-IL2-K43N:Y45T; Fc-IL2-R38N:L40T-K43N:Y45T; Fc-IL2-K43N:Y45T-L72N:Q74T. In addition, Fc-linked wild-type human IL-2 (“Fc-IL2”) and Fc-linked IL2v (“Fc-IL2v”) were also tested as controls.

On day 0 of the experiment, female C57/BL6 mice were subcutaneously implanted in the upper thigh with approximately 500,000 B16F10 cells, which had been freshly thawed from a single, low-passage vial (of 1*10\hat{ }7 cells) and cultured for the minimum time required to establish sufficient cells for implantation.

On day 5 of the experiment, mice were randomized into groups to receive one of the molecules listed above or control. The treatment groups were as follows: PBS; Fc-IL2; Fc-IL2v; Fc-IL2-K43N:Y45T; Fc-IL2-R38N:L40T-K43N:Y45T; Fc-IL2-K43N:Y45T-L72N:Q74T. Fc-IL2 fusion molecules were dosed at 1 mg/kg on days 5, 6, 7, and 8 by subcutaneous injection with the respective control or variant Fc-IL2 fusion molecule at the concentration thereof for their assigned group. Groups of 15 animals were maintained to assess tolerability and tumor growth inhibition of the 1 mg/kg dose. Tumor volumes, body weight, and animal survival were tracked throughout the course of the experiment. Animals were sacrificed once tumors reached approximately 2000 mm\hat{ }3 or 2 weeks post treatment.

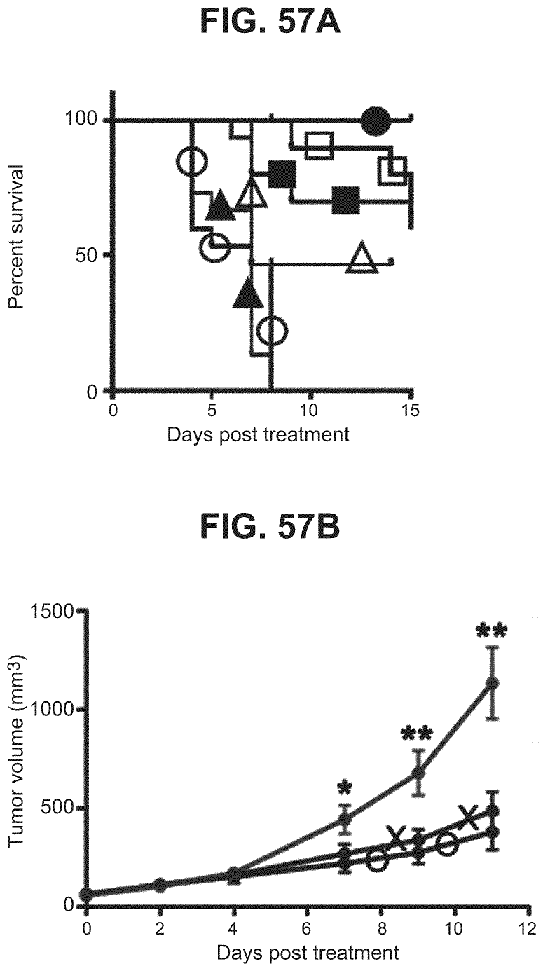

Survival and tumor growth inhibition was monitored over approximately 2 weeks from the starting dose for treatment groups as shown in FIGS. 57A and 57B. Tolerability was correlated with the degree of attenuation of IL-Ra binding as seen in the other Examples. Fc-IL2 and Fc-IL2v control molecule groups were similarly tolerated with no survival by day 8 (FIG. 57A). Fc-IL2-K43N:Y45T had intermediate survival with 7/15 mice. Groups treated with double glycan variants Fc-IL2-R38N:L40T-K43N:Y45T and Fc-IL2-K43N:Y45T-L72N:Q74T had 11/15 and 14/15 surviving mice at day 12 post first treatment. Tumor growth inhibition was assessed for the surviving mice from the better tolerated Fc-IL2-R38N:L40T-K43N:Y45T and Fc-IL2-K43N:Y45T-L72N:Q74T groups, in comparison to PBS control. As shown in FIG. 57B, significant tumor growth inhibition was observed for mice treated with Fc-IL2-R38N:L40T-K43N:Y45T and Fc-IL2-K43N:Y45T-L72N:Q74T.

These experiments show that Fc-IL2-K43N:Y45T; Fc-IL2-R38N140T-K43N:Y45T; and Fc-IL2-K43N:Y45T-L72N:Q74T proteins are better tolerated in mice than Fc-IL2 and Fc-IL2v, and that Fc-IL2-R38N:L40T-K43N:Y45T and Fc-IL2-K43N:Y45T-L72N:Q74T have tumor growth inhibition activity.

### Example 22: In Vitro Potency of VV110 in a Panel of Human Tumor Cell Lines

VV110 and VV12 (a JX-594 mimetic) were tested in cytotoxicity assays in a panel of human tumor cell lines from NSCLC, melanoma, RCC, CRC and HCC indications. Cells were cultured in their corresponding complete media. Cells were plated 24 hr prior to assay in 96-well plates at a cell type specific seeding density to form a confluent monolayer on the day of the assay. Test viruses were serially diluted (1:5) from a starting MOI of 30 in cell line specific media containing 2.5% FBS. After media aspiration, cells were infected with virus (from MOI of 30 to 1.54×10⁻⁵). The plates were then incubated for 48, 72 or 96 hr in a 37° C., 5% CO2 incubator. At the end of the incubation, CCK-8 reagent was added to each well and the absorbance at 450 nm was read using a SpectraMax i3X. The data was normalized to the cells only (100% viability) and the lysed cells only controls (0% viability). EC50 was calculated using a 4 parameter logistic fit. EC₅₀ and % maximum killing were reported for each time-point.

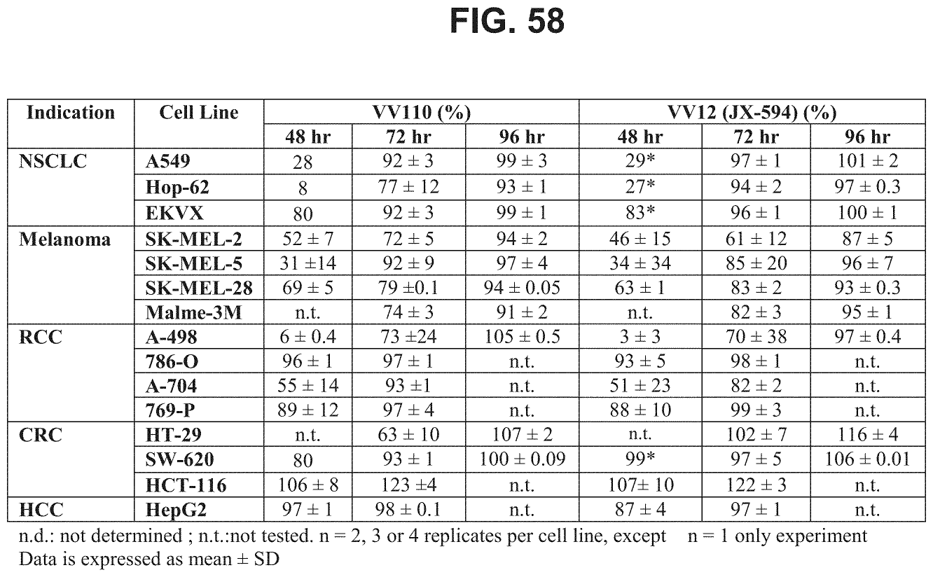

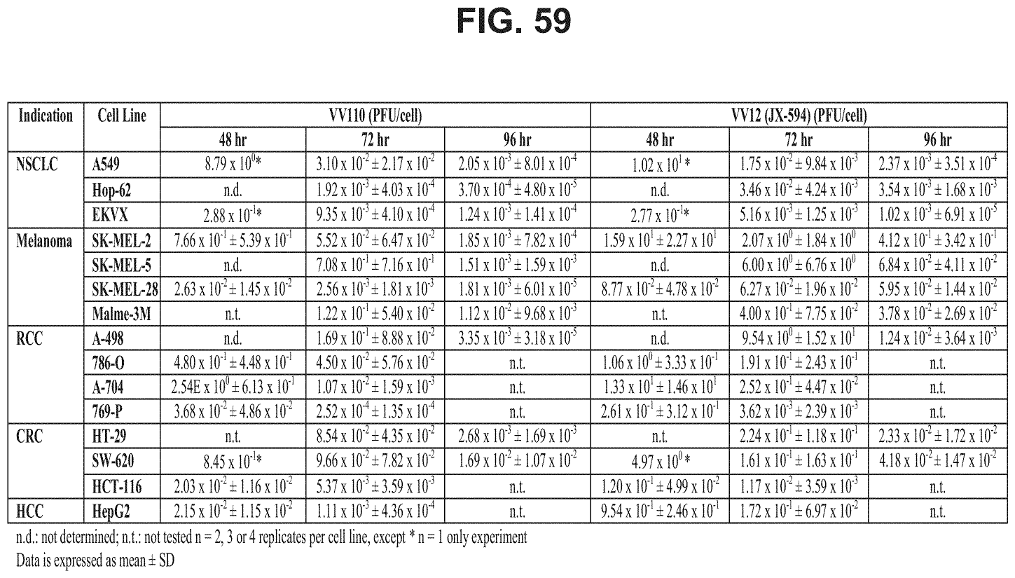

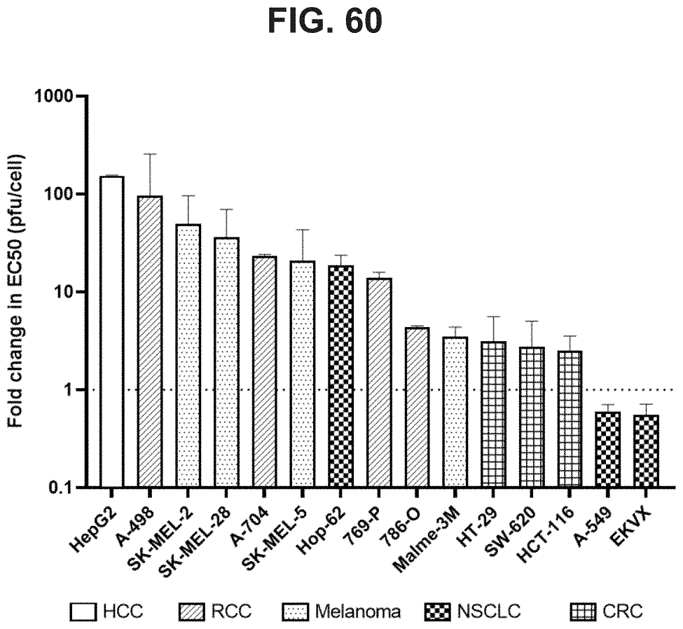

All of the cell lines tested were susceptible to infection and oncolysis in vitro induced by both VV110 and VV12 with ≥90% killing observed 2 to 4 days post-infection, dependent on cell line (FIG. 58). The potency of VV110 ranged from EC50 of 2.52×10⁻⁴ PFU/cell for the most sensitive line tested (769-P) to 7.08×10⁻¹ PFU/cell for the least sensitive line tested (SK-MEL-5) with no tumor indication being consistently more sensitive or resistant to VV110 than the others (FIG. 59). All cell lines tested were also sensitive to VV12, a JX-594 mimetic. The EC50 ratio of VV12 over VV110 was calculated and VV110 demonstrated higher in vitro potency compared to VV12, in 13 out of 15 tumor cell lines (FIG. 60).

FIG. 58: Percentage maximum human tumor cell killing at 48, 72, and 96 hrs post-infection. Human tumor cell lines were infected with VV110 or VV12 (JX-594) for 48, 72, or 96 hr at which point cell viability was determined. Data represented as mean±SD.

FIG. 59: Potency of VV110 and VV12 in human tumor cell lines at 48, 72, and 96 hrs post-infection. Human tumor cell lines were infected with VV110 or VV12 (JX-594) for 72 hr at which point cell viability was determined and EC50 (pfu/cell) calculated using a 4-PL logistic fit. Data represented as mean±SD.

FIG. 60: Relative potency (EC50 ratio) of VV110 and VV12 in human tumor cell lines. Human tumor cell lines were infected with VV110 or VV12 (JX-594) for 72 hr at which point cell viability was determined and EC50 (pfu/cell) calculated using a 4-PL logistic fit. EC50 ratio of VV12 over VV110 was calculated. Data represented as mean±SD.

### Example 23: Topical Acyclovir Treatment of Spontaneous Skin Lesions Occurring Following IV Administration of VV110 in Cynomolgus Monkeys

Cynomolgus monkeys received 5×10⁷ PFU VV110 via IV administration on study day 1. Animals developed spontaneous skin lesions by study day 5 at which an area containing 3 lesions was identified for examination of lesion progression and virus shedding either without (Group 1) or with (Group 2) lesion treatment with topical acyclovir (Zovirax). Animals receiving treatment had topical acyclovir applied to the area 4 times a day (2 hour intervals) for 11 days. Lesion progression was documented photographically. Virus shedding from lesions was assessed on days 5, 7, and 9. Swabs of individual lesions were collected and stored at −80° C. prior to assay for infectious virus titer in a U2OS plaque assay. Briefly, U-20S cells were plated approximately 24 h prior to the titer assay in 6-well plates. One mL of PBS was added to the swabs and samples were sonicated. Media was removed from cells and 700 μL of serially diluted virus/swab samples were added. After 2 h incubation in a 37° C. incubator, the inoculum was removed and 2 mL of 1.5% CMC, 10% FBS, 0.5× McCoy's overlay was added to each well. Plates were incubated for 48 h in a 37° C. incubator. At the end of this incubation period, the plates were washed once in DPBS and the cells were fixed and stained with crystal violet for 1 h, then washed with water. Images were acquired with Immunospot S6 MACRO Analyzer. Plaques were counted using the CTL ImmunoSpot software and titers (PFU/m L) were determined.

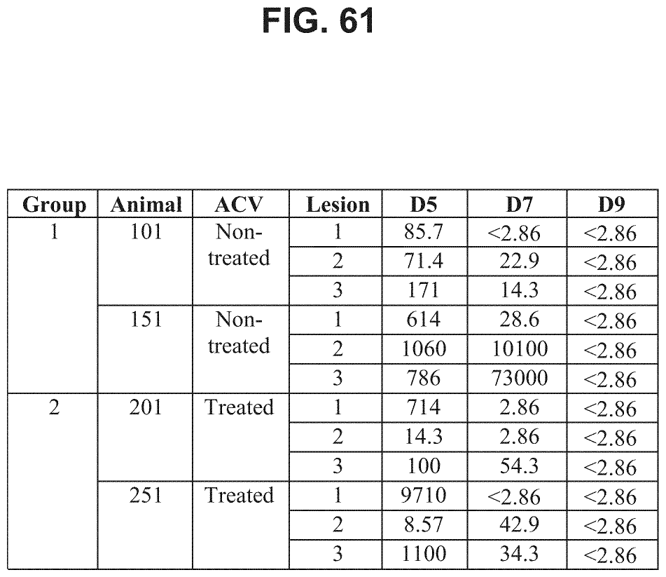

Lesions on Group 1, animals that did not receive topical acyclovir treatment, resolved by approximately Day 15 to 17. Lesions on group 2 animals that were treated topical acyclovir resolved more quickly, by approximately Day 11 to 13. In group 1, lesion swab titers ranged from 71 to 1060 PFU/mL on Day 5 to <3 to 73,000 PFU/mL on Day 7, whereas in group 2 animals, lesion swab titers from lesions treated with ACV ranged from 14 to 9,710 PFU/mL on Day 5 to 3 to 54 PFU on Day 7. On Day 9 and Day 11, none of the lesion swabs had any detectable infectious titers. On average, the infectious virus titers from swabs of lesions treated with ACV (group 2) decreased in quantity over a shorter duration than that detected from untreated lesions (group 1) (FIG. 61).

FIG. 61: Infectious virus titer from spontaneous skin lesions occurring following IV administration of VV110 to cynomolgus monkeys, with or without topical acyclovir treatment. Swabs were collected from individual skin lesions on animals that received 5×10⁷ PFU VV110 IV, either without (Group 1) or with (Group 2) topical acyclovir administration.

These data support the concept that the HSV TK.007 safety “off-switch” included in VV110 confers virus sensitivity to topical antiviral drugs and provides a potential means to reduce the severity, duration, and level of virus shedding from spontaneous skin lesions that can occur in some cancer patients following VV treatment.

1. An isolated human interleukin 2 (IL-2) variant comprising at least one amino acid substitution as compared to wild-type human IL-2 having the amino acid sequence as shown in SEQ ID NO: 1, wherein the IL-2 variant comprises one or more substitutions at amino acid positions selected from the group consisting of:
a) K35,
b) both R38 and L40,
c) both T41 and K43,
d) both K43 and Y45,
e) both E62 and K64, and
f) both L72 and Q74.

2. An isolated fusion protein, comprising: a) an IL-2 variant of claim 1; and b) an Fc region of a human antibody, wherein the IL-2 variant is covalently linked to the Fc region.

3-5. (canceled)

6. A method for treating cancer in a subject, comprising administering to the subject in need thereof an effective amount of the IL-2 variant of claim 1.

7. A recombinant oncolytic virus (OV), which comprises a nucleotide sequence encoding an IL-2 variant of claim 1.

8-14. (canceled)

15. The OV according to claim 7, wherein the IL-2 variant comprises the amino acid substitutions R38N, L40T, K43N, and Y45T.

16. The OV according to claim 7, wherein the IL-2 variant comprises the amino acid sequence of SEQ ID NO:29 or SEQ ID NO:31.

17. The OV according to claim 7, wherein the IL-2 variant comprises substitutions at amino acid positions K43, Y45, L72, and Q74.

18. The OV according to claim 7, wherein the IL-2 variant comprises the amino acid substitutions K43N, Y45T, L72N, and Q74T.

19. The OV according to claim 7, wherein the IL-2 variant comprises the amino acid sequence of SEQ ID NO:33 or SEQ ID NO:35.

20. (canceled)

21. (canceled)

22. The OV of claim 7, which further comprises a nucleotide sequence encoding heterologous thymidine kinase (TK) polypeptide.

23. (canceled)

24. The OV of claim 22, wherein the heterologous TK polypeptide is a variant herpes simplex virus (HSV) TK polypeptide.

25-28. (canceled)

29. The OV of claim 24, wherein the variant HSV TK polypeptide comprises the amino acid sequence of SEQ ID NO: 26, 27, or 28.

30. The OV of claim 7, wherein the virus comprises an A34R gene comprising a K151E substitution.

31. The OV of claim 7, wherein the virus comprises a modification that renders the vaccinia thymidine kinase deficient.

32. (canceled)

33. The OV of claim 7, wherein the virus is a vaccinia virus.

34. The OV of claim 33, wherein the vaccinia virus is a Copenhagen strain.

35. (canceled)

36. The OV of claim 7, comprising, in its genome: (1) a nucleotide sequence encoding a variant interleukin-2 (IL-2v) polypeptide comprising amino acid sequence of SEQ ID NO:29; (2) a nucleotide sequence encoding a heterologous thymidine kinase (TK) polypeptide comprising the amino acid sequence of SEQ ID NO: 28; and (3) a K151E substitution in the A34R gene, wherein the virus is a Copenhagen strain vaccinia virus and is vaccinia thymidine kinase deficient.

37. A composition comprising: a) the OV of claim 7; and b) a pharmaceutically acceptable carrier.

38. A method of treating cancer in an individual having a cancer, the method comprising administering to the individual an effective amount of the composition of claim 37.

39-48. (canceled)

49. A method of treating cancer in an individual, comprising administering to the individual: a) an effective amount of a recombinant oncolytic virus of claim 22; and b) a synthetic analog of 2′-deoxy-guanosine in an amount that is effective to reduce an adverse side effect of the oncolytic virus.

50. (canceled)
